(19) United States
(12) Patent Application Publication
(10) Pub. No.: US 2023/0277654 A1
(43) Pub. Date: Sep. 7, 2023

(54) STABLE FORMULATIONS OF CYTOMEGALOVIRUS

(71) Applicant: Merck Sharp & Dohme LLC, Rahway, NJ (US)

(72) Inventors: Muneeswara Babu Medi, Chalfont, PA (US); Harrison Bradford Davis, Lansdale, PA (US); Lorenzo H. Chen, Hatfield, PA (US); Lynne Ann Isopi, Quakertown, PA (US); Jeffrey Thomas Blue, Telford, PA (US); Heidi Joanne Pixley, Glenside, PA (US); Erin J. Green-Trexler, Green Lane, PA (US)

(73) Assignee: Merck Sharp & Dohme LLC, Rahway, NJ (US)

(21) Appl. No.: 18/060,386

(22) Filed: Nov. 30, 2022

Related U.S. Application Data
(63) Continuation of application No. 16/760,571, filed on Apr. 30, 2020, now abandoned.
(60) Provisional application No. 62/580,230, filed on Nov. 1, 2017.

(51) Int. Cl. A61K 39/245; A61K 9/19; A61K 47/02; A61K 47/10; A61K 47/26; A61K 47/38

(52) U.S. Cl. CPC A61K 39/245; A61K 9/19; A61K 47/02; A61K 47/10; A61K 47/26; A61K 47/38; A61K 2039/5254

(57) ABSTRACT

The present invention relates to stable formulations of a cytomegalovirus (CMV) comprising for example, a genetically modified CMV that is conditionally replication defective, a buffer, alkali or alkaline salt, a sugar, a cellulose derivative and optionally a polyol.

### CROSS-REFERENCE TO RELATED APPLICATIONS

This application is a continuation of U.S. patent application Ser. No. 16/760,571, filed Apr. 30, 2020, which claims the benefit of International Application No. PCT/US18/057914, filed Oct. 29, 2018, which claims the benefit of U.S. Provisional Application No. 62/580,230, filed Nov. 1, 2017, the contents of which are hereby incorporated by reference in their entirety.

### REFERENCE TO SEQUENCE LISTING

The instant application contains a Sequence Listing which has been submitted electronically in XML format and is hereby incorporated by reference in its entirey. The XML file, created on Nov. 28, 2022, is named 24527-US-CNT.XML and is 258 bytes in size.

### FIELD OF INVENTION

The present invention relates to stable formulations of cytomegalovirus (CMV). In one embodiment, the CMV is genetically modified CMV that is conditionally replication defective.

### BACKGROUND OF THE INVENTION

Cytomegalovirus (CMV), also known as human herpesvirus 5 (HHV-5), is a herpes virus classified as being a member of the beta subfamily of herpesviridae. According to the Centers for Disease Control and Prevention, CMV infection is found fairly ubiquitously in the human population, with an estimated 40-80% of the United States adult population having been infected. The virus is spread primarily through bodily fluids and is frequently passed from pregnant mothers to the fetus or newborn. In most individuals, CMV infection is latent, although virus activation can result in high fever, chills, fatigue, headaches, nausea, and splenomegaly.

Although most human CMV infections are asymptomatic, CMV infections in immunocompromised individuals, (such as HIV-positive patients, allogeneic transplant patients and cancer patients) or persons whose immune system has yet fully developed (such as newborns) can be particularly problematic (Mocarski et al., Cytomegalovirus, in Field Virology, 2701-2772, Editor: Knipes and Howley, 2007). CMV infection in such individuals can cause severe morbidity, including pneumonia, hepatitis, encephalitis, colitis, uveitis, retinitis, blindness, and neuropathy, among other deleterious conditions. In addition, CMV infection during pregnancy is a leading cause of birth defects (Adler, 2008 J. Clin Virol, 41:231; Arvin et. al., 2004 Clin Infect Dis, 39:233; Revello et. al., 2008 J Med Virol, 80:1415). CMV infects various cells in vivo, including monocytes, macrophages, dendritic cells, neutrophils, endothelial cells, epithelial cells, fibroblasts, neurons, smooth muscle cells, hepatocytes, and stromal cells (Plachter et al. 1996, Adv. Virus Res. 46:195). Although clinical CMV isolates replicate in a variety of cell types, laboratory strains AD169 (Elek and Stem, 1974, Lancet 1:1) and Towne (Plotkin et al., 1975, Infect. Immun. 12:521) replicate almost exclusively in fibroblasts (Hahn et al., 2004, J. Virol. 78:10023). The restriction in tropism, which results from serial passages and eventual adaptation of the virus in fibroblasts, is stipulated to be a marker of attenuation (Gema et al., 2005, J. Gen. Virol. 86:275; Gerna et al., 2002, J. Gen Virol. 83:1993; Gerna et al., 2003, J. Gen Virol. 84:1431; Dargan et al., 2010, J. Gen Virol. 91:1535). Mutations causing the loss of epithelial cell, endothelial cell, leukocyte, and dendritic cell tropism in human CMV laboratory strains have been mapped to three open reading frames (ORFs): UL128, UL130, and UL131 (Hahn et al., 2004, J. Virol. 78:10023; Wang and Shenk, 2005 J. Virol. 79:10330; Wang and Shenk, 2005 Proc Natl Acad Sci USA. 102:18153). Biochemical and reconstitution studies show that UL128, UL130 and UL131 assemble onto a gH/gL scaffold to form a pentameric gH complex (Wang and Shenk, 2005 Proc Natl Acad Sci USA. 102:1815; Ryckman et al., 2008 J. Virol. 82:60). Restoration of this complex in virions restores the viral epithelial tropism in laboratory strains (Wang and Shenk, 2005, J. Virol. 79:10330).

Loss of endothelial and epithelial tropism has been suspected as a deficiency in CMV strains previously evaluated as vaccines such as Towne (Gerna et al., 2002, J. Gen Virol. 83:1993; Gema et al., 2003, J. Gen Virol. 84:1431). Neutralizing antibodies in sera from human subjects of natural CMV infection have more than 15-fold higher activity against viral epithelial entry than against fibroblast entry (Cui et al., 2008, Vaccine 26:5760). Humans with primary infection rapidly develop neutralizing antibodies to viral endothelial and epithelial entry but only slowly develop neutralizing antibodies to viral fibroblast entry (Gema et al., 2008, J. Gen. Virol. 89:853). Furthermore, neutralizing activity against viral epithelial and endothelial entry is absent in the immune sera from human subjects who received Towne vaccine (Cui et al., 2008, Vaccine 26:5760). More recently, a panel of human monoclonal antibodies from four donors with human cytomegalovirus (HCMV) infection was described, and the more potent neutralizing clones from the panel recognized the antigens of the pentameric gH complex (Macagno et al., 2010, J. Virol. 84:1005).

Whole viruses are one of the commonly used antigens in several vaccine products due to their ability to generate humoral and cellular immune responses. Vaccine products containing whole viruses are challenging to stabilize as these are sensitive to heat, freeze/thaw and other processing stresses leading to significant potency losses. These products are typically stored frozen (below −20° C.) or as dried powder. Frozen products are not easy to store and distribute as they need a stringent cold-chain requirement to prevent potency loss. Drying of whole viruses, especially enveloped viruses, often leads to significant loss of potency due to the freezing and drying stresses encountered during the drying process. Therefore, there is a need in the art to generate stable formulations of CMV.

### SUMMARY OF THE INVENTION

The current invention provides stable formulations of cytomegalovirus (CMV). The addition of a cellulose derivative, for example, a carboxymethylcellulose salt, improved CMV stability and/or yield after drying. The further addition of a polyol, for example, propylene glycol, also further improved the virus stability and/or yield after drying. In one embodiment, the CMV formulation has a shelf-life of >2 years as measured by CMV titer of about 7.77×10E⁴ to 3.8×10E⁸ pfu/ml at 2-8° C.

In one aspect of the invention, the formulation comprises a cytomegalovirus (CMV), a buffer at a pH of about 6.0 to 8.0, an alkali or alkaline salt, a sugar, a cellulose derivative selected from the group consisting of carboxymethyl cellulose, hydroxypropyl cellulose (HPC), hydroxypropyl methylcellulose (HPMC), 2-hydroxyethyl cellulose (2-HEC), croscarmellose and methyl cellulose or a pharmaceutically acceptable salt thereof, and optionally, a polyol selected from the group consisting of propylene glycol, polypropylene glycol, ethylene glycol, polyethylene glycol, a polyethylene glycol monomethyl ether, and a sugar alcohol, e.g., glycerol.

In one aspect of the invention, the formulation comprises a cytomegalovirus (CMV), a buffer at a pH of about 6.0 to 7.5, an alkali or alkaline salt, a sugar, a cellulose derivative selected from the group consisting of carboxymethyl cellulose, hydroxypropyl cellulose (HPC), hydroxypropyl methylcellulose (HPMC), 2-hydroxyethyl cellulose (2-HEC), croscarmellose and methyl cellulose or a pharmaceutically acceptable salt thereof, and optionally, a polyol selected from the group consisting of propylene glycol, polypropylene glycol, ethylene glycol, polyethylene glycol, a polyethylene glycol monomethyl ether, and a sugar alcohol, e.g., glycerol.

In one embodiment, the buffer is selected from the group consisting of phosphate, succinate, histidine, TRIS, MES, MOPS, HEPES, acetate and citrate, or any combination thereof. In one aspect of this embodiment, the buffer is selected from the group consisting of phosphate, histidine and HEPES. In another embodiment, the alkali or alkaline salt is magnesium chloride, calcium chloride, potassium chloride, sodium chloride or a combination thereof. In one aspect of this embodiment, the salt is selected from the group consisting of potassium chloride and sodium chloride. In a further embodiment, the sugar is trehalose or sucrose. In yet a further embodiment, the cellulose derivative is a pharmaceutically acceptable salt of carboxymethylcellulose.

In one embodiment, the polyol is propylene glycol, glycerol and sorbitol. In a particular embodiment, the polyol is propylene glycol. In a further particular embodiment, the polyol is propylene glycol and the cellulose derivative is sodium carboxymethylcellulose (sodium CMC).

In other aspects of the invention, the formulation comprises about 50-600 μg/ml CMV, a buffer at pH about 6.0 to 8.0, about 50-300 mM of an alkali salt, about 40-150 mg/ml sucrose or trehalose, and about 0.3-10 mg/ml of a pharmaceutically acceptable salt of carboxymethylcellulose or hydroxypropylmethylcellulose. In another embodiment, the formulation comprises about 50-600 μg/ml CMV, about 5-500 mM buffer at pH about 6.0 to 8.0, about 50-300 mM NaCl or KCl, about 40-150 mg/ml sucrose or trehalose, and about 0.3-10 mg/ml sodium carboxymethylcellulose or hydroxypropylmethylcellulose with average molecular weight at about 50,000 to 1,000,000.

In other aspects of the invention, the formulation comprises about 50-600 μg/ml CMV, a buffer at pH about 6.0 to 7.5, about 50-300 mM NaCl, about 40-150 mg/ml sucrose, and about 0.3-10 mg/ml of a pharmaceutically acceptable salt of carboxymethylcellulose. In another embodiment, the formulation comprises about 50-600 μg/ml CMV, about 5-500 mM buffer at pH about 6.0 to 7.5, about 50-300 mM NaCl, about 40-150 mg/ml sucrose, and about 0.3-10 mg/ml sodium carboxymethylcellulose with average molecular weight at about 50,000 to 1,000,000.

In a further embodiment, the formulation comprises about 50-600 μg/ml CMV, about 10-100 mM histidine or phosphate or HEPES buffer, or any combination thereof, at pH about 6.0 to 8.0, about 50-300 mM NaCl, about 40-150 mg/ml sucrose, about 2.5-7.5 mg/ml propylene glycol (PG), and about 3-10 mg/ml sodium carboxymethylcellulose with average molecular weight at about 90,000. In yet a further embodiment, the formulation comprises about 50-600 μg/ml CMV, about 10-100 mM histidine, phosphate or HEPES buffer, or any combination thereof, pH about 6.0 to 7.5, about 50-150 mM NaCl, about 60-110 mg/ml sucrose, about 3-7 mg/ml propylene glycol (PG), and about 3-7 mg/ml sodium carboxymethylcellulose with average molecular weight at about 90,000. In yet a further embodiment, the formulation comprises about 100-350 μg/ml CMV, about 25 mM buffer of histidine, phosphate, HEPES or a combination thereof, pH about 7.0, about 75 mM NaCl, about 90 mg/ml sucrose, about 5 mg/ml propylene glycol (PG), and about 5 mg/ml sodium carboxymethylcellulose with average molecular weight at about 90,000. In one embodiment, the formulation further comprises an aluminum adjuvant.

In a further embodiment, the formulation comprises about 50-600 μg/ml CMV, about 10-100 mM histidine or Tris or HEPES buffer, or any combination thereof, at pH about 6.0 to 7.5, about 50-300 mM NaCl, about 40-150 mg/ml sucrose, about 2.5-7.5 mg/ml propylene glycol (PG), and about 3-10 mg/ml sodium carboxymethylcellulose with average molecular weight at about 90,000. In yet a further embodiment, the formulation comprises about 50-600 μg/ml CMV, about 10-100 mM histidine, TRIS or HEPES buffer, or any combination thereof, pH about 6.0 to 7.5, about 50-150 mM NaCl, about 60-110 mg/ml sucrose, about 3-7 mg/ml propylene glycol (PG), and about 3-7 mg/ml sodium carboxymethylcellulose with average molecular weight at about 90,000. In yet a further embodiment, the formulation comprises about 100-350 μg/ml CMV, about 25 mM buffer of histidine, TRIS, or a combination thereof, pH about 7.0, about 75 mM NaCl, about 90 mg/ml sucrose, about 5 mg/ml propylene glycol (PG), and about 5 mg/ml sodium carboxymethylcellulose with average molecular weight at about 90,000. In one embodiment, the formulation further comprises an aluminum adjuvant.

In one aspect of the foregoing embodiments, the formulation is an aqueous solution prior to lyophilization.

In another aspect of the foregoing embodiments, the formulation is a reconstituted solution, reconstituted with water or saline. In one embodiment, the reconstituted solution is performed with 0.5-1 ml of a diluent comprising an aluminum adjuvant formulated in saline solution, water or buffer. In another embodiment, the reconstitution is performed with a diluent (0.5 ml or 0.7 ml) comprising an Aluminum Phosphate Adjuvant (APA) and saline solution. In a further embodiment, the Aluminum Phosphate Adjuvant is at about 400-500 μg/ml or 200-700 μg/ml. In a particular embodiment, the reconstituted solution is the 0.5 ml dose of CMV comprising about 25-300 μg of CMV, about 1.39-1.9 mg histidine, about 6-6.7 mg NaCl, about 32.2-45 mg sucrose, about 1.79-2.5 mg propylene glycol (PG), and about 1.79-2.5 mg sodium carboxymethylcellulose at average molecular weight at about 90,000.

In another aspect of the invention, the formulation is in dried solid form and comprises about 25-300 μg CMV, about 1.9-2.7 mg histidine, TRIS, or a combination thereof, about 2.2-3.07 mg NaCl, about 45-63 mg sucrose, about 2.5-3.5 mg propylene glycol (PG), and about 2.5-3.5 mg sodium carboxymethylcellulose with average molecular weight of 90,000. In a further aspect of the invention, the formulation is in dried solid form comprising a weight ratio of about CMV 1, histidine 6-108, NaCl 7-123, sucrose 150-2520, propylene glycol 8-140, and sodium carboxymethylcellulose 8-140. In one embodiment, the formulation is a dried solid formulation, wherein the CMV titer after 2 years at 2-8° C. is about 7.77×10E⁴ to 3.8×10E⁸ pfu/ml. In another embodiment, the formulation is a dried solid formulation, wherein the CMV after 6 months at 2-8° C. has less than or equal to about 0.2 log 10 infectivity loss as compared to a CMV reference sample. In a further embodiment, the formulation is a dried solid formulation, wherein the CMV after 2 years at 2-8° C. has less than or equal to about 0.5 log 10 infectivity loss as compared to a CMV reference sample. In yet a further embodiment, the formulation is a dried solid formulation, wherein the CMV after 2 years at 2-8° C. has less than or equal to about 1.0 log 10 infectivity loss as compared to a CMV reference sample. In another embodiment, the dried solid formulation further comprises an aluminum adjuvant, for example APA.

In one aspect of the foregoing embodiments, the CMV is a live attenuated CMV, or a killed or inactivated CMV. In one embodiment, the live attenuated CMV is a conditional replication defective CMV (rdCMV) that comprises: (a) a pentameric gH complex comprising UL128, UL130, UL131, gH and gL; and (b) a nucleic acid encoding a fusion protein of an essential protein and a destabilizing protein, wherein the essential protein is selected from the group consisting of IE1/2, UL51, UL52, UL79 and UL84. In another embodiment, the destabilizing protein is either FK506-binding protein (FKBP) or an FKBP derivative, wherein the FKBP derivative is FKBP comprising one or more amino acid substitutions selected from the group consisting of: F15S, V24A, H25R, F36V, E60G, M66T, R71G, D100G, D100N, E102G, K105I and L106P. In another embodiment, the FKBP derivative is FKBP comprising amino acid substitutions F36V and L106P. In one embodiment, the essential protein is IE1/2. In another embodiment, the essential protein is UL51. In another embodiment of the foregoing embodiments, the CMV comprises a nucleic acid encoding at least two fusion proteins, wherein the essential proteins in each of the fusion proteins are different. In one embodiment, one of the fusion proteins comprises IE1/2 or UL51. In another embodiment, a first fusion protein comprises IE1/2 and a second fusion protein comprises UL51.

In another aspect of the foregoing embodiments, the live attenuated CMV is a conditional replication defective CMV that comprises: (a) a pentameric gH complex comprising UL128, UL130, UL131, gH and gL; and (b) a nucleic acid encoding a first fusion protein of IE1/2 and a destabilizing protein and a second fusion protein of UL51 and the destabilizing protein, wherein the destabilizing protein is FK506-binding protein (FKBP) derivative comprising amino acid substitutions F36V and L106P; wherein the wild type IE1/2 and UL51 are no longer present and wherein the CMV is an attenuated strain that has restored gH complex expression due to a repair of a mutation in the UL131 gene.

In one embodiment of the foregoing embodiments, (a) the first fusion protein is SEQ ID NO:1 or an amino acid sequence that is at least 95% identical to SEQ ID NO:1; and (b) the second fusion protein is SEQ ID NO:3 or an amino acid sequence that is at least 95% identical to SEQ ID NO:3. In another embodiment of the foregoing embodiments, the first fusion protein comprises SEQ ID NO:1 and the second fusion protein comprises SEQ ID NO:3. In another embodiment of the foregoing embodiments, (a) the first fusion protein is encoded by SEQ ID NO:2 or a nucleic acid sequence that is at least 95% identical to SEQ ID NO:2; and (b) the second fusion protein is encoded by SEQ ID NO:4 or a nucleic acid sequence that is at least 95% identical to SEQ ID NO:4. In yet another embodiment of the foregoing embodiments, the first fusion protein is encoded by SEQ ID NO:2 and the second fusion protein is encoded by SEQ ID NO:4.

In another aspect of the foregoing embodiments, the live attenuated CMV is a conditional replication defective CMV that comprises: (a) a pentameric gH complex comprising UL128, UL130, UL131, gH and gL; and (b) a nucleic acid encoding a first fusion protein of an essential protein and a destabilizing protein and a second fusion protein of an essential protein and a destabilizing protein, wherein the first fusion protein comprises SEQ ID NO:1 and the second fusion protein comprises SEQ ID NO:3, wherein the wild type 1E1/2 and UL51 are no longer present; and wherein the CMV is an attenuated strain that has restored gH complex expression due to a repair of a mutation in the UL131 gene. In one embodiment, the CMV is AD169 that has restored gH complex expression due to a repair of a mutation in the UL131 gene. In another embodiment, the conditional replication defective CMV has a genome as shown in SEQ ID NO: 14.

### BRIEF DESCRIPTION OF THE DRAWINGS

FIG. 1: Formulation excipient screening for CMV lyophilization yield. Percent lyophilization yield was calculated using the measured infectivity for frozen liquid formulation as 100 percent. The lyophilized vials stored at −70° C. were tested along with frozen liquid control vials (stored at −70° C.) using a cell-based infectivity assay and the lyophilization yield was calculated as a percentage of liquid control sample. Three samples were tested and the average data from 3 tests was reported. Standard error of the mean (SEM) for the samples was calculated and 2×SEM is reported.

FIG. 2: Formulation excipient screening for CMV stability. The stability samples subjected to different storage conditions were tested at 1 week using a cell-based infectivity assay along with lyophilization control samples stored at −70° C. The Log10 Infectivity loss for the stability samples was calculated as compared to the lyophilized control sample stored at 70° C. for each of the formulations.

FIG. 3: Formulation excipient optimization for CMV lyophilization yield. Percent lyophilization yield was calculated using the measured infectivity for frozen liquid formulation as 100 percent. The lyophilized vials stored at −70° C. were tested along with frozen liquid control vials (stored at −70° C.) using a cell-based infectivity assay and the lyophilization yield was calculated as a percentage of liquid control sample. Three samples were tested and the average data from 3 tests was reported. Standard error of the mean (SEM) for the samples was calculated and 2×SEM is reported.

FIG. 4: Formulation excipient optimization for CMV stability. The stability samples subjected to 2-8° C. storage for different times (1 month, 3 months and 6 months) were tested using a cell-based infectivity assay along with lyophilization control samples stored at −70° C. The Log10 Infectivity loss for the stability samples was calculated as compared to the lyophilized control sample stored at 70° C. for each of the formulations.

FIGS. 5A-5B: Effect of formulation pH on CMV lyophilization process yield. Lyophilization yield for different CMV formulations can be found in FIG. 5A (pH 6.0, 6.5, 7.0 and 7.5) and FIG. 5B (pH 6.0, 7.0 and 8.0). Percent lyophilization yield was calculated using the measured infectivity for frozen liquid formulation as 100 percent. The lyophilized vials stored at −70° C. were tested along with frozen liquid control vials (stored at −70° C.) using a cell-based infectivity assay and the lyophilization yield was calculated as a percentage of liquid control sample. Three samples were tested and the average data from 3 tests was reported. Standard error of the mean (SEM) for the samples was calculated and 2×SEM is reported.

FIGS. 6A-6B: Effect of formulation pH on CMV stability. Stability Studies for different CMV formulations can be found in FIG. 6A (pH 6.0, 6.5, 7.0 and 7.5) and FIG. 6B (pH 6.0, 7.0 and 8.0). The stability samples subjected to 2-8° C. storage for 1 month and 3 months (FIG. 6A) or 2-8° C. or 25° C. storage for 1 week (FIG. 6B) were tested using a cell-based infectivity assay along with lyophilization control samples stored at −70° C. The Log10 Infectivity loss for the stability samples was calculated as compared to the lyophilized control sample stored at 70° C. for each of the formulations. Three samples were tested and the average data from 3 tests was reported. Standard error of the mean (SEM) for the samples was calculated and 2×SEM is reported.

FIG. 7: Effect of propylene glycol concentration on CMV lyophilization yield. Percent lyophilization yield was calculated using the measured infectivity for frozen liquid formulation as 100 percent. The lyophilized vials stored at −70° C. were tested along with frozen liquid control vials (stored at −70° C.) using a cell-based infectivity assay and the lyophilization yield was calculated as a percentage of liquid control sample. Three samples were tested and the average data from 3 tests was reported. Standard error of the mean (SEM) for the samples was calculated and 2×SEM is reported.

FIG. 8: Effect of propylene glycol concentration on CMV stability. Stability Studies for different CMV formulations. The stability samples subjected to 15° C. (1 week) and 2-8° C. (1 week and 1 month) storage were tested using a cell-based infectivity assay along with lyophilization control samples stored at −70° C. The Log10 Infectivity loss for the stability samples was calculated as compared to the lyophilized control sample stored at 70° C. for each of the formulations. Three samples were tested and the average data from 3 tests was reported. Standard error of the mean (SEM) for the samples was calculated and 2×SEM is reported.

FIG. 9: Effect of Sodium CMC concentration on CMV lyophilization yield. Percent lyophilization yield was calculated using the measured infectivity for frozen liquid formulation as 100 percent. The lyophilized vials stored at −70° C. were tested along with frozen liquid control vials (stored at −70° C.) using a cell-based infectivity assay and the lyophilization yield was calculated as a percentage of liquid control sample. Three samples were tested and the average data from 3 tests was reported. Standard error of the mean (SEM) for the samples was calculated and 2×SEM is reported.

FIG. 10: Effect of Sodium CMC concentration on CMV stability. Stability Studies for different CMV formulations. The stability samples subjected to 15° C. (1 week) and 2-8° C. (1 week and 1 month) storage were tested using a cell-based infectivity assay along with lyophilization control samples stored at −70° C. The Log10 Infectivity loss for the stability samples was calculated as compared to the lyophilized control sample stored at 70° C. for each of the formulations. Three samples were tested and the average data from 3 tests was reported. Standard error of the mean (SEM) for the samples was calculated and 2×SEM is reported.

FIG. 11: Effect of fill volume on CMV lyophilization yield. Percent lyophilization yield was calculated using the measured infectivity for frozen liquid formulation as 100 percent. The lyophilized vials stored at −70° C. were tested along with frozen liquid control vials (stored at −70° C.) using a cell-based infectivity assay and the lyophilization yield was calculated as a percentage of liquid control sample. Three samples were tested and the average data from 3 tests was reported. Standard error of the mean (SEM) for the samples was calculated and 2×SEM is reported.

FIG. 12: Effect of Fill Volume on CMV stability. The stability samples subjected to 2-8° C. (1 month, 3 months and 6 months) storage were tested using a cell-based infectivity assay along with lyophilization control samples stored at −70° C. The Log10 Infectivity loss for the stability samples was calculated as compared to the lyophilized control sample stored at 70° C. for each of the formulations. Three samples were tested and the average data from 3 tests was reported. Standard error of the mean (SEM) for the samples was calculated and 2×SEM is reported.

FIG. 13: Particle size data for liquid and lyophilized APA formulations measured by static light scattering.

FIGS. 14A-14B: (FIG. 14A) Particle size data for liquid, freeze/thawed and lyophilized CMV with APA in CMV-202 formulation measured by static light scattering. (FIG. 14B) Effect of formulation in the presence of APA on CMV stability in CMV-202 formulation. The stability samples subjected to 2-8° C. or 25° C. storage for 1 month were tested using a cell-based infectivity assay along with lyophilization control samples stored at −70° C. Three samples were tested and the average percent relative infectivity data from 3 tests was reported. Standard error of the mean (SEM) for the samples was calculated and 2×SEM was reported.

FIGS. 15A, 15B, and 15C: show a schematic diagram of the construction of a strain of CMV with restored expression of the pentameric gH complex. FIG. 15A shows a strategy for generation of self-excisable Bacterial Artificial Chromosome (BAC) to manipulate AD169 viral genome. FIG. 15B shows a repair of the frame shift mutation in UL131 to restore its expression. FIG. 15C shows a replacement of GFP with a cre recombinase gene to create a self excisable CMV BAC.

FIG. 16. Effect of buffer species at pH 7.0 on lyophilization process yield in CMV-202 formulation. Percent lyophilization yield was calculated using the measured infectivity for frozen liquid formulation as 100 percent. The lyophilized vials stored at −70° C. were tested along with frozen liquid control vials (stored at −70° C.) using a cell-based infectivity assay and the lyophilization yield was calculated as a percentage of liquid control sample. Three samples were tested and the average data from 3 tests was reported. Standard error of the mean (SEM) for the samples was calculated and 2×SEM was reported.

FIG. 17. Effect of buffer species at pH 7.0 on CMV stability in CMV-202 formulation. The stability samples subjected to 2-8° C. or 25° C. storage for 1 week and samples were tested using a cell-based infectivity assay along with lyophilization control samples stored at −70° C. The Log10 Infectivity loss for the stability samples was calculated as compared to the lyophilized control sample stored at −70° C. for each of the formulations. Three samples were tested and the average data from 3 tests was reported. Standard error of the mean (SEM) for the samples was calculated and 2×SEM is reported.

FIG. 18: Effect of sucrose concentration on lyophilization process yield in CMV-202 formulation. Percent lyophilization yield was calculated using the measured infectivity for frozen liquid formulation as 100 percent. The lyophilized vials stored at −70° C. were tested along with frozen liquid control vials (stored at −70° C.) using a cell-based infectivity assay and the lyophilization yield was calculated as a percentage of liquid control sample. Three samples were tested and the average data from 3 tests was reported. Standard error of the mean (SEM) for the samples was calculated and 2×SEM was reported.

FIG. 19: Effect of sucrose concentration on CMV stability in CMV-202 formulation. The stability samples subjected to 2-8° C. or 25° C. storage for 1 week and samples were tested using a cell-based infectivity assay along with lyophilization control samples stored at −70° C. The Log10 Infectivity loss for the stability samples was calculated as compared to the lyophilized control sample stored at −70° C. for each of the formulations. Three samples were tested and the average data from 3 tests was reported. Standard error of the mean (SEM) for the samples was calculated and 2×SEM is reported.

FIG. 20: Effect of trehalose concentration on lyophilization process yield in CMV-202 formulation. Percent lyophilization yield was calculated using the measured infectivity for frozen liquid formulation as 100 percent. The lyophilized vials stored at −70° C. were tested along with frozen liquid control vials (stored at −70° C.) using a cell-based infectivity assay and the lyophilization yield was calculated as a percentage of liquid control sample. Three samples were tested and the average data from 3 tests was reported. Standard error of the mean (SEM) for the samples was calculated and 2×SEM was reported.

FIG. 21: Effect of trehalose concentration on CMV stability in CMV-202 formulation. The stability samples subjected to 2-8° C. or 25° C. storage for 1 week and samples were tested using a cell-based infectivity assay along with lyophilization control samples stored at −70° C. The Log10 Infectivity loss for the stability samples was calculated as compared to the lyophilized control sample stored at −70° C. for each of the formulations. Three samples were tested and the average data from 3 tests was reported. Standard error of the mean (SEM) for the samples was calculated and 2×SEM is reported.

FIG. 22: Effect of sugar type (sucrose vs. trehalose) on lyophilization process yield in CMV-202 formulation. Percent lyophilization yield was calculated using the measured infectivity for frozen liquid formulation as 100 percent. The lyophilized vials stored at −70° C. were tested along with frozen liquid control vials (stored at −70° C.) using a cell-based infectivity assay and the lyophilization yield was calculated as a percentage of liquid control sample. Three samples were tested and the average data from 3 tests was reported. Standard error of the mean (SEM) for the samples was calculated and 2×SEM was reported.

FIG. 23: Effect of sugar type (sucrose vs. trehalose) on CMV stability in CMV-202 formulation. The stability samples subjected to 2-8° c. or 25° c. storage for 1 week and samples were tested using a cell-based infectivity assay along with lyophilization control samples stored at −70° C. The log 10 infectivity loss for the stability samples was calculated as compared to the lyophilized control sample stored at −70° C. for each of the formulations. Three samples were tested and the average data from 3 tests was reported. Standard error of the mean (sem) for the samples was calculated and 2× sem is reported.

FIG. 24: Effect of alkali salt (sodium chloride vs. potassium chloride) on lyophilization process yield in CMV-202 formulation. Percent lyophilization yield was calculated using the measured infectivity for frozen liquid formulation as 100 percent. The lyophilized vials stored at −70° C. were tested along with frozen liquid control vials (stored at −70° C.) using a cell-based infectivity assay and the lyophilization yield was calculated as a percentage of liquid control sample. Three samples were tested and the average data from 3 tests was reported. Standard error of the mean (SEM) for the samples was calculated and 2×SEM was reported.

FIG. 25: Effect of alkali salt (sodium chloride vs. potassium chloride) on CMV stability in CMV-202 formulation. The stability samples subjected to 2-8° C. or 25° C. storage for 1 week and samples were tested using a cell-based infectivity assay along with lyophilization control samples stored at −70° C. The Log10 Infectivity loss for the stability samples was calculated as compared to the lyophilized control sample stored at −70° C. for each of the formulations. Three samples were tested and the average data from 3 tests was reported. Standard error of the mean (SEM) for the samples was calculated and 2×SEM is reported. FIG. 26: Effect of cellulose type (CMC vs. HPMC) on lyophilization process yield in CMV-202 formulation. Percent lyophilization yield was calculated using the measured infectivity for frozen liquid formulation as 100 percent. The lyophilized vials stored at −70° C. were tested along with frozen liquid control vials (stored at −70° C.) using a cell-based infectivity assay and the lyophilization yield was calculated as a percentage of liquid control sample. Three samples were tested and the average data from 3 tests was reported. Standard error of the mean (SEM) for the samples was calculated and 2×SEM was reported.

FIG. 27: Effect of cellulose type (CMC vs. HPMC) on CMV stability in CMV-202 formulation. The stability samples subjected to 2-8° C. or 25° C. storage for 1 week and samples were tested using a cell-based infectivity assay along with lyophilization control samples stored at −70° C. The Log10 Infectivity loss for the stability samples was calculated as compared to the lyophilized control sample stored at −70° C. for each of the formulations. Three samples were tested and the average data from 3 tests was reported. Standard error of the mean (SEM) for the samples was calculated and 2×SEM is reported.

### DETAILED DESCRIPTION OF THE INVENTION

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization and/or use of the substance or composition; through instrumental error in these procedures; through differences in the manufacture, source, or purity of the ingredients employed to make or use the compositions or carry out the procedures; and the like. In certain embodiments, “about” can mean a variation of ±0.1%, 0.5%, 1%, 2%, 3%, 4%, 5%, or 10%.

The term “bulking agents” comprise agents that provide the structure of the freeze-dried product. Common examples used for bulking agents include mannitol, glycine, and lactose. In addition to providing a pharmaceutically elegant cake, bulking agents may also impart useful qualities in regard to modifying the collapse temperature, providing freeze-thaw protection, and enhancing the protein stability over long-term storage. These agents can also serve as tonicity modifiers.

The “CMV reference sample” has the same CMV formulation as the CMV formulation test sample and refers to the dried solid composition immediately after drying the CMV formulation under the same conditions as the CMV formulation test sample (i.e., lyophilization, microwave dried, lyosphere dried), or the foregoing dried solid composition stored at conditions where there is no or minimal infectivity loss of the CMV virus (i.e., stored at or below −70° C.).

“Inactivated virus” refers to a killed or inactive whole virus, wherein the virus is inactivated by any means, including with chemicals, heat or radiation. An inactivated virus has a low residual infectivity following inactivation, e.g., <5 plaque forming units (PFU's)/mL after inactivation. In preferred embodiments, there is very low amount of residual infectivity following inactivation, e.g., ≤4 PFU's/mL, ≤3 PFU's/mL, ≤2 PFU's/mL, ≤1 PFU/mL, ≤0.5 PFU/mL, or ≤0.1 PFU/mL. The PFU's of a particular virus, or formulation thereof, may be determined, for example, by using a plaque assay, an immunostaining assay, or other method known in the art for detecting viral infectivity.

“Infectivity loss” refers to comparing the loss of viral replication of a CMV test sample to a CMV reference sample using methods known in the art. In one embodiment, the loss of expression of viral proteins essential for viral replication in a CMV test sample to a CMV reference sample is measured. In another embodiment, the infectivity loss is measured using a relative infectivity assay (e.g. IRVE assay) in Example 3. In another embodiment, the infectivity loss is measured using a plaque assay.

The terms “lyophilization,” “lyophilized,” and “freeze-dried” refer to a process by which the material to be dried is first frozen and then the ice or frozen solvent is removed by sublimation in a vacuum environment. An excipient may be included in pre-lyophilized formulations to enhance stability of the lyophilized product upon storage.

“Lyosphere,” as used herein, refers to dried frozen unitary bodies comprising a therapeutically active agent which are substantially spherical or ovoid-shape. In some embodiments, the lyosphere diameter is from about 2 to about 12 mm, preferably from 2 to 8 mm, such as from 2.5 to 6 mm or 2.5 to 5 mm. In some embodiments, the volume of the lyosphere is from about 20 to 550 μL, preferably from 20 to 100 μL, such as from 20 to 50 μL. In embodiments wherein the lyosphere is not substantially spherical, the size of the lyosphere can be described with respect to its aspect ratio, which is the ratio of the longer dimension to the shorter dimension. The aspect ratio of the lyospheres can be from 0.5 to 2.5, preferably from 0.75 to 2, such as from 1 to 1.5.

“Live attenuated CMV” refers to a CMV wherein the ability of the virus to cause disease is reduced compared to wild-type CMV. In one embodiment, the reduced ability to cause disease is measured by reduction in infectivity of the CMV.

“Microwave Vacuum Drying” as used herein, refers to a drying method that utilizes microwave radiation (also known as radiant energy or non-ionizing radiation) for the formation of dried vaccine products (preferably, ≤6% moisture) of a vaccine formulation through sublimation. In certain embodiments, the microwave drying is performed as described in United States Patent Application Publication No. US2016/0228532. In one embodiment, the microwave radiation is in traveling wave format.

A “reconstituted solution” is one that has been prepared by dissolving dried virus in solid form (such as a lyophilized cake) in a diluent such that the virus is dispersed in the reconstituted solution. The reconstituted solution is suitable for administration, (e.g. intramuscular administration), and may optionally be suitable for subcutaneous administration.

“Salt(s)”, as employed herein, denotes acidic salts formed with inorganic and/or organic acids, as well as basic salts formed with inorganic and/or organic bases. Pharmaceutically acceptable (i.e., non-toxic, physiologically acceptable) salts are preferred, although other salts are also useful. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, Choline, tromethamine, dicyclohexylamines, t-butyl amines, and salts with amino acids such as arginine, lysine and the like.

“Sugar alcohol” refers to polyols derived from a sugar having the general formula HOCH₂(CHOH)ₙCH₂OH, n=1, 2, 3, 4, 5, 6, 7, 8, 9 or 10. Examples include but are not limited to mannitol, sorbitol, erythritol, xylitol and glycerol.

As used herein, “x % (w/v)” is equivalent to x g/100 ml (for example 5% w/v equals 50 mg/ml).

As used herein, the term “induce an immune response” refers to the ability of a live attenuated, killed or inactivated CMV to produce an immune response in a patient, preferably a mammal, more preferably a human, to which it is administered, wherein the response includes, but is not limited to, the production of elements (such as antibodies) which specifically bind, and preferably neutralize, CMV and/or cause T cell activation. A “protective immune response” is an immune response that reduces the likelihood that a patient will contract a CMV infection (including primary, recurrent and/or super-infection) and/or ameliorates at least one pathology associated with CMV infection and/or reduces the severity/length of CMV infection.

As used herein, the term “an immunologically effective amount” refers to the amount of an immunogen that can induce an immune response against CMV when administered to a patient that can protect the patient from a CMV infection (including primary, recurrent and/or super-infections) and/or ameliorate at least one pathology associated with CMV infection and/or reduce the severity/length of CMV infection in the patient. The amount should be sufficient to significantly reduce the likelihood or severity of a CMV infection. Animal models known in the art can be used to assess the protective effect of administration of immunogen. For example, immune sera or immune T cells from individuals administered the immunogen can be assayed for neutralizing capacity by antibodies or cytotoxic T cells or cytokine producing capacity by immune T cells. The assays commonly used for such evaluations include but not limited to viral neutralization assay, anti-viral antigen ELISA, interferon-gamma cytokine ELISA, interferon-gamma ELISPOT, intracellular multi-cytokine staining (ICS), and ⁵¹Chromimium release cytotoxicity assay. Animal challenge models can also be used to determine an immunologically effective amount of immunogen.

As used herein, the term “conditional replication defective CMV” refers to CMV destabilized of one or more proteins essential for viral replication. The nucleic acids encoding the wild type, non-destabilized essential proteins are no longer present in the conditional replication defective virus. Under conditions where the one or more essential proteins are destabilized, viral replication is decreased by preferably greater than 50%, 75%, 90%, 95%, 99%, or 100% as compared to a virus with no destabilized essential proteins. However, under conditions that stabilize the destabilized essential proteins, viral replication can occur at preferably at least 75%, 80%, 90%, 95%, 99% or 100% of the amount of replication of a CMV that does not contain a destabilized essential protein. In more preferred embodiments, one or more essential proteins are destabilized by fusion with a destabilizing protein such as FKBP or a derivative thereof. Such fusion proteins can be stabilized by the presence of a stabilizing agent such as Shield-1. As used herein, the term “rdCMV” refers to a conditional replication defective cytomegalovirus.

In preferred embodiments, the immune response induced by a replication defective virus as compared to its live virus counterpart is the same or substantially similar in degree and/or breadth. In other preferred embodiments, the morphology of a replication defective virus by electron microscopy analysis is indistinguishable or substantially similar to its live virus counterpart.

As used herein, the term “FKBP” refers to a destabilizing protein of SEQ ID NO:11. Fusion proteins containing FKBP are degraded by host cell machinery. As used herein, the term “FKBP derivative” refers to a FKBP protein or portion thereof that has been altered by one or more amino acid substitutions, deletions and/or additions. The FKBP derivatives retain substantially all of the destabilizing properties of FKBP when fused to a protein and also retain substantially all of the ability of FKBP to be stabilized by Shield-1. Preferred FKBP derivatives have one or more of the following substitutions at the denoted amino acid positions F15S, V24A, H25R, F36V, E60G, M66T, R71G, D100G, D100N, E102G, K105I and L106P. The FKBP derivative having the F36V and L106P substitutions (SEQ ID NO:12) is particularly preferred. In preferred embodiments, the nucleic acid that encodes the FKBP or FKBP derivative contains at least some codons that are not commonly used in humans for endogenous FKBP. This decreases the likelihood that the FKBP or FKBP derivative of the fusion protein will rearrange or recombine with its counterpart in human genome. The nucleic acid sequence of SEQ ID NO:13 encodes SEQ ID NO:12 using such codons.

As used herein, the terms “Shield-1” or “Shld-1” refer to a synthetic small molecule that binds to wild-type FKBP and derivatives thereof and acts as a stabilizing agent. Binding is about 1,000-fold tighter to the F36V derivative compared to wild-type FKBP (Clackson et al., 1998, Proc Natl Acad Sci USA 95:10437-42). Shield-1 can be synthesized (essentially as described in Holt et al., 1993, J. Am. Chem. Soc. 115:9925-38 and Yang et al., 2000, J. Med. Chem. 43:1135-42 and Grimley et al., 2008, Bioorganic & Medicinal Chemistry Letters 18:759) or is commercially available from Cheminpharma LLC (Farmington, CT) or Clontech Laboratories, INC. (Mountain View, Calif.). Salts of Shield-1 can also be used in the methods of the invention. Shield-1 has the following structure:

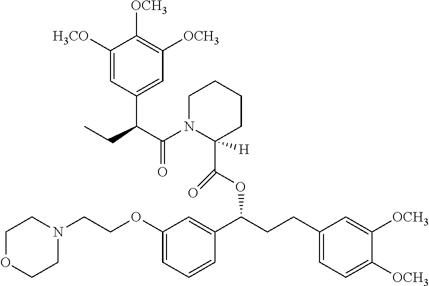 [SMILES: CC[C@H](C(=O)N1CCCC[C@H]1C(=O)O[C@H](CCc1ccc(OC)c(OC)c1)c1cccc(OCCN2CCOCC2)c1)c1cc(OC)c(OC)c(OC)c1]

As used herein, the terms “fused protein” or “fusion protein” refer to two polypeptides arranged in-frame as part of the same contiguous sequence of amino acids. Fusion can be direct such there are no additional amino acid residues between the polypeptides or indirect such that there is a small amino acid linker to improve performance or add functionality. In preferred embodiments, the fusion is direct.

As used herein, the terms “pentameric gH complex” or “gH complex” refer to a complex of five viral proteins on the surface of the CMV virion. The complex is made up of proteins encoded by UL128, UL130, and UL131 assembled onto a gH/gL scaffold (Wang and Shenk, 2005, Proc Natl Acad Sci USA 102:1815; Ryckman et al., 2008, J. Virol. 82:60). The sequences of the complex proteins from CMV strain AD169 are shown at GenBank Accession Nos. NP 783797.1 (UL128), NP 040067 (UL130), CAA35294.1 (UL131), NP 040009 (gH, also known as UL75) and NP_783793 (gL, also known as UL115). Some attenuated CMV strains have one or more mutations in UL131 such that the protein is not expressed and therefore the gH complex is not formed. In such cases, UL131 should be repaired (using methods such as those in Wang and Shenk, 2005, J. Virol. 79:10330) such that the gH complex is expressed in the rdCMV. These viruses express the five viral proteins that make up the pentameric gH complex and assemble the pentameric gH complex on the viral envelope.

As used herein, the term “essential protein” refers to a viral protein that is needed for viral replication in vivo and in tissue culture. Examples of essential proteins in CMV include, but are not limited to, IE1/2, UL37x1, UL44, UL51, UL52, UL53, UL56, UL77, UL79, UL84, UL87 and UL105.

As used herein, the term “destabilized essential protein” refers to an essential protein that is expressed and performs its function in viral replication and is degraded in the absence of a stabilizing agent. In preferred embodiments, the essential protein is fused to a destabilizing protein such as FKBP or a derivative thereof. Under normal growth conditions (i.e., without a stabilizing agent present) the fusion protein is expressed but degraded by host cell machinery. The degradation does not allow the essential protein to function in viral replication thus the essential protein is functionally knocked out. Under conditions where a stabilizing agent such as Shield-1, is present the fusion protein is stabilized and can perform its function at a level that can sustain viral replication that is preferably at least 75%, 80%, 90%, 95%, 99% or 100% of the amount of replication of a CMV that does not contain a destabilized essential protein.

Replication Defective CMV

In one aspect of the present invention the formulation uses a replication defective CMV (rdCMV) that expresses the pentameric gH complex. Any attenuated CMV that expresses the pentameric gH complex can be made replication defective as described herein. In one embodiment, the attenuated CMV is AD169 that has restored gH complex expression due to a repair of a mutation in the UL131 gene (see Example 1).

Conditionally replication defective viruses are mutants in which one or more essential viral proteins have been replaced by a destabilized counterpart of the essential proteins. The destabilized counterpart is encoded by a nucleic acid that encodes a fusion protein between the essential protein and a destabilizing protein. The destabilized essential protein can only function to support viral replication when a stabilizing agent is present. In preferred embodiments, methods described in U.S. Patent Application Publication No. 2009/0215169 are used to confer a conditionally replication defective phenotype to a pentameric gH complex expressing CMV. Briefly, one or more proteins essential for CMV replication are fused to a destabilizing protein, e.g., a FKBP or FKBP derivative. The nucleic acids encoding the wild type essential protein are no longer present in the rdCMV. In the presence of an exogenously added, cell permeable small-molecule stabilizing agent, Shield-1 (Shld-1), the fusion protein is stabilized and the essential protein can function to support viral replication. Replication of the rdCMV in the presence of the stabilizing agent is preferably at least 75%, 80%, 90%, 95%, 99% or 100% of the amount of replication of a CMV that does not contain a destabilizing fusion protein (e.g., the parental attenuated CMV used to construct the rdCMV). In the absence of Shield-1, the destabilizing protein of the fusion protein directs the fusion protein to be substantially degraded by host cell machinery. With no or minimal amounts of essential protein present, the CMV cannot replicate at an amount to produce or maintain a CMV infection in a patient. Replication of the rdCMV in the absence of the stabilizing agent does not take place or is reduced by preferably greater than 50%, 75%, 90%, 95%, or 99% as compared to a CMV that does not contain a destabilizing fusion protein (e.g., the parental attenuated CMV used to construct the rdCMV).

Suitable fusion proteins for use in the present invention retain sufficient essential protein activity to facilitate viral replication in a host cell in the presence of a stabilizing agent and cause a decrease (preferably greater than 50%, 75%, 90%, 95%, or 99% reduction) in CMV replication in the absence of a stabilizing agent. Preferably, the essential protein for use in the fusion protein encodes non-structural proteins and are thus not packaged into the rdCMV virions. Suitable essential proteins identified herein include the CMV proteins encoded by the essential genes IE1/2, UL51, UL52, UL79 and UL84.

Using recombinant DNA methods well known in the art, the nucleic acid encoding an essential protein for CMV replication and/or establishment/maintenance of CMV infection is attached to a nucleic acid that encodes FKBP or a derivative thereof. The encoded fusion protein comprises the FKBP or FKBP derivative fused in-frame to the essential protein. The encoded fusion protein is stable in the presence of Shield-1. However, the encoded fusion protein is destabilized in the absence of Shield-1 and is targeted for destruction. In preferred embodiments, the FKBP is SEQ ID NO:11. In other preferred embodiments, the FKBP derivative is FKBP comprising one or more amino acid substitutions selected from the group consisting of: F15S, V24A, H25R, F36V, E60G, M66T, R71G, D100G, D100N, E102G, K105I and L106P. In a more preferred embodiment, the FKBP derivative comprises the F36V and/or the L106P substitutions (SEQ ID NO:12). In a more preferred embodiment, the FKBP derivative is encoded by SEQ ID NO:13.

The essential proteins targeted for destabilization by fusion with FKBP or a derivative thereof 1) are essential for viral replication; 2) can accommodate the fusion of the destabilizing protein without substantially disrupting function of the essential protein; and 3) can accommodate the insertion of a nucleic acid encoding the FKBP or derivative thereof at the 5′ or 3′ end of the viral ORF encoding the essential protein without substantially disrupting the ORFs of other surrounding viral genes. In preferred embodiments, the essential proteins targeted for destabilization by fusion with FBBP or derivative thereof encode non-structural proteins and, as such, have a decreased likelihood of being packaged into recombinant CMV virions. Table 1 shows CMV genes that meet the aforementioned criteria.

Table 1: Viral genes selected for construction of FKBP fusion

TABLE 1
Viral genes selected for construction of FKBP fusion

| | | Kinetic | Fusion of | Sequence of |
|---|---|---|---|---|
| Viral Gene | Function* | phase | FKBP | Fusion Protein |
| IE1/2 | viral | Immediate | N-term | SEQ ID NOS: |
| (UL123/122) | transcriptional | early | | 1-2 |
| | modulators | | | |
| UL37x1 | Viral gene | Immediate | N-term | |
| | regulations | early | | |
| UL51 | DNA packaging | Late | N-term | SEQ ID NOS: |
| | | | | 3-4 |
| UL52 | DNA packaging | Late | N-term | SEQ ID NOS: |
| | and cleavage | | | 5-6 |
| UL53 | Capsid egress; | Early | C-term | — |
| | nuclear egress | | | |
| UL77 | DNA packaging | Early | C-term | — |
| UL79 | Unknown | Late | N-term | SEQ ID NOS: |
| | | | | 7-8 |
| UL84 | DNA replication | Early-Late | C-term | SEQ ID NOS: |
| | | | | 9-10 |
| UL87 | Unknown | ? | N-term | — |

*according to Mocarski, Shenk and Pass, Cytomegalovirus, in Field Virology, 2701-2772, Editor: Knipes and Howley, 2007

* according to Mocarski, Shenk and Pass, Cytomegalovirus, in Field Virology, 2701-2772, Editor: Knipes and Howley, 2007

The present invention encompasses formulations of rdCMV that comprise fusion proteins with an essential protein or derivative thereof fused to the destabilizing protein. Essential protein derivatives contain one or more amino acid substitutions, additions and/or deletions relative to the wild type essential protein yet can still provide the activity of the essential protein at least well enough to support viral replication in the presence of Shield-1. Examples of measuring virus activity are provided in the Examples infra. Methods known in the art can be used to determine the degree of difference between the CMV essential protein of interest and a derivative. In one embodiment, sequence identity is used to determine relatedness. Derivatives of the invention will be preferably at least 85% identical, at least 90% identical, at least 95% identical, at least 97% identical, at least 99% identical to the base sequence. The percent identity is defined as the number of identical residues divided by the total number of residues and multiplied by 100. If sequences in the alignment are of different lengths (due to gaps or extensions), the length of the longest sequence will be used in the calculation, representing the value for total length.

In some embodiments, the one or more viral proteins essential for viral replication targeted for destabilization are selected from the group consisting of IE1/2, UL51, UL52, UL84, UL79, UL87, UL37x 1, UL77 and UL53 or derivatives thereof. In a specific embodiment, the one or more viral proteins essential for viral replication targeted for destabilization are selected from the group consisting of IE1/2, UL51, UL52, UL84, UL79, UL87. In a more specific embodiment, the one or more viral proteins essential for viral replication targeted for destabilization are selected from the group consisting of IE1/2, UL51, UL52, UL79 and UL84.

More than one essential protein can be destabilized by fusion to FKBP or derivative thereof. In some embodiments, the essential proteins function at different stages of CMV replication and/or infection (including but not limited to, immediate early, early or late stages). In preferred embodiments, the combination of viral proteins essential for viral replication targeted for destabilization are selected from the group consisting of IE1/2 and UL51, IE1/2 and UL52, IE1/2 and UL79, IE1/2 and UL84, UL84 and UL51 and UL84 and UL52. In a more preferred embodiment, IE1/2 and UL51 are targeted for destabilization in the same recombinant CMV. In a most preferred embodiment, the fusion protein comprising IE1/2 is SEQ ID NO:1 and the fusion protein comprising UL51 is SEQ ID NO:3. SEQ ID NOs: 1 and 3 can be encoded by SEQ ID NOs: 2 and 4, respectively. The genome of the rdCMV with the destabilized IE1/2 and UL51 is shown in SEQ ID NO:14.

The FKBP or derivative thereof can be fused to the essential protein either directly or indirectly. In preferred embodiments, the FKBP or derivative thereof is fused to the essential protein directly.

The FKBP or derivative thereof can be fused to the essential protein either at either the N- or C-terminus of the essential protein. In preferred embodiments, the FKBP is fused to the N-terminus of the essential protein.

More than one FKBP or derivative thereof can be fused to the essential protein. In embodiments where there is more than one FKBP or derivative thereof fused to the essential protein, each of the individual FKBP or derivatives thereof can be the same or different. In preferred embodiments, there is one FKBP or derivative thereof fused to the essential protein.

Inactivated CMV

In some embodiments, the rdCMV or CMV described supra is inactivated further using a chemical or physical inactivation. Examples of such include heat treatment, incubation with formaldehyde, ß-Propiolactone (BPL), or binary ethyleneimine (BEI), or gamma irradiation. Preferred methods do not disrupt or substantially disrupt the immunogenicity, including, but not limited to, the immunogenicity induced by the pentameric gH complex. As such, the immune response elicited by the CMV that has been further inactivated is preserved or substantially preserved as compared to rdCMV with no additional inactivation treatment. In preferred embodiments, the ability of the further inactivated CMV to induce neutralizing antibodies is comparable to those induced by rdCMV with no additional inactivation treatment. Inactivation regimen by any one or combination of the chemical or physical methods is determined empirically to ensure immunogenicity of CMV, including the pentameric gH complex.

Evaluation of Viral Replication

One skilled in the art can use viral replication assays to determine the function of a particular essential protein fused to FKBP or derivative thereof. Because gene expression/encoded product function should not be substantially affected by the attachment of the FKBP or derivative thereof to the essential protein in the presence of Shield-1, the rdCMV should replicate at a rate that is comparable to the parental CMV in the presence of Shield-1 (preferably at least 75%, 80%, 90%, 95%, 99% or 100% of the parental virus levels). Replication of the rdCMV is substantially altered from the parental CMV in the absence of Shield-1 (reduced by preferably greater than 50%, 75%, 90%, 95%, 99% or 100% as compared to a CMV that does not contain a destabilizing fusion protein).

In preferred embodiments, the rdCMV in the presence of at least 2 μM Shield-1 replicates preferably at least 90%, more preferably at least 95%, most preferably at least 99%, of the amount that a non-rdCMV replicates.

In one embodiment, a composition comprising the rdCMV of the invention has a viral titer of at least 10⁵ pfu/ml, more preferably at least 10⁷ pfu/ml, in the presence of at least 2 μM Shield-1.

Conversely, rdCMV should not replicate substantially in the absence of Shield-1. The quality of a replication defective mechanism is judged by how stringent the control is under the conditions not permissive for viral replication, i.e., the infectious titers of progeny virions under these conditions. The rdCMV described herein cannot replicate substantially (either in cell culture or within a patient) without Shield-1 present. Its replication in ARPE-19 cells and other types of human primary cells is conditional, and a molar concentration of Shield-1 greater than 0.1 μM, preferable at least 2 μM, in the culture medium is required to sustain viral replication.

In one embodiment, a composition comprising the rdCMV of the invention has a viral titer of less than 2 pfu/ml, more preferably less than 1 pfu/ml, in the absence of Shield-1.

Methods to assess CMV replication can be used to assess rdCMV replication either in the absence or presence of Shield-1. However, in preferred embodiments, the TCID50 is used.

In another embodiment, rdCMV titers are determined by a 50% Tissue Culture Infective Dose (TCID50) assay. Briefly, this dilution assay quantifies the amount of virus required to kill 50% of infected hosts. Host cells (e.g., ARPE-19 cells) are plated and serial dilutions of the virus are added. After incubation, the percentage of cell death (i.e., infected cells) is observed and recorded for each virus dilution. Results are used to mathematically calculate the TCID50.

In another embodiment, the rdCMV titers are determined using a plaque assay. Viral plaque assays determine the number of plaque forming units (pfu) in a virus sample. Briefly, a confluent monolayer of host cells (e.g., ARPE-19 cells) is infected with the rdCMV at varying dilutions and covered with a semi-solid medium, such as agar or carboxymethyl cellulose, to prevent the virus infection from spreading indiscriminately. A viral plaque is formed when a virus infects a cell within the fixed cell monolayer. The virus infected cell will lyse and spread the infection to adjacent cells where the infection-to-lysis cycle is repeated. The infected cell area will create a plaque (an area of infection surrounded by uninfected cells) which can be seen visually or with an optical microscope. Plaques are counted and the results, in combination with the dilution factor used to prepare the plate, are used to calculate the number of plaque forming units per sample unit volume (pfu/mL). The pfu/mL result represents the number of infective particles within the sample and is based on the assumption that each plaque formed is representative of one infective virus particle. In certain embodiments of the invention, the formulation comprises live attenuated CMV or rdCMV at 2.5×10E⁰⁵ to 1.2×10E⁰⁹ pfu/mL after lyophilization.

Adjuvants

Adjuvants are substances that can assist an immunogen in producing an immune response. Adjuvants can function by different mechanisms such as one or more of the following: increasing the antigen biologic or immunologic half-life; improving antigen delivery to antigen-presenting cells; improving antigen processing and presentation by antigen-presenting cells;

achieving dose-sparing, and, inducing production of immunomodulatory cytokines (Vogel, 2000, Clin Infect Dis 30:S266). In some embodiments, the compositions of the invention comprise a rdCMV and an adjuvant. The adjuvant may be added to the formulation before lyophilization, microwave drying, forming lyospheres, or added upon reconsitution of the dried CMV formulation.

A variety of different types of adjuvants can be employed to assist in the production of an immune response. Examples of particular adjuvants include aluminum hydroxide; aluminum phosphate, aluminum hydroxyphosphate, amorphous aluminum hydroxyphosphate sulfate adjuvant (AAHSA) or other salts of aluminum; calcium phosphate; DNA CpG motifs; monophosphoryl lipid A; cholera toxin; E. coli heat-labile toxin; pertussis toxin; muramyl dipeptide; Freund's incomplete adjuvant; MF59; SAF; immunostimulatory complexes; liposomes; biodegradable microspheres; saponins; nonionic block copolymers; muramyl peptide analogues; polyphosphazene; synthetic polynucleotides; IFN-γ; IL-2; IL-12; and ISCOMS. See, e.g., Vogel, 2000, Clin Infect Dis 30:S266; Klein et al., 2000, J Pharm Sci 89:311; Rimmelzwaan et al., 2001, Vaccine 19:1180; Kersten, 2003, Vaccine 21:915; O'Hagen, 2001, Curr. Drug Target Infect. Disord. 1:273.

In other embodiments, particulate adjuvants including, but not limited to, ISCOMATRIX® adjuvant and/or aluminium phosphate adjuvant are used in the compositions of the invention. The Aluminum Phosphate Adjuvant may be added to the aqueous solution prior to lyophilization or added in the diluent for reconstitution of the lyophilized formulation.

Formulations

In certain embodiments, the formulations of the invention comprise a cytomegalovirus (CMV), a buffer at pH about 6.0 to 8.0, an alkali or alkaline salt, a sugar, a cellulose derivative selected from the group consisting of carboxymethyl cellulose, hydroxypropyl cellulose (HPC), hydroxypropyl methylcellulose (HPMC), 2-hydroxyethyl cellulose (2-HEC), crosscarmellose and methyl cellulose, and optionally, a polyol selected from the group consisting of propylene glycol, polypropylene glycol, ethylene glycol, polyethylene glycol, polyethylene glycol monomethyl ethers, and sugar alcohol.

In certain embodiments, the formulations of the invention comprise a cytomegalovirus (CMV), a buffer at pH about 6.0 to 7.5, an alkali or alkaline salt, a sugar, a cellulose derivative selected from the group consisting of carboxymethyl cellulose, hydroxypropyl cellulose (HPC), hydroxypropyl methylcellulose (HPMC), 2-hydroxyethyl cellulose (2-HEC), crosscarmellose and methyl cellulose, and optionally, a polyol selected from the group consisting of propylene glycol, polypropylene glycol, ethylene glycol, polyethylene glycol, polyethylene glycol monomethyl ethers, and sugar alcohol.

In preferred embodiments, the cellulose derivative is anionic and forms a salt, for example carboxymethyl cellulose sodium or potassium at about 0.3-10 mg/ml, 1-10 mg/ml, 3-7 mg/ml or 5 mg/ml in the CMV formulation. Carboxymethyl cellulose salt is available in high viscosity type with average molecular weight of about 700,000; medium viscosity type with average molecular weight of about 250,000; and low viscosity type with average molecular weight of about 90,000. In one embodiment, the cellulose derivative is carboxymethyl cellulose salt with average molecular weight of about 700,000 at about 0.3-1.5 mg/ml in the CMV formulation. In another embodiment, the cellulose derivative is carboxymethyl cellulose salt with average molecular weight of about 250,000 at about 1-4 mg/ml. In a further embodiment, the cellulose derivative is carboxymethyl cellulose salt with average molecular weight of about 90,000 at about 3-7 or 3-10 mg/ml. In yet a further embodiment, the cellulose derivative is carboxymethyl cellulose salt with average molecular weight of about 50,000 to 1,000,000 at about 0.3-10 mg/ml.

In one embodiment, the buffer is selected from the group consisting of phosphate, succinate, histidine, TRIS, MES, MOPS, HEPES, acetate and citrate at about 5-500 mM, 50-300 mM, 10-100 mM, or 20-30 mM. In one embodiment, the buffer is selected from the group consisting of phosphate, histidine, and HEPES at about 5-500 mM, 50-300 mM, 10-100 mM, or 20-30 mM.

The alkali or alkaline salt can provide a stabilizing effect and can be selected from the group consisting of magnesium chloride, calcium chloride, potassium chloride, sodium chloride or a combination thereof at about 50-300 mM, 50-150 mM or 60-80 mM. In certain embodiments, the salt is selected from the group consisting of potassium chloride and sodium chloride at about 50-300 mM, 50-150 mM or 60-80 mM.

The sugar and polyol can act as a cryoprotectant or stabilizing excipient. In one embodiment, the sugar is trehalose or sucrose at about 40-150 mg/ml, 60-110 mg/ml, or 80-100 mg/ml. In another embodiment, the polyol is propylene glycol, glycerol or sorbitol at about 2.5-7.5 mg/ml, 3-7 mg/ml or 5 mg/ml.

The compositions of the invention can be administered to a subject by one or more method known to a person skilled in the art, such as parenterally, transmucosally, transdermally, intramuscularly, intravenously, intra-dermally, intra-nasally, subcutaneously, intra-peritonealy, and formulated accordingly.

In one embodiment, compositions of the present invention are administered via epidermal injection, intramuscular injection, intravenous, intra-arterial, subcutaneous injection, or intra-respiratory mucosal injection of a liquid preparation. Liquid formulations for injection include solutions and the like. The composition of the invention can be formulated as single dose vials, multi-dose vials or as pre-filled syringes.

In another embodiment, compositions of the present invention are administered orally, and are thus formulated in a form suitable for oral administration, i.e., as a solid or a liquid preparation. Solid oral formulations include tablets, capsules, pills, granules, pellets and the like. Liquid oral formulations include solutions, suspensions, dispersions, emulsions, oils and the like.

In one aspect of the invention, the formulation is a solid dried formulation prepared from lyophilization, freezing, microwave drying or through the generation of lyospheres. The formulations can be stored at −70° C., −20° C., 2-8° C. or at room temperature. The dried formulations can be expressed in terms of the weight of the components in a unit dose vial, but this varies for different doses or vial sizes. Alternatively, the dried formulations of the present invention can be expressed in the amount of a component as the ratio of the weight of the component compared to the weight of the drug substance (DS) in the same sample (e.g. a vial). This ratio may be expressed as a percentage. Such ratios reflect an intrinsic property of the dried formulations of the present invention, independent of vial size, dosing, and reconstitution protocol. In one embodiment, the formulation has a d(0.5) μm less than 20, 15, 10 or 5 μm. In other embodiments, the formulation is in lyospheres.

In another aspect of the invention, the formulation is a reconstituted solution. A dried solid formulation can be reconstituted at different concentrations depending on clinical factors, such as route of administration or dosing. For example, a dried formulation may be reconstituted at a high concentration (i.e., in a small volume) if necessary for subcutaneous administration. High concentrations may also be necessary if high dosing is required for a particular subject, particularly if administered subcutaneously where injection volume must be minimized. Subsequent dilution with water or isotonic buffer can then readily be used to dilute the drug product to a lower concentration. If isotonicity is desired at lower drug product concentration, the dried powder may be reconstituted in the standard low volume of water and then further diluted with isotonic diluent, such as 0.9% sodium chloride.

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution. The reconstitution volume can be about 0.5-1.0 ml, preferably 0.5 ml or 0.7 ml.

In another embodiment of the invention, the formulation is the aqueous solution prepared before lyophilization, freezing, microwave drying or generation of lyospheres.

In some embodiments, the rdCMV is administered to a patient to elicit an immune response. It is desirable to minimize or avoid the loss of the rdCMV composition infectivity during storage of the immunogenic composition. The conditions to support such an aim include but not limited to (1) sustained stability in storage, (2) resistant to stressed freezing-thawing cycles, (3) stable at ambient temperatures for up to a week, (4) maintenance of immunogenicity, and (5) compatible with adjuvanting strategy. Conditions that affect rdCMV stability include, but are not limited to, buffer pH, buffer ionic strength, presence/absence of particular excipients and temperature. The compositions comprise buffers to increase the stability of purified rdCMV viral particles suitable as vaccine composition.

The preservation of the integrity of viral particles can be assessed by immunogenicity assays in mice and/or viral entry assays. Viral entry events dependent on the integrity and functions of viral glycoproteins, including the pentameric gH complex. The pentameric gH complex also provides the substantial immunogenicity of rdCMV, thus the two properties are linked.

Processes for Preparing the Lyospheres

Processes for preparing lyospheres are disclosed in U.S. Patent Application Publication US20140294872, the disclosure of which is herein incorporated by reference in its entirety. The method comprises dispensing at least one liquid droplet having a substantially spherical shape onto a solid and flat surface (i.e., lacking any sample wells or cavity), freezing the droplet on the surface without contacting the droplet with a cryogenic substance and lyophilizing the frozen droplet to produce a dried pellet that is substantially spherical in shape. U.S. Pat. No. 9,119,794, the disclosure of which is herein incorporated by reference in its entirety, also discloses processes for forming lyospheres. The unitary volumes containing the aqueous medium mixture are formed on a solid element containing cavities. The solid element is cooled below the freezing temperature of the mixture, the cavities are filled with the mixture, and the mixture is solidified while present in the cavity to form the unitary forms. The unitary forms are dried in a vacuum to provide the lyospheres.

In other embodiments, the lyospheres are formed in a substantially spherical shape and are prepared by freezing droplets of a liquid composition of a desired biological material on a flat, solid surface, in particular, a surface that does not have any cavities, followed by lyophilizing the unitary forms. U.S. Patent Application Publication No. US2014/0294872, the disclosure of which is herein incorporated by reference, discloses similar processes for forming lyospheres.

Briefly, in some embodiments the process comprises dispensing at least one liquid droplet having a substantially spherical shape onto a solid and flat surface (i.e., lacking any sample wells or cavity), freezing the droplet on the surface without contacting the droplet with a cryogenic substance and lyophilizing the frozen droplet to produce a dried pellet that is substantially spherical in shape. The process may be used in a high throughput mode to prepare multiple dried pellets by simultaneously dispensing the desired number of droplets onto the solid, flat surface, freezing the droplets and lyophilizing the frozen droplets. Pellets prepared by this process from a liquid formulation may have a high concentration of a biological material (such as a protein therapeutic) and may be combined into a set of dried pellets.

In some embodiments, the solid, flat surface is the top surface of a metal plate which comprises a bottom surface that is in physical contact with a heat sink adapted to maintain the top surface of the metal plate at a temperature of −90° C. or below. Since the top surface of the metal plate is well below the freezing point of the liquid formulation, the droplet freezes essentially instantaneously with the bottom surface of the droplet touching the top surface of the metal plate.

In other embodiments, the solid, flat surface is hydrophobic and comprises the top surface of a thin film that is maintained above 0° C. during the dispensing step. The dispensed droplet is frozen by cooling the thin film to a temperature below the freezing temperature of the formulation.

Lyophilization Process

The lyophilized formulations of the present invention are formed by lyophilization (freeze-drying) of a pre-lyophilization solution. Freeze-drying is accomplished by freezing the formulation and subsequently subliming water at a temperature suitable for primary drying. Under this condition, the product temperature is below the eutectic point or the collapse temperature of the formulation. Typically, the shelf temperature for the primary drying will range from about −50 to 25° C. (provided the product remains frozen during primary drying) at a suitable pressure, ranging typically from about 30 to 250 mTorr. The formulation, size and type of the container holding the sample (e.g., glass vial) and the volume of liquid will dictate the time required for drying, which can range from a few hours to several days (e.g. 40-60 hrs). A secondary drying stage may be carried out at about 0-40° C., depending primarily on the type and size of container and the type of protein employed. The secondary drying time is dictated by the desired residual moisture level in the product and typically takes at least about 5 hours. Typically, the moisture content of a lyophilized formulation is less than about 5%, and preferably less than about 3%. The pressure may be the same as that employed during the primary drying step. Freeze-drying conditions can be varied depending on the formulation, vial size and lyophilization trays.

In some instances, it may be desirable to lyophilize or microwave dry the protein-polysaccharide formulation in the container in which reconstitution is to be carried out in order to avoid a transfer step. The container in this instance may, for example, be a 2, 3, 5, 10 or 20 ml vial.

Administration

A rdCMV formulated as described herein can be administered to a patient using the guidance provided herein along with techniques well known in the art. Guidelines for pharmaceutical administration in general are provided in, for example, Vaccines Eds. Plotkin and Orenstein, W. B. Sanders Company, 1999; Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990.

Vaccines can be administered by different routes such as subcutaneous, intramuscular, intravenous, mucosal, parenteral, transdermal or intradermal. Subcutaneous and intramuscular administration can be performed using, for example, needles or jet-injectors. In an embodiment, the vaccine of the invention is administered intramuscularly. Transdermal or intradermal delivery can be accomplished through intradermal syringe needle injection, or enabling devices such as micron-needles or micron array patches.

The formulations described herein may be administered in a manner compatible with the unit dosage, and in such amount as is immunogenically-effective to treat and/or reduce the likelihood of CMV infection (including primary, recurrent and/or super). The dose administered to a patient, in the context of the present invention, should be sufficient to effect a beneficial response in a patient over time such as a reduction in the level of CMV infection, ameliorating the symptoms of disease associated with CMV infection and/or shortening the length and/or severity of CMV infection, or to reduce the likelihood of infection by CMV (including primary, recurrent and/or super).

Suitable dosing regimens may be readily determined by those of skill in the art and are preferably determined taking into account factors well known in the art including age, weight, sex and medical condition of the patient; the route of administration; the desired effect; and the particular composition employed. In determining the effective amount of the rdCMV to be administered in the treatment or prophylaxis against CMV, the physician may evaluate circulating plasma levels of virus, progression of disease, and/or the production of anti-CMV antibodies. The dose for a vaccine composition consists of the range of 10³ to 10¹² plaque forming units (pfu). In different embodiments, the dosage range is from 10⁴ to 10¹⁰ pfu, 10⁵ to 10⁹ pfu, 10⁶ to 10⁸ pfu, or any dose within these stated ranges. When more than one vaccine is to be administered (i.e., in combination vaccines), the amount of each vaccine agent is within their described ranges.

The vaccine composition can be administered in a single dose or a multi-dose format. Vaccines can be prepared with adjuvant hours or days prior to administrations, subject to identification of stabilizing buffer(s) and suitable adjuvant composition. Vaccines can be administrated in volumes commonly practiced, ranging from 0.1 mL to 0.5 mL.

The timing of doses depends upon factors well known in the art. After the initial administration one or more additional doses may be administered to maintain and/or boost antibody titers and T cell immunity. Additional boosts may be required to sustain the protective levels of immune responses, reflected in antibody titers and T cell immunity such as ELISPOT. The levels of such immune responses are subject of clinical investigations.

For combination vaccinations, each of the immunogens can be administered together in one composition or separately in different compositions. A rdCMV described herein can be administered concurrently with one or more desired immunogens. The term “concurrently” is not limited to the administration of the therapeutic agents at exactly the same time, but rather it is meant that the rdCMV described herein and the other desired immunogen(s) are administered to a subject in a sequence and within a time interval such that they can act together to provide an increased benefit than if they were administered otherwise. For example, each therapeutic agent may be administered at the same time or sequentially in any order at different points in time; however, if not administered at the same time, they should be administered sufficiently close in time so as to provide the desired therapeutic effect. Each therapeutic agent can be administered separately, in any appropriate form and by any suitable route.

Embodiments also include formulations of the CMV comprising or consisting of said CMV or compositions (i) for use in, (ii) for use as a medicament for, or (iii) for use in the preparation of a medicament for: (a) therapy (e.g., of the human body); (b) medicine; (c) inhibition of CMV replication; (d) treatment or prophylaxis of infection by CMV or, (e) treatment, prophylaxis of, or delay in the onset or progression of CMV-associated disease(s). In these uses, the formulations of CMV comprising or consisting of said CMV or compositions can optionally be employed in combination with one or more anti-viral agents (e.g., anti-viral compounds or anti-viral immunoglobulins; combination vaccines, described infra).

Patient Population

A “patient” refers to a mammal capable of being infected with CMV. In a preferred embodiment, the patient is a human. A patient can be treated prophylactically or therapeutically. Prophylactic treatment provides sufficient protective immunity to reduce the likelihood or severity of a CMV infection, including primary infections, recurrent infections (i.e., those resulting from reactivation of latent CMV) and super-infections (i.e., those resulting from an infection with a different stain of CMV than previously experienced by the patient). Therapeutic treatment can be performed to reduce the severity of a CMV infection or decrease the likelihood/severity of a recurrent or super-infection.

Treatment can be performed using a pharmaceutical composition comprising a rdCMV as described herein. Pharmaceutical compositions can be administered to the general population, especially to those persons at an increased risk of CMV infection (either primary, recurrent or super) or for whom CMV infection would be particularly problematic (such as immunocompromised individuals, transplant patients or pregnant women). In one embodiment, females of childbearing age, especially early adolescent females, are vaccinated to decrease the likelihood of CMV infection (either primary, recurrent or super) during pregnancy.

Those in need of treatment include those already with an infection, as well as those prone to have an infection or in which a reduction in the likelihood of infection is desired. Treatment can ameliorate the symptoms of disease associated with CMV infection and/or shorten the length and/or severity of CMV infection, including infection due to reactivation of latent CMV.

Persons with an increased risk of CMV infection (either primary, recurrent or super) include patients with weakened immunity or patients facing therapy leading to a weakened immunity (e.g., undergoing chemotherapy or radiation therapy for cancer or taking immunosuppressive drugs). As used herein, “weakened immunity” refers to an immune system that is less capable of battling infections because of an immune response that is not properly functioning or is not functioning at the level of a normal healthy adult. Examples of patients with weakened immunity are patients that are infants, young children, elderly, pregnant or a patient with a disease that affects the function of the immune system such as HIV infection or AIDS.

Having described various embodiments of the invention with reference to the accompanying description and drawings, it is to be understood that the invention is not limited to those precise embodiments, and that various changes and modifications may be effected therein by one skilled in the art without departing from the scope or spirit of the invention as defined in the appended claims.

The following examples illustrate, but do not limit the invention.

### EXAMPLES

### Example 1: Restoration of the Pentameric gH Complex

An infectious CMV bacterial artificial chromosome clone was constructed so that the encoded virion that expressed the pentameric gH complex consisting of UL128, UL130 and UL131 assembled onto a gH/gL scaffold.

CMV strain AD169 strain was originally isolated from the adenoids of a 7-year-old girl (Elek and Stern, 1974, Lancet, 1:1). The virus was passed 58 times in several types of human fibroblasts to attenuate the virus (Neff et al., 1979, Proc Soc Exp Biol Med, 160:32, with the last 5 passages in WI-38 human fibroblasts. This passaged variant of AD169 virus, referred in this study as Merck AD169 (MAD169), was used as the parental virus to construct the infectious BAC clone. Neither the parental virus AD169 nor the passaged variant virus MAD169 expressed UL131 or the pentameric gH complex.

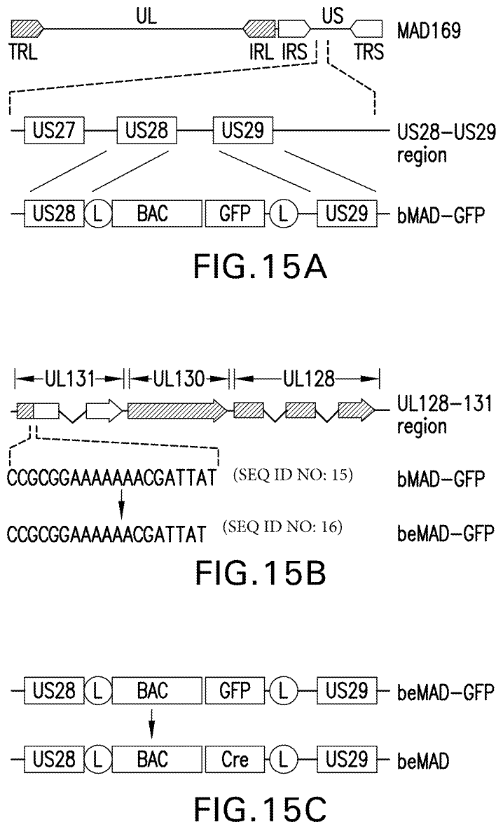

The MAD169 was used as the parental virus to construct an infectious bacterial artificial chromosome (BAC) clone. A BAC vector is a molecular tool that allows the genetic manipulation of a large size DNA fragment, such as the CMV genome (˜230 Kb), in E. coli. A BAC element along with a GFP marker gene was inserted immediately after the stop codon of US28 open reading frame (between US28 and US29 ORFs in the viral genome) with a LoxP site created at the both ends of the fragment (FIG. 15A). Briefly, a DNA fragment containing a GFP expression cassette flanked by two loxP sites and CMV US28-US29 sequences were synthesized and cloned into pBeloBAC11 vector. The BAC vector was linearized with restriction enzyme Pmel, and cotransfected into MRC-5 cells with MAD169 DNA extracted from purified virions. The recombinant variants, identified by green fluorescence expression, were plaque purified. After one round of amplification, the circular form of viral genome was extracted from the infected cells, and electroporated into E. coli DH10 cells. The bacterial colonies were screened by PCR for the presence of US28 and US29 regions. Candidate clones were further examined by EcoRI, EcoRV, Hind II, SpeI and BamHI restriction analyses. After screening, one clone, bMAD-GFP, showed identical restriction pattern with the parental MAD169 virus.

The frame-shift mutation in the first exon of UL131 underlying the epithelial tropism deficiency in MAD169 was repaired genetically in E. coli (FIG. 15B). Specifically, one adenine nucleotide (nt) from the 7 nt A-stretch in the UL131 gene was deleted (FIG. 15B). Deletion of 1 nt was sufficient to rescue the epithelial and endothelial cell tropism due to UL131, and thus the pentameric gH complex, now being expressed. Expression was confirmed by ELISA and western blot (data not shown). This clone was further modified by removing the BAC segment by LoxP/Cre recombination. The BAC DNA was transfected in ARPE-19 cells, human retinal pigmented epithelial cells (ATCC Accession No. CRL-2302), to recover the infectious virus (FIG. 15C). The resultant infectious virus, termed BAC-derived epithelial-tropic MAD169 virus (beMAD), differs from MAD169 only in two loci, (1) UL131 ORF where a single adenine nucleotide was deleted and (2) a 34 bp LoxP site inserted between US28 and US29 ORFs (see Table 2).

The genome of the BAC clone beMAD was completely sequenced. The overall genome structure of beMAD is identical to that reported in the ATCC AD169 variant (GenBank Accession No. X17403), which is comprised of two unique regions, unique long (UL) and unique short (US). Each unique regions are bracketed by two repeat sequences, terminal repeat long (TRL)-internal repeat long (IRL), terminal repeat short (TRS)-internal repeat short (IRS). The growth kinetics of the passaged variant MAD169 and the beMAD derived virus were indistinguishable in MRC-5 cells, a human fibroblast cell line (ATCC Accession No. CCL-171) (data not shown). Because the pentameric gH complex is not needed for growth on fibroblast cells, the differences in the pentameric gH complex expression between the MAD169 and beMAD are not relevant.

TABLE 2
Molecular difference of CMV

| Virus ID | Genetic composition | Proteins in virions |
|---|---|---|
| AD169 | ATCC laboratory strain containing | |
| | frame-shift mutation in UL131 causing | |
| | deficiency in epithelial tropism | |
| MAD169 | Contains frame-shift mutation in | Identical to |
| | UL131 identical to ATCC AD169 | AD169 from ATCC |
| beMAD | Repaired frame-shift mutation in | Identical to |
| | UL131; LoxP sequence (34 bp) | MAD169, with |
| | between US28 and US29 ORFs | addition of the |
| | | pentameric gH |
| | | complex |

### Example 2: Effect of Conventional Inactivation Methods on CMV

The effect of two conventional methods of viral inactivation, γ-irradiation and ß-Propiolactone (BPL), were investigated on the CMV expressing the pentameric gH complex.

The γ-irradiation was performed on lyophilized virions. Recombinant CMV at a concentration of 0.15 mg/mL in HNS (25 mM Histidine, 150 mM NaCl, 9% w/v Sucrose, pH 6.0) formulation was lyophilized using a conservative lyophilization cycle (−50° C. freezing and primary drying at −35° C. for −30 hrs followed by secondary drying at 25° C. for 6 hrs) to obtain dry powder. The vaccine was lyophilized in a 3 mL glass vial with 0.5 ml filled in each vial. At the end of lyophilization, the vials were stoppered in a nitrogen environment and the samples were removed, labelled, crimped and stored at −70° C. until gamma irradiation. The vials were irradiated under a Co irradiator for the desired dosage of irradiation.

For BPL treatment, a BPL stock solution was added to the crude viral culture supernatant from growth on ARPE-19 cells to reach the final concentrations of 0.01% or 0.1% (v/v). The reaction was terminated with sodium thiosulfate at various time points. The BPL-treated pentameric gH complex-expressing CMV were then purified by ultracentrifugation.

### Example 3: Construction and Screening of FKBP-Essential Protein Fusions

A CMV was constructed using the attenuated AD169 strain backbone that regains its epithelial tropism while being conditionally replication defective. Methods described in Example 1 were used to restore epithelial tropism.

The viral proteins to be fused to the FKBP derivative were selected based on two criteria. First, the proteins of interest were not detected in CMV virions by proteomics analysis (Varnum et al., 2004, J. Virol. 78:10960), thus, decreasing the likelihood that the FKBP fusion protein will be incorporated into virus. Second, the proteins of interest are essential for viral replication in tissue culture.

Using beMAD as the parental virus, the FKBP derivative (SEQ ID NO:12) was fused to 12 essential viral proteins individually, including IE1/2 (SEQ ID NO:1), pUL37x1, pUL44, pUL51 (SEQ ID NO:3), pUL52 (SEQ ID NO:5), pUL53, pUL56, pUL77, pUL79 (SEQ ID NO:7), pUL84 (SEQ ID NO:9), pUL87 and pUL10⁵. A virus with two different essential proteins fused to FKBP was also constructed that fused each of IE1/2 and UL51 with the FKBP derivative (the genome of the rdCMV with the destabilized IE1/2 and UL51 is shown in SEQ ID NO:14). After construction, all recombinant BAC DNAs were transfected into ARPE-19 cells, and cultured in the medium containing Shld-1.

### Example 4: Formulation Excipient Screening for Lyophilization Process Yield and Short-Term Stability

Materials: Histidine was purchased from Sigma-Aldrich, St. Louis, Mo., USA or Avantor, Center Valley, Pa., USA. Tris (Tris(hydroxymethyl)aminomethane) and Calcium chloride were purchased from Sigma-Aldrich, St. Louis, Mo., USA. Sodium Carboxymethylcellulose (90,000 average molecular weight) was purchased from Sigma-Aldrich, St. Louis, Mo., USA or Ashland Specialty Ingredients, Wilmington, Del., USA. Sodium Chloride was purchased from Avantor, Center Valley, Pa., USA. Propylene glycol was purchased from Sigma-Aldrich, St. Louis, Mo., USA or Dow Chemical Co, Midland, Mich., USA. Sorbitol and Glycerol and Urea were purchased form Sigma-Aldrich, St. Louis, Mo., USA or Fisher Scientific, USA. Dilute hydrochloric acid and sodium hydroxide were purchased from Avantor, Center Valley, Pa., USA.

### Formulation Preparation:

The rdCMV (SEQ ID NO: 14) bulk prepared as described above and in U.S. Pat. No. 9,546,355 (incorporated herein by reference in its entirety) was formulated in 25 mM histidine, 150 mM sodium chloride, 90 mg/mL sucrose at pH 6.0 or 25 mM histidine, 75 mM sodium chloride, 90 mg/mL sucrose at pH 7.0 and stored at −70° C. The formulation compositions were prepared as a stock at 1.25-2 times higher concentration and used in the formulations to achieve a final composition as indicated after mixing with CMV bulk. The CMV bulk was thawed and formulated in appropriate formulation compositions at 100-350 Units of CMV/mL (100-350 μg/mL or 2.5×10E05 to 1.2×10E° 9 pfu/mL).

The formulations were filled into 2 mL glass vials at either 0.7 mL or 0.5 mL. The vials for liquid control were closed with stoppers, crimped with aluminum caps and vials were frozen either in a−70° C. freezer or nitrogen cooled fast freezer and then stored at −70° C. until analyzed.

The vials for lyophilization were filled at either 0.7 mL or 0.5 mL on a lyophilization tray, partially stoppered with lyophilization stoppers. The vials were frozen either in a nitrogen cooled fast freezer set to <−50° C. or on the pre-cooled lyophilizer shelf at <−50° C. The Lyophilization was performed utilizing a Lyostar II or III (SP Scientific, Warminster, PA). The liquid formulations or frozen formulations were loaded onto the lyophilizer shelf that was pre-cooled and held at −50° C. After soaking at −50° C., shelf temperature was ramped from −50° C. to between −30 to −17° C. at a ramp rate of 0.1° C.-0.5° C./min for primary drying. The shelf temperature was ramped to 15 to 30° C. set point at 0.1° C.-0.5° C./min ramp rate for secondary drying.

### Measurement of Viral Replication (Infectivity):

Imaging of Relative Viral Expression (IRVE) Assay: CMV infectivity was measured using cell-based relative infectivity assay, Imaging of Relative Viral Expression (IRVE) Assay. This stability-indicating method is a cell-based relative infectivity assay based on expression of immediate-early (IE) protein 1 (IE1) of CMV. In this assay, ARPE-19 cells are planted in 384-well micro-titer plates, incubated for 24 hours ±4 hours, and then infected with serial dilutions of rdCMV (SEQ ID NO:14) reference standard, positive control, and test articles. The infection proceeds at 37° C. with 5% CO₂ for 20 hours followed by fixation of the cells with a dilute formaldehyde solution. The method was performed using medium formulated with excess Shld-1. The fixed cells are permeabilized then primary antibody is added to the plates and incubated for 1 hour. After washing the plates, secondary antibody (AlexaFluor conjugate) was added to the wells, incubated at room temperature for 30 minutes, then washed off. After washing the plates, Nuclei stain (Hoechst 33342 DNA stain) was added to the wells, incubated at room temperature for 5 minutes, then washed off. PBS was added to the plates and they were read using a Cytation 3 imaging reader. The potency of samples relative to the reference was calculated (% RP) from the sample EC50 and reference EC50 values via reduced 4 parameter logistic regression, 4-PL % RP=(Sample ED50)/(Reference ED50)×100% RP=(Sample ED50)/(Reference ED50)×100. The CMV titers (pfu/mL) for reference standard used in IRVE assay was measured using CMV plaque assay and can be used to convert the % RP from IRVE assay of the test articles to pfu/mL.

The frozen liquid control samples were thawed at ambient temperature prior to testing. The lyophilized vials were reconstituted with either sterile water or sterile 9 mg/mL sodium chloride solution prior to testing.

### Data Analysis

For each data point reported n=3 individual samples were tested and the average data from 3 tests was reported. A standard deviation (STDEV) or standard error of the mean (SEM) for the samples was calculated for the data and 2×SEM is reported.

### Lyophilization Process Yield:

For the lyophilization yield, the lyophilized vials stored at −70° C. were tested along with liquid control vials (stored at −70° C.) using a cell-based infectivity assay and the lyophilization yield was calculated as a percentage of liquid control sample. The expected variability of the assay is approximately 30%.

### Lyophilization Stability:

The stability samples at different time points were tested using the cell-based infectivity assay along with lyophilization control samples stored at −70° C. and the lyophilization stability was calculated as a Log10 loss compared to the lyophilized control sample.

Base formulation for CMV was 12.5 mM Histidine, 12.5 mM Tris, 75 mM Sodium Chloride, 90 mg/mL w/v Sucrose; pH 7.0 (CMV-098). Excipient screening was performed for improving the lyophilization process yield and stability of short-term virus infectivity stability at 2-8° C. (4° C.), and 15° C. The formulations are listed in Table 3.

TABLE 3
Formulation codes and compositions for excipient screening

| Formulation | |
|---|---|
| Code | Formulation Composition |
| CMV-098 | 12.5 mM Histidine, 12.5 mM Tris, 75 mM NaCl, 90 mg/mL Sucrose; pH 7.0 |
| CMV-142 | 12.5 mM Histidine, 12.5 mM Tris, 75 mM NaCl, 90 mg/mL Sucrose, 5 |
| | mg/mL Glycerol; pH 7.0 |
| CMV-165 | 12.5 mM Histidine, 12.5 mM Tris, 75 mM NaCl, 90 mg/mL Sucrose, 5 |
| | mg/mL sodium carboxymethylcellulose; pH 7.0 |
| CMV-170 | 12.5 mM Histidine, 12.5 mM Tris, 75 mM NaCl, 90 mg/mL Sucrose, 5 |
| | mg/mL Glycerol, 5 mg/mL sodium carboxymethylcellulose; pH 7.0 |
| CMV-182 | 12.5 mM Histidine, 12.5 mM Tris, 75 mM NaCl, 30 mg/mL Sucrose, 50 |
| | mg/mL Sorbitol, 5 mg/mL sodium carboxymethylcellulose; pH 7.0 |
| CMV-188 | 12.5 mM Histidine, 12.5 mM Tris, 75 mM NaCl, 90 mg/mL Sucrose, 5 |
| | mg/mL propylene glycol, 5 mg/mL sodium carboxymethylcellulose; pH 7.0 |
| CMV-189 | 12.5 mM Histidine, 12.5 mM Tris, 75 mM NaCl, 90 mg/mL Sucrose, 10 |
| | mg/mL Propylene Glycol, 5 mg/mL sodium carboxymethylcellulose; pH 7.0 |
| CMV-192 | 12.5 mM Histidine, 12.5 mM Tris, 75 mM NaCl, 90 mg/mL Sucrose, 5 |
| | mg/mL propylene glycol; pH 7.0 |

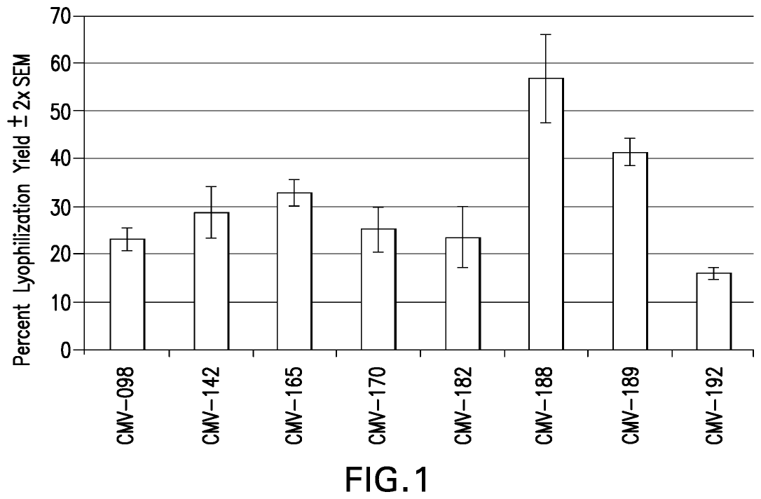

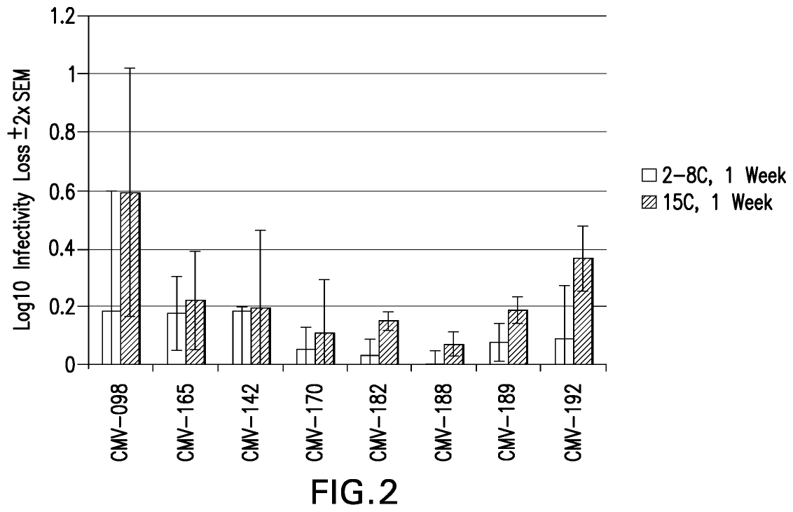

The percent lyophilization yield data (FIG. 1) showed the addition of propylene glycol and sodium carboxymethylcellulose (CMV-188 and CMV-189) to sucrose alone formulation (CMV-098) improved lyophilization process yield significantly by approximately 2-fold. The lyophilized rdCMV (SEQ ID NO: 14) stability at different storage conditions (FIG. 2) showed lower infectivity loss with formulations containing sodium carboxymethylcellulose (CMV-165), glycerol (CMV-142), glycerol and sodium carboxymethylcellulose (CMV-170), sorbitol and sodium carboxymethylcellulose (CMV-182), propylene glycol and sodium carboxymethylcellulose (CMV-188 & 189) compared to sucrose alone formulation (CMV-098). Additionally, CMV-098 formulation showed a greater variability in stability as shown by higher SEM.

### Example 5: Formulation Excipient Screening for Lyophilization Process Yield and Long-Term Stability at 2-8° C.

Subsequent to the initial screening study, a long-term stability study was performed to identify stabilizing formulations. Base formulation for rdCMV (SEQ ID NO: 14) was 12.5 mM Histidine, 12.5 mM Tris, 75 mM Sodium Chloride, 90 mg/mL w/v Sucrose; pH 7.0 (CMV-098). Excipient screening was performed for improving the lyophilization process yield and stability of long-term virus infectivity stability at 2-8° C. (4° C.). The formulations tested are listed in Table 4.

TABLE 4
Formulation codes and compositions

| Formulation | |
|---|---|
| Code | Formulation Composition |
| CMV-098 | 12.5 mM Histidine, 12.5 mM Tris, 75 mM Sodium Chloride, 90 mg/mL |
| | Sucrose; pH 7.0 |
| CMV-099 | 12.5 mM Histidine, 12.5 mM Tris, 75 mM Sodium Chloride, 90 mg/mL |
| | Sucrose; 5 mg/mL Urea; pH 7.0 |
| CMV-165 | 12.5 mM Histidine, 12.5 mM Tris, 75 mM Sodium Chloride, 90 mg/mL |
| | Sucrose, 5 mg/mL Sodium Carboxymethylcellulose; pH 7.0 |
| CMV-170 | 12.5 mM Histidine, 12.5 mM Tris, 75 mM Sodium Chloride, 90 mg/mL |
| | Sucrose; 5 mg/mL Glycerol; 5 mg/mL Sodium Carboxymethylcellulose; pH |
| | 7.0 |
| CMV-188 | 12.5 mM Histidine, 12.5 mM Tris, 75 mM Sodium Chloride, 90 mg/mL |
| | Sucrose; 5 mg/mL Propylene Glycol; 5 mg/mL Sodium |
| | Carboxymethylcellulose; pH 7.0 |
| CMV-202 | 25 mM Histidine, 75 mM Sodium Chloride, 90 mg/mL Sucrose; 5 mg/mL |
| | Propylene Glycol; 5 mg/mL Sodium Carboxymethylcellulose; pH 7.0 |
| CMV-220 | 25 mM Histidine, 75 mM Sodium Chloride, 90 mg/mL Sucrose; 5 mg/mL |
| | Propylene Glycol; 5 mg/mL Sodium Carboxymethylcellulose, 5 mg/mL |
| | Calcium Chloride; pH 7.0 |

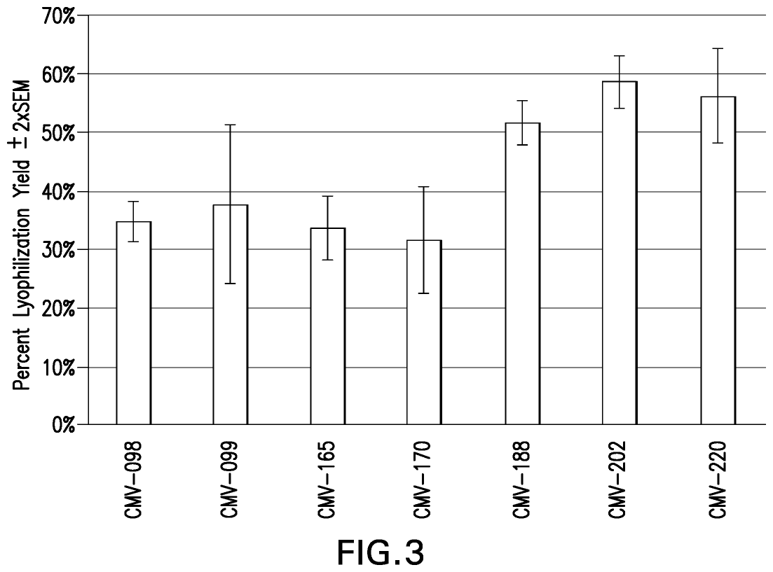

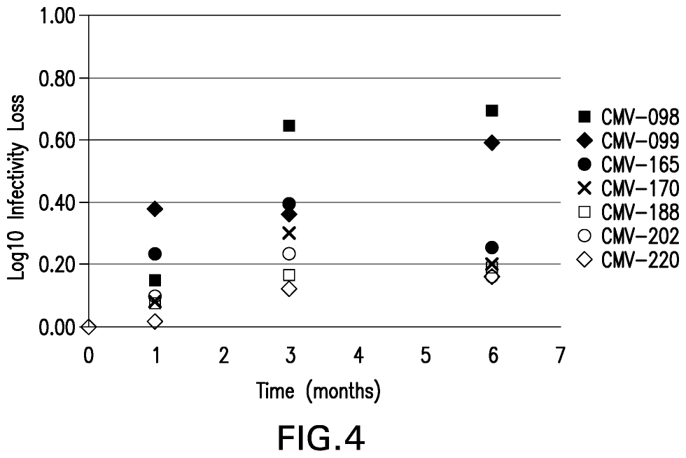

The percent lyophilization yield data (FIG. 3) again showed that the addition of propylene glycol and sodium carboxymethylcellulose (CMV-188, CMV-202) to the base sucrose formulation (CMV-098) showed significant improvement by approximately 2 fold. The lyophilization process yield with CMV-188 is similar to FIG. 1. Addition of calcium chloride (CMV-220) to the CMV-202 formulation showed no effect on process yield (FIG. 3). Addition of urea (CMV-099) did not improve either process yield or stability of rdCMV (SEQ ID NO: 14) as compared to the sucrose formulation (CMV-098). Addition of sodium carboxymethylcellulose (CMV-165), glycerol (CMV-165), propylene glycol and sodium carboxymethylcellulose (CMV-188 and CMV-202), propylene glycol and sodium carboxymethylcellulose and calcium chloride (CMV-220) significantly improved stability of rdCMV (SEQ ID NO: 14) at 6 months by approximately 3-fold (FIG. 4). Formulations CMV-188 and CMV-202 and CMV-220 had less than 0.2 log 10 infectivity loss at 2-8° C. after 6 months (FIG. 4). For formulations CMV-188, CMV-202 and CMV-220, it is expected that after 2 years of storage at 2-8° C., the infectivity loss will be less than 0.5 log 10.

### Example 6: Effect of pH on Lyophilization Process Yield and Stability of CMV in CMV-202 Formulation

The impact of pH was studied in CMV-202 composition in the pH range of 6.0 to 7.5 by adjusting the pH of the formulations. The formulations and the pH are listed in Table 5. The liquid formulations were prepared with 200 Units/mL of rdCMV (SEQ ID NO: 14) and filled at 0.7 mL in 2 mL glass vials, stoppered, crimped and stored frozen at −70° C. for liquid control. For lyophilization, the formulations were filled at 0.7 mL in 2 mL glass vials, partially stoppered with lyophilization stoppers and lyophilized. After completing the lyophilization process, the control lyophilized product is stored at −70° C. and stability samples are subjected to real-time (2-8° C.) stability condition for 1 and 3 months.

TABLE 5
Formulation codes and compositions

| Formulation | |
|---|---|
| Code | Formulation Composition |
| CMV-188 | 12.5 mM Histidine, 12.5 mM Tris, 75 mM Sodium Chloride, 90 mg/mL |
| | Sucrose; 5 mg/mL Propylene Glycol; 5 mg/mL Sodium |
| | Carboxymethylcellulose; pH 7.0 |
| CMV-202 | 25 mM Histidine, 75 mM Sodium Chloride, 90 mg/mL Sucrose; 5 mg/mL |
| | Propylene Glycol; 5 mg/mL Sodium Carboxymethylcellulose; pH 7.0 |
| CMV-214 | 25 mM Histidine, 75 mM Sodium Chloride, 90 mg/mL Sucrose; 5 mg/mL |
| | Propylene Glycol; 5 mg/mL Sodium Carboxymethylcellulose; pH 6.0 |
| CMV-215 | 25 mM Histidine, 75 mM Sodium Chloride, 90 mg/mL Sucrose; 5 mg/mL |
| | Propylene Glycol; 5 mg/mL Sodium Carboxymethylcellulose; pH 6.5 |
| CMV-216 | 25 mM Histidine, 75 mM Sodium Chloride, 90 mg/mL Sucrose; 5 mg/mL |
| | Propylene Glycol; 5 mg/mL Sodium Carboxymethylcellulose; pH 7.5 |

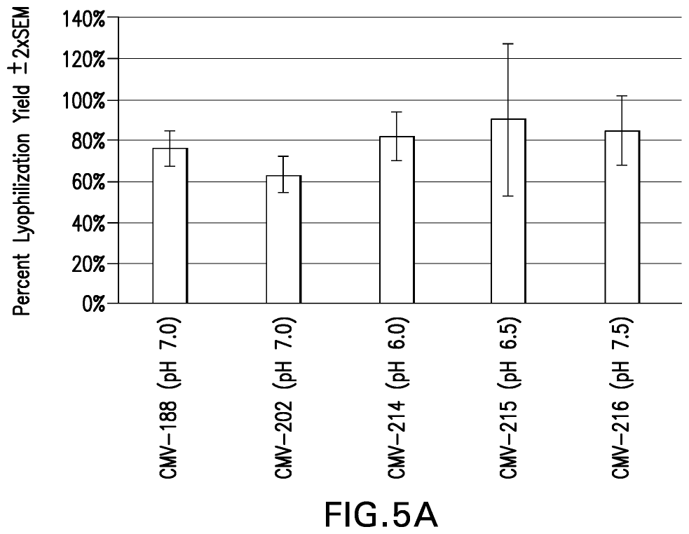

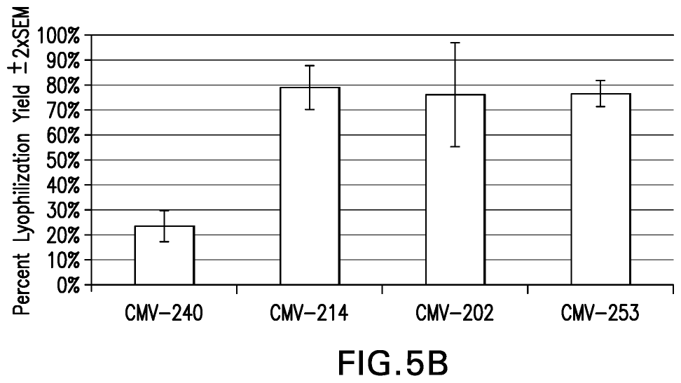

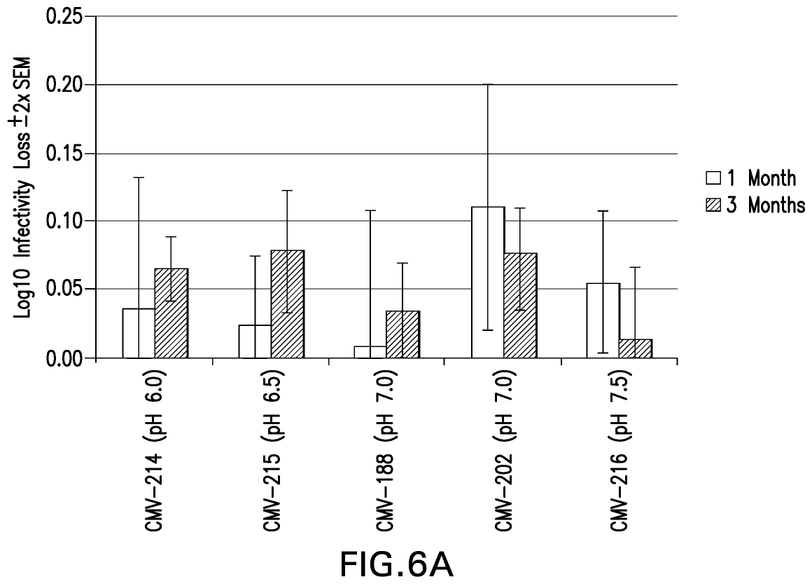

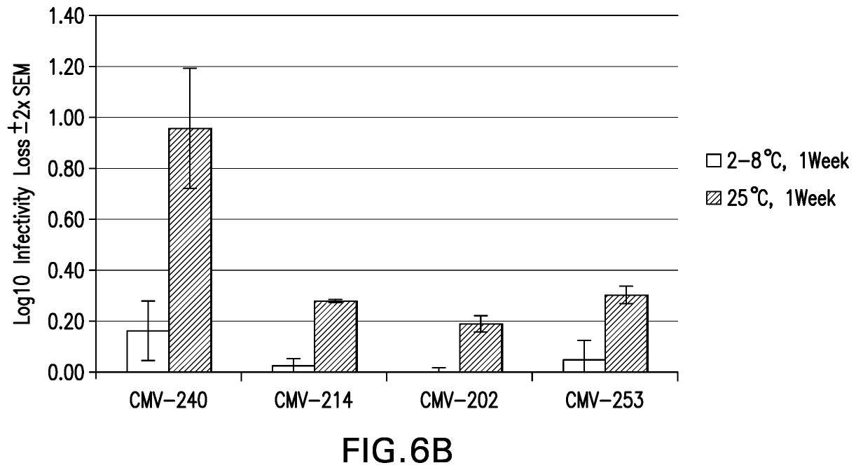

The lyophilization process yield for CMV-202 (FIG. 5A) at pH 7.0 showed consistently higher lyophilization yield as compared to CMV-098 (as shown in FIGS. 1 and 3). The lyophilization yield for other pH levels tested were also higher than CMV-098 (as shown in FIGS. 1 and 3) and similar to CMV-202 (FIG. 5A). The infectivity loss for CMV-202 at all pH levels tested (FIG. 6A) at 1 and 3 months at 2-8° C. was low compared to CMV-098 (as shown in FIG. 4).

A subsequent study was performed to determine the impact of pH in CMV-202 composition at pH 6.0, 7.0 and 8.0. The formulations and the pH are listed in Table 5-1. The liquid formulations were prepared with 280 Units/mL of rdCMV (SEQ ID NO:14) and filled at 0.5 mL in 2 mL glass vials, stoppered, crimped and stored frozen at −70° C. for liquid control. For lyophilization, the formulations were filled at 0.5 mL in 2 mL glass vials, partially stoppered with lyophilization stoppers and lyophilized. After completing the lyophilization process, the control lyophilized product is stored at −70° C. and stability samples are subjected to real-time (2-8° C.) stability condition or accelerated stability condition (25° C.) for 1 week. The samples were reconstituted with 0.7 mL of saline (0.9% w/v sodium chloride) solution for testing.

TABLE 5-1
Formulation codes and compositions

| Formulation | |
|---|---|
| Code | Formulation Composition |
| CMV-240 | 25 mM Histidine, 75 mM Sodium Chloride, 90 mg/mL Sucrose; pH 7.0 |
| CMV-214 | 25 mM Histidine, 75 mM Sodium Chloride, 90 mg/mL Sucrose; 5 mg/mL |
| | Propylene Glycol; 5 mg/mL Sodium Carboxymethylcellulose; pH 6.0 |
| CMV-202 | 25 mM Histidine, 75 mM Sodium Chloride, 90 mg/mL Sucrose; 5 mg/mL |
| | Propylene Glycol; 5 mg/mL Sodium Carboxymethylcellulose; pH 7.0 |
| CMV-253 | 25 mM Tris, 75 mM Sodium Chloride, 90 mg/mL Sucrose; 5 mg/mL |
| | Propylene Glycol; 5 mg/mL Sodium Carboxymethylcellulose; pH 8.0 |

The lyophilization process yield for CMV-202 (FIG. 5B) at pH 7.0 was similar to formulation at pH 6.0 (CMV-214) and pH 8.0 (CMV-253). It also showed significantly higher lyophilization yield for formulations containing propylene glycol and sodium carboxymethylcellulose as compared to CMV-240 formulation which lacks propylene glycol and sodium carboxymethylcellulose. The infectivity loss for CMV-202 at all pH levels tested (FIG. 6B) at 1 week 2-8° C. and 25° C. was similar and significantly lower as compared to CMV-240.

These data show that the formulations of the invention can be used at a pH range from 6.0 to 8.0.

### Example 7: Effect of Propylene Glycol Concentration

Effect of propylene glycol concentration was studied in CMV-188 formulation along with CMV-202. The formulation compositions are listed in Table 6. The liquid formulations were prepared with 200 Units/mL of rdCMV (SEQ ID NO: 14) and filled at 0.7 mL in 2 mL glass vials, stoppered, crimped and stored frozen at −70° C. for liquid control. For lyophilization, the formulations were filled at 0.7 mL in 2 mL glass vials, partially stoppered with lyophilization stoppers and lyophilized. After completing the lyophilization process, the control lyophilized product is stored at −70° C. and stability samples are subjected to accelerated (15° C.) and real-time (2-8° C.) stability conditions for 1 week and 1 month.

TABLE 6
Formulation codes and compositions

| Formulation | |
|---|---|
| Code | Formulation Composition |
| CMV-188 | 12.5 mM Histidine, 12.5 mM Tris, 90 mg/mL Sucrose; 5 mg/mL |
| | Propylene Glycol; 5 mg/mL Sodium Carboxymethylcellulose, pH 7.0 |
| CMV-203 | 12.5 mM Histidine, 12.5 mM Tris, 75 mM Sodium Chloride, 90 mg/mL |
| | Sucrose; 1 mg/mL Propylene Glycol; 5 mg/mL Sodium |
| | Carboxy methylcellulose, pH 7.0 |
| CMV-200 | 12.5 mM Histidine, 12.5 mM Tris, 75 mM Sodium Chloride, 90 mg/mL |
| | Sucrose; 2.5 mg/mL Propylene Glycol; 5 mg/mL Sodium |
| | Carboxymethylcellulose, pH 7.0 |
| CMV-201 | 12.5 mM Histidine, 12.5 mM Tris, 75 mM Sodium Chloride, 90 mg/mL |
| | Sucrose; 7.5 mg/mL Propylene Glycol; 5 mg/mL Sodium |
| | Carboxymethylcellulose, pH 7.0 |
| CMV-202 | 25 mM Histidine, 75 mM Sodium Chloride, 90 mg/mL Sucrose; 5 mg/mL |
| | Propylene Glycol; 5 mg/mL Sodium Carboxymethylcellulose, pH 7.0 |

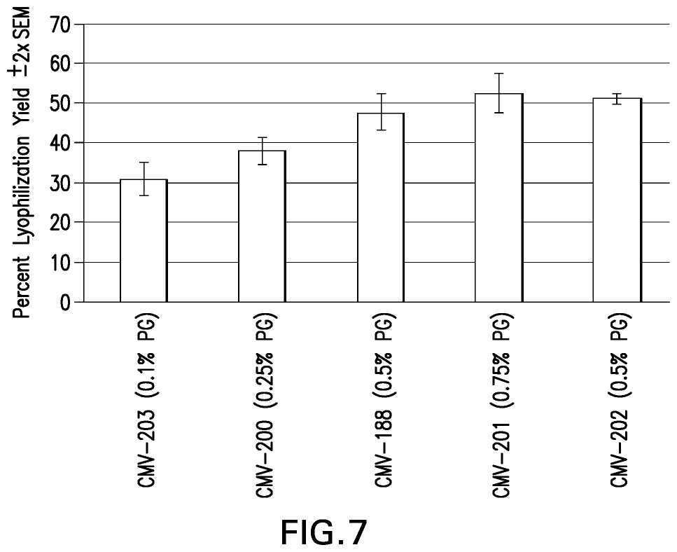

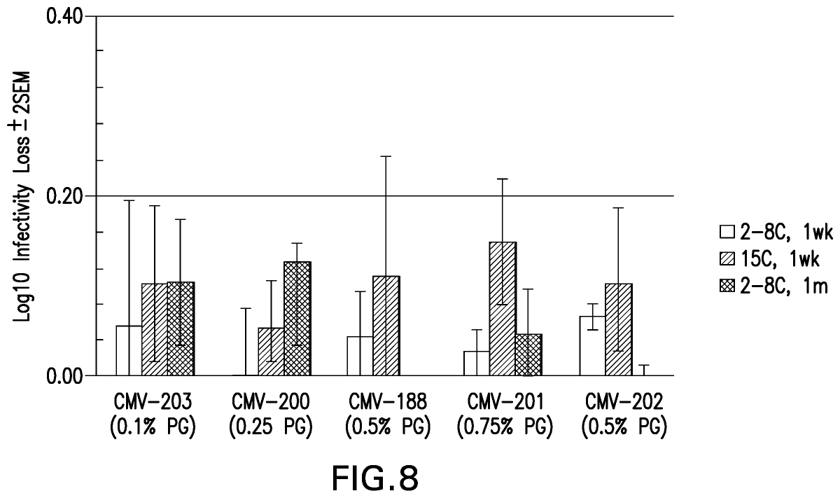

The lyophilization process yield with 0.25% w/v (2.5 mg/mL) to 0.75% w/v (7.5 mg/mL) propylene glycol (FIG. 7) was higher than CMV-098 (as shown in FIG. 1). The infectivity loss for all propylene glycol levels tested (FIG. 8) was lower compared to CMV-098 (as shown in FIGS. 2 and 4). The stability at 2-8° C. and 15° C. was comparable among all the concentrations of propylene glycol tested.

### Example 8: Impact of Sodium Carboxymethylcellulose Concentration

Effect of sodium carboxymethylcellulose concentration (2 mg/mL to 5 mg/mL) was studied in the CMV-202 formulation. The formulation compositions listed in Table 7 were tested. The liquid formulations were prepared with 200 Units/mL of rdCMV (SEQ ID NO: 14) and filled at 0.7 mL in 2 mL glass vials, stoppered, crimped and stored frozen at −70° C. for liquid control. For lyophilization, the formulations were filled at 0.7 mL in 2 mL glass vials, partially stoppered with lyophilization stoppers and lyophilized. After completing the lyophilization process, the control lyophilized product is stored at −70° C. and stability samples are subjected to accelerated (15° C.) and real-time (2-8° C.) stability conditions for 1 week.

TABLE 7
Formulation codes and compositions

| Formulation | |
|---|---|
| Code | Formulation Composition |
| CMV-202 | 25 mM Histidine, 90 mg/mL Sucrose; 5 mg/mL Propylene Glycol; 5 |
| | mg/mL Sodium Carboxymethylcellulose, pH 7.0 |
| CMV-225 | 25 mM Histidine, 90 mg/mL Sucrose; 5 mg/mL Propylene Glycol; 3 |
| | mg/mL Sodium Carboxymethylcellulose, pH 7.0 |
| CMV-226 | 25 mM Histidine, 90 mg/mL Sucrose; 5 mg/mL Propylene Glycol; 2 |
| | mg/mL Sodium Carboxymethylcellulose, pH 7.0 |

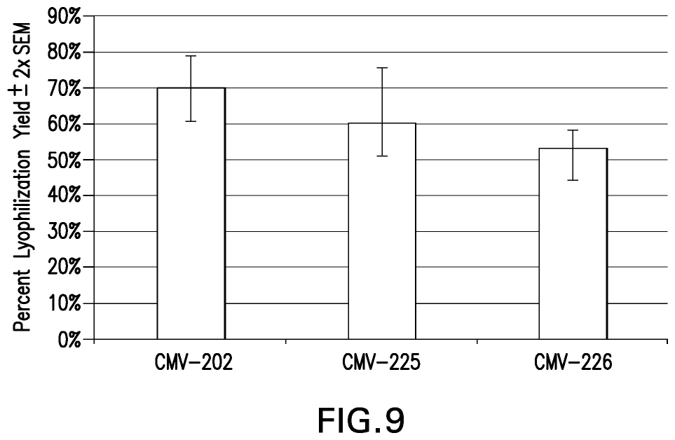

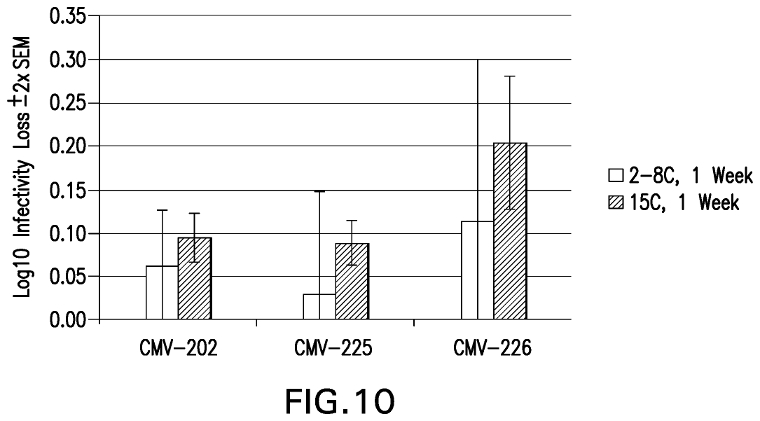

The lyophilization yield trended lower with lower concentration of Sodium CMC and 2 mg/mL showed a lower lyophilization yield as compared to 5 mg/mL (FIG. 9). The infectivity stability of CMV showed a trend of higher stability loss with lower Sodium CMC concentration (FIG. 10).

The lyophilization process yield with 0.2% w/v (2 mg/mL), 0.3% w/v (3 mg/mL) and 0.5% w/v (5 mg/mL) Sodium CMC (FIG. 9) was higher than CMV-098 (as shown in FIG. 1). The infectivity loss for all Sodium CMC levels tested (FIG. 10) was lower compared to CMV-098 (as shown in FIG. 2).

### Example 9: Effect of Fill Volume for Lyophilization of rdCMV in CMV-202 Formulation (0.7 mL Vs. 0.5 Ml)

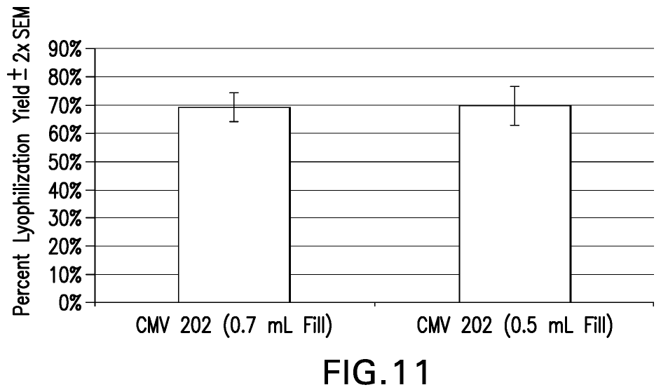

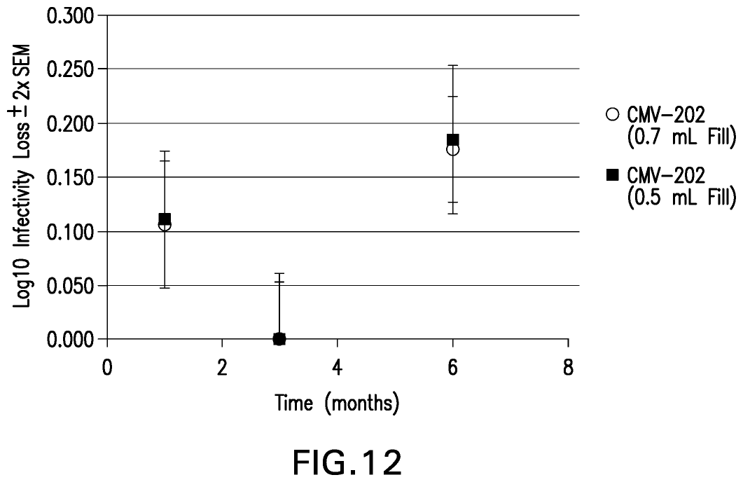

rdCMV (SEQ ID NO: 14) formulated at either 200 Units/mL and filled at 0.7 mL or 280 Units/mL and filled at 0.5 mL in 2 mL vials and lyophilized. The lyophilized vials were reconstituted with 0.7 mL water and tested for lyophilization yield and stability over time at 2-8° C. storage condition. The lyophilization process yield (FIG. 11) infectivity loss at 2-8° C. storage (FIG. 12) was found to be similar for both 0.7 mL and 0.5 mL fill.

### Example 10: Lyophilization of CMV in the Presence of Aluminum Phosphate Adjuvant (APA)

The ability of preventing agglomeration/aggregation of aluminum adjuvants, particularly APA, was tested using three formulations (Table 8) without CMV. The APA was formulated at 450 μg/mL in the three formulations and the samples were filled at 0.7 mL into 2 mL glass vials. The samples for liquid control were stored at 2-8° C. and the samples for lyophilization were frozen using a nitrogen cooled fast freezer and the samples loaded into a lyophilizer.

TABLE 8
Formulation codes and compositions

| Formulation | |
|---|---|
| Code | Formulation Composition |
| CMV-098 | 12.5 mM Histidine, 12.5 mM Tris, 90 mg/mL Sucrose; pH 7.0 |
| CMV-165 | 12.5 mM Histidine, 12.5 mM Tris, 90 mg/mL Sucrose, 5 mg/mL Sodium |
| | Carboxy methylcellulose, pH 7.0 |
| CMV-188 | 12.5 mM Histidine, 12.5 mM Tris, 90 mg/mL Sucrose; 5 mg/mL Propylene |
| | Glycol; 2 mg/mL Sodium Carboxymethylcellulose, pH 7.0 |

### Particle Size Analysis

Aluminum adjuvants are prone to agglomeration during freezing and freeze drying process. The frozen and lyophilized rdCMV formulations in the presence of APA were evaluated for the physical stability by measuring the particle size distribution of thawed or reconstituted samples using static light scattering (SLS). Evaluation of the particle size was done using a Malvern© Mastersizer 2000 system.

The particle size data is presented as D [3,2], d(0.5) and D[4,3]. The D[3,2] is surface area weighted mean and this value is sensitive to smaller diameter particles in the particle size distribution. The d(0.5) is the median volume diameter and shows the particle diameter that divides the particles into two equal halves i.e., there is 50% of the particles are above this value and 50% below this value. The D[4,3] represents volume weighted mean (De Brouckere mean diameter) and shows the diameter of the particles that make up a bulk of the sample volume and it is sensitive to larger diameter particle in the particle size distribution.

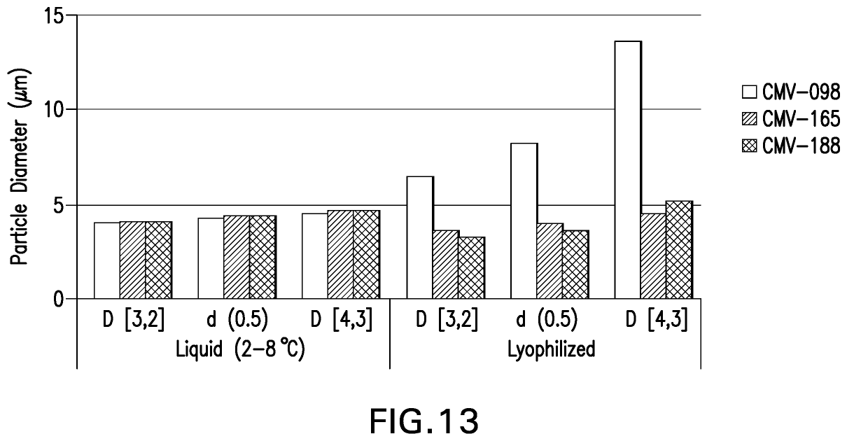

The particle size distribution data showed that rdCMV (SEQ ID NO: 14) stabilizing formulations containing sodium carboxymethylcellulose alone (CMV-165) or in combination with propylene glycol (CMV-188) prevents freezing induced or freeze drying induced APA particle agglomeration (FIG. 13) as compared to a sucrose formulation (CMV-098).

Subsequent study was performed with CMV at 200 Units/mL formulated along with APA at 450 μg/mL in CMV-202 formulation. The liquid formulation was prepared in CMV-202 composition (25 mM Histidine, 75 mM Sodium Chloride, 90 mg/mL Sucrose; 5 mg/mL Propylene Glycol; 5 mg/mL Sodium CMC, pH 7.0) and filled at 0.7 mL in 2 mL glass vials, stoppered, crimped and stored at 2-8° C. for liquid control. The vials for frozen liquid samples were frozen using a nitrogen cooled fast freezer and the samples were stored at −70° C. For lyophilization, the formulations were filled at 0.7 mL in 2 mL glass vials, partially stoppered with lyophilization stoppers. The vials were frozen using nitrogen cooled fast freezer and the frozen vials were loaded into a lyophilization chamber. After completing the lyophilization process, the lyophilized product was stored at 2-8° C. The liquid control, frozen liquid and lyophilized samples were tested for particle size analysis as a measure of physical stabilization during freezing and lyophilization process.

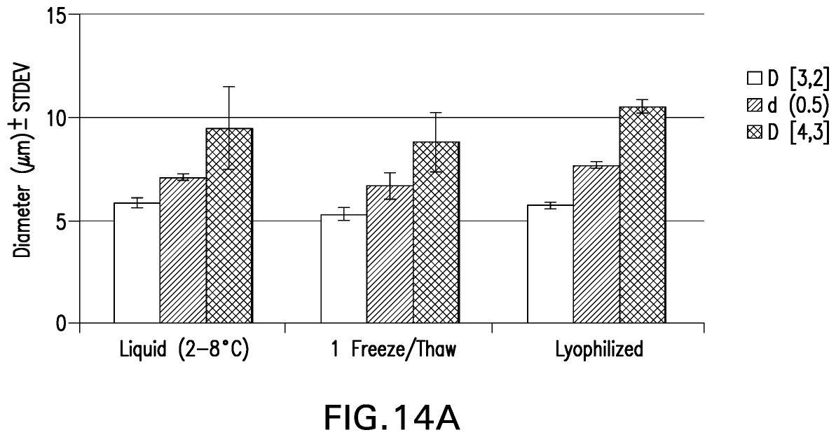

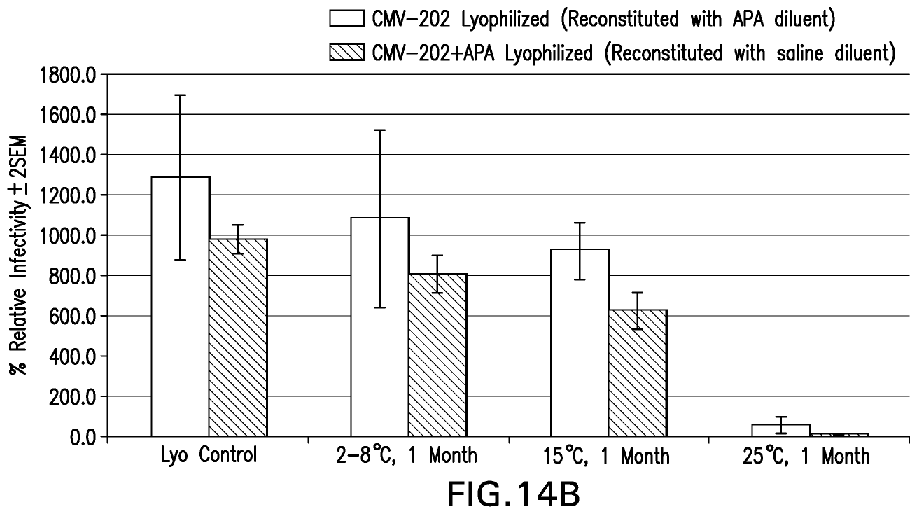

The particle size data for CMV formulated with APA in CMV-202 composition showed no significant differences between liquid control sample (stored at 2-8° C.) and frozen samples and lyophilized samples (FIG. 14A). This data demonstrated the feasibility of freezing and lyophilization CMV in the presence of APA in a composition that is stabilizing to both CMV and APA.

### Stability Analysis

An additional study was performed evaluating lyophilization feasibility and stability of CMV in CMV-202 formulation in the presence of Aluminum Phosphate Adjuvant. In one case, the CMV was formulated in CMV-202 formulation followed by lyophilization and reconstitution of the lyophilized product with APA diluent prior to testing. In another case, the CMV was formulated in CMV-202 formulation along with APA followed by lyophilization and reconstitution of the product with saline diluent prior to testing.

After completing the lyophilization process, the Lyo control samples were stored at −70° C. and stability samples were subjected to real-time (2-8° C.) stability condition or accelerated stability conditions (15° C. and 25° C.) for 1 Month. The CMV samples lyophilized without APA were reconstituted with 0.7 mL of APA diluent (450 μg/mL) solution for testing. The CMV samples lyophilized with APA were reconstituted with 0.7 mL of saline (0.9% w/v sodium chloride) solution for testing. Both formulations were expected to have similar CMV and APA content after reconstitution.

The lyophilization yield and the stability of CMV as shown in FIG. 14B shows the feasibility of adding APA int the formulation prior to lyophilization.

### Example 11: CMV Formulation Compositions

The rdCMV (SEQ ID NO: 14) was formulated in 25 mM L-histidine, 75 mM sodium chloride, 90 mg/mL sucrose, 5 mg/mL propylene glycol and 5 mg/mL sodium CMC at pH 7.0 prior to lyophilization. The formulation was filled at 0.5 mL to 0.7 mL into sterile 2 mL vials and lyophilized.

Table 9 below shows the formulation composition of the rdCMV (SEQ ID NO: 14) formulation prior to lyophilization.

TABLE 9
Target composition of rdCMV formulation prior to lyophilization

| Ingredient | Target Concentration | |
|---|---|---|
| rdCMV | 50-600 μg/mL | |
| L-Histidine USP/BP/EP/JP | 25.0 mM | (3.88 mg/mL) |
| Sodium chloride USP/EP/JP/BP | 75.0 mM | (4.38 mg/mL) |
| Sucrose NF/EP/BP | 9.00% w/v | (90.0 mg/mL) |
| Propylene Glycol USP/EP/JP | 0.500% w/v | (5.00 mg/mL) |
| Sodium Carboxymethylcellulose NF/EP | 0.500% w/v | (5.00 mg/mL) |

Target pH = 7.0 ± 0.5

Table 10 below shows the formulation composition of the rdCMV (SEQ ID NO: 14) lyophilized in final containers.

TABLE 10
Target composition of rdCMV lyophilized final containers

| Ingredient | Quantity (per container) | |
|---|---|---|
| rdCMV | 25-300 | μg |
| L-Histidine USP/BP/EP/JP | 1.9-2.7 | mg |
| Sodium chloride USP/EP/JP/BP | 2.2-3.07 | mg |
| Sucrose NF/EP/BP | 45-63 | mg |
| Propylene Glycol USP/EP/JP | 2.5-3.5 | mg |
| Sodium Carboxymethylcellulose NF/EP | 2.5-3.5 | mg |

The rdCMV (SEQ ID NO: 14) lyophilized cake was reconstituted with 0.7 mL of either water or 9 mg/mL sodium chloride solution or APA diluent (formulated at 450 μg/mL APA in 0.9% w/v sodium chloride) prior to vaccine administration. Table 11 below shows the composition of the APA diluent (0.9% w/v sodium chloride in sterile water for injection).

TABLE 11
Formulation composition for APA diluent

| Formulation Component | Target Concentration |
|---|---|
| APA | 450 μg/mL |
| Sodium chloride | 0.900% w/v (9.00 mg/mL) |
| USP/EP/JP/BP | |
| Water for Injection | Qs |

qs: quantity sufficient

Prior to administration, rdCMV (SEQ ID NO: 14) active lyophilized cake was reconstituted with 0.70 mL of APA diluent to obtain a total of approximately 0.70 mL of the reconstituted virus. The virus dose was administered in 0.50 mL volume that contains the target clinical dose of 100 μg of rdCMV (SEQ ID NO: 14) and 225 μg of APA. Table 12 summarizes the target formulation composition of rdCMV (SEQ ID NO: 14) after reconstitution with APA diluent.

TABLE 12
Formulation composition of rdCMV after reconstitution of rdCMV
lyophilized cake with sterile saline diluent or APA diluent

| | | | Quantity | |
|---|---|---|---|---|
| Ingredient | Target Concentration | | (per 0.5 mL dose) | |
| rdCMV (SEQ ID NO: 14) | 50-600 | μg/mL | 25-300 | μg |
| L-Histidine USP/BP/EP/JP | 2.77-3.88 | mg/mL | 1.39-1.9 | mg |
| Sodium chloride USP/EP/JP/BP | 12.1-13.45 | mg/mL | 6-6.7 | mg |
| Sucrose NF/EP/BP | 64.3-90 | mg/mL | 32.2-45 | mg |
| Propylene Glycol USP/EP/JP | 3.57-5 | mg/mL | 1.79-2.5 | mg |
| Sodium Carboxymethylcellulose | 3.57-5 | mg/mL | 1.79-2.5 | mg |

| | | |
|---|---|---|
| NF/EP | | |
| APA* | 450 μg/mL (aluminum) | 225 μg (aluminum) |

*Only for APA reconstituted CMV.

### Example 12: Effect of Buffer Species at pH 7.0 on Lyophilization Process Yield and Stability of CMV in CMV-202 Formulation

The impact of buffer species was studied in CMV-202 composition at pH 7.0 by using different buffering agents. The formulations are listed in Table 13. The liquid formulations were prepared with 280 Units/mL of rdCMV (SEQ ID NO: 14) and filled at 0.5 mL in 2 mL glass vials, stoppered, crimped and stored frozen at −70° C. for liquid control. For lyophilization, the formulations were filled at 0.5 mL in 2 mL glass vials, partially stoppered with lyophilization stoppers and lyophilized. After completing the lyophilization process, the control lyophilized product is stored at −70° C. and stability samples are subjected to real-time (2-8° C.) stability condition or accelerated stability condition (25° C.) for 1 week. The samples were reconstituted with 0.7 mL of saline (0.9% w/v sodium chloride) solution for testing.

TABLE 13
Formulation codes and compositions

| Formulation | |
|---|---|
| Code | Formulation Composition |
| CMV-240 | 25 mM Histidine, 75 mM Sodium Chloride, 90 mg/mL Sucrose; pH 7.0 |
| CMV-202 | 25 mM Histidine, 75 mM Sodium Chloride, 90 mg/mL Sucrose; 5 mg/mL |
| | Propylene Glycol; 5 mg/mL Sodium Carboxymethylcellulose; pH 7.0 |
| CMV-251 | 25 mM Phosphate, 75 mM Sodium Chloride, 90 mg/mL Sucrose; 5 mg/mL |
| | Propylene Glycol; 5 mg/mL Sodium Carboxymethylcellulose; pH 7.0 |
| CMV-252 | 25 mM HEPES, 75 mM Sodium Chloride, 90 mg/mL Sucrose; 5 mg/mL |
| | Propylene Glycol; 5 mg/mL Sodium Carboxymethylcellulose; pH 7.0 |

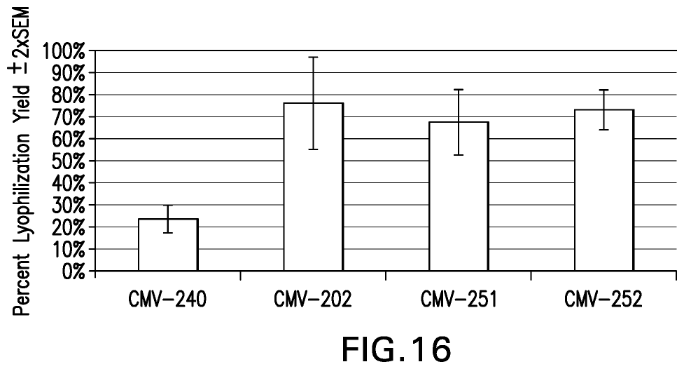

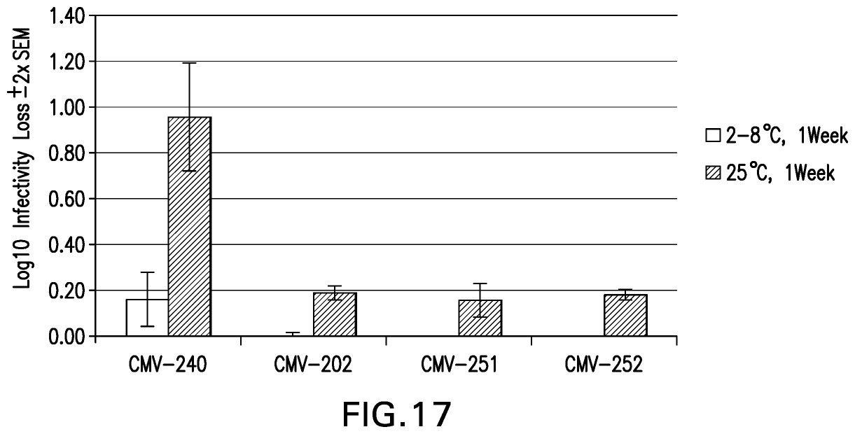

The lyophilization process yield was similar with different buffer species at pH 7.0 (FIG. 16). The data also showed significantly higher lyophilization yield for formulations containing propylene glycol and sodium carboxymethylcellulose as compared to CMV-240 formulation which lacks propylene glycol and sodium carboxymethylcellulose. The infectivity loss for CMV-202 with different buffer species (FIG. 17) at 1 week 2-8° C. and 25° C. was similar and significantly lower as compared to CMV-240.

This data shows that a variety of buffers can be used for the formulations of the invention.

### Example 13: Effect of Sugar (Sucrose and Trehalose) and Sugar Concentration on Lyophilization Process Yield and Stability of CMV in CMV-202 Formulation

The impact of sugar type and sugar concentration was studied in CMV-202 composition with sucrose and trehalose at 40, 90 and 150 mg/mL. The formulations are listed in Table 14. The liquid formulations were prepared with 280 Units/mL of rdCMV (SEQ ID NO: 14) and filled at 0.5 mL in 2 mL glass vials, stoppered, crimped and stored frozen at −70° C. for liquid control. For lyophilization, the formulations were filled at 0.5 mL in 2 mL glass vials, partially stoppered with lyophilization stoppers and lyophilized. After completing the lyophilization process, the control lyophilized product was stored at −70° C. and stability samples were subjected to real-time (2-8° C.) stability condition or accelerated stability condition (25° C.) for 1 week. The samples were reconstituted with 0.7 mL of saline (0.9% w/v sodium chloride) solution for testing.

TABLE 14
Formulation codes and compositions

| Formulation | |
|---|---|
| Code | Formulation Composition |
| CMV-240 | 25 mM Histidine, 75 mM Sodium Chloride, 90 mg/mL Sucrose; pH 7.0 |
| CMV-255 | 25 mM Histidine, 75 mM Sodium Chloride, 40 mg/mL Sucrose; 5 mg/mL |
| | Propylene Glycol; 5 mg/mL Sodium Carboxymethylcellulose; pH 7.0 |
| CMV-202 | 25 mM Histidine, 75 mM Sodium Chloride, 90 mg/mL Sucrose; 5 mg/mL |
| | Propylene Glycol; 5 mg/mL Sodium Carboxymethylcellulose; pH 7.0 |
| CMV-256 | 25 mM Histidine, 75 mM Sodium Chloride, 150 mg/mL Sucrose; 5 mg/ml |
| | Propylene Glycol; 5 mg/mL Sodium Carboxymethylcellulose; pH 7.0 |
| CMV-257 | 25 mM Histidine, 75 mM Sodium Chloride, 40 mg/mL Trehalose; 5 mg/mL |
| | Propylene Glycol; 5 mg/mL Sodium Carboxymethylcellulose; pH 7.0 |
| CMV-258 | 25 mM Histidine, 75 mM Sodium Chloride, 90 mg/mL Trehalose; 5 mg/mL |
| | Propylene Glycol; 5 mg/mL Sodium Carboxymethylcellulose; pH 7.0 |
| CMV-259 | 25 mM Histidine, 75 mM Sodium Chloride, 150 mg/mL Trehalose; 5 mg/mL |
| | Propylene Glycol; 5 mg/mL Sodium Carboxymethylcellulose; pH 7.0 |

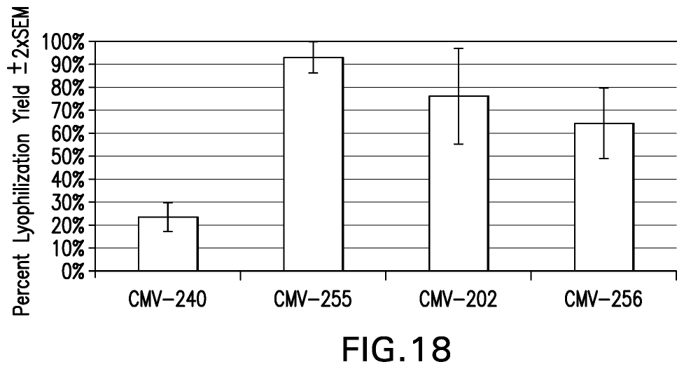

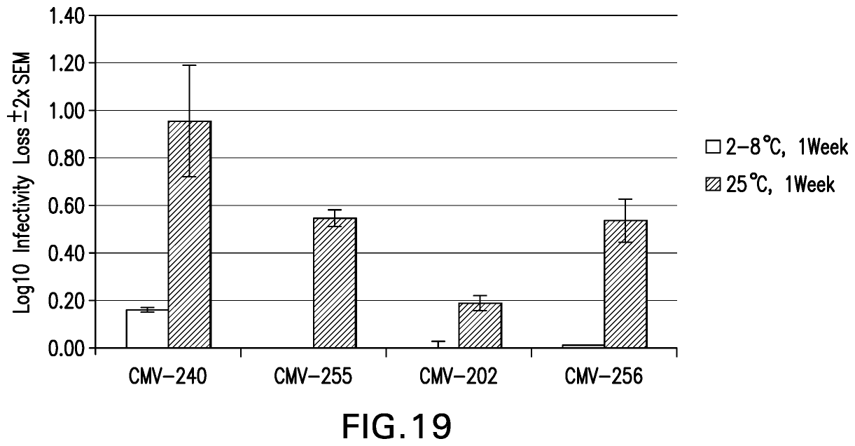

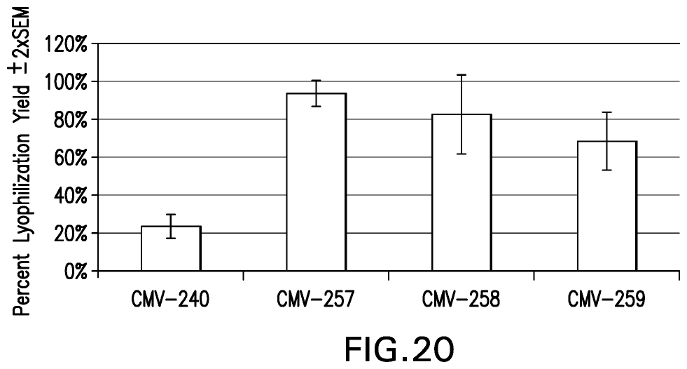

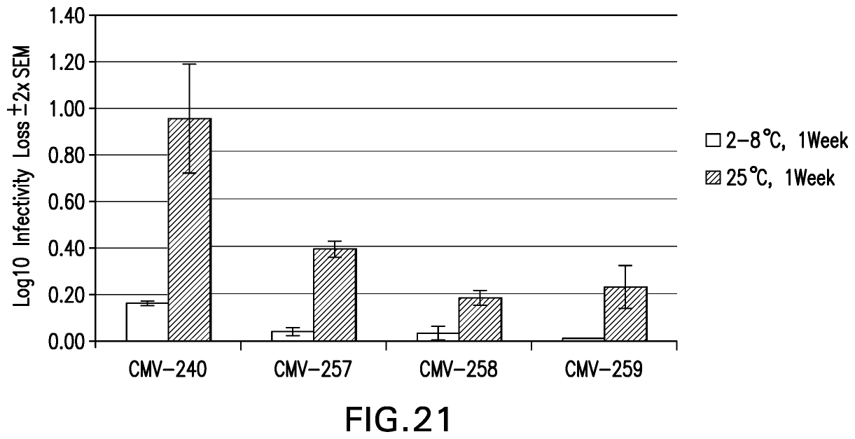

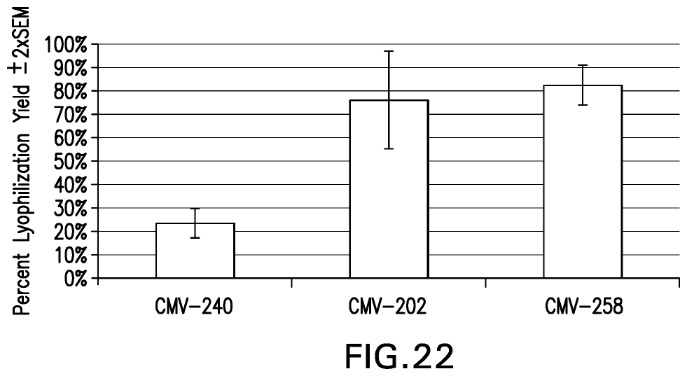

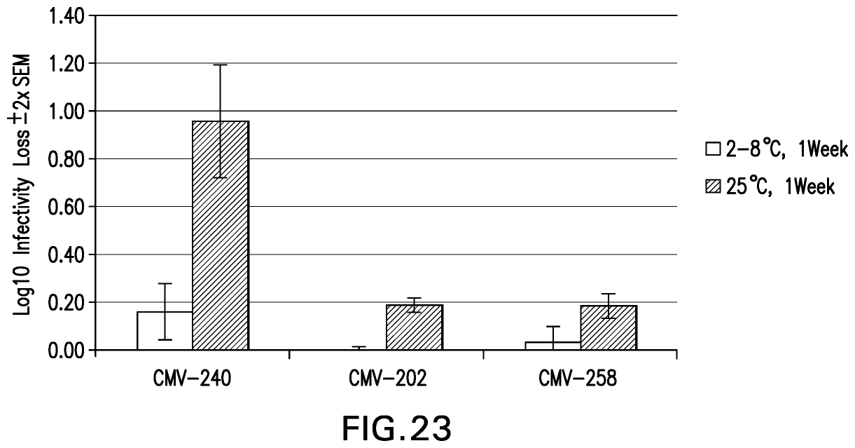

The lyophilization process yield (FIG. 18) with all the sucrose concentrations tested in the CMV-202 formulation showed significantly higher yields as compared to CMV-240 formulation which lacks propylene glycol and sodium carboxymethylcellulose. The stability data (FIG. 19) showed significantly better stability for all the sucrose concentrations tested in the CMV-202 formulation as compared to the CMV-240 formulation which lacks propylene glycol and sodium carboxymethylcellulose. Similar results were obtained when sucrose was replaced with another sugar trehalose (FIGS. 20 and 21). The lyophilization yield (FIG. 22) and stability (FIG. 23) was found to be similar with sucrose and trehalose at 90 mg/mL in CMV-202 formulation.

### Example 14: Effect of Alkali Salt on Lyophilization Process Yield and Stability of CMV in CMV-202 Formulation

The impact of alkali salt in CMV-202 composition was studied by replacing sodium chloride with potassium chloride. The formulations are listed in Table 15. The liquid formulations were prepared with 280 Units/mL of rdCMV (SEQ ID NO: 14) and filled at 0.5 mL in 2 mL glass vials, stoppered, crimped and stored frozen at −70° C. for liquid control. For lyophilization, the formulations were filled at 0.5 mL in 2 mL glass vials, partially stoppered with lyophilization stoppers and lyophilized. After completing the lyophilization process, the control lyophilized product is stored at −70° C. and stability samples are subjected to real-time (2-8° C.) stability condition or accelerated stability condition (25° C.) for 1 week. The samples were reconstituted with 0.7 mL of saline (0.9% w/v sodium chloride) solution for testing.

TABLE 15
Formulation codes and compositions

| Formulation | |
|---|---|
| Code | Formulation Composition |
| CMV-240 | 25 mM Histidine, 75 mM Sodium Chloride, 90 mg/mL Sucrose; pH 7.0 |
| CMV-202 | 25 mM Histidine, 75 mM Sodium Chloride, 90 mg/mL Sucrose; 5 mg/mL |
| | Propylene Glycol; 5 mg/mL Sodium Carboxymethylcellulose; pH 7.0 |
| CMV-254 | 25 mM Histidine, 75 mM Potassium Chloride, 90 mg/mL Sucrose; 5 mg/mL |
| | Propylene Glycol; 5 mg/mL Sodium Carboxymethylcellulose; pH 7.0 |

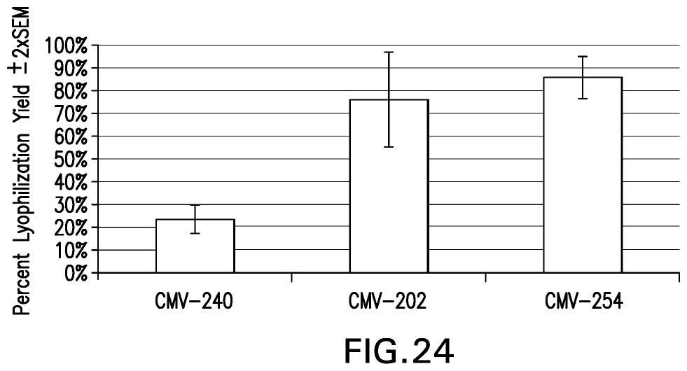

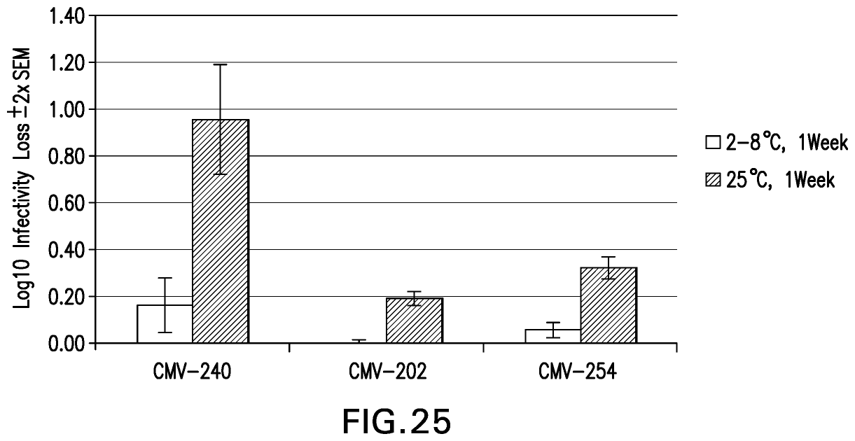

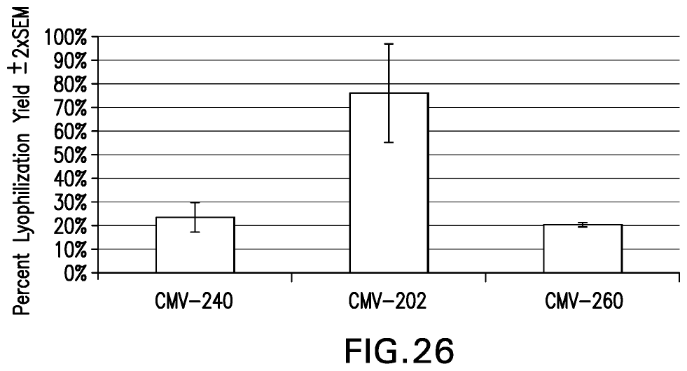

The lyophilization yield (FIG. 24) and stability (FIG. 25) was found to be similar when sodium chloride in CMV-202 formulation was replaced with potassium chloride.

This data supports the notion that alkaline salts can be used in the formulations of the invention.

### Example 15: Effect of Cellulose Type on Lyophilization Process Yield and Stability of CMV in CMV-202 Formulation

The impact of type of cellulose in CMV-202 composition was studied by replacing sodium carboxymethylcellulose (CMC) with hydroxypropyl methylcellulose (HPMC). The formulations are listed in Table 16. The liquid formulations were prepared with 280 Units/mL of rdCMV (SEQ ID NO: 14) and filled at 0.5 mL in 2 mL glass vials, stoppered, crimped and stored frozen at −70° C. for liquid control. For lyophilization, the formulations were filled at 0.5 mL in 2 mL glass vials, partially stoppered with lyophilization stoppers and lyophilized. After completing the lyophilization process, the control lyophilized product was stored at −70° C. and stability samples were subjected to real-time (2-8° C.) stability condition or accelerated stability condition (25° C.) for 1 week. The samples were reconstituted with 0.7 mL of saline (0.9% w/v sodium chloride) solution for testing.

TABLE 16
Formulation codes and compositions

| Formulation | |
|---|---|
| Code | Formulation Composition |
| CMV-240 | 25 mM Histidine, 75 mM Sodium Chloride, 90 mg/mL Sucrose; pH 7.0 |
| CMV-202 | 25 mM Histidine, 75 mM Sodium Chloride, 90 mg/mL Sucrose; 5 mg/mL |
| | Propylene Glycol; 5 mg/mL Sodium Carboxymethylcellulose; pH 7.0 |
| CMV-260 | 25 mM Phosphate, 75 mM Potassium Chloride, 90 mg/mL Sucrose; 5 mg/mL |
| | Propylene Glycol; 5 mg/mL Hydroxypropyl methylcellulose (HPMC); pH 7.0 |

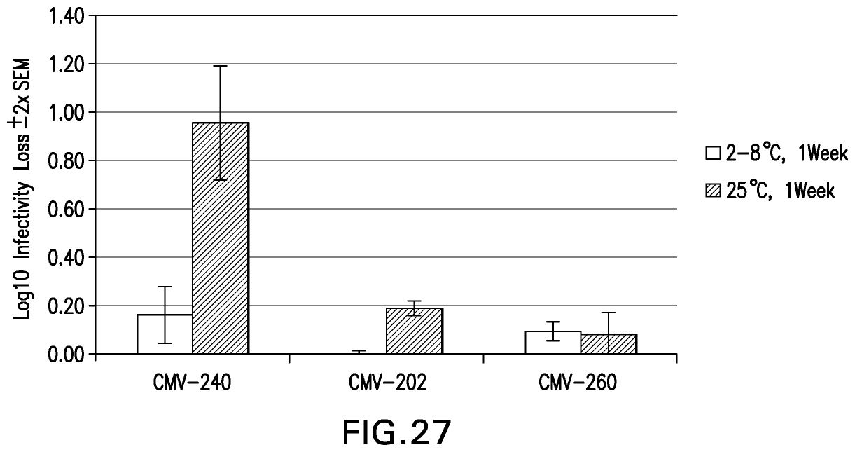

The lyophilization yield (FIG. 26) for HPMC formulation (CMV-260) was significantly lower compared to sodium carboxymethylcellulose formulation (CMV-202). However, the stability of CMV was found to be similar for both HPMC and CMC formulations (FIG. 27). The stability of CMV in formulations containing cellulose (HPMC or CMC) was significantly better than a formulation that lacks a cellulose (CMV-240). This data supports the selection of sodium carboxymethylcellulose for a clinical formulation of a CMV vaccine. Improvements were seen with both lyophilization yield and virus stability. The use of hydroxymethylcellulose, while not as preferred as sodium CMC, would allow for the design of a stable CMV formulation. However, the lyophilization yield would need to be taken into account.

Other embodiments are within the following claims. While several embodiments have been shown and described, various modifications may be made without departing from the spirit and scope of the present invention.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

What is claimed is:

1. A formulation comprising:
cytomegalovirus (CMV), wherein the CMV is selected from:
(a) a live attenuated CMV that is conditional replication defective and comprises:
(i) a pentameric gH complex comprising UL128, UL130, UL131, gH and gL; and
(ii) a nucleic acid encoding a fusion protein of an essential protein and a destabilizing protein, wherein the essential protein is selected from the group consisting of IE1/2, UL51, UL52, UL79 and UL84,
(b) a killed CMV, and
(c) an inactivated CMV
a buffer at pH about 6.0 to 8.0, wherein buffer is selected from: phosphate, succinate, histidine, TRIS, MES, MOPS, HEPES, acetate, and citrate;
an alkali or alkaline salt, wherein the salt is selected from: magnesium chloride, calcium chloride, potassium chloride, sodium chloride, or a combination thereof;
a sugar;
a cellulose derivative selected from the group consisting of carboxymethyl cellulose, hydroxypropyl cellulose (HPC), hydroxypropyl methylcellulose (HPMC), 2-hydroxyethyl cellulose (2-HEC), crosscarmellose and methyl cellulose or a pharmaceutically acceptable salt thereof; and
optionally, a polyol selected from the group consisting of propylene glycol, polypropylene glycol, ethylene glycol, polyethylene glycol, polyethylene glycol monomethyl ethers, and sugar alcohol.

2. The formulation of claim 1 that comprises about 50-600 μg/ml CMV, a buffer at pH about 6.0 to 7.5, about 50-300 mM NaCl, about 40-150 mg/ml sucrose, and about 0.3-10 mg/ml of a pharmaceutically acceptable salt of carboxymethylcellulose.

3. The formulation of claim 1 that comprises about 50-600 μg/ml CMV, about 5-500 mM buffer at pH about 6.0 to 7.5, about 50-300 mM NaCl, about 40-150 mg/ml sucrose, and about 0.3-10 mg/ml sodium carboxymethylcellulose with average molecular weight at about 50,000 to 1,000,000.

4. The formulation of claim 1 that comprises about 50-600 μg/ml CMV, about 10-100 mM histidine, or TRIS or HEPES buffer at pH about 6.0 to 7.5, about 50-300 mM NaCl, about 40-150 mg/ml sucrose, about 2.5-7.5 mg/ml propylene glycol (PG), and about 3-10 mg/ml sodium carboxymethylcellulose with average molecular weight at about 90,000.

5. The formulation of claim 1 that comprises about 50-600 μg/ml CMV, about 10-100 mM histidine, TRIS or HEPES buffer, at pH about 6.0 to 7.5, about 50-150 mM NaCl, about 60-110 mg/ml sucrose, about 3-7 mg/ml propylene glycol (PG), and about 3-7 mg/ml sodium carboxymethylcellulose with average molecular weight at about 90,000.

6. The formulation of claim 1 that comprises about 100-350 μg/ml CMV, about 25 mM buffer of histidine, TRIS, or a combination thereof, at pH about 7.0, about 75 mM NaCl, about 90 mg/ml sucrose, about 5 mg/ml propylene glycol (PG), and about 5 mg/ml sodium carboxymethylcellulose with average molecular weight at about 90,000.

7. The formulation of claim 1 that further comprises an aluminum adjuvant.

8. The formulation of claim 1 that further comprises an Aluminum Phosphate Adjuvant at about 200-700 μg/ml.

9. The formulation of claim 1 that is the aqueous solution prior to lyophilization.

10. The formulation of claim 1 that is a reconstituted solution, wherein the reconstitution is performed with water or saline solution.

11. The formulation of claim 1 that is a reconstituted solution, wherein the reconstitution is performed with a diluent comprising 0.5-1 ml of an aluminum adjuvant formulated in buffer, saline solution, or water.

12. The formulation of claim 11, wherein the reconstitution is performed with 0.7 ml diluent comprising an Aluminum Phosphate Adjuvant (APA) and saline solution, wherein the APA is about 400-500 μg/ml in the reconstituted solution.

13. The formulation of claim 12 that is a 0.5 ml dose of CMV comprising about 25-300 μg of CMV, about 1.39-1.9 mg histidine, about 6-6.7 mg NaCl, about 32.2-45 mg sucrose, about 1.79-2.5 mg propylene glycol (PG), and about 1.79-2.5 mg sodium carboxymethylcellulose at average molecular weight at about 90,000.

14. The formulation of claim 1 that is in a dried solid form and comprises about 25-300 μg CMV, about 1.9-2.7 mg histidine, TRIS, or a combination thereof, about 2.2-3.07 mg NaCl, about 45-63 mg sucrose, about 2.5-3.5 mg propylene glycol (PG), and about 2.5-3.5 mg sodium carboxymethylcellulose with average molecular weight of 90,000.

15. The formulation of claim 1 that is in a dried solid form comprising a weight ratio of about CMV 1, histidine 6-108, NaCl 7-123, sucrose 150-2520, propylene glycol 8-140, and sodium carboxymethylcellulose 8-140.

16. A formulation comprising:
cytomegalovirus (CMV), wherein the CMV is selected from:
(a) a live attenuated CMV that is conditional replication defective and comprises:
(i) a pentameric gH complex comprising UL128, UL130, UL131, gH and gL; and
(ii) a nucleic acid encoding a fusion protein of an essential protein and a destabilizing protein, wherein the essential protein is selected from the group consisting of IE1/2, UL51, UL52, UL79 and UL84,
(b) a killed CMV, and
(c) an inactivated CMV
a buffer at pH about 6.0 to 8.0, wherein buffer is selected from: phosphate, succinate, histidine, TRIS, MES, MOPS, HEPES, acetate, and citrate;
an alkali or alkaline salt, wherein the salt is selected from: magnesium chloride, calcium chloride, potassium chloride, sodium chloride, or a combination thereof;
a sugar; and
a cellulose derivative selected from the group consisting of carboxymethyl cellulose, hydroxypropyl cellulose (HPC), hydroxypropyl methylcellulose (HPMC), 2-hydroxyethyl cellulose (2-HEC), crosscarmellose and methyl cellulose or a pharmaceutically acceptable salt thereof;
wherein the formulation is a dried solid form, wherein the CMV titer after 2 years at 2-8° C. is about 7.77×10E⁴ to 3.8×10E⁸ pfu/ml.

17. A formulation comprising:
a live attenuated conditional replication defective cytomegalovirus (rdCMV) and comprises: (a) a pentameric gH complex comprising UL128, UL130, UL131, gH and gL; and (b) a nucleic acid encoding a first fusion protein of 1E1/2 and a destabilizing protein and a second fusion protein of UL51 and the destabilizing protein, wherein the destabilizing protein is FK506-binding protein (FKBP) derivative comprising amino acid substitutions F36V and L10⁶P; wherein the wild type 1E1/2 and UL51 are no longer present and wherein the CMV is an attenuated strain that has restored gH complex expression due to a repair of a mutation in the UL131 gene,
(b) a buffer at pH about 6.0 to 8.0, wherein buffer is selected from: phosphate, succinate, histidine, TRIS, MES, MOPS, HEPES, acetate, and citrate;
(c) an alkali or alkaline salt, wherein the salt is selected from: magnesium chloride, calcium chloride, potassium chloride, sodium chloride, or a combination thereof;
(d) a sugar; and
(e) a cellulose derivative selected from the group consisting of carboxymethyl cellulose, hydroxypropyl cellulose (HPC), hydroxypropyl methylcellulose (HPMC), 2-hydroxyethyl cellulose (2-HEC), crosscarmellose and methyl cellulose or a pharmaceutically acceptable salt thereof;
wherein the formulation is a dried solid form, wherein the CMV titer after 2 years at 2-8° C. is about 7.77×10E⁴ to 3.8×10E⁸ pfu/ml.that is a dried solid form,
wherein the rdCMV in the presence of at least 2 μM Shield-1 replicates preferably at least 90% of the amount that a non-rdCMV replicates; and
wherein the rdCMV of the invention has a viral titer of at least 10⁵ pfu/ml in the presence of at least 2 μM Shield-1.

18. The formulation of claim 17, wherein (a) the first fusion protein is SEQ ID NO:1 or an amino acid sequence that is at least 95% identical to SEQ ID NO:1; and (b) the second fusion protein is SEQ ID NO:3 or an amino acid sequence that is at least 95% identical to SEQ ID NO:3.

19. The formulation of claim 17, wherein (a) the first fusion protein is encoded by SEQ ID NO:2 or a nucleic acid sequence that is at least 95% identical to SEQ ID NO:2; and (b) the second fusion protein is encoded by SEQ ID NO:4 or a nucleic acid sequence that is at least 95% identical to SEQ ID NO:4.

20. The formulation of claim 17, wherein the CMV is AD169 that has restored gH complex expression due to a repair of a mutation in the UL131 gene.


SEQUENCE LISTING (16 sequences)

SEQ ID NO: 1 - protein, 598 aa, synthetic construct
  REGION 1..598: note = Chemically Synthesized
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEESSAKRKMDPDNPDEGPSSKVPRPETPVTKATTFLQTMLRKEVNSQLSLGDPLFPELAEESLKTFEQVTEDCNENPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGVSSSDSLVSPPESPVPATIPLSSVIVAENSDQEESEQSDEEEEEGAQEEREDTVSVKSEPVSEIEEVAPEEEEDGAEEPTASGGKSTHPMVTRSKADQ

SEQ ID NO: 2 - DNA, 2081 bp, synthetic construct
  misc_feature 1..2081: note = Chemically Synthesized
  source 1..2081: mol_type = other DNA
atgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaagagtcctctgccaagagaaagatggaccctgataatcctgacgagggcccttcctccaaggtgccacggtacgtgtcggggtttgtgccccccctttttttttaataaaattgtattaatgttatatacatatctcctgtatgtgacccatgtgcttatgactctatttctcatgtgtttaggcccgagacacccgtgaccaaggccacgacgttcctgcagactatgttgaggaaggaggttaacagtcagctgagtctgggagacccgctgtttccagagttggccgaagaatccctcaaaacttttgaacaagtgaccgaggattgcaacgagaaccccgagaaagatgtcctggcagaactcggtaagtctgttgacatgtatgtgatgtatactaacctgcatgggacgtggatttacttgtgtatgtcagatagagtaaagattaactcttgcatgtgagcggggcatcgagatagcgataaatgagtcaggaggacggatacttatatgtgttgttatcctcctctacagtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagtaa

SEQ ID NO: 3 - protein, 264 aa, synthetic construct
  REGION 1..264: note = Chemically Synthesized
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPESWAKQRVPFLDDDDGEEENDVQDDVDSPVPTRPLVIDEDAEPAAGTSGGLEGGGGDDEDGEDGHALPDLDDDLLLQFEPMLPRVYDLLLPSLDARLNFVNAGQKYAAFLKYVHGDCATCSHGEILREKTQLLTAIVSKLMDINGILEGKDESAPGK

SEQ ID NO: 4 - DNA, 795 bp, synthetic construct
  misc_feature 1..795: note = Chemically Synthesized
  source 1..795: mol_type = other DNA
atgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaatcctgggctaagcagcgggtgccgtttctggacgatgacgacggagaggaggaaaacgacgtgcaggatgacgtggattctccggtgccgacgcgaccgctggtgatcgacgaggacgcggagcccgcggctggtacgagcggtgggctcgaggggggaggtggtgacgacgaggacggtgaagacggacacgcgctacccgatcttgacgacgatctgctattacagttcgagccgatgctgccgcgcgtctacgatctgttgctgccctctctggacgcgcgcttaaatttcgtgaacgcgggtcagaagtacgccgccttcctcaagtacgtgcacggcgactgcgcgacctgcagtcacggagagatcctccgtgagaagacgcaactattaacggcgatcgtcagcaagctcatggacattaacggaatcctggagggaaaagacgagtcggcgccgggtaaataa

SEQ ID NO: 5 - protein, 775 aa, synthetic construct
  REGION 1..775: note = Chemically Synthesized
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPENPSTHVSSNGPTTPPHGPHTTFLPPTSPAPSTSSVAAATLCSPQRQAVSRYSGWSTEYTQWHSDLTTELLWHAHPRQVPMDEALAAAAAASYQVNPQHPANRYRHYEFQTLSLGTSEVDELLNCCAEETTCGGTQSTVLTNATNTTSCGGAVAGSSNVGPAGASAACDLDAELAGLETSAADFEQLRRLCAPLAIDTRCNLCAIISICLKQDCDQSWLLEYSLLCFKCSYAPRAALSTLIIMSEFTHLLQQHFSDLRIDDLFRHHVLTVFDFHLHFFINRCFEKQVGDAVDNENVTLNHLAVVRAMVMGEDTVPYNKPRRHPQQKQKNNPYHVEVPQELIDNFLEHSSPSRDRFVQLLFYMWAGTGVMSTTPLTELTHTKFARLDALSTASEREDARMMIEEEEDEEGGEKGGDDPGRHNGGGTSGGFSESTLKKNVGPIYLCPVPAFFTKNQTSTVCLLCELMACSYYDNVVLRELYRRVVSYCQNNVKMVDRIQLVLADLLRECTSPLGAAHEDVARCGLEAPTSPGGDSDYHGLSGVDGALARPDPVFCHVLRQAGVTGIYKHFFCDPQCAGNIRVTNEAVLFGRLHPHHVQEVKLAICHDNYYISRLPRRVWLCITLFKAFQITKRTYKGKVHLADFMRDFTQLLESCDIKLVDPTYVIDKYV

SEQ ID NO: 6 - DNA, 2328 bp, synthetic construct
  misc_feature 1..2328: note = Chemically Synthesized
  source 1..2328: mol_type = other DNA
atgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaaaatccgagtacccacgtgagcagtaacggcccaacgactccccctcacgggccccacaccacgtttcttcccccgaccagcccggccccgtccaccagctccgtcgccgccgctaccttgtgcagtccgcaacgacaggccgtttcgcgttacagcggctggagcaccgagtacacccagtggcactcggacttgacaactgagctgctatggcacgcgcacccgcgtcaagtacctatggacgaagcgctggccgccgcggcggccgcctcataccaggtaaatcctcaacaccccgccaaccgttaccgtcattacgaattccagacgctcagcctcggcacctcggaggtagacgaactgctcaactgttgtgcggaagaaaccacgtgcggcggcacgcaatccaccgtactcaccaatgcgaccaacaccactagctgcggcggagccgtcgccggcagtagcaacgtaggacccgccggcgcttcggccgcctgcgacctagatgcagaactggccggcctcgaaacctcggcggccgactttgaacaactgcggcgactgtgcgcgccgctggccatcgacacgcgctgtaacctatgcgccatcatcagcatctgcctcaaacaggactgcgaccagagctggctcctcgagtacagcttgctgtgcttcaaatgcagttacgcgccccgtgcggcgctcagcacgctcatcatcatgtccgagtttacgcatctgctgcagcagcacttttccgatctgcgcatcgacgacctgttccgacaccacgttctcacggtcttcgatttccacctgcactttttcatcaatcgttgctttgaaaaacaagtgggcgacgcggttgataacgagaatgtcaccctgaaccatctggccgtggtgcgggccatggtcatgggtgaagacacggtgccttacaacaagcctcggcgccacccgcaacagaagcaaaaaaacaacccttatcacgtcgaagtgccgcaagaactgatcgacaactttctagaacacagctcacctagccgcgaccgcttcgtgcagctgcttttctatatgtgggccggcaccggcgtcatgagcaccacgccactcacggaactcacgcacactaagttcgcgcgactagacgcgttatccacggcctcggaaagagaagacgcaaggatgatgatagaagaagaggaggatgaagaaggaggagaaaaaggaggagacgatccgggccgtcacaacggcggtggcaccagcggggggttcagcgagagcacgctaaaaaaaaacgtgggtcccatttacctatgtcccgtacccgctttttttaccaagaaccaaaccagtaccgtgtgtctgctgtgcgaactcatggcctgctcctattacgataacgtcgtcctgcgcgagctgtaccgccgcgtcgtctcgtattgtcagaacaatgtgaagatggtggaccgcattcagctggtattggccgatctgttgcgcgaatgcacgtcgccgctcggcgcggcacacgaggacgtggcgcgctgtggactcgaagcacccacctcgcccggaggcgactcggactaccacggcctgagcggcgtcgacggcgcactggcgcgacccgacccggtattttgccacgtcctgcgtcaggcaggcgtcacgggcatctacaagcactttttctgcgacccgcagtgcgccggcaacatccgcgtcaccaacgaggccgtgctcttcggacgcctgcacccccaccacgtccaggaggtgaaactggccatctgtcacgacaattactatataagtcgacttccgcgacgtgtgtggctctgcatcacactcttcaaggcctttcagattacaaaacgcacctacaaaggcaaagtgcacctggcggactttatgcgcgatttcacgcagctgttggagagttgcgacatcaagctggtggaccccacgtacgtgatagacaagtatgtctag

SEQ ID NO: 7 - protein, 402 aa, synthetic construct
  REGION 1..402: note = Chemically Synthesized
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEMARDEENPAVPRVRTGKFSFTCANHLILQISEKMSRGQPLSSLRLEELKIVRLICVLLFHRGLETLLLRETMNNLGVSDHAVLSRKTPQPYWPHLYRELRQAFPGLDFEAAVFDETRAARLSQRLCHPRLSGGLLTRFVQRHTGLPVVFPEDLARNGNILFSLGTLYGHRLFRLAAFFTRHWGAEAYEPLIRIICQKMWYFYLIGTGKMRITPDAFEIQRSRHETGIFTFIMEDYRTFAGTLSRHPHRPHPQQQQHHHPGPPHPPLSHPASSCLSPEAVLAARALHMPTLANDV

SEQ ID NO: 8 - DNA, 1209 bp, synthetic construct
  misc_feature 1..1209: note = Chemically Synthesized
  source 1..1209: mol_type = other DNA
atgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaaatggcccgcgacgaagagaaccccgccgtcccgcgggtccgcacgggcaaattctcctttacttgcgccaatcatctaatattacagattagcgagaagatgtcgcgcggacagccgctgagctcgctgcgtttggaagaactcaagatcgtacgcctcatctgcgtcctcctctttcaccgcggtctcgaaacgctgctactgcgcgaaaccatgaacaacctgggtgtctcggaccacgccgtgcttagtcgcaagacgccgcaaccctactggcctcatctgtaccgcgaactgcgccaggccttcccggggctggactttgaggcggccgtgttcgatgaaacgcgcgccgcccgtctcagccagcgcctgtgtcacccgcgcttgagcggcggactgctgacgcgctttgtgcagcgccacaccggcctgccggtcgttttccccgaagacctggcgcgcaacggcaacatcctcttctccctaggcacgctctacggacaccgcttgtttcgtctggcggccttcttcacgcgccactggggtgccgaagcgtacgaacccttgattcgcatcatctgtcaaaaaatgtggtacttttatctcatcggcaccggcaagatgcgcattacccccgacgccttcgagatccagcggagtcgacacgagacgggcatttttacttttattatggaagattacagaacgttcgccggcacgctgtcccggcacccgcaccgtccgcacccacaacagcagcagcaccaccaccccggtcccccccatcctcctctttctcaccctgcctcgtcctgtctcagcccagaggccgtactggccgcccgcgcccttcatatgccgacgctggctaacgacgtgtga

SEQ ID NO: 9 - protein, 693 aa, synthetic construct
  REGION 1..693: note = Chemically Synthesized
MPRVDPNLRNRARRPRARRGGGGGVGSNSSRHSGKCRRQRRALSAPPLTFLATTTTTTMMGVASTDDDSLLLKTPDELDKYSGSPQTILTLTDKHDIRQPRVHRGTYHLIQLHLDLRPEELRDPFQILLSTPLQLGEANDESQTAPATLQEEETAASHEPEKKKEKQEKKEEDEDDRNDDRERGILCVVSNEDSDVRPAFSLFPARPGCHILRSVIDQQLTRMAIVRLSLNLFALRIITPLLKRLPLRRKAAHHTALHDCLALHLPELTFEPTLDINNVTENAASVADTAESTDADLTPTLTVRVRHALCWHRVEGGISGPRGLTSRISARLSETTAKTLGPSVFGRLELDPNESPPDLTLSSLTLYQDGILRFNVTCDRTEAPADPVAFRLRLRRETVRRPFFSDAPLPYFVPPRSGAADEGLEVRVPYELTLKNSHTLRIYRRFYGPYLGVFVPHNRQGLKMPVTVWLPRSWLELTVLVSDENGATFPRDALLGRLYFISSKHTLNRGCLSAMTHQVKSTLHSRSTSHSPSQQQLSVLGASIALEDLLPMRLASPETEPQDCKLTENTTEKTSPVTLAMVCGDLGVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 10 - protein, 107 aa, synthetic construct
  REGION 1..107: note = Chemically Synthesized
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKKE

SEQ ID NO: 11 - DNA, 2082 bp, synthetic construct
  misc_feature 1..2082: note = Chemically Synthesized
  source 1..2082: mol_type = other DNA
atgccacgcgtcgaccccaaccttcggaatcgggcccgccggccacgagccagacgaggcggcggcggtggcgttggcagcaatagcagccgacacagcggaaaatgccgccgccaacgccgagctctgtcggcgccgccgctcactttcctcgccaccactaccaccacgaccatgatgggcgtcgccagtaccgacgacgacagtctcctcctgaaaacgccggacgagctggacaagtacagcggctcgccgcagaccatcctcacactgacggataaacacgacatccgtcagcctcgggtgcaccgcggcacctaccatctgatccagttgcacctcgacctccgacccgaagaattgcgggatcccttccagattctgctctctacgccgctgcaattgggggaagcgaacgacgagtctcaaaccgcccccgcgacgttgcaagaagaagaaacggcggcttcccacgagcccgagaaaaaaaaggaaaaacaagagaagaaagaagaggacgaggatgaccgcaacgacgatcgtgaacgcggcattctatgcgtggtctctaacgaggattctgacgtgcgcccggccttctctctctttcccgcacgcccaggctgccatatcctgcgctcggtaattgaccaacaactgacgcgcatggccatcgtgcgcctatcactcaatctcttcgcgctccgtatcatcacgccgctgttgaaacggctaccgctacgacgtaaagccgcgcatcacacggcgttacacgactgtctggcgctgcatctgccagaactcacgttcgagccgacgctggatataaacaacgtaacggagaacgcggcttccgtcgctgataccgcggaatcaacggacgcggatctgacgcccacgctgacggtgcgcgtacgacacgcgctgtgctggcatcgagtggaaggcggcatctcggggccgcgtggactcaccagccgtatctcggcgcgcctctcggaaaccacggccaagacattgggaccctccgtctttggacgattggagctagacccgaacgaatcaccgccggacctgacgctgtcgtcactcacgctataccaagacggcatattacgtttcaacgtgacctgcgaccgcaccgaggcgccagccgacccagtggcgtttcgcctgcggctgcgacgcgaaacggtgcgacgacccttcttttcggacgcgccactgccttactttgtaccgccacgctccggcgcggcggacgagggactggaggtgcgcgtcccttacgaattgacgctgaagaactcgcacacgttacgtatctaccgccgcttttacgggccttatctgggtgtttttgtaccacacaaccgtcagggactcaaaatgcccgttacggtctggctaccgcgctcctggttggaattaaccgtactggtgagcgacgagaacggcgccacgttcccacgggacgcgctcctggggcgcctctattttatctcgtcaaagcatacgctgaatcggggttgcctgtcagcaatgacgcaccaagtcaaatccacgctacactcgcggtccacatcccattcgccgtcgcaacagcagctctcggtgctgggcgcttccatcgcgctggaggacctgctgcccatgcgactggcgtccccggagacggaaccgcaagactgtaagcttacggaaaatacgacagagaagacgagtcctgtcactttagccatggtctgcggcgatctcggagtgcaggtggaaaccatctccccaggagacgggcgcaccttccccaagcgcggccagacctgcgtggtgcactacaccgggatgcttggagatggaaagaaagttgactcctcccgggacagaaacaagccctttaagtttatgctaggcaagcaggaggtgatccgaggctgggaagaaggggttgcccagatgagtgtgggtcagggagccaaactgactatatctccagattatgcctatggtgccactgggcacccaggcatcatcccaccacatgccactctcgtcttcgatgtggagcttctagaactggaataa

SEQ ID NO: 12 - protein, 107 aa, synthetic construct
  REGION 1..107: note = Chemically Synthesized
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPE

SEQ ID NO: 13 - DNA, 321 bp, synthetic construct
  misc_feature 1..321: note = Chemically Synthesized
  source 1..321: mol_type = other DNA
ggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaa

SEQ ID NO: 14 - DNA, 230966 bp, synthetic construct
  misc_feature 1..230966: note = Chemically Synthesized
  source 1..230966: mol_type = other DNA
gggccgcgtggtgggtcctcgaggggcgggggggtgtttttagcgggggggtgaaacttggagttgcgtgtgtggacggcgactagttgcgtgtggtgcggaggacggcgacggcgaataaaagcgacgtgcggcgcgcacggcgaaaagaagacgcgtgtctgtgtctgtgtgattccccggggaaaagaggaagttcccgggggacggcagcatgggtccctggggacacacgaaaagcaacgcccgggggcgagggacgacggccctggggaccgcgggggaaataacggccgcgaggccacacactcgttcctgcgaagccgcacaccccgaggccgcgcacaccgccgacacaccccgccaccacaccccgccggcacacccgccacacgcccgcgacacacccggcacgacacacccggcacacgcccgcgacacaccctgacacaccctgccaacacacccccgacacacccaacacacgcccgcgacacacccggcacacacccacccggccgcgccccgacacacccaaaacaccgccggtgcggggccgcgtggtgggtcctcgagggagtgttgagggccgtaagcgtgttgtgtccgacgctgcctgcgcactgccggtgcgtgtcgtcccacggtatttgttgtcggcaccgggcttcgggacggtgtttcggcgcgctgccggtgcgttccacggtccttgcctgtgtcgtttcggcgctgcgcttgtcgggggttttcgagcgttctggccgccggcgatgccggggtgttgcggagacggggggtgtgcgggacggtgttggggccggggacgggggttgcgctggggccggggctgttcgcgccgcgtaggggaggttacgttggggacggggacagtttgcggcgcggaccagggaacccacctcacctatttaacctccacccactacaacacacacatgccgcacaatcatgccagccacagacacaaacagcacccacaccacgccgcttcacccagacgcccaacacacgttacccttacaccacagcaacacacaaccgcatgtccaaacctcggacaaacacgccgacgaagaacaccgcacacagatggagctcgacgccgcagactacgctgcttgcgcacaggcccgccaacacctctacgatcaaacacaacccctactactcgcataccccaacaccaacccacaggacagcgctcattttcccacagagaatcaacatcaactcacgcatccacttcacaacattggcgagggcgcagcactcggctaccccgtcccccgcgcggaaatccgccgcggcggtggcgactgggccgacagcgcaagcgactttgacgccgactgctggtgcatgtggggacgcttcggaaccatgggccgccaacctgtcgtcaccttactgttggcgcgccaacgcgacggcctcgctgactggaacgtcgtacgctgccgcggcacaggctttcgcgcacacgattccgaggacggcgtctctgtctggcgtcagcacctggtttttttactcggaggccacggccgccgtgtacagttagaacgtccatccgcgggagaagcccaagctcgaggcctcttgccacgcatccggatcacccccatctccacatctccacgtcggaaaccgccgcaccccgccacatccaccgcatcgcaccacccacatgcttcgcctcggtcagatcacacgctttttcctgtcccatctacaccctcagccacggttcacaatccccgaaactacgccgtccaacttcacgccgaaacgacccgcacatggcgctgggcacaacgcggtgaacgtggcgcgtggatgccggccgagacatttacgtgtccaaaggataaacgtccctggtagacggggtagggggatctaccagcccagggatcgcgtctttcgccgccacgctgcttcaccgatatccaataaacccatcccctcgccacgacgtctccgcgtatctttgtagcctcaagaatccgtccccacgtccacccatcccgagcactccacacgccataacaaaccacggacacgacaaatgcatgcaaacttctcatttattgtgtctactactctgtgttgctacagggagtgaagagggtgaaggcaaagaaaaaaaaaaggaacaaaataatagattagcagaaggaataatccgtgcgaccgagcttgtgcttcttttcttataaggaggcaaatatactagggaaaacataagaataggaagaaaccgaggtttgggagaaaagctgagataaaatagcgcattttccatacagaggttgttgtttttgtggatcctaagaggtttcaagtgcgaatctcaaagttctcacgagaatattgtcttcaagtatcgacaactgtggtccaagatttttttttggtctttttaggttctgcgagggacatcacgatggatcgttgcgatgaagtcacgcgtacgcctctggtgtggcgcggtgtcgtgacaggagagtgtgttttcagtgcagagctgtcttgattcctatatccgagtatctgttttctcgtaaggacggtaatcttctttggtgtaagtacatctaaaagctgcaaactatattttaagggctgtctctaggtgtactttgatgctggagtttttcgctgtgttgatgtgaataaatctactactactattatatgcagaaagagtgattatgccgagacaagattgcattggctgaactgtttcaaaaacgcctacactctacttatccgtaaacctaaggtaatactatgtgtaagttgtttttttttctttttgtagtaaaatggtgatacgtgcaattaaaactgtattccatgtttccatcctttcatttcaactttaaaggcggctttgagagcgaagaagtgcgaggataaaaatggatgactccttcgtgtccagggagtcgactactgcaacgctgattgattaaaagatggtctccgatgatgatgttgttattgatcgaatcatggtgcagaacggcgacggagaggagcgtgtccgccgccgggaaggtggtctctttctcttttcttttttcaagaaatcttccatgtgtttatcgtagtgatcgaaatcgactgatctcgggttctttttgttggtttcttttcggttaatcatgtattgttttctttttttacagaaagatactttttttcatgagcaattcctcgcccggcgccggcatgccgaggtggggccactgcgatcagcggcatgccgacgccgacccggggatcttggattcaccgttttctctcttctctctctacatacagaccgggtggcaggagcggtaaggaatcatcgtcgtctttcattcttcgatgattatggtaatactaaatcttatctaggagcatatacatctaagattggagtactagtagtcgtttgtggtttctatttttttttatatttatctatgacagtttttctgtttttcgttttgataataatataataaaaactcatggacgtgaaatctggcttggttgtggtgatttcattctcattattgttgttttctttccgtcttgcggatgaagatgttgcgatgcggttgttgttggtgttgctatacaccgagagagatgatctttttgttcttctggttcatttcctatgattgtttggctgctgaccgacgcgtcaggatgtgcagggcatgcggggaatcaggaccggacacgggataatttcatctacctatacggagatcgcggtcctcgccatgaggatcgcgacaggcgcgtcgagggggcaggaacacccttgcggattgacattcttggtggtgtttcgttgttgtcggtagttgttgttgacgatgaggataaataaaaatgaccttgtttttgttctgttttctcttgttgggaatcgtcgactttgaattcttcgagttatcggaaagctgaggtacccaaatgtctgtagcttttttctttttaccctcttgtttatcatctgcgattcgtggtaggtaggagagggaaatgataatccgagattaaggaaaggagaagataaaaaataaaaaaaaataataaaacagaagccgaccggccgccgacccgttccccaggaccagcctacgaggaacggataacgcggtggcgacggcagcggtggtggcgctgggggtggcggcagtggtactgctgatggtagtcgggacggaggagaggcgatgcatacatacacgcgtgcatgctgcatgggtggatggtacggccgggagacgcggaagagaaactcacataaaaaggtgacaaaaagagcggttgaaaaaagaaaacaagattcgaccagacagaagagaaggaccggggcttggcgacccttccacgactgctgttgtcatctcggctcctccgtcttctcccggccacgggcggctaagtcaccgccgttctccccatccgtccgagcgccgaccgaccagccggccgattcgcccgccggggcttctggagaacgccggggcagcagcgatctggggaagctgctaaacccctgcgtttttatatggtagctctgccgagcgcgggctgacgcgttgggtaagcggaaagacgtgtgtgacgaaaaggggtcccatggtatttcacgtgacgatgaggagatacggtttggagcacatacggtttagaaaaagggagttgtcgtgacaagggctgagggacctctgtctccatgtgtgtataaaaagcaaggcacgttcataatgtaaaaaagaacacgttgtaaacaagctattgctgtatcattcggctgactatgcttcattcggactgattttcttttcctaacggcgtaacttaaagtgattaacgtatgatatttgttccccagagttatactatagtcatcatcctaaaattcagatataaatgaacacatgtcgtatgggattattaagaaaccgaaactctccacagttcaccatcttcttcgtcattcaaccgatgacccactccgtacaacgaatcagtctgctgcgtcatattgcaaagcacaagcgacgtatgcgaacaacttgaaacacaggctgttgtattgacgaccgttgtaccattattagtcaccaccgttatcccatgtttcccacccgatggaaaaccgtcttctatcatcaactgtggtaagatttcgaccctgcgaggtattcagtttcctcatatccataacctggattttatcattaaaccccaatattaaacacttttttagtaccccccacccaccaaaaaatgtgactggaccggttcctagcagctctgggagccatgttcaggttgaaccacagctacagcgaaaccgagtccagtgaccggtaaccacgtccagcccctgcgtatgtaccagtccaagcacgtccggtcattgttctacacaggaaatctaactaggtcaacgcaattttattccaccgttacgcagaatactaacaaaaaaacacacaaatttaacgaattacacgtagtttattacatgaaaactgtaagaacaccaattcactaagcgatacaacatttagctgacttccaagtgccacacatcaccactgtattcatccatgttttcaccgaaccaacgagacagatcgaagaagccagaatctcccgactttaaattacataaatccaacgtattatgaccacagctcgacacacaaatagttgcgttactattcacagtagcattacctatacccgtaacgttgcacaaccactgatcaccattgttaccaaaaacggttttccacttagttgtcaacggatctttcctatgcgtaatggtaaaattactaccagtcgtcgcttttagctcattacgagtattatccgcatccacatatatcaacgtcatagctaggcacgctataagtaccccccccccacaatggaatgttgccaaaccggttctttcccgttatagccatagcgttcccaggcaaaagcaaacgccaaacctaatgcagtgaaaagcgcttgcagccagaaccagcttatgtaccagccacaatcacatccggttattgtttccacaggaaatcctaccaggcaaagccccgcttgttttgttcctatcttgtttagcaattcgtaaactgtcagcctagcgacgtccgtttagatcaaaagtcacgtatatagcgacgctgtttccatccgtttccccgtcccgccgtttccgaacaacccacccgggttcagacaaccgaccaccaacagaaatatacacacagaccactgggagttcagttaaagatttcatcaggtttattttggctgctgctagtcttttgcttcttagaaaaaaaatacccatatagagaaataatgatagtttgacaacacatatggcagggatttcttcttcatcaataagatatgcaattcccccagggagagactttcaacaattgaatttacaaaaacaaaattacatcaggagaaagagaggatacattaataaatatattatatctggtgtatatactgaatgctgctggttcataaggtaacgatgctactttttttaattccaagatggtttttctttgttagtcttttgttgacttgctggttcctaaaagttcgcaaaaacgattgtgtgaagattttatgacgttggttgactagttcatgagattctgctgtacgtgtgatggttattcgctggttcgttctaagatgagtatcgtactgtgtctgcgatggtcgtctcttactggcattctctcggctgcctcttgctttcatgattgaaaaggaaaaaaggactccgagggcgcggtcatcttttacttttcggttttctcgttggcgggtcagaggtagtcagatcatgagactgtcgtggtcgatgaaactgtgtctgctcaagtgacgtccatttcttgtacggagaaaaaagtcatcgggataaataaggctatacaaggcgttgtcaagcgtgcggctctaaacaaattaagcgatacaaaattacagtaatacgaataataaattacccccctccccctgtggtcccccgagacgagagccacccatcgtgtactctcgcaccacccacgaccacagagggagacgggacgaagagacgacgcacagcgccatctcctcctggaggccggcgacgttaactgctacagctgcggcggcgaagacagctgcgatttgtcggccgacatgccgatggtatgggcggcggcggcaatggccgcggcagcggggaggagaggagagagaagaggagcggggcgtccgaaggcgaggatggcatggtctcgccggagcgcccggcttttatggaacactcgcgtccggttgggtatcacccacaggaagatgagtcacaacttccaaaccatcttgagacccgagtaacggtttacaggtcgcacgccagtcagctaaaaacagcggacagtcccacgctgtttctgttgtggctctctccagtttcctcatcaccgtcccggtctccgtcgtcatcggaagaataccacccgctctcatgcggcagtcgatcggcctcgacgaacgagacgcggcgacgcctctccacggccgactggttgtggtggtgaaagaagagcaccagcaatcccaggaggagcaacaagccctcacatgtccaggaggtcggggagagggcctgtcggagatggccgtgaggcatcacgtacggcagctgaggagaaacggagaagaaaggaaaattaccgtcaggggccggggttcttattagagaaacagcacgtaggtcaggatccagatgctaatggcaatcatgatgacgatgatcatgcaggccaagacgcggcgcaccaatgccgaatccaatagccgccgtgcctccggttggtggccggcggcatctagagacatgatttgggggggaccggcggcgcaaaaagacagggagatggacagtgtcacggtgttttgttataattaggacatggggaccggaagccgagacagagtactacagggtgttgaagggtaacgtgagggagatcatgtcatgggcgggctgaagaccgtgcggggaggattgacgtgtgcggtgcttgtggaacacggtgttttaatatgtatccgcgtgtaatgcacgcggtgtgctttctggcactcagcttggtaagctatgtggccgtctgcgccgaaaccaaagtcgccaccaactgtctcgtgaaatcagaagatacccatttgacgtgcaagtgcagtccgaataacacatcatctaataccggcaatggcagcaagtgccacgcgatgtgcaaatgccggatcacagaacccattaccatgctaggcgcatactcggcctggggcgcgggctcgttcgtggctacgctgatagtcctgctggtggtcttctttgtaatttacgcgcgcgaggaggagaaaaacaacacgggcaccgaggtagatcaatgtctggcctatcggagcctgacacgcaaaaagctggaacaacacgcggctaaaaagcagaacatctacgaacggattccataccgaccctccagacagaaagataactccccgttgatcgaaccgacgggcacagacgacgaagaggacgaggacgacaacgtctgataaggaaggcgagaacgtgttttgcaccatgcagacctacagcacccccctcacgcttgtcatagtcacgtcgctgtttttgttcacaactcagggaagttcatcgaacgccgtcgaaccaaccaaaaaacccctaaagctcgccaactaccgtgccacctgcgaggaccgtacacgcacgctggttaccaggcttaacactagccatcacagcgtagtctggcagcgttatgatatctacagcagatacatgcgtcgtatgccgccactttgtatcattacagacgcctataaagaaaccacgcgtcagggcggtgcggcgttcgcgtgcacgcgccaaaatctgacgctgtacaatctcacggttaaagatacgggagtctacctcctgcaggatcagtataccggcgatgtcgaggctttctacctcatcatccacccacgtagcttctgccgagccttggaaacgcgtcgatgcttttatccgggaccagggagagttgtggttacggattcccaagaggcagaccgggcaattatctcggatttaaaacgccagtggtccggcctctcactccattgcgcctgggtttcgggaatgatgatctttgttggcgcgctggtcatctgcttcctgcgatcgcaacgaatcggggaacaggacgctgaacatctgcggacggacctagatacggaacctttgttgttgacggtggacggggatttacagtaaaagatgcgtgtcgcctgccgaagacctcaccatctcacgtacaggcatacggcgtatacaatcataatattctatattctgcatagagttacatgcaacagtactactaccaatactgcatccatcacatcacccaacactgcttctaccacctttgtgaccagcgtattttctactccgaataacaacacatcaacgacgccacacacatctgtcacctcacaagcgtcaaccattggcaacatcaccaacgttacctccgacttgagtactttcacaaccgtatattctacattcaatacatcatatgctaatatatccaatacggctgccactacagaattgatttcaacaaataccaacactatattatcttttaccaacgtaacagcaaacgctacatcatcttataacacaacaatcaccgtaactatcacgtcagatgaaacttcgcacaacgtatccactaatactgcacttataagcacgccatggcttacaaattgcagcgccacaacgtacaccacgtacaaccgtactaactcttccaacgcttgtcacacagagacaacaatcatacgtttcaaagaaactaatacaacaggaatagaagggagtaatgtcaccataaaaggtaattctacgtgggattgtctttcagtcgcctggatacgacattacaatcgatccacacacggacatcatctaggtcatcgtaagaacgcacatacccaatcttggtattggttacgcatccttacctctcatactgtatgtcattctcaacatgaaagaccttcactgtaccatgacttatgtcgttcgtgcaacaacacagaactacatctgtacgatctaaatatcaccaattccggcaggtacagcagacgttgttttaaagaaaattacttcacaggacatcacgaagatgaaaatttctacctattagtaacaccaaaaaatcatactgaagctattaatgctactttcgtttgccctagatacaacaccgatatcgaaaatgaagatagagagaaaggaagtcaacatactaacaatacacatcaccacaaacgtaatctctatcatagctcgcaaagaagccgcaccgtatggaccatcgtgttggtttgtatggcctgcatagttctgttttttgcacgacgagcctttaacaaaaagtaccatatgttgcaagacaccgtcagtgaatcagaattcattgttcgatatcacacagaacatgaagattgagctacgtttccgggcagacatcttatgaagctgaacaataaactaaaacattctgtaaggctcagcgttcaaaggaatattaatgcccattgagcgagaactaatattgcaatggactggcgatttacggttatgtggacgatactaatatccgcgttatcagaaagctgcaatcaaacctgttcctgtcaatgtccctgtagtactaccgttaactattccactagtactgagacagccacatcaacatacagtacaacagttatcagcaataaaagcacttcagaatctataaattgctctactgcaactgcaccagcaaccaccgtttctacaaaaccgtcgaaaacaaccacacagatatccacaacgacaaatacaaacgttgagactaccacatgtaccaacaccaccacgaccgttacttgtgatggtttcaattatacagtccataaaagatgcgaccgcagttacgaggtaatcaacgtaacaggatacgttggtggcaacataactctaaaaaatgcaatcagactgagaaatggcacaatgtagactggattcattatgagtaccccacgcataaaatgtgcgaattaggcaactatcaccaaacaacaccacggcacgacatatgttttgactgcaacgacacctccctaactatctacaacttaaccacaagaaacgctggaaaatataccaggcatcaccgtgataacggtcaagaagaaaattactacgtaacggtgttaattggagacacaacgttatccactcttggcacatgccctgtaagatataaagaatctaggaacactgaaaacaccattggaagtaacatcataaaaaccattgagaaagctaacattcccctgggaattcatgctgtatgggcaggcgtagtggtatcagtggcgcttatagcgttgtacatgggtagccatcgcattcccaaaaaaccgcattacaccaaacttcccaaatatgatccagatgaattttggactaaggcttaacatgcacatcaataaactttttttaaccaataacatgtctctgtttttttttgttaacaacctatgatataaagcggtatattcaatcattactaaacaaaaaaacatgggcatgcaatgcaacactaaattgttattgccagtcgcactaataccggttgtaatcatcctaattggtactctagtgcccatacttttacatgaacaaaaaaaggcgttttactggcgactttttctgcaaagtcaacatgtagaagcacccattacagtaacgcagggagacacagtctacctagatgctagcaataatccctgtaattattccagcttttggtaccacggtaattgcgaactttgtggatggaacggatatctacgcaatgttacacattactacacaaacacatcgtgttccccgcaattcatgtgcataaacgaaactaaaggtctgcagttatataatgtaacattaaacgattcaggtgcttatactgaacacgtttacgaatgtgatctttcatgtaacattactacttataacgaatatgaaatactcaattacttcgataactgtaactacaccataaatagcaccaagcatattatcaccgtggtgtcttcacgtcattctaaacaaacaaattcccacgtatccactcacgctggttgggcagccgccgtggtgacggtaattatgatctacgttttgatccactttaacgttccggcaactctgagacacaaactacgaactagaaacaacgtaaatcgcatagcgtgattacaaagtatcgacactaatttatccaagataaaatttgattactccgtgcggttctcaaaaactgtaaggtcccgcttttctactccatcatgaaggatcgcaatagaatactgctatgtatcatctttatttgcatcatgtgcctcatttgtatttactttaaacgtcgttgtgttcttactccgtctccagacaaagcggatctgcgagtggaatttccctcgttacccccgtgtatcggcatacaatgtgctgcatgagaacacgcgtgacacatagcgtacccctggacggtacagtttatgataacgtcattcaggggaagtatacattactatcgacgtgttatcacagaacacacagattttctgcgtgttttataaaagagcgtctcgaagcagcttgagccacactacggtccagatgacgagcgtaatcaaaaatatgccgcgcagtagtcgaaagccgtactgagcgtgcgaggcgggtagggtgccgaacgacggatatgcgtcgttgtcatcttcgactataaggatcgcgaccgagtcttcggccatggtaaacgtcaccctgtgtggctggtatgtagcgtatccggtttggaattgttctgctccagctcgggggatagtgaggaattctcaagggatacgggacccaatgactggataagagaagggtttttccccgtaagatgatcctcgtatcacatgaggtctggatatgtataaatgaagagtgaaataggcacagggaatcagatgccagcctcgtgatgcagccgctggttctctcggcgaagaaattgtcgtctctgttggcttgcaaatacatcccaccttaagcgatgagtccataaagcaccgttgtccgggtacggtgaaagtgactcggattgtagcacgtcccttttttttgtttttgtatcgcttatcgccactgacagtgcaatattttgatcgtgaggctgagtatggttatgatgcttagaacgtggagattattaccaatggtactacttgccgcgtactgttattgtgtttttgggacttgttcaatcggcacgacgactgctcccgtggaatggaagtctcccgaccgtcagattcctaagaatattacttgcgctaactactcagggaccatcaacggcaacgttacatttcgaggtcttcagaacaaaacggaagactttttgcactggttgttagggtggggtcataagtccatctgttcgttcttcccgaaactccagggcaactataacgaacaacattacagatatgaagtagcgaacctgacgtataactgcacctataaccgcttgacgttgctaaatctgacgacggaaaacagcggaaagtactattttaaaagggaagatgcgaatttcaccttttattactcttgttacaacctgaccgtgtcctaaagaacgcacgtgaagttccacagagccgcgtggctgtagctattgtgtttacgttgcttttgaaatgttaagcgtccctacggcgctaacatgtttctaggctactctgactgtgtagatcccggccttgctgtgtatcgtgtatctagatcacgcttaaagctcgtgttgtcttttgtgtggttggtcggtttgcgtctccatgattgtgccgcgttcgagtcctgctgttacgacatcaccgaggcggagagtaacaaggctatatcaagggacaaagcagcattcacctccagcgtgagcacccgtacaccgtccctggcgatcgcgcctcctcctgatcgatcgatgctgttgtcgcgggaggaagaactcgttccgtggagtcgtctcatcatcactaagcagttctacggaggcctgattttccacaccacctgggtcaccggcttcgtcttactaggacttttgacgcttttcgccagcctgtttcgcgtaccgcaatccatctgtcgtttctgcatagaccgtctccgggacatcgcccgtcctctgaaataccgctatcaacgtctcgtcgctaccgtgtagctagttagccagctgtgtatagtttgttgtgttttgcttttgcatatttgttttcagtcagagagtctgaaacggggtgggagggacttttacgggtaatgcatgctaagatgaacgggtgggctggggtgcgcttggtaactcactgtttgaatacgcgctcacgcacatatgtagcactcaacatgttagcttttgcccgcacgccccggggcgtgccgagctgcctttttaataaagtctgggtttccagatacgcgctggttctgattttgatggtttgtgcctctgaaagctctacgagctgggccgtgacatccaatcgactgcctaactgtagcacgataactacaacagcgggtcaagacgctgaattgcacggtccggcaccgttaagctgtaatgtgacccagtggggacgttacgagaatggaagcacacccgtattatggtgcactttatggggatcacgcacgcgagtctcattaggacaccgtgtagcgtttggctgttcttggaaaacattttttatttataacgtttctgaaagtagtggtggcacttattatcaaaaaggttacaactgcaccgacaaacatataacactatcttgtttcaacctaacggtggttcctcgagcggttcaaagcacaaccaccgtaatgacacccacggtggttacaaactccacattcagtgtgtcacttgttgcgtcgagactgacgacaaattccagcgcgtttagacacgctagttatcaacggcaacagcgtgtcggaaacgggacgttatccaagaacataactaacttggcattcacctacggcagctggggcgtcgcgatgctgctgttcgccgccgtgatggtgctcgttgatttgggtttgcctcaatcggcttggcgacgctggcgaagccacgtggacgatgaagaacgtggtttgttaatgtaggaaataaaaggcactgtttgagcatgactgtttccaaaccgtaacgtggtaaataaatcatggcttccgacgtgagctcccatcttctaacggttacacaatcccgttggacaatacatcatatgtacaataaactgttgattttggcgttgtttacccccgtgattctggaatccatcatctacgtgtctgggccacagggagggaacgttaccctggtatccaacttcacttcaaacatcagcgcacggtggtttcgctgggacggcaacgatagtcatctcatttgcttttacaaacgtggagagggtctttctacgccctatgtgggtttaagcctaagttgtgcggctaaccagatcactatcttcaacctcacgttaaacgactccggtcgttacggagcagaaggttttacgagaagcggcgaaaatgaaacgttcctgtggtataatttgaccgtgaaaccgaaacctttggaaactactacagctagtaacgtaacaaccatcgtcacgacgacaccaacggtgatcggcacgaaaagtaacgttacggggaacgccagtttagcaccacaactacgtgccgtcgctggattcttaaatcagacgcctcgggaaaacaacacgcacctggccttggtaggtgttatcgtatttatagctctaatagttgtttgtattatgggatggtggaagttgttatgtagtaaaccaaagttatagtgatgtgctttttatcagggagaaggttttgtgccaacaatgactaaccctgggctatatgcatcggaaaattataacggaaattatgaacttacggaagccgccaatacagcacgtacaaatagcagtgactgggtaacgttaggaaccagtgcgtcgctgttgagaagcacggagactgcggttaaccctagcaacgcgactacggttactccacaacctgtggaatacccagctggggaagtacaatatcaaagaacgaaaacacattattcttggatgctaattattgccataattctcatcatttttattatcatctgtctgcgagcacctcaaaaagtctacgatcgctggaaagacaataaacagtacggacaagtatttatgacggacacggagctgtgatgaactacaatgtatagatacacgtggctgctttggtggataacaatattgcttcgtatacaacagttctatcaatggtggaaaccagatacaacgtcatgcattcagaaaacgggatatgaaggtcaaaacctcagtctgcctcctagtaatgcattatcatctaaagactatactttttcatggtataaagattcacttaaagcccttaacatgttatgttattatactgaaaaacttgaagaaatagatagcaagccagatactatacgacgatgttttttgaatcatacattgtttcttattaatttaacaagtcactatagcgggatttactacttcgattctctatacacatatggttgggtattacggacacctctatgttacaatgtcactgtatattccatatatcaaacacacatccacacaactatattgctctatccgcctacgtccacatataattcattaactatatcatcatttacctcaaccaacttaacacataccgcggtccactatgccgccggtaacgttgaagcacaacacgatactgccaccccacatacaatgtggatcatacccttagttatcgttacaacaattatcgttttaatttgtttcaaatttccccagaaagcttggaataaattcacacaataccgatacaacagtatgctcaccgccgcttaaagaatcaccgtcgagaaaactaaaacgtaaaaagaatggccatgtacgtttatttttcagctcactgtttgaataccgtaaacataatgacgtacatatacgtgattatacaacaggtgtttgtgttatgcggcgactgattaaccatatcgtgaaccatgatcttttccgatggtccgtcatgaccgcaatgatattttacaggtattccgaaacctgtatggaggtcactgtcagagtaggtgatccagttaccctcggtagtggacatggttatcatccaggacaaaaagtacactggtataaccagtcatgcgtcggcattagcaacggcgaaaatacgcatcctatctgcacctacgaccctcctaaacctggtagacgaaagacaatgaaaaccactccgttaccatcaccactgttgtacgagtgtcacaattccacattaagcattcttcatgtaaacgtctcagatcccaaaaactattgcaggcgaaaatgtccaccaaacggtaactgtgaatttcccacatgttttacgttatcactgatttccagaacgacaaccaccagaaaacccggacaaaaaactacgttgtcgcgattaaaaaccacgccaaataaacatacgcagcacaaaagatccacgcgaagaacgtcacctagagattacaatgtaacgggtctgccgaaaggctttgcggactcgtttaccggtaacgtagaggcacatagagccaaagatgccgcacacagcgcatggatcctcattgtcatcatcattatcatagtcgtcattttatttttcttcaagattcctcaaagactcagagagaaatgggacaccagaggatacctttacaaaggaaccgacggcctgcccactacggactacttatcgtgagcggacggatatctccggtttcaaacccactgtttgaatatagggacagtccctacggaacctgagaacatgtggaaatcacctgtggtagaatgctgctcaggtacattacctttcatcgtgaaaaggtactttacctagcgatcgcatgcttctttggtatctacatcagtttccacgacgcatgcattctggtacctgctaaagtaggtactaacgtcacattgaacgcggtacatgtgcatgacggtgactatgtgtactggtcttttggtggaggtggagctaatagattaatgtgtcgctatacaccaaggctagacgaaattcacaaaaacaccaatcgaagtttttcatgtcttacaaatcacagtctccttctcatcaatgtaacggaagaatatactgattactatcgcaccatgaccacattcgtacatcagtcccataattggcacaaccacggcaacaaatggactttagacacatgttattatgtatacgttacccaaaacggaacacttcccactaccaccaccaaaaaacccactacgaccacgagaacgacaactaccaccacaacaaagaaaacaaccaccacgagcacgacaacgaccaccactaccaccaagaagacgacgacaagcactacccatcatcgacactccaatcccaaagaatccaccacccctaaaacccacgtagaacttcacgtcggtttaggagccacagcagcggaaacaccgttacaaccaagcccacagtaccaacacgtggctacacacgccctctgggttttagcggtcgtaatcgttattatcatcattatcattttctactttcgaataccgcaaaagctgtggctgctctggcagcatgacaagcacggcatcgtgctcatccctcaaaccgatctgtgagcaagtcgcgtaggaaatgattgcatgaaatcactgtgaaacgccaactccgtgccagctggcgcggcggacaggcctttgacgtatttgaagccaggcgcgctctcgataccgaaaggatccgagggggctttccaaagccgacgtccctgattcccttcataaagctgttgaccggccctagaaagaccaagagcatgctgtgggcccactgcggtcgcttcttgcgttatcatctgctcccgctgctgctgtgtagactgccattcttactccttttccagcggccgcagtgggcccacggcttggacattgtcgaggaggacgagtggctacgggagatacaaggagcgacgtaccagctgtccatagtgcgccaagccatgcagcacgccggattccaagtcagagcagcgtcggtcatgacgcggcgaaacgccgttgacctggaccgaccgccgctttggtcgggatcgctcccgcatttgcccgtctacgatgtgcgttccccgcggccgttgagaccgccgtcatcacagcatcacgccgtatcacccgaactgccgtcgcgagacgggatacgttggcagtaccaagagctgcagtatctggtggaagaacaacggcggcgaaatcagtcgcgcaatgcgattccgagaccctcgttcccccctccggatccaccatcgcagccggcagaggatgcacgagacgcggacgcagaacgtaccgaatcaccacatagtgcagaaagcaccgtcaggcacgacgcgagtgagaacgcagtgcggcgacggcacgaaagacggcgctataacgctctgacggtccgcagccgggactcgctgctcctgacgcgaatacgcttctccaaccaacggtgtttcggacgcgggcgtctgagacatcccgcgggaagcggtcccaacaccggcggaccgcgacccggcggtgcgggactccgtcaactacgccaacaactgacggtccgctggcagctgttccgcctacggtgccacggttggacacagcaggtctctagccagatcagaacccgctgggaggaaagcaacgtcgtgagccagacggccacgcgagtacgtacgtggttcgtggaaagaaccacgttttggcgtcgcacgtgggttccgagacagaacccggcggccgaagcgcaagaactggccgtcataccgccggcacccacggtgctccggcagaacgaggaaccacgtcaacagcttacgggagaggagacaagaaattcaacgcacactcaacgtgaagaagtggaggacgtttcgagagagggcgcgagagaagggaatgatgggagccgagcaagtggaaacgacgagagaaggaataatgcgggaagatatgatgatgatcatgaggttcaagagccgcaggtcacttatccagcgggacaaggagaactgaataggaggtcacaggaggagaacgaggaaggtggaccgtgtgaatcgccgccaatgacgacaaatacgctgaccgtggcctgtccgccccgagaacccccgcatcgtgccctgtttcgtctatgcttaggactgtgggtctcgagctacctggttcgacggcccatgacgatttagaatacaccgagccattcctttatttccccccatccccggtcgcttatgcgtgttaaacactaccaataaagataatctgccaatcgcaccttatatataatatgtggtcgcgtgtggtctttttaaggagctctgaaacacagacaggtatgggcggtggtcggctgccgccgctgtggctgccgctactgatcgcctggagcgagtggggcaactgctgcctcgatgcgcctccggtggtgcgttcgccctgtctgcagccggtgcgcgaccgcaaccgcgagcggaacccgggctcaccgcagttgctgccttacggcgaccgtctggaggtggcctgcatcttccccgcgcacgactggccagaggtctctatccgagtccacctctgctactggcccgagatcgtgcgttcgctggtggtggacgcacgcagcggtcaggtgttacacaacgacgccagctgttacatcgccggcgggcgctggcgcttcgaggacggcggcgcggcgcagcggctgagcctctcgtttcggctcatcaccgagaccgcgggcacctacacctgcgtgctgggcaacgagacccacagcctggcgaccgagaccacggcgctggtggccgacgtgcacgacctgcgccactcggaccgctcctgcgacctggctttcggatcgcgctcacagacgcggtacctgtggacgcccgatccctccaggttgcgcagtataaactgcggttgggagggtgaacggcaccgcgtagtccactacatccccggcacctcgggtttgctgccctcgtgcgaggaggacgagcgcgaactgtgcgtgcccttcatcagccagagcattgcggacaacaactgcagccgccggcatcgagttgacggcgctaggcggcgctatcatctacggagggattactggctgacggatccgaagatcgggttgctggccgcgggatcggtggccctgacctccctctgccacctgctgtgctactggtgttccgaatcgtaccggcgtctgaacaccgaggaggaaagcgaggcggcggaggaaactgccgcgggagaagcctctgcggtagcggcggcggccgtctctgaggaagagcagcggcgggagtaaacgaggagagccatgaagcggatgattcgcagtcacggcaggaaaacggaatgtcagatgacgagcgccggcgagcgacgcggctccgccgtcggtgcgcccatctgcggcagcggtacccgacgcggcagcggcgccaacgaacgccgcgactccgacgtcggtcccatcgcccacagtagcggtaccagacgcggttcggcgaatgaaacgtccgcctgtacgcggaccgatcaccagaaggcggacattgggctgtggttcatgtttctggtttttggactgtgttcgtggttggcgatgcggtatcgcgcacaataaattttgaatcgatgtcaaggaacgcgtgttttgtattttattgggaatattggcggggataaacctgtttcggatgtttacccttaatcttaccggggacctcgttgtcctctcctccttcttcctcggacaccgggctccatgctgacgtaggtaccgactggggtcaaaagcctgggtacttatgaggagcgcgcacaaaggaccgttaggcgccggcatggagcgtcgccgaggtacggtaccgctgggatgggtgttttttgttctttgcttatctgcctcttcctcgtgtgctgttgacctgggtagcaagtcctccaactcgacctgccgcttgaatgtgacggagttggcctcgatccatcctggggaaacgtggacgttacacgggatgtgtatttctatctgctactacgagaatgtgaccgaggacgagatcatcggcgtggcttttacttggcagcataacgagtctgtggttgacctgtggttgtaccagaacgacacggtgatccgcaatttcagcgacatcaccactaacatcttgcaagacggactgaaaatgcgaaccgtccctgtgactaaactgtacaccagccgcatggtcactaatcttaccgtgggccgctatgactgtttacgctgcgagaacggtacgacgaaaataatcgagcgcctctacgtccgattgggctcgctatatccgagaccgcccggatccgggctcgccaaacacccctccgtaagcgccgacgaggaactgtccgcgaccttggcgagagacatcgtgttggtctcagccatcactctgttcttcttcttgttggccctacggatcccccagcgactgtgtcagcggctgcgcattcgcctgccgcatcgataccagcggttacgcaccgaggactgaacggataaccgcaaaggccacgtgcaacgttcacgctgctataagaaggccatgtcccccgtggacgggtctctttgacacgagcgcggcacgccgttgccacgagcatggatcacgcgctcttcacacacttcgtcggccgaccccgtcactgtcggttggaaatgttgattctggacgaacaggtgtctaagagatcctgggacaccacggtttaccacaggcgccgcaaacatctacctcgacgtcgcgctccgtgcggcccccagaggcccgccgagattcccaaaagaagaaaaaaggcggccgtccttctattttggcacgatttgtgctggctgtttcgacgacttttctttcctcgggaggactcagagccactgatgtcggatccggcacggtctcccgaagaggaggagtaaacaacacacggctaagaggatacatcatcaaagaagataggaggggtcaaaacgcggactgaaagtatataacgccgatcatgtccgaggaactgttaataaaacgccatgatgacaatgtggtgtctgacgttgtttgtgctgtggatgttgagagtggtgggaatgcacgtgttgcgttacgggtacacggggattttcgatgatacatcgcatatgacgttgaccgttgtggggatttttgacgggcaacacttttttacctatcacgttaattccagcgataaagcgtcaagtcgggccaacggtaccatttcttggatggctaacgtctcggcggcctaccccacctacctggacggggaaagagccaaaggtgaccttatttttaaccaaaccgagcaaaacctgttagagctggaaattgcgttgggttaccggtcacagagcgtgctgacgtggacgcacgagtgtaataccacggaaaacggtagttttgtagccggttacgagggatttgggtgggacggggaaactttaatggagctcaaggataacctgacactatggacgggccccaattacgaaattagttggttgaagcaaaacaaaacgtacatcgacggtaaaattaaaaacatcagcgagggggatactacaatacaaaggaactatctcaagggtaattgcactcaatggtccgtcatttatagcgggtttcaaccccccgtcacccacccagtggtaaagggcggtgtccgaaaccagaatgacaacagagctgaagcattctgtacatcttacgggttctttccaggggaaattaatattacttttattcattacggtgataaggtgcccgaggatagcgagcctcaatgcaatccgctacttcccaccttggatgggactttccatcagggatgttacgtagccatcttttgcaatcaaaactacacctgccgcgttacacacggtaattggacggtggaaatccccatcagcgttacctcacctgacgacagttcctcgggggaggtccctgatcacccgacagctaacaaacgctataacaccatgaccatcagcagtgtcctcctagccctgcttttatgcgctttgctattcgcgttcctgcactactttaccaccttgaaacaatacctacgtaacctggcctttgcgtggcgctatcgcaaggtccggtcgtcatgaccagcaacgccctgtatgagctgtttcgacgtaggttaccgcgtgcccccgtcaacacggtcatgtttctcacgcgacgcactcgtgatgggttctgcggtcggttgacgtccatcgccacgaattcccactacactatgttcgtgttagatcacgggtccgtgcgcatcgagcgaccgagtcagtcagaagtggattgcgccagtttaatggaaacgctgaagcggattcggttacgaaattcgtgggtagcgtcagaagacgagctagatgtgagtcgcggggacgcgtgacacaaaacgcgttcaggattaacgtaggttttcgaaataacctacgtccgtgagtgacgcggtttcgtgttgaaacccgcgccggttctcacggtggtttatgatgaaaccggcgttggggatctacgcgggttcctcattcaacctgcgaaaagaggaagttgcggtaaaaccacgtcaataaagacgtcaatgacacctcaatgttgcgttggaacggtctttatatatacaaacgccgttatgttcagtgtccggcaagatgctcgggatacgggctatgctggtgatgctggattactactggatacagttgataacaaacaatgacactcgaagcaacaataccgataccatctttgtatctctccttaccggggccaacggagttactcgcacagccatcgggggtctgcattcaaactacaccaacttaaccgaggcattcagattcactccagcaaacacaacaactaactcttccacggagggtaattggagcgtgactaacctaacggagagttgcatcaaccgcggtgagtcctatctaactaccatctggcttctgaactgcgctgacaacaatacttattggtactctggaaatgcctataaccatacaattgacacttgtaaaaatacagtttcgggatatctcttcttcggcatgtgccagctatggaaagattgggttactaatgcttctcacgacactgtcagaattcagtcgttgggaaatgaaatacgctgcatgctgctccctagacagtataccctcaacgccacggtggaatggtacaacaaatctgaaggtgacgtaccagaagaatttatggactatgttatcctgacccccttggcagtgcttacatgcggactgcaggaagcttatatactcgacaagggtcgtagatacatgtatttgttttccgtgtcctgcgcgggaatcacaggtaccgtatctattatactcgtctccctatcgctgctcatcctcatctgttactatcgctgtggccggcttctgatatgcccacgcggctttgaactcttgccagaattcactgaggaagaggaggaaaaagaaaaattgttaacgtacaaggacattgaagtccaggtgcctatccgcacgcggcggctgctcgtcccttggatccgggagagcaaaatgtgggtactaccacccccgctgcctccacgacctccccacttaatagaattcccgccgtctcctccgccgtcgcctgggcccatgcacatggtggtctgcatgccagcatgacggactttgaactttgagccccaagcggtacggactacatattttccataaatctacactgaacttgagcacaaaaatactgacaatggactgaatatacagacttttatatgatccttgtacagatgtaaataaaatgtttttatttaaaactggtcccaatgttcttcgggaatcatggggtggggacgggggacgcggtaaggagcaaaaccgggtacatgggggggaacatcgtccagcagtagcaccagcggattgggtaggggttgctgcggaggtcggtcgatgacgatgtcgatctccatcggcagatccggcaacatctcttcgtctccctcaccgaccagcactcggcgctgttctggatgtatatgattttggaaaagcctccgacgagctcgcggcgcgtagaaagccaagcggcgcaagggccggcgagcccgaaagtccatgcgcacagatggcatgagtccttgagtgacggtggtgagctggggaacagggctacctcccatcgcgacggtgacagtggatccatgagagaggcgccgcacgctgcatggctaaataccgtgaatcccctgacgtcgtctttcgtcccgaacgcgtcatgttgggggcgaggcgtaaaccgtcgaggttgaaaaaccgcgtatctgcgacccgtccggactacgttgtttttcagaagcggccacatgacctcgagatgtcgtcacccaaggtatttaacggcacacagccagacgcgttcgtcagcagcgacgccgacaagacctcagcatggctcggaggctatggatcttgagcttactagccgtgaccttgacggtggctttggcggcaccttctcagaaatcgaagcgcaggtaaacggaatctggggaattcaacacaggtaagaaatacaaaaaataacgtgattgtgaacgcggttatcgtgtttttgcagcgtgacggtggaacaacccagtaccagcgctgatggtagtaataccacccccagcaagaacgtaactctcagtcagggggggtccaccaccgacggagacgaagattactccggggagtatgacgttttgattacagacggagatggcagcgaacatcagcaaccacaaaagactgatgaacacaaagaaaatcaagccaaagaaaatgaaaagaagattcagtaacagcagaccccaagggttaacgattatgttgactaccttgttttttattaaaaagctgtaaggttttgctctaaaaacaccccgcctccggtcttttttcttttgtattcggcacgcgaaacacggtttcttcccatagcctgtctaactagccttcccgtgagagtttatgaacatgtatctcaccagaatgctagtttgtagaggctatgcgggatgctgcggcggcgcgaccttccctctccacccagccccgtcaaaacacacgcgactcgagcggttcgtatgaaaaataaaaaacagctttttatttacaggaacggggaaaaaaaaggcacacggtccgtgggagacgcgggttcacgcgtcgtcaaaaagttggtggtccactccgtaaggacaggtaggcttatttagcttccgcatgctcctggttccgtaataaatgccgttttcgtggcagcgtgtcatgccgcgagtcacaaactccatcaaactgtcggccacgatgcaaacgtgctgattgttggcagcaaagacgcgcatacagtcgtccacgaagaggttgatcacgtcgtaggggctcaccaaccagcctaaaggttccacgtggttactgccgaccatgaccctccagtcgttaatctcgctccagtcgtacagccgaatcgtggagacgcgaatgacgctgtaatcacccatgaccatgagtcggccgcgatacgtagcacgccactgcgcgaacgcgtggatgtgcatgcagccggccagcgctctaagcgaggcggtgtgcggcagctcctctgggacggtgatgaagttgcagcgtcgcaaaccgatgttgagaaattcagtgatgctctcggccacaaaggtcaacgagtcagagtagatgtggtcggtccacaggtacatggcgcccgaggcgcccaggtacagttcagacggcacgttgtgatcgcccttgtgtttaagaaagttgtaggtgcagatgctgccgacgaaacgcagcggctcggggcagcagaggtagctggccagacgctgtgcatcccgtccttcgtcgcgcaccaagcgccagcgacgccggataacgaggcagcggtctttgggccagaccagggccacgcgttgcccgggtttccacggtcgcgacgtcttaggaggcctccagcggtcgagcagattgagaaaacagtccttgattaccgacatcgcggtcgcgcgtcggtggacaaaaagaaatcgggccgatccggaaaaaaaaaacgacggcaaaacaccgccgtgctcgagcgaagggtggcggagggccagaagaggcggccttgacggcgttggcagcgaaaaaattggcacgcgagtcaaacgggaagtagcgtcggtgttttatgccccaagcagcgtcgtcgtcactcgtggcgtcacagtcaacggtgctgacgtcctttggggcagtcgggcacgcgatcgtagatgccgttgtggccgctgaaacgtcggttttcaaacagcaggttaagtcccagacacatgaacgtgttgagattatctcccacccggatgtagcggtcgtcgcgcacgtcgcaggcgtagacggccccggtataggcgacgacgatggggataaggtcgacgggccagcgcaagtgaggaaagggcgcgttctcgcccttgaggctgacggttcccaggccgagaacgcgcattccgaaagcggttttgatgttgcgcagcaagtgaccgccttccacgctgttttcgaaacacctgaggttgcatagacgcagttccgttcccggcgggtacgtcaacggcatgaactgcccgtggtggcggatgatgaatcgcgccatggtatccaaaccgaggctccaggcgcgcaacagcgggcgaaagtagcgcttaaccaacgacgaggtcaggtagcgcatgcagtgcagggtctcgacggcgcgcagcccgacgcgcgcaaactccatgaggttgcgggccaggtagtagacggcggtgtcctcgcgtacatagcaaaaaacatagccctcgtccgagatgaggcacacagcggtcttcttctgctgatccggcgacaacacgccctcgttcacgaagcgacccacgaaggccaggcgcgtctggcaacacaggtagtgactccaagccttcacgtcctccggtttgaagtcctcgtccgtctcgatctcctgcagcactaggttccagcccggcggccagaccacgggcaacacctggcctgcgttgatgcgcacgtaagcttccagacagcccaggccgaactcggccgtgagcgccaggctagccagatcgctcatgtgacgcgccgagtcagtgggcgagcccgggggcccgtcgcacaccacgctccgtcttcttgtcctcaccgcggccagcgtggcgaggacactttccgcgcccgaggctgtatcttcggtttgcccgccggagccggccctcactatataacgtcccgcccgggtctcctccatgtatgcaggtaagcaactgagccgaacgcacctcagcagacgagaggatgtcgtcgcggcgtcgcagctcgtcacgtcgctctggcgaaccctcgacggtgatttatatcccctcgagcaacgaggacacgccggcggatgaggaggcggaggacagcgttttcacgagcacgcgggcgcgcagcgccacggaagatctggatcgcatggaggccggtttgtcgccctacagcgtctcctcggacgctccgtcgtccttcgagctcgtgcgcgagaccggcggcaccggcgccgccaagaaaccgagcgaaaagaaacgatcgtcgtcgcgtcggcaaccgcagatcgcagcgggcgcgcctcggggctcgccggcgacacccaaggccggcaagtcgcctaaagtctcgcgaccgcctagtgtgccctcgctgcccgagaacggcgccggcggcggtggcgacgataacagcagcagcggcggtagcagcagtcgcaccaccagtaacagtagcagaagtaccagtcccgtggcgccaggtgagccgtccgctgccgagggcgatgagttttccttctgcgacagcgacatcgaagactttgagcgcgaatgttaccgggtcagcgtggccgacaatctgggcttcgagcccagcgtggtcgcgccgcagcacgtcgagtatctcaaattcgtgctgcaagactttgacgtgcagcacctccgccgcctcaacgaatgcatacccatgccggccttcgcgctcaccagcctcgtcgaccccgtcttaaacaacgtagcgcctggcgagcgcgatctcacgcgtcggataatcacgcacgcggtgatcatcaactattactacgtggcgcaaaagaaagcgcgccacatggtggaggccatacggaccaccgtgcggggcgacacggtacgccgggtagccgcgcaggtcaacaaccagagccgttcggggcgtgcggccgcgctagcgcttcattttctcacgtcacgaaaaggagtgacggacggccagtacgccacgtctctgcggcggctggacgaagagctgcggcatcgcggcacgcccgaatcgccgcggctcaccgaggtttaccagacgctacgcgattacaacgtgctcttctataccgcccactacacctcgcgcggcgcgctctacctctatcggcaaaacctgcagcggctcaacgagaaccaccggggcatgctccggctgctttcggtcgaagagatatgcgaagagcacacgctcaacgatctggcgttcctagtaggcgtcgagcttatgatcacgcactttcaacgcaccattcgcgtgctgcgctgctatctccagcaccagctgcagagcatctcggagctgtgttacctcatctatgtacaactgccgtcgttgcgcgaagactacgcgcagcttagtgacgtgatctactgggccgtcagtcaaaactacgactacgcgctctacgcgagcacgccggcgttgtttgactttttacgcgtcgtgcgtcagcaggacgccttcatttgcaccgactacgtgtactgcgccctgcgtctgctggcctgtcccgacagacctattatcggtgacaccggcggcagcagtagctcccaacgcctcgtaggcgagtttatggtgcgcgatccgctgttgcgcgacccgcgcgccacccacctgcgccagaaactcatcacccgcgacatatgcgtggcgcggttgcaagcgcagccctcgagtcgacacattccggtcgaacacacgggtgtctcctccgtcaccctgctcaaaatctttagccaagtcccccccgacgaacgcgaagaagacacgttacgcgagatggctcttaaagcgtttatggaagcgaacggtaatcaccccgaacaaatctgccgatccccaccacccccgctgccaccgcgcgactatcctcaacgcgacgagcgggaccgtcaccgtcgcgaccgccgcgacagcggggaatactgttgctgatggtgggacgaaacagcagggcggaacagtttatgatagaaagtcacaggaaagtatgtgttgttttttttttaatgtaccaagaataaaaagtgcgtctacgaccaaagcggtgtgtggacgctcgtcctctctgtcttctccgggtttttttttcacgtgttttttccttcctattttgttacggcaacagcgctgatggcacgttgccggcttcgaacatcgcgtcggtgatttcttgcttgcccggcgtcacacggtgacgtagcagcacgcggctcacgtagcaggccgactcgcggatgacctggccgtcggcgtcgcgtcgcaggcccgagcggttgccgtgacgcagtctgccctgcgcagcgcgctccacgtcttcaaagtagctgtgtagcaggccgcgctccagcagctgcggcagcgagtcggcggcgcgcactacaaagttctcacggctgatctcgtagcacagcacgctgccgtcggccgccacgccggccacgctgcggtcccaactgaaaaggttggcgagtccgatggtgccgatgacgcgcaactgaccctgggtcaccaccagcagcttccagtattctacgtcgcgcggggtgaggatggtctcctccacgtcgcagacaaacaacgtgtagccgcgcggatagggcagatccaggtggcgaccgcgctggcggcgcataaaatcgtctaaattcaaaccgccgtcgggtgcgcgcctactcgtcatcgccgcgccttgtcggtcgatgaccccacggtgcttataacgcgccgccgcggcttcatgtggcgtgacctccgacctcgtgaggccgaaaacggcgtacatgaagacgctcaaacttttgaatgtgggcccggtagcgcaccgagggccccggggcggcgacgacggcgggcccgagttccagcggggccttgcggcggcagcggttggcgtggttgctcagctcggcgtccgagagcgccgagctgaactgcggcagccgcgtgcgatcctgcggcgcgtccccgtgtcgcagcgagtgccagagcaggcgctggacgcgcgccgtctcgggcgtcggcggcgcgcgacagccccggcgcagcttgaaaacgtgcaggcacagcagctcgcgcttgatgcgcagcgacacgctgcggtagtcgggaatccgctgcaccagctcgagaaagtcgcagaaggtctccacgaacgtgtcctcggtgaagcgaatgcgcttcagatcgtggacgtgtttgcgaaaccgcgacagttctcgacgttgcacggggttctgagcgagtcccttgcgcagcagcgcagcctcgcctttaaacagcctgatgagccgctgcacgtccccgctcaacatacgtatacacgccgtgtactcgtgacgtatactggcgcgcagcagccgaatgatacgcagggccagcacggcgttagaggccaggtacatggcgtagccgcgacgcgggttggcacaggcccagcccgcggggagcagaaagtagtcgtcgatcagcgtctgcgaccagtcggcgaagcccaggtcacgtgatacgctgtcctggacgcgggccacgtcgccggccgtgaggtggcggatcgccggcaggtgaaacgcgcccaggtgtcggttgcgctccagcctcagctcggcgtgctccaaacgggaatggtgggacgccaccgcggagggcgacaaagaggagtggtcattgccgtcgtggttaccgtcgtggttaccgccgttgtcgcgcccgtcgccgcactcgcaaaaggccgcgtagaggtccttcaatgccgcttcggctcgcgccataaacgtggcgtggaaaaaaacggcggcgcggtgcgtccggtacttgacgggcaacccgcggcacagggccgccggcaggcagcggccgatgagttcgcgctcctcgggctccagaaacaggcacagggtgccgtccaggcgcaggtacagctcctcggtcatcgagcatagctgccgcaagtaatgggtgcgcgtcccaaaggtcttgtaatcgagcaacgtgcacaccacgtattgccccgtggccacggccagagcgatgcgtttggcggcacgactgatctctggcaagtactgcgcctcgtgcaccagacggcggaaagcgccggcgttgagccagcgaaaatgctgcggatcgggcggcaagggcacgcctcgaagcgcggcccagacagcgaggtccgactcgagcgtcagaccgcggatgtcgtacttgccgtgcgccgtagcgcaggctgaatggaccagacagctgcggcgaatgtacaccatggcgtgcttgggatgtttgggcgccggcgttttctttttctgaccgccggcggccgccagatcctcgggcgtgcgacacaacaggccggcgcgcacagcctcctgtcgattacgaatcggcgtcaggtaggcgcgcaggaactggtgacaaaactcctcatcatcacgacagtcgtcgagatactcgtacgtggtgagcggatcgcgaaataggcgctcgtcaccgtcgtcatggtcttctttagcctgctcctccggctgctgggttggcagtggaggcggcggctgatccacggggttcatgactgagaggaagaagaaggtggcggcgaagcgacgcggagcgacggcggtaaagccagacaccggctatatagctagtcatcacagtctcctccttcacgacgcccccgtgccgctcacgctatccagcacgctacggcccgaaaacacgtactcgctgacgtcgtacgcgggcgatgtatggctgctcaccggtttcgcggcgacggttgcgctcgagtccaacggcgagaagcaaaaacgccgtgggcaacgaaaccagaaggagccctgacggataaaaccgcgcagcgtctcggccaacttaaccagcatcgtaccgtacagcagtacgtgaatgccgccatgcgcgtccataaatacggctttgttcacgggttccatccatccgatgactacaaaatgggcctgttctagcacgccgatcacgaaattgttggcctcgtcggcctcggccacgttccacgagccgaaagtgaaagtacaagcgggcgagccgcccaggcggatcttgctaccggcgtggagctgacatacgcgcagcagattggcgcggtcgtgcagtatctgggagagttcgtacatgcccgcaaaggtgtgcttaaaccacgcgccctctacgatctcatccacgtagtcgcgctcaaagaagctgtacacggcaaagaggccgttctcaaaaaactcgccgaacgagagccccagcacgtacaccttgtcctcgccgggcaggtacgcaaaggcgtgcccgtgcccggagacccagatctcgggcgccgtgtttgcgtccggcacgcattcgtacacactgacgaggccgataaagtacaagcggccagcctggcgcaggcacgagaagcgccggtaggtcttgtgatcgcgcaccaccccaaagtactgagtgtcgcccagcatgatgccgtgcagcggcggccagcacagcgggagccaacgacccgccgtggcgcgcacgtagcgctgcaggtgaaccccgctcgcacgctcgcgcggcttcgggcgcttgtgggtccaggcatcacgcagaccgcgccagatgctgctgaacttgggctgcccgcgcagatagagcgacgagagcgagtcaaagtagcccacgacgagcctgtcgggagacacaagagcgcgaaaatcaaacctagagcgacgacggtgaaaaaaccgaccagaagcgcgtgtctcaaacacgctactttcggttataaaaacaccgtcgccctatttctgggcgcgtgtacactgatgactcacctacgctttttgaacggcagtctcagctcgggattggcctcgtacagcgagctgcggtccacggggccgatgctctcgtagcgaaagtcgtcgatgagcagcgccagccccacgcgcacgaagcccctgaggtcgcgcgccagccgcaccaacttatcctgccccaccagcgccgcgtacacggtgcccgtgtcgccgcagagaatccgcacgcggtgaaagaaggtcttgtcctcggcgccctcaatttcgcccagcggcatgacgggctcgcgcgtgtacaacgaacgttgaaagcggcgcagcatcgaggccgagagccccagatcgcgcgccgtgcgcagcactagggaatgcttctcgggccagatgagggtcagttgcgcctcgcggtgcgcctctacgtaggcgcaacgagcggcggtgtcctcgcaggccagcaactcgcggaaagccagcagcgaacgtaggtagcggccgcgagcggaggcgcgcgagcggcggcacagctcggcccgatggtcgggatgcaccaagggcacgttaggttgcagacgcgcgcagatggattcgtgcaccgggtcgcagcggatcatgcccttggcaaaaaatccggccagatccgaggccaactcgtacaggcagtcctcttgcgcgtcgtaggcgaacacggcgccgtacgcgtccacgaacacctggtaccggcaggtggcgtgcgagaccgtgccaatgagatgcagagctcggaattcgccgaaaaagtcgttctggcagtgctccagatcgatctcggtcagcgagtgcggcgaatgctcgcccccgaccacgtagatgcactgcgagggccagcccagcgacacgcacgagccctcgaagcgccgcaagtaacgccgcaggccctcatagtcgcgtcgcacgcacaggtcggccaagtcgcgcgtgcaaaagacctcgggtaccaagcagcgtttgcgacgcggccgacgcgcgtgcccaggcagaggaggaaggcgcgacggcggcgacgacgaggaggaagacgccgtggccgccgagcagcccttgcgacggccggacatgccggcagtccgcgacgatccacaggagacaaaaaagcagaagcagcagtagcctcggcgacccgctccaccccgtcctccacacgctcagccgcgactgaacgccggggcgcgccgctacttgggtttttatagccatctgccccccgtctcgggcacccgggagcgatctacggagacctgacagcagttgggcaacacaagatagggaaatacaaagacacttttaataaaaaacgagactactttgtgtgtgtgctccgtaaactgtttattctccccctccgcttcgctctggatgggctccgggtccgtcaacacgcgactcgcgcggcaaaaggcacgctgttgacggcgcgagagcccgtcgtgatagtccatcatgccccggagatcgtgcacaaagcagctgtcgccgcgcagaaaccgacgcagcgtctccacgtgctgcagctgccggcgcgtatcaggagccgtcatcgctgatgtcgtcatcgccctgacaggcgcgtagatggcttcgcgagatcatgcgcgttttcaaccgccgtgacacatcaggtccatcttgagctggcgccgggcctcgcgcaggtgttgcacgcgttgtgagcgggaggcgagttcggcttcttgctcgaactcctgctgctcactgtccgagagggtgcgataaaaggcggcaaagtcctccaagtcggctacatgcgccctgggtctgacgctccaaagcgtacgcagtctgatgaagcggacccatcgagcgtcacggcacgccgtcttgaacgcggggcccgggaagaggttcttctccccggcgcgctcgggccggcgaggccgacgcggtttatatacaccgtctcggacggcgggacgccgagcccgcgccgcggccgctcatccggagacggcggaaaccgcgacgccggaggaaacggggaccggcaacgacggcggtggcggcgaccagattatgggggacaaacccacgcttgtgaccctgttgaccgtcgccgtgtcgtcgccgccaccgtcgtcgccgctgccgctcgtcagcttcacggagctgttgttaccgccgccgtccgtcgccgccgctgcggtggcggcgacagcgacgagcgaggtgggcgagaaaaccgcggagcaagaggtagcggctgcggatccggagaccgggaatgagagaagagaaaacagggagaacgaaggaggggagacgaggacgacaggcaccaccgcggtcaaaaggtcgcacgacggtatccctcgccaactggcagagcgcctgcggctgtgccgccacatggaccccgagcaggactatcgtctgccggcgcaggacgtggtgacctcgtggatcgaagcgctacgcgacgcggaccgcgacaactacggtcgctgcgtgcgccacgccaagattcaccgttcggcctcgcacctgacggcctacgagtcgtacttggtgtccatcaccgagcagtacaacacggcctcgaacgtgacggagaaagcttcgtacgtgcagggctgcatctttctctcgtttcccgtcatttacaacaacacgcagggctgcggctacaagtacgactggtccaacgtggtgacgcccaaggcggcgtacgccgagctcttctttctgctctgctccaccagcgagagctccgtggtgctgcaaccgctcatcaccaagggcgggctctgctcgtccatggcggtttacgacgaggaaaccatgcggcagtcgcaggcggtgcagatcggttttctgcacacacaactggtcatggtgcccttcgtgccgcacgcctgcccgcattacgccgtgcctttcacgacgccgggaaagccgggctgcggcggtgctccgagcggcgttgcggggttggaggaggcggcgccctttggacgggtcagcgtcacgcggcatggcgcgacgctgctatgtcgcgtggaccatctgacctggatcagtaagcgcgtaaccacgtacggacacaaaaaaattacgcgctacctcgcgcagttccgcggcacgatggacgacgacgaggcagcgctacccggcgaggacgaagcgtggatcgcgtccaaaaacgtgcagtacgaattcatgggtctcattttcaccgtcaacgtggattcactatgcgtggacgcggaacagcgccaactgctgggcaccgtggccacctccttctgtcaccgcgtctcggacaagatcacggcgcgcaacatgccgcgcgctttttccttctacctgctgacgagcgcgcagcgcgggtacgacctgcgattcagccgcaacccgtcactcttttttagcggcgacgcgctcaactgtccgcttctcaatgagcccaacgtgttttcgctcacggtgcacgcgccttacgatatccacttcggggtgcaaccgcggcagacggtggagttggacttgcgctacgtgcagatcacagaccggtgtttcttggtggccaacttgccacacgaggacgccttttacacggggctcagcgtgtggcgcggcggcgagccgctcaaagtcacgctgtggacgcgcacgcgttccatcgtgatcccgcagggcacccccatcgccacgttgtatcaaattaccgagggcgacggtaacgtgtactcgtacaaccaccacacggtgtttcggcagatgcacgccgccggaacaaccacgttctttctgggcgacatgcaattgcccgcggacaactttctcacgtctccccatccctgaccctccgtccgtcctcctttcccgacacgtcactatccgatggtttcattaaaaagtacgtctgcgtgtgtgtttcttaactattcctccgtgttcttaatcttctcgatcttttggaggatgttctgcacggcgtccgacggcgttttggcgccccccatgccggcagaacccggttgcggccccgtaccgctcttctggggcgacgataggtcgaaagccaccgttttcatgcccgtcgtgctcttgacgggggaacctacggcggcggtccccgtcgagcggcgtgattgcaaagccgcgctcgcccccggtttcaggatggagggggaggccacaggcggcgcattcgatacgctgcttttggccgtagacgacggtgggtaaacggtggttaccgcgggatacgtcggcgtggtcgaggcggcccggctgctgccggacaggcgacccggcgcgctaccgctcacggggaccgagggcggtcgacctaccaccgccttgccgcccaaagtaggtttcaaggaaggaacaccgacgcggctgccccggcctttcaccggagacgggggggcactcttggccggggacggagaggctgacgaaagcatggacagcggcgatgtggcgggggacacgacatcatcctccgtgggcgacaaaacggacgccgaggctgacggctgtcgagccgaagaagcggaagaggttcccgcgccagaagtcacgttccttgatgacgtcgttttagacgaagccggttgaggttgcaacagcgtggcgggtaccgtcgacggcgtgcccgacacctgtttctctagccttccctgaaccggcgtcgacgtcaccgtctgcgctcgggcggacgcgtgcggcgtcgcgactcgcttgcccagcaccggtttctggctcgtggatgtcgtcgtcattggagacgataacttagctttacgtattctggacggcgtcgactgctcgggcgtctgactgggaggcgaaatgacgtcgttgttgtaatcggacgacggtgttgtgtgtcccaggctgacgacggagccggtgtccgaggagtcgtcgtcttcctcctcgctgtcttcgaccggtgactctgcagtttggtcccttaaagcccaaacctcatcagcggcgtcccgagacgctgtttgtgtcaccgcggcgcgtggagtcgacggcctccgaggggtggtggacacggtgttttgagaagccgtggaagtcgtaggcatcctgaagggattgtaagccaggtgaggattcttgagggcccacgcgcgttcgcgcggccagttggcggggttcatatccccgggcaacggcgccgtcggagcccagggcgagttaccgttgaccggggtttgggtacccgcgaaggtaggtgtcggggccggagcgggggccgtggaaggattgacaggcgtcggcgtgaggatggcagcgccggcgccagcaggaacgttaactccggcgccgaacgtcaacgtcggttgctcgaacttgtacgcggtggtgacgggcggtttggcgctcgtctcggtatccgtgatgtccaccagcgtgtcggtgaaacgcggatcttgacggttggggggatagccatccgagctgtcggaatcctcgtcgcccgagaaaagatcccctctggtctccgtgagcggcctcacgtcccacgcgctgtcccgacggacccttcccgggctggccttggtcacctgcggggagacgagactgaaagccgcgtgacgctgttgttgttgcgggatgttcaagggaccgctggtcggtttctgactgcccgaggataacaggccgctgaaaatgctggaaacaccgccaccactagcggcgcccttgccgctagttcccggtttcttgatgggcgtaaagatgtttttctcgtcatcatcatcgtcgtcgtcctcatcggcactggagccaaagagcctccgggaggcgctcggtttacgtgccgggggcggtggttgctgctgacgttgctgcaggttctgctgcctctcctcccaagccttcagctgctgtttctcacgctgcaccacctcgtcgtccacccgtttctgccgctcgcgacgcttttcctcttcgtcgtaatagccgacggccgccgaacgggcagcgtgggcttcggcggccggtgccagagaaccatgggcctcgaagcggaacggtttgtgtcccttccagggactggcgatccagctccagccgtccagcggctgcgtggggacatgtttcttgggtaccgacgagaaggccgaaccgccgccgagcgagaggagattggcgtcatcgtcaaactccaacgacggcgagcgcgcgcccaaaaacgtgtgcgccgactgtgggaagctgtccacgtagatatcaaagtcctcgatgagcagctccaacagcgtgtcggccgagtcgccgttttccacggcgtgcttgaggatattgcgacagtagttggaatcaaaggaaaggcacatgcgcagctccttgaccagcagcttgcagcgctcctgaatgcgcgccagacatttgcgctccagctcctcccaagacctgcgcacgttcatgatgagacggcccgtgtacacgagcttgttgacggcgttgaccagcgccgtgttggcgtgccggtccaggttaaggtcgagcggtttcacgcagaacatgttacggcgcacaccctccaggttttcttcaatgcgctgcacctccgtatccttgaggtgcacaaaggcgatgggttccgtctggccgatggctgtgaccagcgtctcgcgcaccgacatcttggccagaatgaccgcgcttacgagcgcgcgttccacgatctcggcatcgtggcgcacgtccgtatcgaattcggtacggtctagcacagccaggtggtcacgcgccttaccacgatcaccgaacgggtaagtgtagccgcgacgcgccacggccacgcaacgcacctcgaactcctcgagcactgaggagaggtcggggttgtgaaaacgcagctcgcggtagtatcccaaccaaagcatgagctcgttgaacagcaccgtacgccggtgcaggcgtttttcgccacattttttcaggatcttggggtgtgcctcgagatccacgtcgggcttttgcgtgagatggcgcagaaagttgaccagggctaccacatcgcgccgctgtagaccgataaactgcaaactcatgctggcttttctccagaacccggaagcgtcgtcgccccggactgcgcccgcggtctgctattcgcccacgatggacaccatcatccacaactcggtgagcgccccacctagagggagggggggtagtttaatagcggaggcggatacgcggttttcttttaagcgccgctgacttgtttcttctgttttttcgccccgtgtgctgttccgcccagacccgcaacaacactcctccgcacatcaatgacacttgcaacatgacagggccgctattcgccattcgaaccaccgaagccgtactcaacacattcatcatcttcgtgggcggtccacttaacgccatagtgttgatcacgcagctgctcacgaatcgcgtgcttggctattcgacgcccaccatttacatgaccaacctctactctactaattttctcacgcttactgtgctaccctttatcgtactcagcaaccagtggctgttgccggccggcgtggcctcgtgtaaatttctatcggtgatctactactcaagctgcacagtgggctttgccaccgtagctctgatcgccgccgatcgttatcgcgtccttcataaacgaacatacgcacgccaatcataccgttcaacctatatgattttgctattgacatggctcgctggactaattttttccgtgcccgcagctgtttacaccacggtggtgatgcatcacgatgccaacgataccaataatactaatgggcacgccacctgtgtactgtacttcgtagctgaagaagtgcacacagtgctgctttcgtggaaagtgctgctgacgatggtatggggtgccgcacccgtgataatgatgacgtggttctacgcattcttctactcaaccgtacagcgcacgtcacagaaacaaaggagtcgtaccttaacctttgttagcgtgctactcatctccttcgtggcgctacaaactccctacgtctctctcatgatcttcaacagttatgccacaaccgcctggcccatgcagtgtgaacacctcacactgcgacgcaccattggcacgctggcgcgtgtggtgccccacctacactgcctcattaatcccatcctgtacgcgctgctgggtcatgattttctgcaacgcatgcggcagtgtttccgcggtcagttgctggaccgccgcgctttcctgagatcgcagcagaatcagcgagctacagcggagacaaatctagcggctggcaacaattcacaatcagtggctacgtcattagacaccaatagcaaaaactacaatcagcacgccaaacgcagcgtgtctttcaattttcccagcggtacgtggaaaggcggccagaaaaccgcgtccaacgacacatccacaaaaatcccccatcgactctcacaatcgcatcataacctcagcggggtatgagctttcctgttactttattcagaaagcaccagaacccgtcgccatttcccctcatatacggtacacgtccccctgatctgtcatcacggtacacagatttcgcccgactgcggacgccgacggccaatcgcgtggcgtaggagtggcgccccggcttcattataacgccacgtcggagcccctgcgcgccacaacgccgtccggcgcaacttctgtctcggcacggtacgataaaaacaacgtcccccgtcgacgttgttttctccgagcggtgatcgttcccgtccctctcctccctccgcggcccccacggcggcggcctgctcgcacggacctatactattaccgccccaccgccgtcgtcgtcatgaacttcatcatcaccacccgagacttctccaacgacgattcagtcctgcgagccgccgagatgcgtgacaacgtggcaggctcgatttccaaagcgtacaagggcacggtacgcgccgaaggcaagaagaagctgctgctgaagcacttgcccgtgccgcccggcggctgctcgcgccgcaacagcaacctcttcgttttctgcaccgagcgcgactaccgcaagttccaccagggcatcgcacagctcaagcgcgcgccggccgaactggacccccacgagatccagcaagtcacggccagtatccgctgccgcctgcagcccagtctccgcgagccgcccacgccggccgacgagctgcagacggctgtgtcgcgcgtgtgcgcgctcttcaaccagctggttttcacggcccagctgcgccactactgcgagcaccaggacaaggtggtgagctacgcgcgcgacgagctgactaaacgctgcggcgaaaaatcggcgctgggcgtggaagtgcatcaactggtagccctgctgccacacgagcgccaccgcgaactgtgccacgtcctcatcggcttgttgcaccagacgccgcacatgtgggcgcgctccatccgtctcatcggacacctgcgccactacctgcagaacagcttcctacacctgttgatgaactcaggtttggatatcgcacaagttttcgacggctgttaccacagcgaggcctaccgcatgctcttccagatcggtcatacggactcggtgtcggcggccctggaactctcacacggcgcggcggccgggccgcccgaggccgatgaaaacaacgacgagggagaggaggacgacgacgagctccgtcacagcgacccggcgccgcttcacgagtccaagaagccccgcaacgcccgtcgtccccgcacacgcgtgccgcctcacgagcaaaagcccgaagaaaacgaggaggaagaagaggagctgtttccctcctgcaaggcaaccgcagcattcctgcgggcagaaccctccgtctccaacgacgacggcaacggcggcgaacgctgcgacacgctagcgaccgccctgcggcatcgcgccgacgaagaagacggacctctagccagccagaccgctgtgcgggtcgccgcgaccccctcaccttcagtcaccccagcccttacccccgtcacgtcccccataaccccgttgtgtatttaacgtcactggagaacaataaagcgttgatttctcaagttccgctctggttttggtttcgttttcaaagggagccccatcatggcccaaggatcgcgagccccatcgggcccgccactgcccgttctccccgtggacgactggctcaactttcgggttgatctatttggggacgagcaccggcgcctgctgctcgaaatgttgacccagggctgctccaactttgtggggctgctcaactttggcgtgcccagccccgtatacgcgctggaggccctggtggacttccaggtgcgcaacgcttttatgaaggtaaagcccgtggcccaggagattatccgtatctgcatactcgctaaccactaccgcaacagccgcgacgtgttgcgggacctgcgcacgcagctcgacgtgctgtactcggatccgcttaagacgcggctgcttagagggctcatccggctctgccgcgctgcgcaaaccggcgtcaagcccgaggacatcagcgtgcacctaggcgccgacgatgtgacattcggcgtgctaaaacgagcgctggtccggctgcaccgggtacgcgacgcgctggggctgcgcgcgtctcccgaggccgaggcgcgctatccgcgcctcaccacctacaacctgctgttccacccaccgcccttcaccacggtcgaggcggtggatctgtgcgccgagaacctgtccgacgtaacacaacgtcgtaaccgaccgttgcgctgcctcacctccatcaaacgcccgggctcacgcaccctggaggacgcgctaaacgacatgtatctgttgttgacgctgcgacacttgcagctgcgacacgcgctggagctacaaatgatgcaggactgggtggtggaacgctgcaaccggctttgcgacgcgctttacttttgttacacgcaagcccccgagacgcggcagactttcgtcacgctggtgcgtgggctggaacttgcgcggcaacacagcagtccggccttccagccgatgctgtacaatctgttgcagctactgacgcaactgcacgaggccaacgtgtacctctgcccgggatatttacatttcagcgcgtacaagctgctgaaaaagatccaatcggtctcggacgcccgcgagcgcggcgagttcggggacgaggacgaagagcaggagaacgacggcgagccgcgcgaggcccagctcgatctcgaagccgatcccacggcgcgcgagggcgagctctttttcttctccaagaacctgtacggcaacggtgaggttttccgcgtgccagaacagcccagccgctacctgcgccgacgtatgttcgtggaacggcccgaaaccctgcagatcttttataacttccacgaaggcaagatcaccaccgagacgtatcacctccagcgcatctatagcatgatgatcgagggcgcctctcggcagacgggcctgacacccaagcgcttcatggaactcctcgacagagcgcctctgggccaggagtcggaacccgagatcacagaacatcgcgatttatttgccgatgtttttcgccgtcctgtgaccgacgcggcttcttcgtcgtccgcgtcttcgtcgtcgtcctcagcatctccgaattctgtttcgctgccgtctgccaggtcgtcatccacacgaaccaccacgcccgcgtccacgtacacctcggccgggacttcttctaccacaggtctcttgctctcttcttccttgtcggggtcgcacggcattagctccgcggacctggagcagccgccccggcaacgacgccgcatggtcagcgtgaccctcttttcgccctactcggtagcctacagccaccaccgacgtcaccgaaggcgacgcagcccgccacccgcaccccgagggccggcccacacacgcttccagggacccgacagcatgccgagcactagctacggcagcgacgtcgaagacccgcgggacgatctggccgaaaacctacggcatctctgaaagcggtttttcctctttttctacgtgtctgtctcaagatgagacgtcgatatcaataaaaataccgtcgacgtggtttttttaacagtgtggttttctttattgactagcgaagtacacagtttacgagtagaaaagacagggaaaggttatataaaatgctgtattatatacaaaaacatgcacataaacaaacgggaccatcgtgctcatcatcccctccttgatcagttgttcatgtaaacgtgtggcggggtgaggggcggcatgccgttggcggcgccgggaataatgtgccgtcgaccgacgtcgcacaccttgaaacgccgtcggcgcacgcagcggtcgcaggacgggatatcccagaggaagcccatgtaggtctcggggtcctcgtcgtgaaagcggtaggagagttcaaagtggtgcaacgagcccgtccgagctcgcagcttctggcgaacaccctccacgtcatcggtgcacagcgacagtgctgggctgtcacacagggcctgaagctcctgcggccacaggtgcgtggccaggggcgagtccgtcgtcaccagtttgacgcagtgcatcaggttctcggtgatggcgtcgtacaggcgactctcagcctcctcgtgcgtcatcacgtttcgaggcagcgacagctcgtcgtcgtcatcctcgtcaaacatgatcatggggtcaggggtttttttgggatgttgacaggtgggtgtcttttccagacgcacgatggcctcacgccggccgctgaaacggtggtttcggtgtcccttctttcccatgacgcaggtgaacataaccacgtcctcggccaaacggtagacggcgtccatggcggggtcgtagccgtagacgacgccgaaagtgtccaccaagacgtactggcgtacgaggaactctttgcgttctggcacctcgtggcccagcgcgcccaacaactggtggtaacaggtgatgcgcggcacggtacggatcatgagctccatggtctggatgctgccgcccgcgcggacgacgctgaaggatgtttccttgaacttcataacctctgtgttgtgggtccagaaggcgaaatgggtgtcgggacactcatcgaaagggtcgtcgatggtgtaggaagcgtagcctcgcttggtcacctcggccgacaggctctccacgtcaccgcggtagagcatgacggcgttccagtagtcgtcgtactgcaccatgggccgctggtagtcgcgcatagtgtggaagtggtcgcggtgacgaaagccgttccgcagaaagtccttcatggtgggtgccagctcgtagacgcagtcgcgcaggtcatcgtagcagtagatgccgccgcgctgcccgatgagcacgatgagttggtagcgcataaagcccggaccctcgacgaagccaaaggggtgcaggtactcctgacagcagacgtaagcacctggtagagaatagaaaaaatccacgcacgttgaaaacacctggaaagaacgtgcccgagcgaacgtcctctttccaggtgtcttcaacgacgtggggcttaccttgcgaacagacggtgcccatcttgcccacgaagggccccagggcgctgcgcgaacggagctggatgaagcagcgttcgggccaggccacgtgcagccgggtgccgcattcctgctccagaaagtcgttgagaccgttaaagtccccggctcggatggcgatgcagccgtaggccatcagcgtgtcccgtaggtcgtccatgacggactcctctaccttcgctcgccgacgctgcgcttctccagccaccgctgcggtcgacagactcctccgtccgccttcggagaactacggcgcggcggcacggcctttatagacactatcagcgttgacgtcagacgatccgatgaacgtcgttttttgtgctggaacttccctcgtcccgacaaatgtagcggaaatcttcaagcaaatcgcgacgaagtccgatgaggaggatgcaaaagaggctgagcaacgcgatgctgcccgccgccacagtacatatgctcaacaacgcccagtgtcccaaggcgcgacttttggctcggaggagagccgaacggcggtttctccacatgacggacaacgtggtccagtacgtccatcctttgcattccggtgtccagacgggaagcgttgtcatgttatttcccgtaactgtcacgttatgttttgttttgtttctcgtgagcttaacggtcctcttgagaaatcgcgggcacatgtcttgtagaaagatataatcactttccgcgtatttcgtcagtgttgacatcacggtggtagtgttttctgaagaagtagcgttgtcagtgacgtttgtttcttcccaacgtacgtatgattcgaacggactcgtgtgcgctattgcccgcaacacgtagctgtggccggtgaagttgagcgtcagttgtcccacggtcacgttcgtgtcattcctaaaacatgctacttctccgtgaacttccgtgacgtttatctcacgactctcgttcaagacacgcaggggaaaccagccttccaggtgatactgaaaaccaaatttaagcatgacgctgtgccatttccgtcgtgattgattaaacgttacattcaagggcagtctggcttcggtcccgagacaggggccgttgtagatttgcgtgtgattgcgtgtgcagtttaggtggcagttcatgctcgtggtgttggaagtgcgattaacgtccgtaccgtggtacgtacatcggaccgaaacaccgtgtcccgtgctccaaagcagcgtcaacaacagccacacagaaacctacgtggagacgacacgggactttttattgacggagactcacgtttctaccctcccctttcccgtaggtaaaaacccacgtttatcacacacgttgtttttacctgaaacccgcgcagcccgtggacgcgacaaaaaaccgcggcactagaaagaaaatgaaacaagtatgtttattaagcagcatgtggggctaataggggggataactgaggtatagcaactatgaaaaaatactacaaaaaaaaaagctgaacatggtcatctagcagcaaagttctccttctagaccacgaccaccatctgtaccacgtcgccctccccggccgtgtacacgacatccttcaccacgaccggcggcagcggcggcgacgaggacaactcgctctcgacggaggccgggacgacagaggacgggggggtggtggcggcggaggacgaaggggtggcggcggcagcgggatcttcttccgacacgggcaacggcaggctcggcggcgcggacagcacccgttgcgccggggcgtgagaaggctgagccccggtggcctggatgtgggccaacgaattggctcgcagcgagtcgcgatccacgaaggtcataggaattttcccttcgcggatccgccgctcagattccaggatggcgcgcacgtagctgttcaccgacttggcaaaagtgcgcggcccctccgtattcttgtcgcgacgcgcttccagcacctgcttttcgtagtccagctggtggaagaccatcaccaggtcgtccatagtgtgcgcgtgctgacggacgtgggagcgcacctccaccgggaacaaagcgttccaatactccagcacgatagcaccgtgccagaactgcgccatgctgggcgccaggaaaaacaggataccggagtcgtaggcgaacacgtcccacttgggcgtcatgaacaacaccagctgacgcgtgggccgcaccgaagcttcctcccaggcctcgatgaccccgaacatgatgagctcctggtccaacggggggcagtgtcgctccagccaactgatcttgctcaggttcatctgcagaaactcgtacgaagggtcgcagatgcacacgtagagacccgagtcgtgccgcagcctggctccgcgcttcatcagtttcctcaccgcgtagcgaagcgccaccttgcccaacgccgacgcctggatcagtccccccacgtccatctgcgtctgtcgccactcggcctcgtccagcaggctcatgatagcggcagtgctatgcgtggtcgtagtcatcctttctatccttctctatgaatagcagcaatagcggtaaagtcccttcttatactatcccggagtctgtggtttttttgtttacccctgcttactggtgagactgctgggggccgttgtgctgcagcatccgagctcgttgccgccgttgccacaggaaccggtgtctccgcagggcctttttgagggcttcgcaggcttctcgcgcaagtcctgagaggccctcggcgtcgatggggttcacctcgggcgtccgagcctcgttttcttcttcttcatcctccctttcctcctccgtgtcctcccgctctgtgtcctccgttacgctctcctccccggcctcggccaagagcgcggccaccaagtccacggaccgctcggtctccgagttctcaccgtcaattacgccatgttggcggcgtaaccggtgccgagaacgccgggtgagcgcacatgcttttttctttcttaaccaaggcgggagaggatcttcaaggcgttttcgctggatccagcggtagctaaagtaccaaaaggccagcaggcccacgctacctaacagattcacgtagactggagacataattaaagaaagaagtgaaacccgcgtgtgggtctcacgtcgtcttgaaacaccgtcttatatacatgaagatgccggacatgacgcgcccaagacacgtggggttttccccttaggcgacccggtttcttaagatgtttttcatcttcgcacgcgatgtactacatcaaagggtcggctgaccgaccgcattgacgcacagtttccgagtacgcgcgtctcggagcacctgacggtgagccacccaactcacgcggataggggacaacactgacgtgaggggcgattcacgtcactgacgggaataagacgggtgagggatttccacctttttcttaagtgtgactctctttacggtaaatcgcacctgtgacctcttaacccctcctccctggtacccgataaccgtgaaaaacacacaccacacgtcacgacaccgatcgattttctttattcttagtgtgatgataggtaagggcactcgtgaggatgtgcaattatcattatcaagcctttttcaaggcgtagtgatgatcgttgggcagaacccccaggctcctagcgatctgggaatagaaggaggagaacgagcccagggccagaatgcccacagtgtacatggcccaggtctccagaccgaacgtggcgggtcgcagcttcagatggtaggccacccgctccgagagttgtgaatgctcgttcaggcaacaggactgcaggtgggtgagcccaaaagcgctttcgtttacgccgcgcacgtgcaccgtctgggccgggcaatcctggtgttgcgcgcgaaagtggtcctgacaggaaattccgtctacgtggcggcgcgtgttgttacccacttcgatcaacaacgtgttatcggcaggatgatgcgagaacgcgacgacggtgttgttggaggtctggcggcaacagtacacgtcgagcgtcatgagggccatgtcgccttggtggtacacggcgtacgcccaaccctggaacacgagcggacataacggaccgtgagcggacgtcacggcggcggttgttaccgtcgtctcggcaggagaacacaataaactcctgatcctcatacacaggagtccaaccgtcagaattaaagtccgcggagccataaccgcgcaagtgaagccgatacgagtgttgctgaatttgttcattctgccgactgttgctcacgagcgttcggaggcggtgccacaggctgttggccattaaaaagtcctggcccgaatgacgacaagacagagcccgaggcgaagaaaaaggcgcccgtcatgaagacgtaggcaggggaattcccatatttttatggcttcttttaaaagtctgtatccgactccatccggcgcttttcccaaaccgtggtctcctcgtcgtccgactcggtacccaggaggtggtaagtcttttgccgcacgtagaaagctttcaacgtggagcaaaagatgagaataaagaccccgaaaacgaaacaaaccacgccgatcatgccgatgcagacgttcatgtcgacgtagccggcggtgctgttggcggtgcggcaaaagagtgtcatgtcgtgcgtgcacaaaaaacaacacacaccacaggccaggtcgtagcgtagttattattccgtagcagcaatgatggtacagtcaagcacatgctctatccccgttaccccgatgatgcttgcgtccccgttgttatattggcactgtcccggttaatcaccacggtgaacaccacggccaagaaaatgatccctaatatagcgaccactaagagagcaaaagtccatttccagccgttgtcaaagtacgcccccgtggtgggatgcatggtggcgggcatttccatcatgtccatgtcgaacgtgtgtcgcggcgacggcgaactaaccaggcagtacgggggtcgatagggcggtgggctgcagtcgggtggtggcggcggtggcgtggaaaccgtcgtcgggcacagacccatggcctgctcgtaggtggggggcgcgtcgtcgtgatcccggtcgcggagcatcggcgtgggctccatgtcggtggcagtgacggcgacggtggtaactgtggtggagacggtaccgacggcgtccgcggttcaccttcgagcaaagagccccttctttttgcgcaaacgacggcaaaacagttctctgggacaaccggtggcgcggtaagcgggtgccacgctttcagggtgggtaaaacagtcgcgggcgaagcagtagttgttgcagaaccgcaagaacccgacgcgaaagaagcccaggagtccgcgcgccagaaagtgcgcctgccgcgtctcgggatgcacgccgaagacggcgccgctctcgttcaccagtatggagatgtccaggcgctgctgcgactccaccggcacggcccgcaccacaaatacctgcagcacgttcagcgagcacgtctcttttaaccagttgccgtgggccggatcctcgtaagtctggctcccgttcaagacgaccgtcgtcagcgcctcattaccgtctcgccagctgaagatggaaccctcgcgcttcatgcacaggcgccacaaggccagcaggtcgcgcgccaacatgaactcgcgacccacgtcgccgccggtctcgaagcggacatagcccagttcttcgcgcagcggcgcgtagttgcgcaggccctcctgcacgaagccgcggaaaccggaccgcgacaccaggtacagcgattccaccacgggcgagtagacgtagacgcgaccgccttcgccgatgagtacgggtagcggtgggcggccgatggcttcgcaacgactcacagtgcccaccggcagcaggaacttgtcgcagcacaggaaggtcttctccaaacctttaatattgagatgtccaaagtaaccaacgcgtaacaggtcgcagtaggtgaagaaccaaccgtttggccagctgagacgcagcaccgtgccgctgacgcgacgaaccagcttctgcaggtccttgcgagcgtcggaggtgacagagcagcggaaggtctcgttaaccagctcgacagccagcgcgtcctccagcgtgcgttccttcatctcgtcgttgatgctctgacggcgccgccggatttcgtcgaaacgggccgcggaggcggcgaccgacgcggaggtcgtccgaacgccctctgtgacgctgccgtccggccagtcaagaaagctaaggctggcgctgcgccgcctaaagtgtccgatccgcgcgggacgtcgctgagggacggtggctggtctgctggggcgggtacggccgcgggtgtccgcggacacgttagttatacacggaattgagtcacgtggcacgttgccagctgaaaccgccgtcgtctccgccggcgttttctccatcacgggaccgcgccgtgcgcgcgttcccaggcacgcggcccgcgctctagccgcacttttgcttcttggtgttagggacgaactcgaacgttacagaatcctcgctgtcgctctcctctttcgcgtcgttgaagtaattgccggagttgcgatccaaaccgccgcctcctcctcctccgccgccgcccgatccacctttggacgtcaggtagctggtgatcttgtgctgctcgtatttttccttggaggaaagaccgtggtcgtgatcaccgccgccgccaccgctgctcattttccgcgtaccggaaccaccgccgccaccgcggtcgtgcttcttgccgccaccgccgccacctcctcccagaccgccgagacccatgggctcgttcatgagatcgttatccagacccgggccgtcgtcatgcagaccgccggcattggccagcgaagagaggctgccgccaccaccgccgccgccacgcgacttgccgctgttcccgacgtaatttttgtcgaagggatcgccacgctggaaaggttcctcggtgagaaaattctccacggcgaacagaccgttgcggctggccacgtacaacagcgtgtcgtgctccgtaactatacgcaacgtgcacggcagtttggtgacggcgcaattgagcagcgtctggtagaagttcttcagctgcacgttgatacgcatgtttttcacgccgtggaaactgacgcggttattggctgtgaattccagctcgctgccgttggtcaggataaacttgatggccggtggaccggcgtgcaccagaatctgcacggtgcccgtagggcagggcgcttttttaacgttacgcttgacgcgggtatgcggcccgatccacttaagcaggtcggccaccacgccgaaatctagatccacgtgcacggccgaattctcgctttcgcgcacaatgtcttggccgtgcacgcaggccgagctgaactccatattgaaatcgggcgcgcacatggagatcttggccgacaggtccgagatgtcctgcacgtagaacttggtcaggtccttgctggaagtcaggtacatgaaattacccagcagcggcgtggaattgttaatggtcttgggctgaaacgacttgtcagtgatgtagaggcatgagctgttaaaagtgatttttgacacgcagtgactgcgtaccgtttgcaagataagcgacggcgtgggcaagaaggtaaccgtggtgttctccttgagcgcacggatcacagatcgcagctgctggatagccgtcttgtacggcttcagccgcagcgccagcgtcggcggctccgagaggcgcgtcttgcgatccatcccggacagcgtgcaagtctcgactaaggagcgggcgcgagcgagcgaaagttttatagagagcacacacgacgaccgggaacgctgcgaagacgcccggcgtctaataatacagccgcgccgagccagcgggcccccgactaagaggcacagtacttatatactccgaccttaaagcgccagtggtaccacttgagcatcctggccagaagcacgtcgggcgtcatccccgagtcatagtagaaaaccagggccacgcactggtccacaaacacgctcaggttcacggccgccatttccacgtcgttttggatcgccggtgccgcctggaacagacactgcgtcgccttgccctcctcctggtgctgctccaaccacgcgtaattcaccacgggcacgcgcagcggcctccgcaccacggtggggaagtaacactcacggttgggcgggcacaatgaccacaccgtctcctcctcgaacacggtgccgcgcgaagcccacactgacggcgtcacgccccacagatgcgccacctcgtcgtcgggacccaccgccagaaactgacagttgcgcaatccgaactcgagcatgtcggcgcgcagcgcttcccagcgcgcgctggcgatggagagccgcggcaaccgatacaattcgaaaatgaatttgccctcttgatagatggtgcgttcgaaccactcgcagcgcggcaaacccgacttgcacaaatcgacgctagcgcgcaccgcggcaaagtacatgtgctcaaagatgcgctcgatcaagtcccaagaggcaaagtacgtgaaccctaaccgcatgagcgccgtgtgcaagccagccacgccgatgtgcagcggacgcagtttttccagcgcgctctctacccaccattcggacgccgacattagcgcgtccaagcgcgcgttgccccaaaccaccgcctcggtcaccaactcgcgcagcacgctcaaatcaaagtaacgtcgcgtgttccccaaaaccacgtcgggtagatgcagcttctgctcgtcgctacgcgcaaacacgcagcgagccacgttcaccgtcagccgctgcaccggcatgtcacactcgccaaagtggcacgacgccatatcgggactcaagcacggcggcaggcacacgctgtcggccataatcgagtacttgactacgtgatggacaaagaccaccgaggcacggcccttgagcgcgcacagcaacatctttttcagaaaatcgtccgtgttcacgaccaccttggggcacgattgctcgcagcgcgaatactctttctcgaaagccgactcctgacccaggtccgagagccgccgggagacaggccgcccaaacagcgagtagcgctgctcacgcgcacggtagcgcttcattaacacgctaggcacgttgaaagcgtagcaaacccccgtcaactccgacgtgctttctttgagaataaagttaatcacgcggatagcggccacgtcccacatgtccacaaacacacgtaccacgggtcgatgcacctccttctcgcgtatcaaatcgcagtatccccccaggcaacgaatcacgctgttcacatcggcgttaagtcgcgttacgttcaccgacacagaaacgccgcaactcaaggtactcatccacttgcacatggccgcccaactggcgtcacgcgagaaagggtcggccgagatcagaaagtcgtactgcggcacgcgatcgaaacccacggtagacatggtgaaggtggacagcgacagctgcccatcgcgacagcgcttcaacaccgattccaacacctcgccctcgaaacgcgcatccagatggaaacgatagatgcgcgagtgcctactgttctcgatagcggccgtcaacgccacggcgatgcgcaaaaacacgccgcccgggctctcgtcctgtccgtgcagttggcgacacaccttatccaaacacaaaatggccgcgtacaagccccagcaaccggccaattccacaaaacgcgccgtctcctcggccagcttgggtagatcctccatgtgacgcagcacaaaacggcgcaccgactcatcgcacagctccgaagcgtaacacagtggcgtgcggctttcacgcgcccagttggctttgaaataaaagcgacccaacagcaggtcgcaacgcggcgagtgacgaatcagacagggaccgtggcgcataatgagctgaaatagcctgaaactgcccaaaccggcactgtgccgcgacacggtgtccatctcgcgccacagcgcgttcctgtcggacggcagctcccgcgccggctcctgtacgccgcaaaagcgaaacttgccccagtagccgtgacaatgacactttttgcccatcaacatgcgcgtagcttgtatcggcggcgatactttgcagagcgaagccccgaaatcgtcctcctcctcgacactgtccagctccatcctggtcgcgccggccggattaaaggtgctcagaccgctactcacgcgtccaccgcgactgggcacggcgggaccgctgtcacgcgtcaacgacagcacagacggcgtgccgtcgggagacggcgactcgggacgccaactgacgacgccgccaccactcgtaaaacccgctacacatgctacaccgctcgatacgttggtatttccagcggacgcttccttgtcacccccgggcagcggcccctcctcgagctcgctgtcatctcccccgatagtatcagcggcgacctctgccgacgattcctccgtctcggtttccgcgctgcggcttggaatcctacctggccggcaccgatgtgcgggcaccgaggacacccgctgttcctcgtccgcgtcagccggattcataagtttacgaggaaaataacaaagaaatcaggtagatttcaataaagtgagtctagatggcgccgacaactacggtttataaagtctgtgtgcgatgtgtttttttcttctgtgtctcctccccgtatgctgtcagcgccgctcagacgaattctcgaaagtctcccaattcgacgctaaagttgtccaaacggacgacggacagtttgagttctttgtgtaccaggaacgaggtgtgaatgtcgtcagccaggcaccagcccagcttttgtatgaccccggtacacagagggatctggcgcgggcgcgtgatgcgacggttgacaaagctacagcgctcgcgggcgaactttccgcgtgcaacgtcgaccaaggtctgccagtgtgcgatgctggaggtgagcacgtagatgccgggacgtgtttcgggcccgtcatagtcatagacgatgattaaatacacgtattgcagccgtccccgggtctcttcccacgtcaggtacatgtctttcggtatcatcaacgcgaacacctccgttttgagcgtgttgtaaaggtagccgcgcatgacgcaggtgagcaacgaggtgatgcccagcgagacggtcttgacgcagcccagcgtctcgaggcggcggtgcagcagatgcgggcccaggtccagccactgcagcgcggcgcgcgcggccgaggccgtgtacacgctttcgagcaggcagcgcgtgctggccgagacgttggaggcgcgaatgcctaacaggtaaaggctaatgtagaggtgtcgcggcgagtcgcaacccgtctccatgcggatgagcagcgcgcccggctgcgcctcgaactctaccaggccctcgggcacgaagaaacgcgccgtgagcgcctggtgatcggcgtggtagagatagcgaaccgatatagtatttacctcgcgtttggctttgagcgccgtcactagttcattgtcctcgtcggccgggtcgcgcggccgtttggccaccgcgcgcgcgtccatgatggcgaggcgcacggtagatttcaaaaagttgatagagcagctgcgggcacgggccacggacaaagcggaggcgttaaataccgtgagccaattggagatcggcgcggtggatgcccaggacgtgaccgcgagcgccgtgcgcgccttcgtgggtgcgttgccgagctcgggctaccactttggcttcgtgcgtcagaacgtggtcttttacctcctaagccacgccacggtacagacggcgcgcgacccgctgtacgccgccgagcagttgcacgaacagctggaccgcttcctgcgacaccagcacgacggcggcggagacgaggaccggttgccgttctaccacaacggggccacgctgacggctttccagaagctgttgcagaccctgcgcgagatccagaccgtaatagccgaacagagcggcggcaccgcggcggcggcggacttgatcgccagtaacaacgcgtcgaccgagcgccgcggcaagaagggcggttcgagttccgggggccagcagccgctggtccgccgggtgatcacgcagctggaaacggctgccacggaggcgcggccctacgtcaattgtcgcgccgtggccgaactcctggacctgacctaccagcggctcatctactgggcctgcacgctcatgccctacgtgttgtttcggcgcgacaccgacaccgaactggacacggtgcttctgatgcattttttttacacacactaccgttcggttaacggcgatttggccgtggagtttcaaaactacgtcaagaacagcgtgcggcacatgagctctttcgtcagttccgatatcgacggcgaccagaagcccggtgccgaacacatgcgtgacgtcagctacaagctgttcgtgggtaatctgcaagcgcgtgacgccagcggcctcatgtttcccatcattagcacgcgcatctccaccgtgaacctttacctgtcgcccgaacgtatgtttttccacccgggtctgatctcgcgtctgttgagtgaggaagtttcgccgcgcgccaacctagacgcttacgcgcgcgtgtgcgatcgcgtgctggaagaccacttgcatacgccgcgacgcgtgcaacggctactagatctgacgcagatggtaatgcgactggtggaactgggtttcaatcacgatacctgcgcggcctacgcacaaatggcgctgatccagccggccagtcagaagagctcgctctttgtcagcgagattcgcgagaaactcatacagatcatctacaatttttacacgtttttcatgtgcctctatgtgtacagccccacgttcctgttcgaccaccggcggcggttgattttggagcagcatcgatccacgttgatcggctccaaggaggaactacagcacgtctggagcaacgtgacactgaacgtcaatacgcactttgcggttcagtacacggaagaagactttgaggcacatacgaagggtgccacggaggcggagcgcgagtacctgtatcgggacctgcacagcaagtggggcgtgcacctgtttaccttgcgtccgtctcgcggcgcggccggcgcggcctcgcctttgcctccgcttgacggcgtcacacgctccgacatcttacgcgaatgcgcgctcgttaatctgaacgaaggccgcgtcaactacgcctccctgctagccttcagccatcatcccgagttccccagcatcttcgcgcagttggtggtggtaactgagttctcggagatctttggtatcccgcagggcctgtttcaagccgtgggttcgccgcgtcttttcgcactcattcagctgtgccgtgtattgttgcccgagcaggtgacgctgtaccagaacctggtctccatctacaacctgaccaccttcgtcaagcacatcgacgccgcggtttttaagacggtacgcgattgcgtcttcgacatcgccacgactctcgagcacctcagcggtgtacccgtcacgcccaatgtggacctgctggccgagctcatggcgcgctccgtagcgcataacctgtacaccaccgtcaacccgctgatcgaggacgtgatgcgcagcagcgccggcagtctgagaaactatctgcgacatacgcgactctgtttcggtctggcgcgtggccgggcgcgcctctcggaggacggcgtgacggtgtacgtggaggtacaaggtcaatacggactacgcgtacccaccacgcgtttcgtagaacagttgcgcgagctggttcgccgcgatcggctgttggccgagaatctgcgcggcttgaacgagcgcctgctgagtgttcgcgtgcgcgtacgtcagatcagcagcgacacagaggaagtaagccgacacgccaagggtcaccgcacggtggcccagatgagcaaggcgctcaaaaagacggcctccaaaatcaaagtgttggaaacacgcgtgacattggcgctcgagcaggcgcaacgttccaatggcgccgtcgttaccgcggtgcaacgcgcgctagccgtctttgacgtactaagtcgcgagaacttggaacgccgcggcgcacagctctgtctgacggaagcgacgagcctactgcaccgacatcgcgcgctagcgccgatgacctggcccgcgggcacgggcgttgcggcggcggccgaagcggatcgcgccttacgcgagttcttggaggcgccctgggaatcggcgccccaaccgccgcgactccgcatgacgcccgacaccgatcacgaagaatcaacggcaggcgcgacgtccgtaccggaggtcctgggtgcgcgctacgaacccgcacacctggccgcgagcgacctattaaactggtacatcgtccccgtaagccaggcgcagcaggacatcttgtcttcgatcgacccgcccgccggctcgacatcggtgtccctgccgccggcctcgccatgaaagtcacacaggccagctgccaccagggcgacatcgctcgctttggagcgcgagcgggcaatcaatgcgtctgcaacggcatcatgttcctacacgccttgcacctgggtggaacgagcgccgtcctgcagaccgaggcgctggacgccatcatggaagagggcgcgcgtctggacgcgcggctagagcgcgagttgcaaaagaagctgcccgccggcgggcggctgccggtctacagactgggcgacgaagtgccgcgccgcctggagtcgcggttcggccggaccgtgcacgcgctctcgcggcccttcaacggcaccaccgagacgtgcgacctggacggctacatgtgtccgggcatcttcgactttctgcggtacgcgcacgccaaaccgcgtcccacctacgtactcgtcaccgtcaactcgttggcgcgcgccgtggtcttcaccgaggaccacatgttggtctttgatccgcacagctccgcggaatgtcacaacgccgccgtgtatcactgcgagggtctccatcaggtgctgatggtgctcacgggcttcggcgtgcagctgtcgcccgctttctactatgaggccctttttctctacatgctggatgtggcgaccgtaccagaggctgagatcgccgcgcgtttggtctccacctatcgcgaccgcgatatcgacctcaccggcgtcgtccgagaaagcgcggacacggcagcgacaacgaccaccgccgcaccttccttacctccgctgcccgaccccatcgtcgacccgggttgccctcctggcgtggcgcccagcattcccgtctacgatccctcgtcctcacccaaaaaaacacccgagaaacgccgcaaggacctcagcggtagcaaacacggaggcaaaaagaaacccccgtccacgacgtccaaaacactggccaccgcctcctcctccccctcagcgatagcggcggcctcttcttcgtccgcggtaccaccgtcctacagctgcggcgaaggggccctgccggccctgggccgctaccaacagctggtcgacgaggtagagcaggagttgaaggctctgacgctgccgccgttgcctgccaacaccagcgcctggacgttgcacgcggcgggtaccgaaagcggcgctaacgcggcaacggccacggcgccgtccttcgacgaagctttcctcaccgatcgtctccagcagctcatcatccatgccgtcaatcagcgctcgtgtctgcgtcgcccctgcggtccgcaatcggcggcgcagcaggcggtacgcgcctatctgggcctatccaagaaactggatgcctttctgctcaactggctgcaccacggcctggatctgcagcgcatgcacgactacctgagccacaagaccaccaaaggcacgtactcgacgctggatcgcgcactgctggagaaaatgcaagtcgtcttcgatccctacggacgtcagcacggcccggcgctcatcgcctgggtggaggagatgctgcgctacgtggaaagcaagcccactaacgaactgtctcaacgactgcaacgtttcgtaaccaagcgaccgatgcccgttagcgacagcttcgtctgcctgcgacccgtagactttcagcgtctgacgcaggtcatcgaacagcgacgtcgggtgttgcaacgtcaacgcgaggaataccacggcgtttacgagcacttggccggcctcatcaccagcatcgacattcacgacctagacgccagcgatctgaaccgacgcgaaattctgaaagcgctgcagccgttggacgacaacgccaagcaggaactctttcgcctgggcaacgccaaaatgctagagttgcagatggacctggaccgtctgagcacgcagctgctgacgcgcgtgcacaatcacatccttaacggctttttgccggtagaggacctgaagcagatggaacgcgtcgtcgagcaggtactgagactcttttacgacctgcgcgacctgaaactgtgtgacggcagctacgaagagggattcgtcgtcatacgcgaacaactgagctacctcatgacgggcactgtgcgcgacaacgtaccgctactgcaagagatcctgcagctgcgacacgcgtaccagcaagccacgcagcaaaacgagggtcgcctcacgcagatccacgacctgcttcatgtcatcgagacgctggtgcgcgacccgggcagccgcggctcggcgctgacactggccttggtacaggagcagctagctcagctggaagcgctaggcggcctgcagctacccgaagtgcagcagcgcctacagaacgcgcaactcgcgctaagccgcctctacgaagaggaagaggaaacgcagcgtttcctcgacggactctcgtacgacgatccgcccaacgaacagaccatcaagcgacacccacaattacgcgagatgttacgtcgcgacgaacagacgcgtctgcgactcatcaacgccgtactgagcatgttccacacattagtgatgcgactggcgcgcgacgagtcgccgcgaccgacgttttttgacgccgtcagtttgttgttgcagcaactgccacccgactcgcacgaacgtgaggatctgcgtgccgccaacgccacgtacgcgcagatggtcaagaaactggagcagatcgagaaagccggtaccggcgcatccgaaaaacgtttccaagcgttacgggagttggtttactttttccgtaatcatgaatatttctttcaacatatggtcggacgactgggcgtcggacctcaggtaacggaactctacgagcgatatcaacacgagatggaagaacagcacctggaacggctagaacgtgaatggcaagaagaggccggcaagctcacggtaacttctgtggaggacgtgcagcgtgtcttggcccgggcaccgagccatcgtgtcatgcatcaaatgcaacaaacgttaaccaccaagatgcaagactttttagacaaggagaaacgtaaacaggaagaacagcaacggcagctactggacggctaccaaaaaaaggtgcagcaggatttgcaacgcgtggtggacgccgttaagggcgagatgctctccaccatcccgcaccaaccactggaggccacactcgagctgctcttgggcctagatcaacgcgcccaaccgctactagacaagttcaaccaggacttgctgtcggcgctgcagcagctgagcaaaaaactagacgggcgaatcaacgagtgtctgcacggcgtgctgacgggtgatgtagagcggcgctgtcacccgcaccgagaagcggctatgcaaacccaagcctcgctaaaccacttggaccaaattttgggtccgcaacttctgatccatgagacgcagcaggccctgcaacacgccgtccatcaagcgcagttcatcgagaagtgtcaacagggcgatccaactacagccatcacgggcagcgagttcgagggcgactttgcacgctaccgcagcagtcaacagaagatggaggaacaattacaagagactagacaacagatgaccgagactagcgagcggctagatcgctcgctgcgccaggatcccgggagcagctccgtcacgcgtgtacccgagaaacccttcaagggtcaggagctggcgggtcggatcacgcccccgcccgccgacttccagcagcccgttttcaaaacgctgctagatcagcaggccgacgcggcccggaaagcgctcagcgacgaggccgatctgctgaatcagaaagtacagacgcagttgcgacaacgcgacgagcagctgagcacggcgcagaacctgtggactgatctggtcacgcgccacaaaatgagcggcggactggacgtgaccacccccgacgccaaggcgctgatggaaaagccgctggagacacttcgcgagctgttgggcaaagccacgcaacaactgccgtacctgtcggcggaacgcacagtgcgctggatgctggcctttctggaggaagcccttgcgcaaatcaccgcggaccctacgcacccgcatcacggaagcaggacccactaccggaacctgcaacagcaagctgtcgagagcgccgtgacgctagcgcatcaaatcgaacaaaacgcggcctgtgaaaattttattgcacagcatcaagaggcgactgccaacggcgcgtccacgccgcgggtcgacatggtccaggcggtggaagcggtctggcagcgactggaacccggacgcgtagccggcggcgccgcgcgtcatcaaaaagtgcaggaactgttgcagcgcttgggtcagacgctaggcgacctagaactgcaggaaacgttggcgacggaatactttgcgctgttacacggaatccagaccttcagctacgggctggactttcggtcgcagttggaaaagatccgcgatctgcggactcgttttgcggaactggccaagcgacgcggcacgcgtctctccaacgagggagtcctgcccaacccccggaaaccgcaggcgacgacttcactgggcgcctttacacgcgggttgaacgcgctggaacgacacgtccagctgggtcaccagtatctgctcaacaagctcaacggctcatcgctagtctataggctggaagacattcctagcgtgcttccggcaacacacgagaccgaccccgcgctgataatgcgcgaccgcctgcgtcgcctatgcttcgcgcgtcaccacgacaccttccttgaagtggtagacgtcttcggcatgcggcaaatcgtcacgcaggccggcgaacccattcacctggtcaccgattatggcaacgtagcctttaagtacttggcgctgcgagacgatggtcggcccctggcatggcggcgccgctgtagcggcggaggactcaagaacgtcgtcaccacacgttataaagccatcacggtagccgtggccgtctgtcagacattgcgcactttctggccacagatctcgcagtacgacctacgaccctacctcacgcagcatcagagccacacgcaccccgcggagactcacacgttgcataaccttaagctcttttgttatctggtgagcaccgcctggcaccagcgcatcgacacgcagcaggagctgacggccgccgatcgcgtaggcagcggcgagggtggtgacgtaggggaacagagaccgggccgcggtaccgtgctgcgcctgagtctgcaagagttttgtgtactcatagcggctctgtaccccgagtacatctacaccgtcctcaaatacccggtgcagatgtcactaccctccctcacagctcacctacatcaggatgtgatacacgcggtagtcaataacacacacaaaatgccccccgaccacctccccgaacaggtcaaggccttctgtatcacccccacccaatggcccgccatgcagctcaataaactgttttgggaaaataaactggtacagcaactgtgccaggtaggcccgcaaaaaagcacaccgcccttaggcaagctatggctctacgccatggccacgctggtctttccacaagacatgctgcagtgtctgtggctagaactgaaaccccagtacgccgagacatacgcctcggtgtccgaattggtacagacgttgtttcagattttcacgcaacaatgcgaaatggtgaccgaggggtacacgcaaccgcagctccccaccggagagccggtgcttcagatgatccgcgtgccacgtcaggacacaaccaccacagacacaaacacgaccacggagccgggacttttagatgtttttattcaaacagaaaccgccctagactacgcgctgggctcctggcttttcggcatacccgtgtgtctcggcgtgcatgtagccgacctgctgaaaggccaacgtatactagtagcgcgccacctcgaatacacgtcgcgagaccgcgacttcctccgcatccaacgctcccgggatctcaatctcagtcaactgctccaggacacgtggaccgaaacgccgctggagcactgctggctacaagcccaaatcagacggctacgcgattacctgcgtttccccacccgcttagagtttattcccctagtcatttacaacgcacaggaccacaccgtcgtacgcgtgctgcgaccgccctccacgttcgaacaggaccacagtcggctggtgttggacgaggccttccccaccttcccgctgtatgaccaagatgataactcatccgcggacaacatcgctgcgtctggcgccgctccaacaccgccggtacctttcaaccgcgtgccagtcaatattcagtttctgcgtgaaaacccgccacccatcgcgcgagttcagcagccgccgcgccgacatcgtcatcgagcggccgcggccgcagacgacgacggacagatagatcacgtacaagacgatacatcaaggacagccgactctgcattagtctctaccgcctttggcgggtccgtctttcaagaaaaccgattgggagaaacaccactatgccgagatgaacttgtggccgtggcgcccggcgccgccagcaccagtttcgcctcgccgcctatcacggtgcttacgcagaacgtcctcagtgctctagaaatactgcggctagtgcgattggacctgcgacaactggcgcaatccgtacaggacactattcaacacatgcggtttctctatcttttgtaaccgacactgacagtagcgggtaataaaaacaataggatttttatcgtttttttatgttacaaaacaacgtatcactttcacggtgatttattcttgctattccttttccccttgggctgtcagcgccgggtgcgcgacacggctaccatgcgcaacaggtccagcttaaaggcgcacttgtcattaaacaggctggacatgcgcgtgtacttgctcagcatggtggccaacaccgggtgggtggcctctgatatctcggtcggcagctccaaaacgacgttaacgacgtgacggtgtttttcgtcccgcttgttggccaccgtgggtcccggcgcggtgttagacatggggcaggccgtggggggaggacgaagaggaagccgctgctaaaccgccgcgcgcctgctgcacaatgtggccgccgacgtggcaggcggtctgtttaaccagcgcgcagccccgacacagcggggcgccgtcctcgctttccaaacagctgtcgcggtactcgcccgtctgacagcgcgcgcacagcaggccgtgcccgtgcgaagtgaggcgcaggagacgcgggaccgtcacgtcgcgtaccaccacagtggagtcgcaggtgcgtgccgcgcagggcagaatgacgtcgaaagccagccggtgatcgtacacggcacaagccgcgttgaggcccagcacggctttccagcccacgcgtacgcagcgctgtccaaagagcgtctcggagacgagctcgtagacgcgctgccgcaccacccgctgactgccgcagagcgagcagtgcacgagctcggcgtgcgtgttgaagatgacgctcttttcttgacggtcccgataatagaacatcgagttgagcggaaagttttgctggcagtgtagcttttccttacccaggttgaggcagtgtccgcactgccgacagaccacggccaccagcgagcgcgcgtccagatggcgctcgcacttgagtcgacacagacaccagagcggcaggtcgatgacgctgccgatgaggccgccgcgcagcgcggcgctgagtgcaaagaggacgatcttggtgggctctacgtgacgcgcctgctgtccggcgcccgcgtgtcctaccgccgcagctgccgccgtcgagcctcctccgcgcgtctcgtcgtgcagacccagtgcccgcaacggcaccaggtatcgcggacacgtgtcgcaaaacgtctgcaccgcttgtcgggccagtacgtagagcgggtttccgcagggtaccttcccggcgtaccggcgcaaggctgcgatgaggccccgcaactgcggcgaccgcggctgccgttggtgacaccactggttacggtggtatacggccaaatcagcgcgggcgtcgaagcgcttggcgcgtagtaatgctaggcacggcgagctggtggggtgaagcacgggcagccgaaggtccaccccgaaaaggaaacggtgaaggtcacctagcagcgaggcggtgacaccgtccaacaacgcgtgcagccgctcgggcgggtagagccgcagacggcgcagcaggtagtcggtgtcgtagcgttcgaaacgcagaaaggccatcgtgcggacggccacggtgtgcagacagtccatgctgtagacgtaagcgagaaacacaaagtagggcttggtcataaccatacgctgaaagagcgccgtcaccgcctcccgctcggcttgccgacacaccagccattcgcgcaggaagcgttggtagagacggtcgcccagctcgcgattcagaaagcgcttatccgtcacgaagagatgaaggacgcaagaacgtggcacgtgatgcaccagctgctgctggaggaccgccgacgtctgcgccgcaaactgcgccggtggctgcgacgtttctaccgccgcttcctccggctgcagcgcaccgcggccgatcaccagctgcacatggaaatggtcctcgtgaacgcagaggggcgcgaagagacggcgcagagcctggtggaactcatcagtcgcggtgtgcggagcgtgtcggagacgacgactggccatgaccgcgccacagcagagccagcaccagcagaagagccagcaccagcgggcccagagtcgcaaagcgcgcgggcagccacggcccagactgcggtcgcgatggcccggagcgcgctcgccaccacgatgacggtgcccaacgataaccagtccgctccaaggacggcgcgcacggcggagacggcggatgacggtgatgggtcgacacccctcgccgacgactcacgtgctcctccagaggccgacgcgcggaccctccgacgtcctggcccgccgctgccgctgccgccttcccttctcccgccagagccagcaactcctcctcctcttcatcagcgtctccctcgcttgcgcatccgcatcgtcccatacaggcctcacaacgacacagccgccacgaccccgccgccatgggtggcggcggcggccgaggcccggcagcggcgccgccagcggcgaccatggtgggagagcaactcggatgacgaggaggaggagggggagatgcggtccgagaggaccgctttcccgccgttcgcgtaagcgcggccgacatgcgggcgcgccacagggacggaccgctgccgctgtgactgcttacggtgacgtggttccggaccgccaacgacgtcgacgcggctttcttggcgtacagctcgcgcagcagattctcgtactcgccctcgttttcgggtccgaaggcgatgagctcgatgttgaagaccgacgccgaattggatttgcgcaccacgcacttcgtcagcactccgtaggccgagggcttgatctcctcgatgtccttgagcgtgacgatgagcgactcgttcaccttaagcacattgaactcacctacgtggcgcgccggcgaaacgagcttgacgggcgctcgtacaaaacagcagagggagacggcgcagccagtgtttttaaagataaaacaaggcacgtggtctgtgcggctctcccagtagctgagtagatactcgacacaatagaccgtgtctgtcttgagcatggcgtcgcacaccgagtaattggggtttttacagatgaggccggcatcggtgacgcgcagctcgctgggacccaacttgaggatacgccgcgtggcctgcaccagatcctgatggagaaccttgttcatctccatcgcaccgacgccaccgccgatttatttacccggcgccgactcgtcttttccctccaggattccgttaatgtccatgagcttgctgacgatcgccgttaatagttgcgtcttctcacggaggatctctccgtgactgcaggtcgcgcagtcgccgtgcacgtacttgaggaaggcggcgtacttctgacccgcgttcacgaaatttaagcgcgcgtccagagagggcagcaacagatcgtagacgcgcggcagcatcggctcgaactgtaatagcagatcgtcgtcaagatcgggtagcgcgtgtccgtcttcaccgtcctcgtcgtcaccacctcccccctcgagcccaccgctcgtaccagccgcgggctccgcgtcctcgtcgatcaccagcggtcgcgtcggcaccggagaatccacgtcatcctgcacgtcgttttcctcctctccgtcgtcatcgtccagaaacggcacccgctgcttagcccaggattccggttttagaagctccacatcgaagacgagagtggcatgtggtgggatgatgcctgggtgcccagtggcaccataggcataatctggagatatagtcagtttggctctctgacccacactcatctgggcaaccccttcttcccagcctcggatcacctcctgcttgcctagcataaacttaaagggcttgtttctgtcccgggaggaatcgactttctttccatcttcaagcatcccggtgtagtgcaccacacaggtctggccgcgcttggggaaggtgcgcccgtctcctggggagatggtttccacctgcactcccattcttttttccgcgtcctcaatcagcggcgccgatcgccatgaatccgagtacccacgtgagcagtaacggcccaacgactccccctcacgggccccacaccacgtttcttcccccgaccagcccggccccgtccaccagctccgtcgccgccgctaccttgtgcagtccgcaacgacaggccgtttcgcgttacagcggctggagcaccgagtacacccagtggcactcggacttgacaactgagctgctatggcacgcgcacccgcgtcaagtacctatggacgaagcgctggccgccgcggcggccgcctcataccaggtaaatcctcaacaccccgccaaccgttaccgtcattacgaattccagacgctcagcctcggcacctcggaggtagacgaactgctcaactgttgtgcggaagaaaccacgtgcggcggcacgcaatccaccgtactcaccaatgcgaccaacaccactagctgcggcggagccgtcgccggcagtagcaacgtaggacccgccggcgcttcggccgcctgcgacctagatgcagaactggccggcctcgaaacctcggcggccgactttgaacaactgcggcgactgtgcgcgccgctggccatcgacacgcgctgtaacctatgcgccatcatcagcatctgcctcaaacaggactgcgaccagagctggctcctcgagtacagcttgctgtgcttcaaatgcagttacgcgccccgtgcggcgctcagcacgctcatcatcatgtccgagtttacgcatctgctgcagcagcacttttccgatctgcgcatcgacgacctgttccgacaccacgttctcacggtcttcgatttccacctgcactttttcatcaatcgttgctttgaaaaacaagtgggcgacgcggttgataacgagaatgtcaccctgaaccatctggccgtggtgcgggccatggtcatgggtgaagacacggtgccttacaacaagcctcggcgccacccgcaacagaagcaaaaaaacaacccttatcacgtcgaagtgccgcaagaactgatcgacaactttctagaacacagctcacctagccgcgaccgcttcgtgcagctgcttttctatatgtgggccggcaccggcgtcatgagcaccacgccactcacggaactcacgcacactaagttcgcgcgactagacgcgttatccacggcctcggaaagagaagacgcaaggatgatgatagaagaagaggaggatgaagaaggaggagaaaaaggaggagacgatccgggccgtcacaacggcggtggcaccagcggggggttcagcgagagcacgctaaaaaaaaacgtgggtcccatttacctatgtcccgtacccgctttttttaccaagaaccaaaccagtaccgtgtgtctgctgtgcgaactcatggcctgctcctattacgataacgtcgtcctgcgcgagctgtaccgccgcgtcgtctcgtattgtcagaacaatgtgaagatggtggaccgcattcagctggtattggccgatctgttgcgcgaatgcacgtcgccgctcggcgcggcacacgaggacgtggcgcgctgtggactcgaagcacccacctcgcccggaggcgactcggactaccacggcctgagcggcgtcgacggcgcactggcgcgacccgacccggtattttgccacgtcctgcgtcaggcaggcgtcacgggcatctacaagcactttttctgcgacccgcagtgcgccggcaacatccgcgtcaccaacgaggccgtgctcttcggacgcctgcacccccaccacgtccaggaggtgaaactggccatctgtcacgacaattactatataagtcgacttccgcgacgtgtgtggctctgcatcacactcttcaaggcctttcagattacaaaacgcacctacaaaggcaaagtgcacctggcggactttatgcgcgatttcacgcagctgttggagagttgcgacatcaagctggtggaccccacgtacgtgatagacaagtatgtctagcgtgagcggcgtgcgcacgccgcgcgaacgacgctcggccttgcgctccctgctccgcaagcgccgccaacgcgagctggccagcaaagtggcgtcgacggtgaacggcgctacgtcggccaacaaccacggcgaaccgccgtcgccggccgacgcgcgcccgcgcctcacgctgcacgacctgcacgacatcttccgcgagcaccccgaactggagctcaagtaccttaacatgatgaagatggccatcacgggcaaagagtccatctgcttacccttcaatttccactcgcaccggcagcacacctgcctcgacatctcgccgtacggcaacgagcaggtctcgcgcatcgcctgcacctcgtgcgaggacaaccgcatcctgcccaccgcctccgacgccatggtggccttcatcaatcagacgtccaacatcatgaaaaatagaaacttttattacgggttctgtaagagcagcgagctactcaagctctccaccaaccagccgcccatcttccaaatttattacctgctgcacgccgccaaccacgacatcgtgccctttatgcacgccgaggacggccggttgcacatgcacgtcatcttcgaaaaccccgacgtgcacatcccctgcgactgcatcacgcagatgctcacggcggcgcgcgaagactacagcgtcacgctcaacatcgtgcgcgaccacgtcgttatcagcgtgctgtgtcacgccgtctcggccagcagcgtcaagatcgacgtgactattttgcaacgcaagattgacgagatggacattcccaacgacgtgagcgagtcctttgagcgctacaaagagctcattcaggagctgtgtcagtccagcggcaacaacctatacgaggaggccacgtcgtcctacgcgatacggtctcccttaaccgcgtcgccgttgcacgtagtttccaccaacggctgcggcccctcctcctcgtcccagtccacgccgcctcatctccacccgccgtcgcaggcgacgcagccccaccactactctcaccaccagtctcagtctcagcagcatcatcaccgtccccagtcaccaccgccgccgctgtttctcaacagcattcgtgcgccttgacactgtacggcagaaaagccggctccaagtgcaagcgccgcggcagcaccatgtgcaaaaacttgtccttgcgcgcggtttcgccgccgggaaagacgggcgacagcacgttagttacagccttgagaacctgctcaaagtacttgtcggcgtgaatgggcacgccgtgctcgcgcacgtagctcggatcttcggctacctcgtagttgcacacggccgacggtggtttccgcgccctcttctttgccggctctcctcctctcctgttgctctcctctaccccgccgccgtcagcgtcgtcgtccgtgccatcaatcgcgtccgaccgggaaaccacgccggcggttacagaatcaccgttgtcggaggaaccctgcggcgccgtccggacaccgggcgccgtcagaacgtaaaagacccgatccccgaccgagggtagctcctcagaacgggccgccaatcgcttaatgacggcaatgtgcggcaggttagattgacggtacagcgagatgtccttagagagcaccgacgaaagcaccaggtcctcgacacgcacacggtgcaggtacagatcgtcgcgggcctgcaccaagcggcgtaagatacgccagaaaccgcgtggcacgccgtacttcttgacttcatcgagtgagaggcgcgacaggcgcacggctgcttccgagacctcgcgatcctcaaagagcagcgagaggacgtcacgcgtgacgcccttgacgaactcgcaggccgtcttgcgcaccagatccacgcccttcatgctcagacccgaggcgccctccactttgccgatgtaacgtttcttgcagatcatcataagagagacgaagaccttttcaaactccagcttgacgggctccacaaaaagacaggccgtcacgtagtgcgccaggctgggcccacgcgccaccagagcctgcggcgtcaggccacgaaagcggacaaacacgctgtccgtgtccccgtagatgacccgcgcctccacccgccgttcgttcgagccccctgacgatgtttcgagcccctccggtaacgcgctgctctcctccgaatccccctcccgcgttcccactacatagtcttcctgattaaaaaaattgtgcaaaaaacacggctctgaaaagttgtctttgatgaaccgcgccgtgcgctctagcatgtcgcgaccgatgcgcgtgatgctggcggcgatgggcagacacggcatcataccgttgaccacgccggtaaaaccgtagaaagcgttgcacgttactttgagcgccatctgttccttgtcgagcagcatacggcgcacagggtcttgacactcgcgcatgcattcgcgcacggcacgccgctgcgaaacccacttgttgagcagttccgagagcaccgagacgcgcaccgaagcacgcacaaagcggtgggtcacgccgttctctagcgtgacgctgtatacgtcggcggggtccacagggtactcgccacccggcaccagcagggtggagtagcagaggttgtgggccatgatgatggaagggtagaggctggcaaagtcgaacacggccacggggtcgttgtagtaacccacctcgggctcaaacaccgtggcgccctggtacgaaaccgccgcagtaccgccggcgccgtgattgtcgttggaaacgccgacgccgccactactgccggagccgacgctgaaaacgccgacgctgctactactgttactgccggagccgggtgaaacgccgtcctgactggacggcgcagattgcaagggcggcgacatctgaaacatagccgccacagaacccgcgtcgccgggcacagcggcggtagagatgatagcagcgttaggtgacacagcaacgctattcgtttcgggcaccgtcgtacctttgctgtagtggttgggcaggataaaatcgcggcaggcgcactcgtccagcagcgaggtgtagatacggatctgctgtccgtcaaagatgacacgccgcaacggaattttagccagccgcgcgatggccccggcctcgtagtgaaaattaatggtgttgaacagatcgcgcaccaatacggcgtcctgcagacagtaacggcctacctgggcgcggccctcggcattagccacgaaacaacgcgggatgtccttgtaagacaggtcatccttgcgttgccgcaggtaaagctcggccatagtgttgagcttatagttgggcgagttagtcttggccatgcatacagggtacatgtcgataaccaccgaacccgcaatatacaccttggtggcggccgtgctggccggattgttgtgagaagccgagggaaaagcggcggcgtactgccgcttaaaacccacggcggggctgtgtaaaaagaaacggccgccctgcgccgtaggcaacttgcagaagcgctgcgagtccaccttatacaggtactcgagacgcgtgaggatgtacttcaagtcaaaagagttgatgttgtaaccggtcacaaaggccggcgcgtaccgttgaaagaaaagcataaagcccagcagcagctcgtattcggaagggaactcgtagacgtccacgtctgggcccacctgcccgcaggtgccgatcgtaaagagatgaagacccgagtgcccaaagatcacaccctccgaagtgcagccccgaccatcgttcccgtttgggatcccctgatccacggcggtgtttccccccgtctcgtagcacacgcacgagatctgaatgacaatgtcatcggacttctcggcgcagggaaaaccaccctcgccgctcatgcactcgatatcgaaggacaggcatcgatagcgcggccacgagctgtcgtcgggcacagccaccaggtcagagacatcgcagtctacctcgatatcacaagtcgacgcgcgaccctgctgccgccagtcgtaacgattcacggagcaccagccgaacgtggtgatccgccgatcgatgaccaaacgcgtcagcggatccacacggacctcgtacacgggaaaaccctgctccagcagatactcgccgatttttctggccatggtccagttgctgatagacacacactgcaaatcgggcacgggtcgcgtcccgtacccatagatggaggtcttggtggccggcgtgacagacacggcgtatggcgtccgcggttcgggcactagttcgcccacgctggcaatgacctcacgcagcctatcggtgtcgctgtactcacagtaaaagtagctgcgctgcccgaaaacgttgacgcagatactgtagccgtgttctgtggccccgaagaaacgcaacacgttccccgaaggcaccagatgctgacgatagcgcggcgacacgttttcgggcgagtcgaagaagagcacggcgtccgtctgatcgtaggtgtgaaaacgaataggtcccaccacgcgacccaccagggtctcgcgccaaggacacggccaaaccatgtcatgactcaacaaatgtttaatctctcgatagaacatgagaggcagccgtcccgtcttatgcttgatcaaccccgtctgaccgtcgaacatgacacctcgcggcacgatctgcaaaaactgtttctgtggcggccgcttgcccgagccctgcgcggagccgggctgcgaacgctgacgccggccacccgcgaccgcaccgccggtcacgccgccgctcagatacgggttgaaaaacatagcggaccgtgagaggctgacagcttacgaagcaaaatcacaaagaaaatacacatgcagcacctagatatccagtttaaccccgtatatcacaagtctctgtgtcaatattttttgtctagtttttttttcctcctggttcagacgttctcttcttcgtcggagtctttcaagtgtctgtagccgtttttgcgatgtcgcagccggtctagcaggttaggcttctgtcccttgtcctgcgtgccagtctgtccgtccaaagaatctgtaccgttctgctgcgctcgctgctctgcgtccagacgggccagggccagaagcatctggtaagcctgctcgttggtgtaaggcggagccgccgtggatgcatcagacgacggtggtcccggtcctttgcgaccagaattataaacactttcctcgtaggaaggcggagcctgtaacgacgtgtctttggtgctgcccgacgtcacggtggtcccgtcggcggacaccagatagggaaagaggttctgcagcggctgcgtgcacagacgccgctgtcgagtatagatcaaataagtgataatgactacggctatggccacgaggatgatggtgaaggctccgaaggggtttttgaggaaggtggcaacgccttcgaccacggaggccaccgcgccacccacggccccaatggctacgccaacggcctttcccgcggcgcccaggccgctcatgaggtcgtccagacccttgaggtagggcggtagcgggtcgactaccttgtcctccacgtactttacccgctgcttgtacgagttgaattcgcgcatgatctcttcgaggtcaaaaacgttgctggaacgcagctctttctgcgagtaaagttccagtaccctgaagtcggtattttccagcgggtcgatatccagggcgatcatgctgtcgacggtggagatactgctgaggtcaatcatgcgtttgaagaggtagtccacgtactcgtaggccgagttcccggcgatgaagatcttgaggctgggaagctgacattcctcagtgcggtggttgcccaacaggatttcgttgtcctcgcccagttgaccgtactgcacgtacgagctgttggcgaaattaaagatgaccacgggtcgtgagtagcagcgtcctggcgattccttcacgttcatatcacgcagcaccttgacgctggtttggttgatggtcacgcagctggccaggcccaagacatcacccatgaaacgcgcggcaatcggtttgttgtaaatggccgagagaatggctgacgggttgatcttgctgagttccttgaagacctctagggtgcgccgttgatccacacaccaggcttctgcgatttgcgccagcgcccggttgatgtaaccgcgcaacgtgtcataggtgaactgcagctgggcgtagaccagattgtgcaccgattccatgctggacaaatgagttgtattattgtcactcgtacttcttctggtcctatgagtgatattcagactggatcgattggccaaacgttccaattccaccaaagatttttgcttgatgccttgccagaacaccaccagaccgccgctggtttcgaagacggacacgtttccgtatttttcatatgtttgattgtatgaagtattgaaaatctgctgtaacttatttatagcctcatcacgtacgcagtccagcgcggagtcggacatgttcacttcttgtttcttagacagaaaagttgcagtcattttggcagaagaaaagtggtacgagtcttcggcttcggaacggatagtacgttccgaggcttcccagaaggtgagctggcaggtgacattcttctcgtcctgtatatcccaagagatcaccgagtcggcacgttcgagaaaagccaccaacctatgggtttctggcgcagcgttgggtcttccaaagtcggaaacgatggtgtagttcgggaaaatgaaaaacttgtcggcgttttctccaaagtagctggcattgcgattggttccgttgtagaaaggagaaatgtaaaccacatcacccgtggaagttgcaaaaaaatgataaggatacttggagcgcgcagtagtgatggtcagcatacagttcagattacaggtctcacgatagagccaggtgctgccgcggctgtgccactgatccttgaccgtcacgtaacgggtactgtgggtgttggaataatcgtcgggaattaattgcatggttttgttttcataactgtccctatgatatgccacgaaaaccgtgcctcctataacgcggctgtaggaactgtagcattgagcaaacttgttgatgtgatgaatctcccacataggaggcgccacgtattccgtattgctgcccagcagataagtggtgtagatgtaagcgtagctacgacgaaacgtcaaaaccttttggtagacccgtaccttaaaggtgtgcgccacgatgttgcgcttgtagaccaccatgatgccctcatccaagtcttcattgataggcttcatcgaggtgcagatgatattacgttcaaagcgaataagatccgtaccctgggccatagaacacacgcgataggggtacttggtagtgttgactcccaccacatctccgtacttgagggtagtgttgtagatagtctcgttggctctatgactgacggcttcagaagacgttacgtgttgagaatagactgaccgggtttgagcagacgtcgtacgagaagtatggcttccattgtgagtagaagaagttgcatgggaagtactagaagaggaaaccgcagcacccagacagacgatacacaggttaacgcagactaccaggcaccagatcctggattccatgttcgtcgcgggccaaatccagcagcgatgaggcgcgtcgtggtctcttgcgtgtcgcgcggaccctccgggaaacacccgcagtcgaggaggagggatacggacttggcagccaaggtcggtccggctccctgaagacacccgagacggccgcggcggccgtcagggtggagggcttggccacgggagctgttggcacgtcgccactctcatccggtctggacagatgcctgtagaggaggagatatagatctttggacttataaagacttccttcgtgacgaagcagcagcggccactctttgttatacgtgagaatcacatctctgtccgggtgcagttcgtcgcgcaggcacgcgatcgagagttgtttcccgaaagtttcattatatagtgcgacggagagcacgagctcccgcacgtgcatccacatctccttctgcagcacgtttaggtcctgacagtccgaaaaattgaaaaaacccatatacttcaccaccatccactcactgggatacacggtaccttccgcgcatttgaccaaatcgtccttgacgtggggtagtacgcccgcgttgtcgcaggcataggccatgtccacattgtgagagaggggataacgatcggtgcagtgggtgaagaggggcccgttacacaactcgtagatctgctgacccagtagcgggagggattccacaggcagactcttgtggatcaggttattgaccacatacaggtgctcatcgtaggtgaactgatcacccacgtccaccacgtcttggtcctggtggtattggctgcggtacagaaacccattcatgagcttagagataaagtccagacacaagggccccactagattgacatcgatgagcttgctagtcagacgctcctgcgttttgatgcaacggatcaccttgccatagcccacctccgagaccttctgcaggtaggcgcgtttgcgcacgttcacctcgcgagtgacgttgtggatgcgggagcgcgcgtccaccaagtcgagagcctcgtgttcgtcgcagttgcgcacccgtaagccgttctcgctgccgtcgccgtcctgcccattcacccctccccctaccactttcttgcctcctccacgagcccggccgccgccaccgttattcctctgactgtgagtactgctgttgctgctgttgctggccgtcatcaaagtcgtacccgtccccgacatcgcctcccgtccacgcaggtgaatagcctcgccctcggggccgtcgccccccgtgccatctggcagcggacgtcgaatctcctcgagaatatgcttgattttggtgtacatctcgttgctttcgtggagcttgttgaacaccgggttgtcctcgaaagcttgaatgctgagggatgtgatgaggtcgatgatcctgttgggggcggcaaagaccgaccccacgaacatgcgctcctccccgtccaacgccttttccccgagcacgaagatgtcctccacgtcctccccgtacagatggcgactgatgccgttcatgagcgcccggcacagctggtgatacacatttagctgctggatggtgatgcccacccgcttgacgataacctccgaggtacgggaccagtaggtaaaatccgacaaggaatatattcgttccggtatatccgtaaacaggttgtactccctcagcgcctcctccgcctcctggatgtagctgtggtaggccgatgaagaagagaataggcttttgagggccgaaaggactccagccaagtgggggatgcgcgttgtcaggtccagcaggtcctgctccaccgtctggatattcacatcggactggcttgacggacggtggaccgctatatggttgcacagcaagccctgcagccgcttgttcagcgagcggccctgattcgggatgatggtcagctcctcgtagcattgggcgcatgtcgtcccttcgacgtacacttcctgacgcgccaccggcgagatgccgcataggcgacggaggagctccagcaactgcgcgcagacctccaggccggcctccggcgccaggatcccgtacacgtagttcattttgcacaggaagcgctcgatgtcgttgagtgtggccagactgacgctgaaacggacgttgtccgtaaactggagctccacggtgtgatggcgatcgcagcgatccaaacggaggacggtacggtagaaggccgcccggtccggctggcgcgagtaggccatcagcgcccgatccagcaaagccgtatcctcgtgcagcgccttcagcagcatctccaggtagagcgtcagcaacgaactctgcgtacgattctgcgccaccacctccgggtagatcttccggtacagatacactatagccgccgcgtttctcttgaacggcgtggactccgccagtaacacgttcggatcgcagtactttagacactccagctccatggcgtattcgttgcatttcgaacacactacgcatagtttctgtaacaaattcatctccatgactcgactcgctcacgtacgagacgctgtcgtccggtctggcgccggccagagacatggagtcggtgcacaaataactcgcgggccgctcgctatgccgactgacgttgacgttaatatataacgacgtcgtcgacgacgcgggttctgctcccgaagctgttgccgccgcttgcggcgcaacctcctccaccaccgccgccgccggctcctccgcctcgggcgacgggggctcggagatgaccggctgtgtctgacactcctccccttcctcaggcggcccgggcgccgacgcgaatgtcggagtttgccagcgcggcggcggtctctgtctctggtgccgcggcgctaaccttcggggctgttgctgctgttgatgatgcgacgccgtctgtcgccgctgttgcggcggtagctgatacggtgtcgcctggtgctgctgtgtcggtggctgctgttgctgctgttgttgcggtctgaaaagcggccacgggggctgcgactgttgttgctgttgttgcgatgctcgtggctgcggcggccgttgtcgcggcgtttgctggcggttacaaccggctgcgtttggccggcaataacccgctgcccccgccgcccccgctgctcccgccgacgccgccagcctcgtcttcgccggcgttcacgagaaagcagccacctcccgtctcgccgggcacgccgaagcaaatggagttgcccgcgacggactcgccgagaagaagaccgccacccccgacgccggacgccgcgccgacgccactgggcgcgaagagcgccgacaggtcgtgcacctcccccccggcggcgtccgttaatcgctgggcgtcggcgtccagcacgcgtcgcaagttctccagcgaaaagtcctccacgccctgctcctgcaacgcggcaaacttgtccatcagcgacgcggccagcgcctcgcagccatccacgaagaagagcacatcgtcggacgcggggatctcctcgcgcacgctcagaatctcgtacacggccatcacttcggggtcgcaatccaagttctcggcgtccagcgccagcatgacgcggttttttataagatccgcgtcaaaaagcacgttctcgcggcgcgagcgtttgatgagcacgtcggccagacgcgtagccaagaggtagcgctggcgcatgaaacgataatcttggccgctcatagagctcacgttaaggctgcgttccacaccgttgcccgaaaagtagccgatctgcccaaactgatagatctccttgctgttgttgatacccgcatatttttccacgctcacgggcacggtcaccaaggaacgatgctcaaaaacgctccgtaccaacgattcacgcgccacagtggcggccatgggcgccggcacgcctgcggtcttcaagcccttgacatgcaacgcaaattcggcgggcgacgagaaccgcggactagcacctaacacgtgaggaaactgcgcgtggttctgcgtcgttaagcgcgtcgtcaacccgtgcagcgagccgatgtagtctttgaagccataatagcagaggaatttgttatggaaacggctttccacgtaactcagcacacagtctggcgccacatccagcagatcgtgctcctgatagtcagccgtcacagccaccagaaatttgacgaaagcattgaactcgcccatgtcacctatgggcacattcttgggcaacgcgttggaacagaccttctgccaaaactgtaagcaggggagaccacattcaggaaagagtcgctcgtgatgtcgatacagcagaaatcccaagcagcccttagccggattacgacgcggaacgtgatcgcggcgaaaaaacacgctacccgcgttgcccttgcccgcgcggtagatgggtcggtttttcacccgcaccatgatcaacgtgggtaccgacagccgcgagagcttgatctccatgggcaccacggcgtacgtgccctgcgcgtacagcctaaagtccagcaggcggtcgtgatccgaattcttggacgacttgatctgcttggtgaagagaaagcccttgcgcgacgacgtggtggagaacgcgccgtgaatggattgaaaatgctgcgtcatccatttggataccaagttggtggtcaacggattgtccacaatgtatgaggtagcggtaataagcgccacgttctggatcacgtaaaagacggatctgaaataggcgtaggctagcagcggctggaaggccacggcgtagggattcagatccaggttgaaggcctgcgtggcgcccgccacctcgtcgcggctgctcttgaggcgcacctccgaaacgaaacccagggcctcgtcgtccacaaacttgttgagcgccgaaaagacggccacaaagtcgcttttgccgtgcgcgctaaaggtatcctcgcccgtcacggggtcgatgagccgcatcttgcggcagtaatccaagatgcgattgagccgataggtacggtccacgctagcgcccaacatgcgaccgccgcgccccatcattcccccggaatccccaccacccccaccaccacgaccgccacccagaccgtcgctcgggcccccgctcacgtctcgtccaccacccccgccagcaccgccgcccggaaccccgtcgtcacctttgccgtccaaacccccgtccttggcgtcgacgttgtaacgccgaccgaagctgcccaaaatatccacgtcgttgagaaaacgcgactgcacggtgatcacgcagggctccttcttgggctgcttgggcaccacgggcaagcgggtgcgcacccgcacgaaggccgtctgataacacgtgtggcaacaagtacccccacaggcctcgcacagccccgcggcgcagcccaccaggtgattcgtgagcgtcgacgaacccgacaagcccgtgttgtacaccgagacacgattcagataccagacgaagcccgaaactagctgcggacacgtgccacacaccaacgccaaatgctgcggcccatagcgttcgtccttgagcggcgcgccctgaaacttgagcaccttgcgcgcgtcgttgtagacgtcttcgcaggccgccgacaacccgttggtgaactgaatagccttgagcaacgtctcctgactggccgtaccgccggcgctgggatgccgcgccgacgactggagatacaccagcctgtgctggtagagcaccgaattagcgctgaagaccaaggcggccacgtgcgtcgagagatgcaacttgagctcggtcagcgcgcggatcagatcgcggtgatcggttgcgttggtcactaaaggccactcggaaaagagcatagattcggcaggttggtaagccgaatcgaaaaataccgaggcaaaactgaaggccaactcgcaaaccaccgcgtcactcagcatcagatgatccttttccagactgctgagtcgctggctcatgtaccccaagtagcgcttatgtggcgccagcttcaccgactgctgactgtcgtgcacaaactgccgcaacgccgcctcgatcagcacacgcggctccgagaagcgcagcgattgacaccatgacgtgtacacgtagtagaaaagcgtctcgcttacggccggcacgtagagccctcgcgcctccacaaaagcgctgcgcgcatccagcgagacctcgtcggcttcggcgtcaagctgcaacgaattaaagagcgtaggcgggtacaacggcacgcgcaccgcctcgccgccgtgcagtcgcaccgtggtcgcctcctccacgcatggaatcagctgaccggcaaagagaaactccttcaagccgttgcccaccaccacgtgcacagtcgtctcggacgcctgacagcccaccgccgcgcacaacgccgccagatcggtaggcacgcgatccgcctcgggcatgtaagcctccaacgcgtacttctggcgggcgtcctcgcacagccgatgcacgtctccgtgatcctcggtaaaagccacgatgccttgcgtatgatgaaagtagagcgcaaaaggacagaaggacgtgactttcgtgagcaccccgccgtcgtaacaaagcacaggcgtgcgcacagagacgccgaaatccgcctccaccgtgagccccgccaacaaaggagcgatcaccacgctcgaggaacggtcgcatagcgagagagtggccagaatctcctgcgtttctgcgttcaacctgctgaagtagagaaaagccgcgggccccaccggcgctagcgcggttagttcctcgtggctcatggtggatgaacggaagacaatggctacgccgccactgagtgaattttataccaaggaaaagttcagcacgtcatgtttgacgcacgacgtctgagacaccaccgtggccaccactgcggtctggctgcggttgcggaccaccaaaggcgacaaccgcaacgatcccagcaattcgtaagaaaagctaaccgctacggtcaggtagcctctcgcagccagaccgctagccgacgcacccgcccgcgaaaatagcgtgatgttcgggacggctttgcgtcaccgccaactaacgtcggtagtcgagcacgtcgtttatcctcagcacaccgtccgatcacaatccgttttcccactcagtcgcacaagcagcacataaaaaccccacacagggcacgtgaaaacaccgtccctagaaaacggcgttttctgtcctaccgtcacccgtatacacaggcaaatcccaatcccgatccccgaaaacaccgtacggtgtttgtgacctccaaaatcacatcagctaacaaaccgtgaaaagtcacgtttcacgaacacggtgtttttaaatcacaaagaaccgcctgacggtttacaagcagaaacaccgcaccacggtggtacaagcgcgatgaatctggtctcgcaacctcaatcgccgctatcaccaccgattttcgctgcgctccgccgacaaaacgccgtacaagctacacaccccaaaaacccgcgcgcctacgggcgccaaacctgtgtgttatctcaacgtcacaacacgacacaaaccgcgtaacgtggtttcccgaacacgtacgcggcacagacccccgacacgtactcgaagaccttacagtttacgagtcaataaaacaggaaaagatccgaactttaaaattgtgtgtttttattttcccatccccctctttttaccaaaaaacacatttttcgtcttgtaaaaagtaactttcgcccattgccatgaaacaccgtgatggggaacggtgttgtgtgtcgactgacgtcactacggcgatcagtatcgacgtcgtgtatacataacggtgcccggtgtttttattcggggcgttgtcgcgtcttgatgtaatgtaacctgaaaccgccgtgcccaagaatgcggaagccagcgtgtaatcataacggggttttgggtacaatctgacgacatctggcggcgagcgtacaccatcgaatgtggcgatcgccggctctacgtcacaatgacgcaaaaacacactgtaaaacccgcgtagacagctttcctggtcaacgagcgccatctggtgtcggcataagaacaggcatcaaccccgtggccggcgaggcggtgagcacttttgttggtcacgtgaccatcagcgcaggaagcgaggcccgtagaaccgcccaagaggcggtgccagatgccaacgtcataatcacaaggtgatttgttacgtcacgcgcgcgcacgcacgcgcgcggtagaatacagcgatccctagtgaagccacacccattacgtgtagccatatccgcttacgtatacagccacacccctaggtacgccaccttatctaccaatcacagaaacggatatacaatgacccctccctagactccaccccttgtacggaaatttcagataggtggaacccgttagggttccaccgtcctcggtgtacgtacaggcttctccgtctaccggaaatatacacctgctgacgtagacgctactcccggatacgcgtcataagctactggaccctaggggggagtgtctacagggctacgtgcacgcccccttacctagggtatccgcccccttcctctgttttggcctagtaaacttaacgccgccgcttctcacgtgacccctgacaagcctacgtcacactcgcgtgaccacacccactccggatatacgtcatcctgtggaattccggacatacggtgacgtagcgagcgtagcgagctacgtcacgtatgcgtgcgtcatctccggcggaaatcatctctgatgacgtagcgagcgaagcgagctacgtcatcagtccgttttacgtataccggatgctaggcgacgccccgtaggggcggagcctagcttccacccctaggatgcataccctatatagcataattcttctaacgaaacgttctacgaaaacggactggcggaacgggaaccaccgtaacccccccccctcacccccccccttctcctccggaaccggggggggcaaatttttaccaaatttgggcaaccatgatttccaatgggacggcgtttccgtgcgcatgcgcagtcggggcgagtttttggttgtcagggcgttgccacgcggattatgggatggtgactcgagtgcgcatgcgccggggatgccgcatggaaaacctatatataaggaggggtgaaccaggggccccggggcgcatgcgcgggccagggcccgcgggagggtcgccctgcgcatgcgccggtaaaattccactgtgtgtgtcgtgcgcatgcgccagtatttttccactagaggcggtcagtgcgcatgcgtcggtaaaattccactagatgtgcgccgtgcgcatgcgccggtatttttccactgggcggccgcacctagggagcgcgagccccgtgccgggcatgggccgcggcggtggaaaattaccgctccgcccacctaggcggggcatctgaaaacctataaaacccggcgtgcccgccgccccccggcgcagtccgcggcagggttccggccgtgctgcggtccgcacgctgcgcccgctcccgcctgcctcccgccctaccccccaccctccccggccgaggcccggcgccggtccgtccgcgggcccgtcccaccgccctggagcaccatccggggccgtgggccgggcaccgggcgcggcccgctccggacctcggccgggggtccctcccctccccccgctcgacccccccatccgacggcccggccgggctgggacccccgcaccggggtcccggttcccgtccgtggcccggggggacccgagcgggggcttcccacccccaccccgctcctccccgggctccggcccgggatccctcgctgctcccggcgacctccgccggcttcccggtccacccgccgcggaatggacgggacccggggtccgcgcccttcccctccccccacggggggctgggtcgcggaccccggttcctaggctcgttccgcggtgggcgaccggggatcccccacccagctccccttcccggcccgccttgctggcttttgggcccctgcgggctttttttttccggctgggggtcgcggcggtcggccgacgacgacggtaggtgggccgggtggacggtggtggggacgggcgacgccccggctcgacggcaatcggtcccggaaggttgggggctgggggcccggtcaggagctccgggagcggggtcgaccgcgacggcttccgggtctcgcggcggctccctctcggcggctccggttgggctcccctcccccctctcgagggtccggccgccagtcgtgaccgggggtccctcggcctagccgccggctctcggtccgccttatcctgggcgttggcctgtcccgtgacgctcccctcccccgctgctccccaaaaaaactccgcccgaaccgtcgcggcttgctggccctgggcgtggtcccccactcccctccccccatcggccgcccagccggggtcggcgcctcggaccccaccaggctgtggcgtgtgtgctggccgatgcggcggcgaggttgggtgtggccggaagcgctcggggtcgacggtgggccgccatgacacctcaattgtcgtcagtacgcccctccacaatcaccgtccccacacgatgggcccggcaggtcacccaacgttggttcaggcccagtcgggttttttccccggcacgaacgcacgtccccgtgggctccacgcgttttccaccctttcctggaggggtccggaacaccgtgaatccacggggagggtcccggcacgggccgaggagaccacgaccgtcccacccggcgtgtcgactcgtccgagacccgggaagggaacaggccccacctttttttcccttctccgattttgccgtggaaaacccgtgaaccgatacgggtgcagacggccgaaaaaaatcgagacgacaatatgacggcagggcgcgatcttctcccccatccgacaaaaccgtgtcccttaaaattccccacctttctctgttcaaatggccccgaaactgtaaaacaccgtttgaccgcaccccaaccggcgccatcttggtgaccttctcgacggttctctcgctcgtcatgccgttctgagctccgacatggcggacgagagaaaatggcgtcgagagcctaggagcgtttttgctccaggcgggtaaaaaaatagcacgataacttttctgtgcttttttttgagacgttttagaagagcttttttctgctcagagcgaaaaaatgatagccctgaaaatctcgacgagtctggccgagcggcgccatcttggaggaggggcgagtcgcgggcaccgcctcggtaccccctggccgaggcgagtccgcggtcgccgcctgttccgtgatgctacctagagggcgctgtcgaggcgactcttcctgttttcgccctgagggctaacggtcgctgacgtcaaaccatctcgtgctcgctgagtcacatccggttgttgacaagcgatggaggaccgcacccaaagtgcgccctctagtcatcgcgcctgaccccttttataaactgctcgaagaaaagaacaccttatgtgaaaaaatacagaatgatgacaagttcatccaacacaaccgctcaacaacgccatatctatcagtgtccaaaaactatcttctatcctttgaaactataaatgctgcctatatacatatttagtatccaagactcttaccacgtagacgaaaagaagtgatacaatgatcttgacgtgtatcgtctatatcgtgctagatatattcagataagacgcgcaaaccatagatttctcatcagtatcatgaaagacctatagctctatatacgaacctagtcattttaggacagccgccggagaagccgacgagggatcgggcgggtgcagccagaacctcacgcccgatcccgcctccggtaggcgatttgcatctgtttggtaaaaagctcataagtctgtatgtgacctatatatattatacgctatgtacaccgaactgtcgctgttgtataagaagaaaaaactctccatatttatatcgtctgaatttttgcttgatagacacgtgtttggaactctgtccccccacgttttcactgtgtataacaaaaatatgtgtttctcaaaagatcttgaggtgtttgaaaacgggggaaacctgcgtttgggtgctctaagccccggactgggacgtagccggcgtccggcacctatatttttctatttttttttacaaaatatatgatgaaccaagaataaaactctagctctcgtctatttttaatatgctctacttagaacctttttaatgacagaatgaactccatgttatacgctctttatatagtttctctgcactaacctttaaaaccgtatccttccctgttgtacaaatcatcttttgatacacaatgatgacctgatatccctccatatatatgatcggatattattccgttagacttgtcctccttttttttcctcatctcctgtatctggagatatatgttgaccaccaccgccatgaccaccaaaaagctagccgtcacgactagaaatgtgtaggattcggactttccgttcgagaagaaaaagagaccgcgtctctggacgctctttttgtcagtctgaatcgacccgggatacgtaagagagcggccctacatcggggggcgctcgagaccgacgacgttccatctgaccagaaaaaaaaaggcacccctcggtggcgacctctcaccatcgtttgcccgtccgcccgtccttcgtagccatcatcatctcaggctctatcggtaccatcgttgtcatctgaaaaaaaaactgcctcacccacctgcgtaaaaacaccatctttccggaggtgcggtaagacgggcaaatacggtcgtgccgaggcaaaaaaaacgcaccatcgacaccacaccctcatgagcaccacctgtcggtgttggtcgtcctccatcgttctctacgaacatctcgacgcccgggtgacggacgacggcaagacgtcccggagaagacggtgttctctcgggcggtacgctctctggatctataatatctatagtagctaaacgagactgtgagtacgacgaaccacatcatcttttttttatgttgcttctttagaaaatgacttatgtcgacgacactcggcatcagccatctcgtgaaacacgctcgcttttcgtctctccaaggaacactgggtccgctgaaagggaccgtgtaccgaccaaagcaaaaaacacacacgtagtaacatgatcaaccacgtctgaatgacacgaaaacacaatcgtataacgctctattcatggaacgaacttggaataaaaaaaccatcgcaggccagaggctaagccgaaaccgtccggggaagcgggcgcgagttttccgacttagcctttggtgctcgttgagcctcttttttttttctgattctctgaagaatcaccgtcacagccctatgacgcgaaatcaattgctagaacataaacgttctcaacaggtatgaaatgaacaaactagatgatgctataaccttatattgtgtgtatatagataggtgtgaaatttgtaggataaaaagtgtcgttgtatgatgcacaacgatcgtgaaactggagactgtagctctctaccgaatgcaaatacacaaatgacatcgattcccgtccccacataaagaaatgtgctttactgtgaaagaatgaagaagattcttgttcctcgtacgacggggccctcgctcgtcgtgcctcttcccccctccgggagaggggacgtcggggccctccgtcgcaccgggccgaagccagtgaaatgtttactacactgtcatcagaatatatgatgtatattatttcctccaaactcctcaccatagccaccaattcgcatcacttaagaaagtagtagcaaccgcggcggcggcgaccggccggtcgtcgtctcctcgtcctcaaatgttgtacatgtgcagaaaaatgtgtaaatacgtgttatttatcccatgcgtcttgtacatagatatatgtttttatatacgctatttatactttatatatccttttgcataaccatagacagtcaaggattttaatgatttgctcatccgcctttgagccatcgcttaggagttagttcctctatgttctcggcccaccttttcgactacagtagcaaacccttgtactaccaccccgataaaaaccacatcatcatcgtcaccacgacctggaaacgacacacgttcccccccaatcttgggcatgtgtatatataaaaagaatgggagggagaggacgtggggctcgagaagaaataaacgccaagctcgattcgaaccaaaaaaccacatgtgtattgtgctttgtttttttttttacggtgggggaaaaggagggggccgtcattaacggaaaccgtgtatggggtccggacacgaacagtacacagcttatggggaaaaaagctcacagagagaaaaaacaccaagctcaggcacgcgtacatcattattatcatcatcggatatctcaccacgggtcatagtagtaccaaggagtgtgtaacaccattttttcttttctttgtaacgggataagggacagcaatcatcacgcacaacacccttcactctctttttagtcatccatatcatcgctgtaacacagcatgtcctcgtaatcgggcgtctggcagcgcattaccaccgagtcgtcttcttgcggtaccggtggtggtggtggtggcggcggcggctgctgctgctgggttgccgtcgtactgtgattaccgttggcggactgcaccgggatgatgggctgcttgtggggaacctggggtggactgccgccgtgagaaggcgacggcgtcatcaagttaagctcaccacggtgactccggacaccggcgaggggcgccgggggactgggagggaccgcggtcgtcttgtagacgacggtgtccccgtgtcgatccgtggctcgtaccagatcttgactgctagcgtcgtcactgtcttcgtcctcttccagctcgccctcagagtagtgctgctgtggttgcgacggtggctgggcgggaggagcggcggcgatcattggagagggatgtcgatgactcccttctctgtcctttttatcgtaggctgtcagcgttgctgggtccgtcctgctttccatatttgcgtattgctcatcggtgggatgaatttggtctcctccccgctgttgtccgccggcagtggcgtggttgctggcggttgtcgttgtcgtaccggcaaagacggtgagatccaatagcgactgctcgtcgaagggacagtacgctatcatgaaacgatagggtgccaacgcgcgttggatgcgcagttcgcacatctcgttctgacactcgtggcactgcagggcgcctaggatcaggtccgagacagcgccgcagcggtaggtacccatggcgttgttagtatcgaactggtcaaaaaattggggcgtaccggtgacttgcaacgcgcgacggcgtagcgagacggccacgcgcgagaaagagcacacataggccatggcgcggtgcatgggttgcgagaaggtctcgggcggacgcttctgcagatcgcagacgtcgtcgcgtagccaggcgctcatttgaccgggcttcttgactagccgtttgagcgtgctgcaatggtcgccccagccgtcctggtggtccaggatgcagcccaggtccaggttgttgagtttgttgaagagtagctgacgcatgccgcccaccgtctccagatagggatcgtgcgggttgacgggtagcccgtgcaggtggtggtacttcatgtagctgagcgtttcgtcgatgatggccagcaacgtgtgcaagttgggagcgttgtacacggcgaagatcttttccaccaccagcttgcgcagcaacggttcctccagccaatcgaactgttgacgaatgtgcaacaggtagtcggtgtgcatgagctcgtcgtgtgacagcaggatgcgaccgcgcggctgatgatcttgcgggaaggcggtggggaccttgagatcggcggggtagggtgccagacgtagactctcggccgtgtagcgctgaaggtcgtaaacgggcgaggtagaactcggtgaggtacccgacgaggcggcgccgcgctgcagacgcgctctttttttcttttcgatcaaacggctgagttgctgtagttcgtcctcgtccatggcgtccagttcgtcgtcaataagcgccagcatctgttgttgttgcggtccggcggacgatccgtgatgattattggctgaggaggggtgagaagaaccgaaagtcgtaggacaactgggaactcggcgacgaagatgcgtcgaatcgccgccgtgatggtgcggttcgccgtcatcgttgtcgtaagacttaccgtagtgggggttaaggggcaccgaggcggacgcggccacgcgtcgcttgaaagaggaggacgccctatgtccgccacggaagcccgcggtgcccatgatgatgtgtccgccggtgcccccgagtgcgtggcgggaggagggtggaaggggaggaggatagtggtccggatcgccttcggtatcatcgtctttgctgtagcggggtcgtcgtgcggggacgcagggtcggtgatgatgcgaggcggcgccgacggtatcttccgcgagatggtgttcgctggcggctgctccgttccgtgtcgacggcgaggttggacttcgctcgcgtcggaacttccgtggcacgggttcgtaatccagacagaagcgccgtgcgcgacgggcgcggcgttcgcgctcgctcagggaagataacgacggagcgtcgtgacggccgcgtgagtgcagctccatggccgccgtcgctaggaaggtcacgttcgggcacgctgatgtatatatagatgagaccgctgccggggggcgggtcaccggcgccgtggaaagtgaggctcagacggcggtcgccggcggcacgggcgcgtcgggcggtctgattttgatggaaatgtggacgtttttggcgttggagtgacactttttggtgaaacagcggctccagaggctggcccagagcgcgtagctgtgctcggtgcgcaggtcgatgaacacctgcacggtctcttgcgggttgcggtgcgtgtagttgagacagcgaaaatcccgcgtgcgcgcgccgtcgcgccgcttgacggccacgcagcaggcgccgtggggctgaaagaggaggacgtggggcgcggtaaactgctcgctgacgtgcggttcgtagtgttgcgtgaggtgctcgagcagcggcggccacacgcgggtgacgacgagccgctgcaagtccgtgtcggaaatcgcagcggcagtggcgccgtcgccaccgtacaggtgataggcgagcacctcggtgagaccgcggcgtcgataacgcgtcacgttaagcgagcgcgtctcgataaagttggcttcggtcgaggggcagattttgtcgcgtacgctgagaatgacgcgtggcggcggcgacaggggcaacgcgggcaggtcgtgcggcgggtggtggtgaagcaggttacgcagatccagttgggcgcgcacaaagcctagcgggtgttcgcggtaggcgtcgggcacgatgaacagcggcaacagacggcgatgcatgaaatagccgtcgtcttggtccattttatacatgtagggcagacgtacagagcgtccatggtggtagatgcctgtgtctaggctgctctcgggatgcgagatggggtccagcagcgtgtgcagttcggcgtcgagacagacggcgtgattgagcacctgcgccacggcgcgtaaaacgctggggtgtacggcgacggtgcaggcggggaacggcgtgatgatgcgcagccccagtttgcccttgcagcggcagtaagggggtgacgtgtcaacggaggacgttgttttttggaaaacgccgttatccgggacgttatttttatcctctttcccgtcttcgtcttcctctgtgtcgcgctcgtcccggtaatcgagatagtcgtcgtcatcgaaaggcgcgccggccgcgtccacgggcacgctgttgggtgggcacgcgcttttgaagaaatagaccgggtgccggtcggggtgcgtgtagccaaagaggctcgcccatacggtcatccagacgcgtcgtagtccgcgacatagctcaaagacggtgtgtcgcgccagaccggagacgccgtcgcgcagccgtaaatcaaagtcggccacaaaattgaagacgggcagacgttcgttgaagacttcgtgtcgcgtgtagtagaactgtgtctcggggctggtgctggccacgtcgtcgtcgtgtagccacacggtctcggtcagggcctcgtccgagaaacggctgtcgggtacgtgacggagcaggtcacgcggaaagaggctgcgatgccaggtttcggaggccacggcgcagaagacgtgctggtcattgggcaggtgtacgcggtagacgggcagcggtcgctccagcagcggtgccagcgcgggctcgggtagcaggtagcgacgttgcgagtaacgcgttagcgtgccggtggtgtaagtctgggctgtgcgtagcgaggcgcatagacgtaacaagccggacagggagcgttccagcggggagaagacagactcggaaagcgtgttgatgcgttcgagctggcgcgccagctgcgtggaggtgccgaagaagcccgccaggtgcgtgccgtcgatgcggccgccgtagccggccagccccaagccgtgcgggctggtcgccgagtggggggattcgtcgagacgcagtaggtgcgtctccacgtagtcgtgtagaaagttgtcgagcgagaagtatttttgcatgacgtccagcagctcggtggaaagccggcggcccagaaaacccggttcgcgcgtgcactgcgcttcgggcgccgcgtcagcgtcgtaagccaccacgcgccggtactcgagcaaccgcgcgcgtgccagcgccgtgcggtaggccaggtagacgtagtgcacgcagaccgtgtcgggcagacgcgcacgttcgcggaacgcgttgatctgcgtgtccacctgctctagctcggtgtagtcgcggcggttgcgcgcgacggcgtacgccacgaaagcggacacgcgctgacggaagggcgagcccagtagcagacgcgcgaactcgcccatggaggcgtgcgtggggatgatggtgcccaggtcgcgcgtgcagaagctgcgcacgtactcctccacggtggagatggtgctgtactggccctcgaataggtagtaggccatggtcagcagcacctggccctcggtgtgcccgaagacgctgatgaaccacgagggcgaggtggggcagaggaagacctggttgagatgacgtagcacggccgcgtggtgaaagtacaccaggtgcttgaattcgcgcacctcgccgccgtgttcgggcgagagcacgggcgtgcggaaaagatgccggtagagcggttgcgtctcggcctcgtccagactggcgatgagcgccgagagggggatgggctggcgcgcggccaggtagcgcgagagctgcagcgtttcgttgttcacggcgaagacgggcgccacccgccgcgagtccgagcacttttgcgtctgtaggcagaagtaaacacgtcgcgagacctggtgtttgaccagcagggggaagacgcagtggtccgtcggtgtctgcgagagtacgttggcgactatatgagcagaatcatactctgttgcgaacagaacgagcgtcatcgtcgcgccggcacgatgcagctggcccagcgcctgtgcgagctgctgatgtgccgtcgcaaagccgcgcctgtggccgattacgtgctgctgcagcctagcgaggacgtggagctgcgcgagctgcaggcgtttctggacgagaactttaagcagctggagatcaccccggccgacctgcgaaccttttctcgcgacacggacgtggtgaaccacctgctgaagctgctgccgctctataggcaatgccagagcaagtgcgcgttcctcaagggctatctctcggagggctgtttgcctcacacgcggccggcggccgaggtggagtgcaagaaatcgcagcgtatcctagaggccctggacattctcatcctcaaactggtggtgggcgagtttgccatgtccgaggccgacagcctggagatgttgctggacaagttctccacggatcaggcctcgctggtggaggtgcagcgcgttatgggcctggtggacatggactgcgagaaaagcgcgtacatgctcgaggccggcgcggctgcgacggttgcgccactgacgccaccggcggtcgttcagggggaaagcggcgtccgcgaggacggggaaacggttgccgccgtgtcggcctttgcctgtccctcggtttcggactcgctgatccccgaggaaacgggggtcacgcgtcctatgatgagtttggctcacattaacaccgtctcctgtcctaccgttatgaggttcgaccagcggctgctggaagagggcgacgaggaggatgaagtgaccgtgatgtcgccgtcacccgagcccgtgcaacagcagccgccggtcgagcccgtgcagcagcagccccagggacgcgggtctcaccgtcggcgctacaaggagtcggcgccgcaagagacgctgcctacgaatcacgaacgcgagattttggatctcatgcgacacagccccgacgtgcctcgggaggcggtgatgtcaccgaccatggtcaccatacctcctccccagataccctttgtgggttccgcgcgtgaactcaggggcgtgaagaaaaagaaacccacggcggcggccttgctgtcctccgcgtgaacagcctggcacgttttggaaaacgtacgtgatcacggacacgacgagtacggggtttctcatagacgtactttattaggtcagggatgacggggaggtttcgggccgacgtcaaaaataacgtcattcgtgttgacagggctttctgcgtcggagctcttttcatcttcttctgtctcgtcgacgtcatcgtctaccggcgagggtgtccgttgcagcaacgcgtgctcgggcgtgtgggtgaaaccgatgtcgggggtgggcggcacgatcatctgtcctagggggtgactgcccaccggcagataggtaaagcggtgggtggtaaaaaccgctttggctacggtggtgtgtggggagatgcagacggtggtgtgcgaagtgttgaccaccgtcacgccggccgcggtacccgggagccagatggtgggtcggatgatgagatccgattgactaaactggcgcacgcccactatgagggcgcagataccgggcgcgtgcacgtaggccgcgtcaaaatagacggtttgcgtgtgacccggaccgatcaccagcgtctgacgggtacgtaacgaaaagaaacggtgttcgttgggcggcggcaagttcatgagctgccagggttctggtacaaaacaggggaaaacgccgatatcgccttcgatggtgcccggaaagatggactgaaaagtgtcgttgaggttgacgacatccaactgcgggacttgcagcctggattccagcagctcgggcatgcaaacgaattgcgcgtccaggcatttgtaaaaggtaatgccgaaaaaaccttcggggatatagaggctgacgcccagcgaggtgggcactttgcgctcgcgtgatagccaaatgatgtgtttattgtaaaaggccagctgcgtgtggcattgtttgacgatgaaactggaaggcatccacttgtaaggaactttgagcggtgacggtaatggcgacgacgcttcatcctctcccggatgctgctctttgtcgtatttctcctcggtcgattggggcagcgtaaatgtggtttgaaaatcgctatcgctagcgaaacgcacgcagtaacgcatgttgacggatttctcggctaggatgatggagcctgatgacgatgcggactcttccttcattattaacgtaggggtctcccagaatcgctgaaaacgggagcgcggcagccgcgacagtaccagttgagagtcgattcggtcggtcaacatcgtaagcatcgtggcggtggtgtgatggagtggaacacactagtattaggtcttttagttttatcggtagtggcagagagttctggtaacaattcatccacgtcaacctctgcaactacatcaaagtcttctgctagcgtatcaactaccaaactaacaacagttgcaacaacttctgcaacaactacgacgactacgaccttatcgacaactagcactaaactcagttctaccacccacgatcctaatgtgatgagacgacatgcgaacgatgatttttacaaggcgcattgcacatcgcatatgtatgagctctcactgtccagctttgcggcctggtggactatgcttaatgctctaattctcatgggagctttttgtattgtactacgacattgctgcttccagaactttactgcaaccaccaccaaaggctattgagggtggacagatttacagcccggcggtgttccggcggggtaaggtttacatacgtgggtgaccggaggctaaagttacgaatctcatctagaaacagcagcgagtctagatagtcccacaggggatctataaatgttctctgaaaccccattgatggtgacgtaggtgtagttttgttactatcggaagctgttttgttttccacgaacatggtttcgttgtaatataaggagctcatgtcgagagtaccgtaaatagtgtacggcgtttcgttacggattagtacgtgcgtgtttttcataaattctgacacggcggttcggttgcggcttggttcacaaaaaggattttgccggtaacgtagagtggtatacacccacgttgctaggtcccttaactgtgtggccataatggacttcataaagctgctatcaggacgataagcaattgtagacgtggaaacccgccttgcggcggtagtaatactataagtcacgttagtagtgacgttgagagcggcagacgttgtataggaaaagtatggcgtagtagtactctgagttttcttagcttttttttcgaattgttccttaacgggcgcttgtttacgttttagttttcgcatagtgttttttaacttggtgccgttaatatacttggggacgcgaaatagattccggctcatggcgttaaccaggtagaaactgtgtgtacagttgcgttgtgcgtaacgtaaaagcagggcggttaaacctagaaaataaatcgtttgactatctacgttaaccttagtcggacccacgtacaatttggtgttccaacgcggtacattgaaaaacatggggttgaacgtggtgaaattaccgcaaccttgttcgccagtatcattacgtttggaaacgtttagcatttcggaaagacaagtcatggaaggcacagtaccacaaggtgggggtctgaatgttatcgttttagccgtatgattgtactgtgagtaaacgtattttgcgggttttctaagctgggtactataaaaatcaaaccacagataggttatactataattctgaatggggcccgctaaaatgtagtattgtggaaactctgtcatgttcatagtgagatttttaaccggttgtttacttacattgtattttgtagaaatagtcgtttctagttgtctcaaaatttctaacttaagctgatctaatttatatttgcctatcttagacagtaccaagcccctccaaggacgattataaagcgcttttgacataactttacagtttatgaaagaaacaagcaagaaagatatagatattagaaacaccatcttagggacgtctctcaccatcatctcttttctccccatgacagaggaggagaccccgcaccgtccgtctgccttgtggtttggcttgcctgcgtgtactcactgctgattctggtcgttttgctgctcatctaccgttgttgcatcggcttccaagacgacctagtctcccgcaccttggctgtgtaccaagcttgtatccagggcccgatatgtaaccagacccataacagtacctcgtaaataaagacgcacagacctcacgcatatagtaccatcacaccgtgtggcgtgtactttattacaacgagcaagagtgcccctaagtattggggcccgtaccgttttagaagattttgtgtgaatgtctttaacttttctgtcccttttctcataaactgtcaggttctacagtcagcatgtcttgagcatgcggtagagcagatagatgccgatgatggccgatagcgcgtagacggacatcatgaggagacgactgtcggtggcgtccacgacgacgtcagttacttctaggaccgtaccgtttttcaaaagcatgaggtagtgagttcgcggagatgagaccaccacttcgttgtagggatccagggcgaaaaggacgtcgtccgagtcgtgcatgtacatgatgttgatgacgccttgcgtgtcgtcgtattctagcagggcgctttggcaaaaggcgcagttttctagtgaaatgttgagcgccgctgtgatgctgtgtgtggtgtgcatgttgcgcgttagttcgcatttagtttgactgtccgtttgggtgatgatgaggctctggcctacgacggtggtggagacagggtaggagatacctttgatcaggtactggtttgttacgacataactgacgtgttcggagacggttagcgcggagaaggattcgccgagcggcagacaaaacaggtcggggaaggtttccagcgtgcttggttgcatggtagataggatggagagggcggcgggaacggtagcaggaacggtggcatcggggaagagacgcgtgaggcgttcgagcgagtgatcgcgtcgcccgctactggaacagggtgtgtacaggtcgctgaggtattcgtggtgcggatgagctagcaactgcgtaaagtgtgatagctcggccaatgaacagaggcccgtttctacgatgaagatttcgcgtctctccgtcgtatgtaccaacatggagtggacgaggctgcccatgaggtagagttcttggcgcgcgaaggctgaaagaaaagaggccaggtgcgttttgtgtaattgtagggcaaagtcggcgatctgtcgtagtgcccactggggaatgagatgttgctgattctgtttagaaagtatgtagaccaggcgtacgaggctggtgatgtcggtgatctggtccggcgtccagagggctcgtttggccaggtccacggctgtgggatatagcagcaatgtggtgcgtggtggtgtttgtgagaggcaggtgatcataaattcttgtatttgtaagagtgcggcctggcggtctagggctcgtgggatggagatttcggtgccggcctcttcttgtcgggctgccgcgaacagtgctaatgcgtaggcgaaggccatttctaccgtgcggcggtccaacatttgacatcgaccgcttttgagtacgtctacagcgtaacggtgaaagctgttacgtaacagtgcgctgaggtccaggtagttgaagtcgagtgcggcgtcgagaaagtccgagtctttgagataggagtgacggtttagttgagctttcttaactagtaccaggagctcgtgtttttcagtttgtcgtagtataaagttgtcgcgttgatagggcgctttgaagagtacgcgtggaagatgaccgaagataagcagcatgggtgtgtcgtcgtctatagataccgtaactacgaagaagtcctcggtcagtgtgattttaacgtaacgtagttcgtccatgaggtaaaagccctggtgcagacagggcgtaacggtgctgaaaagcagatcgtgtccatcaaagaggatacaggtctggttaaagtgtggccgatgtagtcccgaggtggtgtgcgatcccttccagtcgtgtggagtggtttggggtggcatccaaacgtgaggtattgacagatcaatgggcggtggcacggtggtgggctgctgacccaggctgtcttgtgccttcagctgctgcgaaaaagatcggtagctggccaggtctttggataccaatgcgtaggtgttaagtctctgttggtatctttctagggtttcggtcagatctacctggttcagaaactgctccgccagaggacccgcaaaaagacatcgaggcatatggaatacatagtattgattatagctttggaaaaagttgaaactgatggcgttttccctgacgaccgtgctgttacggaggctgctgttgtaggtgcactgggtggtgttttcacgcaggaagcggatgggtctcccgtaggtgttgagtagtaggtgaaatgcgtgagggtccagcgcttcggatgcggcgtccgcgccatatcgttgcgaaggtaggtgactgaggaggtagacggtgaagacagtgaggtagggggggaggccgggccgcatagcgcggctgcgccgctgggttcagcggcgtgatccaggtggtggttggcgttacacccgagagaaggagagaaaggatcccaggaaggggcacccgggtgtggcgctacgggttacaaaagtcgcgtctctgtctatttaatacgatgtcattggccgctgcgaaggaagaagaggggacacgcgggtaagccatgccgtccgggcgtggggacgacgctgattcgacggggaacgctctgcggagattgcctcacgtgcgtaagcggatcggtaagcgtaagcacctggacatctaccgtcgcctgttgcgggtctttccctcgtttgtggccctcaaccgcctgttgggaggccttttcccacccgagctgcaaaagtaccgtcgccgtcttttcatcgaagtacgattaagtcggcggattcccgactgcgtgttggtgtttttaccgccggactctgggtcgcgcggcatcgtgtattgctacgtgattgagttcaaaactacgtactcagacgccgacgatcagtccgtgcggtggcacgccacccacagcctgcagtacgccgagggcctgcgccagctcaagggcgccttggtggactttgattttctgcgtctgccgcgcggtggcggtcaagtctggagcgtagtgcccagtctggttttttttcagcaaaaggccgatcgcccatctttttaccgggcttttcgttcgggccgtttcgacttgtgtaccgattctgtcctggactatctgggacggcgtcaggatgagtctgttgcacaccttttggcggctacccgtcgccgtcttcttcgaaccgcacgaggaaaacgtgctgcgctgccccgagcgcgtgcttcggcggttgctggaggacgcggcggtgacaatgcgcggcgggggctggcgcgaggacgtgctcatggaccgggtgcgcaaacggtatctgcgtcaggagctcagggatctgggtcacagggtgcagacttactgcgaggatctcgaagggcgcgtgtccgaggcggaggcgctgttgaaccagcagtgcgagctcgacgaaggaccgtcgccgcggacgctgctacaaccaccgtgtcgtccgcgttcttcgtccccagggaccggcgtggcaggagcttctgccgtcccacacggtctttatagtcggcacgatgccatcacgggacccgccgccgccccgtctgacgtggtcgccccgtctgacgcggtcgccgcgtcagcggccgccggtgcttcttctacctggctggcgcagtgcgccgagcggccgttgcccgggaacgtacctagctactttggaatcacgcagaacgatccctttatccgctttcacaccgattttcgcggcgaggtggtcaacaccatgttcgagaatgcctctacttggactttctcctttggtatctggtactatcggctcaagcgggggttgtacacgcaaccacggtggaaacgagtgtaccatctggcgcagatggacaacttttccatttcgcaggagctgctgctcggcgtggtcaacgctttggaaaacgtgacggtgtatccgacgtacgactgtgtactctccgatttggaagccgccgcctgtctgctggccgcctacggacatgcgctttgggagggccgcgatccgccggactccgtggcgacggtgttgggtgagctccctcagctgttgccgcgtctggccgacgacgtgagtcgtgagattgccgcttgggaaggccccgtcgccgcgggtaacaactattacgcgtatcgcgactcgcccgatctacgctactacatgcccctaagcggtggtcgtcactatcacccgggcacttttgatcgtcacgtgctggtgcggcttttccacaaacgcggcgttattcagcatttgccgggctacgggacgataacggaggagctggtgcaagagcgtctgtcgggccaggtgcgcgacgacgtgctttctctctggagtcgacgtctgctggtcggcaagctgggtcgcgacgtgcccgtctttgtgcacgaacagcaatatctgcgttcgggcctgacctgcctggctggcctgctgttgttgtggaaggtgaccaacgcggatagcgtcttcgctccgcgcacgggcaaatttacgttggccgacctgctgggttcggatgccgtagccggcggcgggttgcccggggggcgcgcgggcggcgaagaggagggctacgggggacggcacgggcgggtacgtaactttgagtttctggtacggtactacatcgggccgtggtacgcgcgcgaccccgcggtcacgctgtcgcagctctttcccggcctggctctgttggccgtgaccgagagcgtgcgcagcggctgggatccctcacgtcgcgaggacagcgccggaggtggcgacggcggcggcgccgtgctcatgcagctcagcaagagcaaccccgtggccgactacatgttcgcgcagagctccaaacagtacggcgatttacgtcgcttagaggtacacgatgccctgctctttcactacgaacacgggctagggcggctgttgtcagtgaccctgccgcgtcaccgtgtgtccactctgggctcgtccctctttaacgtcaacgatatttacgaactgttgtactttttagtgttggggtttcttccgagcgtggcggtgttgtaatttccaccacgtgtcgctcgctgcataaagggcgaacgtcctcggagagggtatattcgttcggcgagagcgggcggcggtggtgggtatgtccccttctgtggaggagactacctcagtcaccgagtccatcatgttcgctattgtgagtttcaaacacatgggcccgttcgaaggctactctatgtcggccgatcgcgccgcctcggatctactcatcggcatgttcggctccgttagcctggtcaacctgctgactatcatcggttgcctctgggtgttgcgtgttacgcggccgcccgtgtccgtgatgatttttacttggaatctggtacttagtcagtttttttccatcctggccaccatgttgtccaagggtatcatgctgcgtggcgctctaaatctcagcctctgtcgcttagtgctctttgtcgacgacgtgggcctatattcgacggcgttgtttttcctctttctgatactggatcgtctgtcggccatatcttacggccgtgatctctggcatcatgagacgcgcgaaaacgccggcgtggcgctctacgcggtcgcctttgcctgggttctttccatcgtagccgctgtgcccaccgccgctacgggttcactggactaccgttggctaggctgtcagatccctatacagtatgccgcggtggacctcaccatcaagatgtggtttttgctgggggcgcccatgatcgccgtactggctaacgtggtagagttggcctacagcgatcggcgcgaccacgtctggtcctacgtgggtcgtgtctgcaccttctacgtgacgtgtctcatgctgtttgtgccctactactgcttcagagtcctacgcggtgtactgcagcccgctagcgcggccggcaccggtttcggcattatggattacgtggaattggctacgcgtacccttctcaccatgcgtcttggcattctgccgctctttatcattgcgttcttctcccgcgagcccaccaaggatctggatgactcctttgattatctggtcgagagatgtcagcaaagctgccacggtcatttcgtacgtcggttggtgcaggcgttgaagcgggctatgtatagcgtggagctggccgtgtgttacttttctacgtccgtccgagacgtcgccgaggcggtgaaaaagtcctccagccgttgttacgccgacgcgacgtcggcggccgttgtggtaacgacaaccacgtcggagaaagccacgttggtggagcacgcggaaggcatggcttccgaaatgtgtcctgggactacgatcgatgtttcggccgaaagttcctccgtcctctgcaccgacggcgaaaacaccgtcgcgtcggacgcgacggtgacggcattatgagcggcggcgctgtacggcagcggggagaaaagtggcagataaatcacgtcaggttcacacgtcgttagccagcgtcggcatatgaagggcgcgggcggccagtacggcctctgggctgagacaggacgaggcagggtgagaaagaggaggatgggggggaccggggtggtggtgctgctgctgttgtgggtgcggacggtgcgggtgccgggacagcgtgccggcgaacgttctgtaatcttccataataaaagtaaaaatgcccgtctcgtgtcgactccgctggatctcgaaggcgtcgggggtaatgcgcatcttgccggtgccgatgagataaaagtaccacattttttgacagatgatgcgaatcaagggttcgtacgcttcggcaccccagtggcgcgtgaagaaggccgccagacgaaacaagcggtgtccgtagagcgtgcctagggagaagaggatgttgccgttgcgcgccaggtcttcggggaaaacgaccggcaggccggtgtggcgctgcacaaagcgcgtcagcagtccgccgctcaagcgcgggtgacacaggcgctggctgagacgggcggcgcgcgtttcatcgaacacggccgcctcaaagtccagccccgggaaggcctggcgcagttcgcggtacagatgaggccagtagggttgcggcgtcttgcgactaagcacggcgtggtccgagacacccaggttgttcatggtttcgcgcagtagcagcgtttcgagaccgcggtgaaagaggaggacgcagatgaggcgtacgatcttgagttcttccaaacgcagcgagctcagcggctgtccgcgcgacatcttctcgctaatctgtaatattagatgattggcgcaagtaaaggagaatttgcccgtgcggacccgcgggacggcggggttctcttcgtcgcgggccatcatcgttcgctcggtgagcgggtagcgacggtgacgacaatgacgatggacgagcagcagtcgcaggctgtggcgccggtctacgtgggcggctttctcgcccgctacgaccagtctccggacgaggccgaattgctgttgccgcgggacgtagtggagcactggttgcacgcgcagggccagggacagccttcgttgtcggtcgcgctcccgctcaacatcaaccacgacgacacggccgttgtaggacacgttgcggcgatgcagagcgtccgcgacggtcttttttgcctgggctgcgtcacttcgcccaggtttctggagattgtacgccgcgcttcggaaaagtccgagctggtttcgcgcgggcccgtcagtccgctgcagccagacaaggtggtggagtttctcagcggcagctacgccggcctctcgctctccagccggcgctgcgacgacgtggaggccgcgacgtcgctttcgggctcggaaaccacgccgttcaaacacgtggctttgtgcagcgtgggtcggcgtcgcggtacgttggccgtgtacgggcgcgatcccgagtgggtcacacagcggtttccagacctcacggcggccgaccgtgacgggctacgtgcacagtggcagcgctgcggcagcactgctgtcgacgcgtcgggcgatccctttcgctcagacagctacggcctgttgggcaacagcgtggacgcgctctacatccgtgagcgactgcccaagctgcgctacgacaagcaactagtcggcgtgacggagcgcgagtcatacgtcaaggcgagcgtttcgcctgaggcggcgtgcgatattaaagcggcgtccgccgagcgttcgggcgacagccgcagtcaggccgccacgccggcggctggggcgcgcgttccctcttcgtccccgtcgcctccagtcgaaccgccatctcctgtacagccgcctgcgcttccagcgtcgccgtccgttcttcccgcggaatcaccgccgtcgctttctccctcggagccggcagaggcggcgtccatgtcgcaccctctgagtgctgcggttcccgccgctacggctcctccaggtgctaccgtggcaggtgcgtcgccggctgtgtcgtctctagcgtggcctcacgacggagtttatttacccaaagacgcttttttctcgctacttggggccagtcgctcggcagtgcccgtcatgtatcccggcgccgtagcggcccctccttctgcttcgccagcaccgctgcctttgccgtcttatcccgcgtcctacggcgcccccgtcgtgggttacgaccagttggcggcacgtcactttgcggactacgtggatccccattatcccgggtggggtcggcgttacgagcccgcgccgtctttgcatccgtcttatcccgtgccgccgccaccatcaccggcctattaccgtcggcgcgactctccgggcggtatggatgaaccaccgtccggatgggagcgttacgacggtggtcaccgtggtcagtcgcagaagcagcaccgtcacgggggcagcggcggacacaacaaacgccgtaaggaaaccgcggcggcgtcgtcgtcgtcctcggacgaagacttgagtttcccaggcgaggccgagcacggccgggcacgaaagcgtctaaaaagtcacgtcaatagcgacggtggaagtggcgggcacgcgggttccaatcagcagcagcaacaacgttacgatgaactgcgggatgccattcacgagctgaaacgcgatctgtttgctgcgcggcagagttctacgttactttcggcggctcttccctctgcggcctcttcctccccaactactactaccgtgtgtactcccaccggcgagctgacgagtggcggaggagaaacacccacggcacttctatccggaggtgccaaggtagctgagcgcgctcaggccggcgtggtgaacgccagttgccgcctcgctaccgcgtcgggttctgaggcggcaacggccgggccctcgacggcaggttcttcttcctgcccggctagtgtcgtgttagccgccgctgctgcccaagccgccgcagcttcccagagcccgcccaaagacatggtagatctgaatcggcggatttttgtggctgcgctcaataagctcgagtaagagagacgctatatttagggcttccctctcttttttttctacaccgtgataccctaataaagcacaccgcggttattatcaacgtctctgtgtttttattatttagaaataaatacagggaatgggaaaaacacgcgggggaaaaacaaagaagtctctctctagatgcggggtcgactgcgtggggtgctggaagtggaagcggtgctgatgggtgagggtcgtggcgcgggcacggaccgcaacgtgctgctgatgtctgccgcggtacgcacgtcgccgtccatgtcgctgcgcagataagaggtaggtcgtagtgcggcgtgctgcacgctcaccgttaatggtaccaagtcgtcaaggctcgcaaagacgtgccacgaggggatgacgagcgtgagagccccgttgttaccgcttcgacgtctttgtccggtcaggatcagtgccggggacagtccggcttgggtgtccgagtcctcgtcgccgctggcttcctcgaagccggcaaacatggcttcggacaggggggtcggcgtcggtgtggaggagaggtcatcttcgtcgtcctcttcctcttcttcctcctcttcctcggtgggtggtaatccgggggactgcgggagaaactcggagacggcgccgcgcatgacgttgctccgtggaaagagaccggcgcgcagctgcacctggggacgcttgattttgtccggtttaccgggtgtgagagtccaaaacccacggcggaaaaagtggatgcggcctagcggctgtcggtgttccaaatgaacggcctgatcgccggtcagcgtgacgcggagggtgattcgcacacgatcgggtagcgggccggcttctatggagacgcccgggatgttttccgggaaaaagatggtgtcgtgagtctgattggtctcgaaagcattctggatctgcacgatgtactcgggatgtatgcgcgtcagcgtaaaacttttgggaatcaacagctggaagccgttgtccggcaagcgtcgtaggtgcgggtacggattgtgtcgcgccaccacctcggcgcgatgcgtgtaaaccgaaaagtgcagaaacacgctggtcggcgggtgcggtgagtcgtgatgcagaaacagcatgatccattggcctcgttcgtccgtctccgttttgtggatgtacgtgttagggtccgaacaggccagctgctccagggcgtctaccagcgtcagcgggatggcgccggcgcgaaaggcgaactggctgacaaagatctgccctgcctccaaactgctgtcggttctgcggcgccagttcggcgttacggtcagtcgcacggcccagtggtgagccgtgcggcggatgatggcgcgcgcttccattcgcggccgattttcttcgccgccgcgccgctggctctgaaagaggtgcagtccgctaacgggcacgcggtccagcggcagcgcaaaggccagtaccgagaccgtgttgttttctgagcctggcgtcaggcgtcgtgggccaaagttgttgaggtccaccagcagtcggtcctgttcgcccaccacgcagcggcccttgatgtttaagtcggtcaggtctacggtgtcgtgcggagatttgttctcctgaaaacagcagagaaccgagggccggctcacctctatgttggtacgcaggtccaggagtcgcagacgaccggcttccagcgagccgccttccacgttggtgatgagccgaagcacctggcagtgcaggcgaccaaagcttccgctggcggcttcggcctcgctgatcgcggccgcttccgacgagggtccctcaccgggcgaggacgatgcctgagacattgcgaaggcgggatggggggagggtcaggggatgcgcaaaggtgaacgggtcttcgtgggaggtcgggaagggttccggcaactgtcgcaaatatagcagcggtgacaggtgtggcggccaaaagttgcgtgtctgagtggacgtgggtttttatagagtcgtcctaagcgcgtgcgcggcgggtggctcaacctcggtgctttttgggcgtcgaggcgatgcatggcccgggcaaggcgtcttgccggtggcggcgacgtttgggttgcgcagcgggctgccatacgccttccaattcggcgaagatgcggtagatgtcgttggcgtcccagaagaattcctggtacttcagattctgaccctgaaccgtagccaccatgggcaccaggttgcgggccaggatgccggcctgccagggcggccaggtgaacacggccggattgtggatttcgttgtcggaatcctcgtcggtgtcctcttcgggcgcgacggtggactcggccttaaggcggccgcgtgtcataacgcccgacgtgcacgccgtcgccgaggatgctgatttgcgtttgcggcccgcggaagtggaggcgcccgccatggcgccgccgccggtgacgcggggcgtcttgcgctcggtggttacgagttcttcgtcggagtccgatccgctggtccagacgtcgtcgtcgccctgggcggcaccctcgtcgtgccggtcccaggtgtgtcggtactcaagcttgccctggatgcgatactggctggtgaaggtggggtgctcgctgtactgaggcccgcgctgcagcagcaagtcgatatcgaaaaagaagagcgcagccacgggatcgtactgacgcagttccacggtctcgcgtatggcttgtacctccaggaagatctgctgcccgttcatcaacaggttacctgagatgctcaggcccgggatgctcttgggacacagcagcccaaaatgctcgtgtgaggtaaaagccacatccagcatgatgtgcgagatcttgcccggtttgattatcatatttttgggacacaacaccgtaaagccgttgcgctcgtgggggcgcatgaagggttgcgggttgcgggtcatcgtcaggtcctcttccacgtcagagcccagcgtgacgtgcataaagagcttgccggagggcacgtcctcgcagaaggactccaggtacaccttgacgtactggtcacctatcacctgcatcttggttgcgcgcgtgttctccatggagcaaaccagctcgtgcgcgcacaccacgtgccgcagtgccacgtccttggtgggaaacacgaacgctgacgtgtagtagacgtcgggctctttccactggttctgctgacgcgtccaggccagtcccgagaccgtgagacgcgcctgccacatctgcttgcccgacgcgtgaatcacagcgtcagctacgggcaggtgtcggtgtttgcgctcggccgccgacgggtagtggtgcacgttgatgctggggatgttcagcatcttgagcggcagcgcgtacacatagatcgacatgggctcctggctggggcagatgcttcggcccgtggggttgtgcacgttgaccgacacgttctccacctcgctgcccgtaaagtacgtgtgctgcacctgcagctgattgtcgccgcggtggcatggcgtcgagtcgggcgtgtactgcgataccaagatcagcgagggctggctcacgcgtacgtggatacccgtctgcaggagtcgcgtctcgtgcggcagcaccggcgtatcgccgcgactaaacacggctttcagcacgtgccccgaaatgggacccagtacggatatcatttcgggacaacggcgaccgcgcgactccatgctgcctgcgcgtacgggtgtaggcgactgagcggcgcgccctctgcggccgccgccttacataggcaggcgaccaaacgcggaacccgaaataaaaacgttctacacagagacaaccgcggattattgagtgtctttttttattacaaaaaaaagaggcgaagccccaccgtcaccacaccccatcacacaccaccaccgatttttttttgttttaatcccgtatggcgcggacgcctagtgtccgtttcccattatcagggtcctctgtttagagatcgccgcagaccatggctaaagtgacaggactcgtcttctctgtcgtattttccgtaagcttacagtcttgcggttccgtctccggggacgccagtcgcatgggcagcaggtcctccagcgcgatggaagcgcccagcaccgagagctgctgttgcgacggcgaatgggatgtggaccgcgagtgtagcgtggatttgacttggtgcgtcattgctgacaggcaaccccgattcagcgtatgctttgacgagataaaatagaggcgccccaggagcgcgtcccgtgggaacgtggcgccgttctcgtcgctcaccagtacggttaattccaaccaggagcgcggtagccagaccgtaacgggcattttgagtccctgacggttgtgtggtacaaaaacacccagataaggcccgtaaaagcggcggtagatacgtaacgtgtgcgagttcttcagcgtcaattcgtaagggacgcgcacctccagtccctcgtccgccgcgccggagcgtggcggtacaaagtaaggcagtggcgcgtccgaaaagaagggtcgtcgcaccgtttcgcgtcgcagccgcaggcgaaacgccactgggtcggctggcgcctcggtgcggtcgcaggtcacgttgaaacgtaatatgccgtcttggtatagcgtgagtgacgacagcgtcaggtccggcggtgattcgttcgggtctagctccaatcgtccaaagacggagggtcccaatgtcttggccgtggtttccgagaggcgcgccgagatacggctggtgagtccacgcggccccgagatgccgccttccactcgatgccagcacagcgcgtgtcgtacgcgcaccgtcagcgtgggcgtcagatccgcgtccgttgattccgcggtatcagcgacggaagccgcgttctccgttacgttgtttatatccagcgtcggctcgaacgtgagttctggcagatgcagcgccagacagtcgtgtaacgccgtgtgatgcgcggctttacgtcgtagcggtagccgtttcaacagcggcgtgatgatacggagcgcgaagagattgagtgataggcgcacgatggccatgcgcgtcagttgttggtcaattaccgagcgcaggatatggcagcctgggcgtgcgggaaagagagagaaggccgggcgcacgtcagaatcctcgttagagaccacgcatagaatgccgcgttcacgatcgtcgttgcggtcatcctcgtcctcttctttcttctcttgtttttccttttttttctcgggctcgtgggaagccgccgtttcttcttcttgcaacgtcgcgggggcggtttgagactcgtcgttcgcttcccccaattgcagcggcgtagagagcagaatctggaagggatcccgcaattcttcgggtcggaggtcgaggtgcaactggatcagatggtaggtgccgcggtgcacccgaggctgacggatgtcgtgtttatccgtcagtgtgaggatggtctgcggcgagccgctgtacttgtccagctcgtccggcgttttcaggaggagactgtcgtcgtcggtactggcgacgcccatcatggtcgtggtggtagtggtggcgaggaaagtgagcggcggcgccgacagagctcggcgttggcggcggcattttccgctgtgtcggctgctattgctgccaacgccaccgccgccgcctcgtctggctcgtggccggcgggcccgattccgaaggttggggtcgacgcgtggcatgcttggtgtctgcgggcgcgagagggccggctcagcctttaaatatgcaggtcgcggatttgttatcgggtgaaacgtcacacaccgtgaagacgacctgttcgcggatgaggtcatccagctgtcgcagcatgacgaaaagcgccgacagccgcgcgatctcgtcgtcgggcgacacgtgctgcggccgcgcgggcgtgcgcggctcgccgacgctgcgctcgcggtccagccgcatcagcagctcctggcacttgacgagcagcatggagctgtcctctagcgccaacttgcgcacgtaggtcatggtcagctccgaggctaggttggccaccatggacatggagaggcaggcggtcttcatgtcgatcagcaggtgctggtcgatgaccggatcggggatggtgaaggtggcgtcgcgaaaagtaatggtctgcagctgctgcacggcagcctttacctcctcgtacgaacggtcgagcgagaagaggcccatgatgagtagtcgctggttgatttccagcgccagtggcatgggtacgatccagggcagcaccagctcccactggcccagcgtcagcaggttctcgcgcgccagcggtccgtggaagagcggcggcagcacgcatagcgcgtcgcccttctcccaagtcacgggtcccgtgttgaggacggtgtagagcagtccgtgcgtgggtacgtgtaggaggatctggttgccttctacgcgccgcatcaacgtcagcgtcatattgcgcagcaggccgcgcagtcgtacgtagccgcgggtgtgatctacgaactggtgtaggcccagctggtagtgcttgatgagatgtagacgttgcggaatgggcacaacggccgctactagcttggtcagtttgcctacgtcggcgatgctgagcttgtggtcgaaagtgcagaagatgttggcctccatggccgccatagcggcggtgaaatcctggccgcgacggaggagaggcagagacgaacaacgtctgcaccgggcgcggcgtcagagcgagcgtggcgcgtccgggcccgcgtttgcgtctaggtgactcgccgctaacctgcggtcgtcgccgtcctcctcaccggacggcctcacgagttaaataacatggattgctgcagcgggatgatttcgcctacgacgtagttaccaaagtgcgtttcggacgtagcaaaagccccggcgccacccttgagtttggtctccatcagcgccagcgtggtggtgctgaggatcggtagcgcttcctgcgtcagacggcacgggttttcgatgagttgttccgtgccttcgacgcagacgtactgcgtgtccgtgtcgccgcggatgcagtccttggcgcgtagcaggtactcgtcgatggttttgaagagcgttttgttggccgcgataatctcttctgtgttaaagtactgcgcgcaagggctgtagaatttggagttgtagcctagacgttcgcgatgtcgggtgttgtagagtacgtcgctcagacagccggcttgcgaggcccaggggttgtgtgtggccgcgaaagtctgtgcgtccgcttcgcgatggtcgtagatggccttggtggcggcctccgtgtcgtacggatcgacggccagcatgcaggaggcacgcccgcgcgggttgttggggatcttaaagtaattaacgtccatcgtcaccggcgtaaggattagttcgcacgcggccttttgtccgtgcaccgtggcggcggcattgcgctcggacatgctgccgaacgtcagcatagagatggtctccgtgtctaacagttgcggccgttctacgccggccgcgtgccggatccagcggtccacctcgtcgtgccggtacacgttcatagggaagacgcgaaagaggtcctgcacgcggacgcccatgtcggttcgcacgcggtttacgtaggctacgcaggtatttgacgtgtaacccagacccatgtctacggtgttaatgttctgcgtgacgtggtacgtagtgctgatgtcgcgttcctccttggtcacgatagggttgttgatgataactgacgtgcatgatttgccgctgtagagcagcatgtccacctcgaaggtgtcggtgcgtacggccgtgagtgcgaatcccgggtggatgtgcgccttggtctgcagcaccagtgaaactggtgagattttgtataacatggcggccagcgtcatgactgagtgcaacacgttgggacaggtggccgagtaacgcgaaaagggcgagcgcagccagttgtggtactcgtgcgcgaaggctgtgggtagcgggaaaccaccgtcgtgacggtgatagtgcgggaactcggtcacgtagcgtttaatgtcgtcgctcaacgccgcgcagatggtggggtttgagtagaaacggtggaaaggtacgggtaggctgtactcgatcaacgtcttaggcgccgtcacgacgcagcagccgttgtaaagcacgtgctgacgtgagataaagtccggcaggccctgacgctgcgcgtggtccagaggcgcgcgcacttcgagcaccttgacgtgctcgcccacgaattgcacggccaaaaacagttcacgacaggcctgcagcagcggcgtatgtgcgtcggtggcgacgtcctccaccagctcggtcagcatctcgcctacggcttgacgttgcgccgctatcgagtcttcgggggtgacaccgcttgtgctctctttcgacgtcgtacctgacgtggagaccgcggtggcggccggcatcaggagaaacgccggtcggtaaaagaggtctactagcagcgtcttgaggttgagtcccaggccgcaggcccggttgttggtcatggcgggcatgaggcagagataaaagaccttttgtaacgtccattcgtcgtcggtggcacggtaatcgtccacaaacagcggctcgtcggcatccatggcgcccaaacgcggtacgtccgaaacgccgtggtgtcgcgcctcgatgttggccgggttcaacggttgccggtcggccactacctgtacgccttccatgttacgcggcaggtgcgtaacgaaggggggccacagccggtggtcgtgcagcgcgttcacgtaagccgatagcggttcctcagccagttgaccgttgttaagtcccggcagcgctgagatgcgcgttaccagacgcagcacggcgaccagattgcggtagtgaaagagcaactgcggtggtagggcgccatcagccaggtgttcggcgatcaacgtcaccagcgcgtagctgtgcgcaaaaaccagcagctgacgtgtgtgaaacatgttgacgatacaacgtgctacgaaagtgcggattagcaaaaaagcgtcgacgttgccgtgtaccagcacgtcgaccaggtagcaaagctcggggtaattggggcttgtcacggtggttttgaaaagtcgcaacgtctcttcgtagtcgggtggtggccgcagtcgcatgtgttccatgatctcccaggtgcgcagttcgtggaaggggcccggtgccagtccatctggcaaattaccgatgacgatacgcggtgtacacagcgccaccgtttcgctgttttcctggcagtgcgtaaagtcgaagaaggggtgcagctcggtgtagagcgtgatgttgcccaccttgtagaagtcggtgaccacaaagtcctgcttcatttcgttcaccgtgcgcgggacctcgcgtcgtacgcggtaaaaatgcggtatgcggcgcgccgcaccgcccatgggttcctgctgaaaacgacactcgagcagtcgttgcatggcgggttccgagggcggtccgcgttccgtgaaggtctgtagacagggcgcgggctcgtgcagcaccgggtggcacagcgtcttgagcgcgtccacaaagtctattttttgtacggcacggtcccggtttagcaggtaggccgtggtgggcaacgcgttgcgaacggtgtcgttaagcttaactttgctttccaccgtggtgtaaccgcgatcctcgggcagatacagccctacggggaagaaaaacgtcaggtccacgttacgttctagcggatctttggtatcggtgtttttgtagacgcgccgcaagttttccataatcaccgttttttcgcccagtcggatcacgtccatgctcagcggcgttaagctgtgcgccccggcctgcgaaagcgagtcgttgggcaaatgcggttggcccgaagtcagatgagccttgtacgagttgaaatcggccaggatcgagtgataggatatggcagtgacggcattttcgggactgagtacaaaattgccgtaggtggccggcgccgagaccgtttctttggtgatgtggcttgagagcagcgacatgatgatctgcataacgttggccgtgcttaccatcacgccgctgatcttggcccccgagctcgtggtgtacgtggtggggttgtctaggatgctatcggtggccgcttcggccagacgcgtgaggaacttgagcacatagtcgcgatcgcgcgtgcgattcagcaaaaagagcgtggccagcattttggccttgaagctctgcaagatgttgcttcgctggatgcggttcagtgcctgtcgcgccagtgtggcgttttctaccagcgtctgcaccacaaagtacggcggcgccttgcgtagcagtgtctgtaaaaagctgtgaatcaagccgcgctccatggcgtcggccgtgtttttaagcgcgcgcagcaccgtgtgcatggcttccacgttgaggatcttgtccaagatggtgccctcgaatgtctcgcgcagatacgtgaggcaggctgcgctgagctcgaaggggatggtgatgggggatttttcactgtatttggtgaccataatggtggtctgacgactagtgggcaaaccggcgccgctggccacacgcggcacctgcacgtggaacagcattttgcccgtagtcagtttattgaggtcgtggaacttgatggcgtgcgccgccgcggccaagccgctggtcaaaaaataaacccattccaggcgattgcagaaggtgccgaagatggcttcgaagtgaatattgtaacgctcggggtcatcgccgtagtagatgcgtaaggcctcaaacatctcctcgccggcgctggtcttgacgtgcgtcagaaagtcagtgggaatgcctactttaggcaggagctcgagcgccgaccagttctccatcgcggcggcggcgtgagcgcgaggcgtcggagctcggggaaagcagcgcgacccggagaatggccggcgctgcgccgcgccgcctcggctgtgacgctctaatagtcgttggcggctccgctatgccgcgccgggttttacacgtccccgtgcacgttcgcgcctgcaacctcacccaagagctatcgacgggcgaggacgcccgcttttgtcgtccgcgacccgttaacgtcgaacgggtgcgcgctgtttttgcggctctctaccgtgcctgtccgatacacgtgaggaccgagcccgagcgtgtcaagctggtactgggtcgtctgttactgggacccgtggccgtaccctgtttttgcgacggtgaagtggagggccacggtgaacatctggtacctacgacgcagttttgtcgcgggccgctgctctacgtgcaccgacgttgttgttgcggatccgtgaccgccgggcgcgcgctgtcctaccacgttctcgaaaaccacgtggccacgcatgtgctacgcggattgctctcgctgacggaatggaatcgagaattgccgagcctcttttgcgactgtcctggcggcggtggcgcctcgggaaccgaggaacgctacgctatggcctgcctgccgcgcgacctcagcctgcacctggacgactatccttacctgatggtggaaatcggacgcgtactcagtgtcagcgaggtagacgactacgtaaccgccgtctccggctacctgggcgaggccgcggcgccgcgcatccaggttcactacaagctgctctttggactcaacgtgcgtccgcaagcgccgtgcgcgttggacgctacacgcgacttttttctgctggagctgcaaaagctttggctgggcgttgaatatcaccacgaagtcacgtcggagtttttcggtcgcgtactggctcagctgcatcgcgaccgcgcccgcgtcatgatggcgcttcgcttgcccgagcagacggtgtgccacctgagcaccttcgttctcagtcgcttcaagcgacaggtactgtacttcaagctacaggtgagctacggcaagtgccggactggtcacgctgacagaagtgggggaggggggaacggtggaaatcagggacaccacaacctactgtgttatcgacgccttagcgtcacatttgccgacacagacacggtgtggagaaaccttttctacgtttattacgaactagctcgggatctggggtcccatgggacggaggaccgacccgtaagccgcggttacggtgtttcttgcgcttcgaggacgtcgcgactgtcaccgtcagaatcgacggtggtttcggcgaacggacacgcgctgtcttccaccgcgctcccgacgacgagcgcgggtcacaagctgtcactgccgcgcgacccggccgcagatcgcgttcgacgttacgtatgcattatctcgcgtctcatgtacgctcggtacggggagagatggcgtaaacactgtcaacggcggtcggagacgggagaagaggaggaggaagagacgctggaatcgggggagactgacgccacgccgccatttgactttacggggcagcagctgcgccgggcctatcaggaacaccgacgtcgtaaacatctagccgtgcagcgttacgcgccgtgccgtcgtaagctcatcggcgggatggagtttgccgaggtgacgggcgtgagtctagaccgcatcgccgtcaacgctttcaacaccaaccgcgttatcaatatgaaggctgcgctctcgtccatcgccgcgtcgggtctcggcgtacgcgcgccgcggcttcccaagaacatgacccacagttttgtgatgtacaagcacacctttaaggagcccgcttgcaccgtcagcacttttgtttccaacgacgccgtctacatcaactcgctcaacgtcaatattcgcggttcctaccccgagtttctgtactcgctgggcgtgtaccggctgcacgttaatatcgatcacttttttctgccggccgtggtgtgcaacagcaactcctcgctggacgtgcatgggctggaggaccaggcggtgattcgctcggagcgcagcaaggtgtactggaccaccaactttccgtgcatgatctcgcatactaacaacgtcaacgtgggctggttcaaagcggctacggccattgtgccgcgcgtctcgggcgccgacctggaagccattctgctcaaagaactctcgtgcatcaagaacatgcgcgacgtgtgcatcgattacggtctgcaccgtgttttcacgcaactagagctgcgcaattcgtaccagatccccttcctggccaagcagttagtgctgtttctgcgtgcttgcctgctcaagctgcacggtcgagagaagcggctgcagttggaccgcctagtatttgaggcggcacagcggggtctctttgactacagcaagaacctcacggcgcacaccaagatcaagcacacttgtgcgctcatcggcagtcgtctagccaacaacgtgcccaagatcctggcccggaacaaaaaagtcaaattggatcacctgggccggaacgccaacgtgctgacggtgtgtcggcacgtggaagcccacaagatccctcgcacgcgcctcaaagtgttagtcgaggtgctgggcgcgttgcagagtatcagcggtacgccgcacacgcgcgaagtgatccaccagacgttgtttcgattgtgctcggcggccgcagccacatcgggcctgtgttcatcccctcccccattgtgtgtgtcctcatcttcctccgtcccttctgtcccaacctccgtcagcgttgacggcagttctgaacccacgtcgccgcgagcgcggtttgcatcacgatgatggaagccgcggccgctgccgccgcggcgtttcgtccggaggagcgtccgacgccgggttggcacgacgcggcgttgttaatggacgacggtacggtgcgcgagcacgcgtttcgcaacggaccgctgtcgcaactgattcgccgtgtgttaccgccgccgcccgacgccgaagacgacgtggtttttgcttccgagctgtgtttttattgcagcggtcgttttaaccgcaggtcgtccgtcttctccatctattggcagaagcatagcgatctggtgtacgcgcttacgggcattacccattgcgccaagttggtggtggaatgcggtcagttggggagtagtaggctacggtggcgcgacggtgatgcgagtggtgaggagcgccggggagacgacgacagcagggacgagctgtacgacgtgccgggcatttatatgattcgcgtcaacgacggcggcagcaccggccccagacacgttatttggccgggtaccagcgtgctttgggcgccggacgttgtgatcactacggtgcagcgacgaatctcggcggcgcgcgccctggtgaacacgttccgccaatatttttttttgctggaacggcgctcgcacgaggagctggttctttgtccgcccgagatggaggagcgtctagcgccgctgttgcagagtgccacgcgcggtgattcggacatgtttgacggtgtggtggccagcgcttatcaccgtttgcgaatgagtaatattccgcgttcatccgcccgtctgctggaacactgcgtggggctggcgggtgctaagaagctgctcttgctcgacgtgccgcgtctggagaactattttctttgtcaagtctgtctttacgagctggacgaggacgagatgggcgaggagatgctgggcatgttggccggaaagcccgaggatgccgccgtctcgggcgcaagcggcggttttctgctacatcgcaagacgatgaagctggccgcctgtctgtgtttgttgctcaattcgctgcatttgcaccaggaggcgctggaggccttggatcctccgccgccgcgcgtcgaggagaacgaccttgtcaacgtggtgctgcgccgttattatcgcagtcacggcggcgtgcaggcgcggacgctggcggcggcccgggctttgttagccgactacgccgaaacgttttcgcccttggggagttttacgcgcctgggttacgatcgtctcgtttctgccgatgccggcgtcagtcgccggcacctggtggctctgctgcgtgcctagctgaccctgaaacggatggcgtgtatatcgtcacacaggtaggtggccatgatgacggcgatgataagatcgtccgagatacgattctggcgcttggccgagtaacgcgccgtcgtgccttcggccagcgtgacgcggtgcaggttctgaatctgctccagaagatactcgatggggtcgtggctcagcttgatggtgtaggagacgagctcttgcgaggctttgatgtagcccgagttgaaacgcgagatgaactgttccacggccagcgccttgtcgcggcccatgaggtagaagggctgttcgatgtggttctggtcgggcgtgtggtagaagagcacgcggatgagcgtgctgctctgcacgctctgtcggatgaggcaggcgatgcgcacggccgccgcctggttggtgttgccctccacggcgatgcgcagttcgtccaggtaagggtgcaggctcagcaccgagatgatcatgtgcgccgcgcactcggcgatggctacctcagaactctcggagaggtcgcgcaaaaagaaatgctctaggccgtaaatgagaaactggtgtcggtaggcgcctacggccgccacgcccgtgcccgaggccttgcggttggtggtgaaggccgggtccagatacacgtaaagcgtcttgccgaaataatcgtaggcgttggtgttgagcgtgctgtaacgcaaaatatcgaactcttcgcggctctggtccgtgatgagcacggtgttctgcgagattttattggtaccgccgatgatctcgtccatgaaagcgcccggcataaacatgttggccgtcttgcgcacttgcgagttgaggctgatgaaggtgggcttgtgcagtcggtagcaaggacacgccgtggcgtcgcccttctccgtgaagctgtgcaggtgctcttcgcacacgtaagagaccacgttgagcatgtcaaagggcgcattgttaaggcgcgtcaagaaacacgtggagtcactggtagtgttggtggacgatatgaagatgatcttggtggtattctgggccaggaaccccagaatggtgttgaaggcctctttcttgatgaagtgcgcctcgtccaccagcagcaagtggaagttttgtcctcggatgctctgtgtagagaggagacagaaaagggactcttatgattacgcacgctcgactggaagcctacagagtcggggtggggccggacaggtgagccaggtgagccgccaggtgaggcgggatcgccgtgtgccaaccgggctgcgacctgaaaaccggaaccaatccgccgacaccggcgccgcgtgacgcgcgcccataaaaacgaaagtgtcgtcgtcgcgacccgccacagccgccatgaactcgttgctggcggaactcaaccgactaggggtcgcgcacgccactacggaggatgtttttatctttgtcgaccgcctctttcaacacttttccttccttttccaggccgaggagtcaggcccgcgccgcttggaactggtcgcgtccgtgttcgagcacctgacggtggagtgcgttaacgacatcctggacgcctgcagtcacccggacgtgaacgtcgcggagacaagcaacacctgtcgtccctgcccttctcctgttccctccgcccccaaaactgtcagcggcgctcagacgtcatgtgcgacgcctcgggcgcctgtgacatgaggcacgtccagaacgcgtttaccgaggagatccagttacactcgctctacgcgtgcacgcgctgctttcgcacgcacctgtgtgatctgggcagcggctgcgcgctcgtctccacgctcgagggctccgtctgcgtcaagacgggcctggtatacgaggctctctatccggtggcgcgtagccacctgttggaacccatcgaggaggccgcactggacgacgtcaacatcatcagcgccgtgctcagcggcgtgtacagctacctcatgacgcacgccggccgttacgccgacgtgatccaggaggtggtcgagcgcgaccgcctcaaaaagcaggtggaggacagtatttacttcacctttaataaggttttccgttctatgcataacgtcaaccgtatttcggtgcccgtcatcagccaactttttattcagcttatcatcggtatctactcaaagcagaccaagtacgacgcgtgtgtcatcaaggttagtcgtaagaagcgcgaggacgcgcttctgaaacagatgcgttccgaatatggaaacgcacctgtattcggatctggcgtttgaagcgcggttcgctgacgatgagcaattgcctctacacttggtgctcgaccaggaggtgttgagtaacgaggaggccgagacgctgcgctacgtctactatcgtaatgtagacagcgctggccgatccacgggccgcgctccaggcggagatgaggacgacgcaccggcctccgacgacgccgaggacgccgtgggcggcgatcgcgcttttgaccgcgagcggcggacttggcagcgggcctgttttcgtgtactaccgcgcccactggagttgctcgattacctacgtcaaagcggtctcactgtgacgttagagaaagagcagcgcgtgcgcatgttctatgccgtcttcactacgttgggtctgcgctgccccgataatcggctctcaggcgcgcagacgctacacctgagactggtctggcccgacggcagctatcgtgactgggagtttttagcgcgtgacctgttacgagaagaaatggaagcgaataagcgcgaccggcagcaccagttggccacgaccacgaatcaccgtcggcggggcggactgcgtaataacttagacaatgggtcggatcgccgtttgcccgaagcggctgtggcttctctggagacggccgtcagtactccattttttgaaattccgaacggagcaggaacctcctccgcgaacggcgacggcagattcagtaacctggagcagcgggtagcgcgtttgttgcgcggcgacgaggaattcatctatcacgcgggtccattggagccgccttccaagatacgcggtcatgagttggtgcagctgcgcctggacgtaaatccagacctcatgtacgccaccgatccgcacgaccgcgacgaggtcgcgcgtacggacgagtggaagggtgccggtgtctcgcgtcttcgcgaggtctgggatgtgcagcatcgcgtgcgcctccgtgtgctgtggtacgtcaattccttttggcgcagtcgcgagctgagctacgatgaccacgaagtcgaactataccgggcgttggacgcttatcgggcgcgcatcgccgtcgagtacgtgctgattcgcgccgtgcgcgacgagatctacgctgtactacgacgggacggcggcgcgttgccacagcgtttcgcctgccacgtgtcacggaacatgtcctggcgcgttgtttgggaactttgccgtcatgccttggcgctctggatggattgggcggacgtgcgtagctgtattattaaggcgctaacgcctcgtctgagccggggtgccgccgctgccgctcagcgagctcgtcgccagcgcgagcgctcggcgcccaaaccgcaggagctgcttttcgggccgcggaacgagagcggtccgcccgccgaacagacttggtacgctgacgtggtgcgctgcgttcgcgcgcaagtggatttgggcgtggaagtgcgcgcggcgcgttgtcctcgcaccgggctttggatcgtccgtgatcgccgcggacgcctgcgacgttggctctcgcagcccgaggtgtgcgtgctgtacgtcacgccagacttggacttttactgggtgctgccgggcggctttgccgtctcttcgcgcgtcactcttcatggcttggcgcagcgggctttgcgagaccgattccagaactttgaagcagttcttgcaagaggaatgcatgtggaagctggtcggcaagagccggaaacaccgcgagtatcgggccgtcgcttgccgttcgacgatctttagtccggaggacgacagctcgtgtatcttatgccagttgctgttgctctaccgcgacggcgaatggatcatctgtttttgctgcaacggccgttatcaaggccactatggcgtgaaccacgtacatcggcgtcgtcgacgcatctgtcatctacctaccttgtaccaactgagcttcggaggtcctttgggtccagccagcatcgatttcttgccaagctttagccaggtgaccagcagtatgacgtgcgatggtattacgcccgacgtgatttacgaggtctgcatgttggtgccccaggatgaagccaagcgtatcctggtcaagggtcacggtgccatggacctgacctgtcagaaggcagtgacgctaggcggcgccggcgcctggttgctgccgcgtcccgaaggctacacgcttttcttttacattctgtgttacgacctgtttacctcatgcggcaatcggtgcgatatcccttccatgacgcgcctcatggcggcggccacggcctgcgggcaggcgggttgcagcttttgcacggatcacgagggacacgtagatcccactggcaattacgtgggttgcacccccgatatgggccgctgtctttgttacgtgccctgtgggcccatgacgcagtcgctcatccacaacgaggaacccgcgacttttttctgtgagagcgatgacgccaagtacctatgcgccgtaggttctaagaccgcggcgcaggtcacactgggagacggcctggattatcacatcggtgttaaggattctgagggccgatggctgcccgtcaagaccgatgtgtgggacctggtcaaggtagaggaacctgtgtcacgtatgatagtgtgttcctgtccggtgcttaagaacctagtgcactaacggggtctgacagttcacggggagaagaaacaagaaacaacaaaaaaaaggaggacatggactcgccacggtttgtggcaaggcgtatgttatcatcatggagctactcacgttggtgttgtagcaactggcaaaaagcgccgtgctcttggcgccgcggtggtcgatgctgatcacgttgtccttgttctcgaccacgtagtcgcgcgcgaaggtgtggcggcagcggaactcgacctctttgagcacaaactgcgacacgtgcttttggtgcgccacgtagccgatgctgatgccgatcatgtgcttaagcagaaacgagataatggggatgatgaaccaagtcttgccgtgacgtcgcggcaccaggaacacggtggctttctgcttaaagatgtcgatggaggtctgcgagaggaagtcgatctggaaggcgtggatgaggtactgcagcacgcgattggccagcacggggatcttggtcacggctataaaaaagatgacgtgtatcaataaattcttttgaaacggttcgagtcggatggcttttgcgtcgccctcgacggcggtactgaagccgccgtcgagccactttttaaagtcggtcatgaagttgttgatctgctgaaactgcggatcgcggtagagctcggtcaacgcgtccagcttctggtaggaggcgcgctgctcctcggagcacgggcgaaacgtcagtttatcgagcgcgctcttgaggcgctcgtgaaacagcagctcgcgctggctttcctcgggcgagttgtagtcgcggtggcggccgcagaaggccatgagcggcaggaaggcctcgttgcacgagtgggccagcccgagttcggggtgcatcatctggtagcgcttgcggcacagcgtcgccacattggtgaaggccgtggagatgcaggaggtggggtggctcttgcgcttctgcagctccgcgtagcgctcctggatcttggcggccgagtctccgcgcaacatgatggcggcggcggtggtgcgagcggaggttaggcggcagcggcgagaggagaggaaaaagatggcggccgcgaggacgacggaggatccacccgaaaaccacgttgttgcggacgtggctcgtgggacgggcgccgtcactcgttcgtcttcgtcgtccctagtggtgtcgtcttcctcggcgtcaggctcggacgaatcttcctccgcctctcctctcagtttccccgtctcctccccctcaactgccgtcaggtctccggggtccgccggggtttcaacgtccctgtgctcggtggaacggatggtcgagctgtcggcgcagtctccggccgccgatttctcggtctccgaggcttggcgcttcgaggaggccgtaaatatggcgctggtggcctgcgaggccgtgtcaccttacgatcgctttcgcctaattgaaacgcccgacgagaatttcttgttggtcaccaacgtaattccgcgcgaatcggccgaggtgccggtgttggatagcagtagcagcggtggcgatagcgggccggaggacaaaaagaaaaacgtcgggaataaaaccgcgggggaaaagaacggcggtgggtctcgggccaaacgccgtcgtagacgacgcgctccgaaaaacgacgccgccacgccgtcttttctacgtcgacacgacgtgctggagcgtttcgcggccgcggctaagcctttgccgtcgctttgtgtgcgtgattatgcgttacgcaatgctgaccgtgttacctacgacggcgaattaatctacggcagttacctgttgtatcgcaaggctcacgtggagctgtcactctccagcaacaaggtgcaacacgtggaagccgtgctgcgacaggtgtacacgccgggcttgttagatcatcacaacgtgtgcgacgtggaggccctgctgtggctgctgtactgtggaccgcgcagcttttgcgcgcgtgacacttgtttcggtcgcgaaaagaacggttgtcctttccccgcgttgttgcccaaactcttttacgaacccgtgcgggactatatgacctacatgaatctggctgagctgtacgtctttgtttggtatcgcggctacgaattccctgcgccgacgccgcaggcgacgacggcgggtggtggtggtggtagtggtggcggcggcggggccggcgcttgtgcggtcgagacgagcgcgtcagcaggccgggtcgatgacgccggcgacgaggtgcatttgcctttaaagcccgtctcgctggaccgtctcagagaggtgttgcaggcggtgcgcggccgcttctcggggcgcgaggtgcccgcctggccggcctcgtcgcgcacctgtttgttgtgcgcgctctacagtcagaaccgtctctgtttagatctcgcgcgtgacgaggcgcggaccgtgagttatagccccatcgttatccaagactgcgccgcggctgtcaccgacgtcactttgagccacatcttgcccggccagagcaccgtctcgcttttccccgtctaccacgtcggcaagttgctggacgctctctcgctgaacgacgcgggtctcatcacgttgaatctatgacgtcggtcaacaaacagctcttaaaggacgtgatgcgcgtcgaccttgagcgacagcagcatcagtttctgcggcgtacctacggaccgcagcaccggctcaccacgcagcaggctttgacggtgatgcgtgtggccgctcgggaacagacccgatacagtcagcgaacgacgcagtgcgtggccgcacacctgttggagcaacgggcggccgtgcagcaagagttgcaacgcgcccgacagctgcaatccggtaacgtggacgacgcgctggactctttaaccgagctgaaggacacggtagacgatgtgagagccaccttggtggactcggtttcggcgacgtgcgatttggacctggaggtcgacgacgccgtctaacaggtatagcaatctccgtcacgcctctgttcagattttattaaaaaaaaaacacaacataacgacagtgtcggtgtggtagctagtgcagccttaggaacagggaagactgtcgccactatgtcctccgcacttcggtctcgggctcgctcggcctcgctcggaacgacgactcagggctgggatccgccgccattgcgtcgtcccagcagggcgcgccggcgccagtggatgcgcgaagctgcgcaggccgccgctcaagccgcggtgcaggccgcgcaggccgccgccgctcaggtcgcccaggctcacgttgatgaaaacgaggtcgtggatctgatggccgacgaggccggcggcggcgtcaccactttgaccaccctgagttccgtcagcacaaccaccgtgcttggacacgcgactttttccgcatgcgttcgaagtgacgtgatgcgtgacggagaaaaagaggacgcggcttcggacaaggagaacctgcgtcggcccgtagtgccgtccacgtcgtctcgcggcagcgccgccagcggcgacggttaccacggcttgcgctgccgcgaaacttcggccatgtggtcgttcgagtacgatcgcgacggcgacgtgaccagcgtacgccgcgctctcttcaccggcggcagcgacccctcggacagcgtgagcggcgtccgcggtggacgcaaacgcccgttgcgtccgccgttggtgtcgctggcccgcaccccgctgtgccgacgtcgtgtgggcggtgtggacgcggtgctcgaagaaaacgacgtggagctgcgcgcggaaagtcaggacagcgccgtggcatcgggcccgggccgcattccgcagccgctcagcggtagttccggggaggaatccgccacggcggtggaggccgactccacgtcacacgacgacgtgcattgcacctgttccaacgaccagatcatcaccacgtccatccgcggccttacgtgcgacccgcgtatgttcttgcgccttacgcatcccgagctctgcgagctctctatctcctacctgctggtctacgtgcccaaagaggacgatttttgccacaagatttgttatgccgtggacatgagcgacgagagctaccgcctgggccagggctccttcggcgaggtctggccgctcgatcgctatcgcgtggtcaaggtggcgcgtaagcacagcgagacggtgctcacggtctggatgtcgggcctgatccgcacgcgcgccgctggcgagcaacagcagccgccgtcgctggtgggcacgggcgtgcaccgcggtctgctcacggccacgggctgctgtctgctgcacaacgtcacggtacatcgacgtttccacacagacatgtttcatcacgaccagtggaagctggcgtgcatcgacagctaccgacgtgccttttgcacgttggccgacgctatcaaatttctcaatcaccagtgtcgtgtatgccactttgacattacacccatgaacgtgctcatcgacgtgaacccgcacaaccccagcgagatcgtgcgcgccgcgctgtgcgattacagcctcagcgagccctatccggattacaacgagcgctgtgtggccgtctttcaggagacgggtacggcgcgccgcatccccaactgctcgcaccgtctgcgcgaatgttaccaccctgctttccgacccatgccgctgcagaagctgctcatctgcgacccgcacgcgcgtttccccgtagccggcctacggcgttattgcatgtcggagctgtcggcgctgggtaacgtgctgggcttttgcctcatgcggctgttggaccggcgcggtctggacgaggtgcgcatgggcacggaggcgttgctctttaagcacgccggcgcggcctgccgcgcgttggagaacggtaagctcacgcactgctccgacgcctgtctgctcattctggcggcgcaaatgagctacggcgcctgtctcctgggcgagcatggcgccgcgctggtgtcgcacacgctgcgctttgtggaggccaagatgtcctcgtgtcgcgtacgcgcctttcgccgcttctaccacgaatgctcgcagaccatgctgcacgaatacgtcagaaagaacgtggagcgtctgttggccacgagcgacgggctgtatttatataacgcctttcggcgcaccaccagcataatctgcgaggaggaccttgacggtgactgccgccaactgttccccgagtaaccgggacgcggaacgtgacggttgctgaggggaaaggcaacagagaaggtacaaacccaccggcggggaaaataccgaggcgccgccatcatcatgtggggcgtctcgagtttggactacgacgacgatgaggagctcacccggctgctggcggtttgggacgatgagcccctcagtctgtttctcatgaacacctttttgctgcaccaggagggcttccgtaatctgccctttacggtgctgcgtctgtcttacgcctaccgcatcttcgccaagatgctgcgggcccacggtacgccagtagccgaggactttatgacgcgcgtggccgcgctggctcgcgacgagggtctgcgcgacattttgggtcagcggcacgccgccgaagcttcgcgcgccgagatcgccgaggccctggagcgcgtggccgagcggtgcgacgaccggcacggcggctcggacgactacgtgtggctcagccggttgctggatttagcgcccaactatcggcaggtcgagctcttccagttgctggaaaaggaatcgcgcggacagtcgcgcaactcggtgtggcatctgttgcgtatggacacggtctcggccaccaagttctacgaggccttcgtcagcggctgtctgccgggcgccgcggcggcggacggttcgggtggcggcggctcgcactacacgggttcgcgcgccggcgtctcgccgggcatccagttcggtatcaaacacgagggcttagtcaaaacgctggtggaatgttacgtgatgcacggacgcgagccggtgcgcgacggcctcggtctgctcatcgaccccacgtcggggctgctgggcgcttccatggacctgtgcttcggcgtgctcaagcagggtagcggtcgcaccttgctggtggaaccgtgtgcgcgcgtctacgagatcaagtgccgctacaaatatttgcgcaaaaaggaggacccctttgtgcagaacgtgctgcggaggcacgacgcggcggccgtggcctcgctgttgcagtcacacccggtgccgggcgtggagtttcgcggtgaacgcgagaccccgtcggcacgcgagtttctgctttcgcacgacgcggcgctcttcagggccacgctcaagcgcgcgcgcccgctcaagccgcccgaaccgctgcgcgagtacctggccgatctgctgtatctcaataaggccgagtgttcggaagtgatcgtgtttgacgccaagcacctgagtgacgacaacagcgacggggacgccacgatcactattaacgcgagtctcggcctagccgcgggcgacggcgctggcggcggcgctgatcaccacctgcggggcagcccgggcgattcgccgccgccgatacctttcgaggacgaaaacacgcccgagctgctgggccggctcaacgtgtacgaggtagcgcgcttttcactgccggcttttgtcaatccgcgtcaccagtattactttcagatgctcattcagcagtacgtgctcagccaatactatataaagaagcatccggacccggagcggatcgatttccgcgacctgcctaccgtctacctggtctcggccatcttccgcgagcgcgaggaaagcgaactgggctgcgagttgctggccggcggtcgcgttttccactgcgaccacatcccgctcctgctcatcgtcacgcccgtggtctttgaccctcagtttacgcgccatgccgtctctaccgtgctagaccgttggagtcgcgacctgtcccgcaagacgaacctaccgatatgggtgccgaactctgcaaacgaatatgttgtgagttcggtaccacgcccggtgagcccctgaaagatgctctgggtcgccaggtgtctctacgctcctacgacaacatccctccgacttcctcctcggacgaaggggaggacgatgacgacggggaggatgacgataacgaggagcggcaacagaagctgcggctctgcggtagtggctgcgggggaaacgacagtagtagcggcagccaccgcgaggccacccacgacggctccaagaaaaacgcggtgcgctcgacgtttcgcgaggacaaggctccgaaaccgagcaagcagtcaaaaaagaaaaagaaaccctcaaaacatcaccaccatcagcaaagctccattatgcaggagacggacgacctagacgaagaggacacctcaatttacctgtccccgcccccggtcccccccgtccaggtggtggctaagcgactgccgcggcccgacacacccaggactccgcgccaaaagaagatttcacaacgtccacccacccccgggacaaaaaagcccgccgcctccttgcccttttaactcataaactttcaggtctcgcgtacgattcgcgagtcgggaatgggacacccgtgggtgtttctccgtgtgtatattattttttttttttgtgtgtgtttgcgcccccgtgtgtctaatgtgctgtttgaaacacgtaaagtagctggtggaagaacagataaacctttaataaaaaaaaaagtatgtgctcccgacccacggtctgcgtgtctcttttttatgtccatgtctccaagtctggtgcgggtggcggcggggttaagcgtcctcgaagtcttcatcatcgtcgtcgtcctcttcttcgcggaggcgacggctttccaagctgtcgtggtgactgagcacagcgacttcttcgccggaggctgtggccagcgcctggtacttgacactgccgctaccgcgtccgcgaaagtagcggacggcgcgacacgtcgtaaacatggcccatatgaaaaagagcatgccgaacgaccagctgatgccggtgcggtattcgttgctgaggaaggtatcgtactgcacgatggggtagatgaggccgcagagtccaaagaaggcgcccaggtggtagccgaattgcaccttgacgtattgaaaaaagacggcctcgatcagtaaaaagtagatgatggagatgatagcgtagaccacgaagacggctaacaccatgtggcctgtacgcacgaaaaagttgtttccgaagccgtagcacagggccatggctaccacggtggtgttgaaaccaagcgctacctctaccaggttgacgatgagcgtgcggaactgcaccgtacctttgagcttggggtgcagacgcgagaagaaaaagagtgagcgtttgtagctgcggtactgcgtgaccatgctcacgttgaaaatggtcaggcagaaaaagtgcacggcggccatgaaggcgatcatgctgggcagccgaaatgacatggtcagtgtgaatagttggaacgtgtccatgctgagaatgaagaggaaggctgtgaggctgtcgcccatgtacgaaatgtcgcgtgtcgactggtttaggctcatgcctttgtccttgcgcatgctgatcttgatccagcataccaggtagtagatggtcacggctaaaaagacgagctgcatgaacacggcgtagcacaccaactgcaccgagtctaagaaaagcataggcgtgtgcaggtgcattacgttgtaggccgacatgttgagcctttcaaagtccacgacgtgatagtagacgcaggggtagcccaggtgcggaaaattgctcagcactagatgcacgctgacgttgacaaaagtcagcaccatgaaaacgatagaagcgctccatgtccgtgtattcaccttatccacgtgcgagggggccatggcgatagcggcggcccgctcgctcgggaggcgatgggggcgcgccgatgacgacaggctcgcgggtcgttaaatactacgatgggagccgccgcggctcacgacgcggtttgagcacgtccgggcggtcggtgaaaaaagaccccgcgggccttcgcgactctcttctgtccgaggatgaccgctcagccgccgctgcaccaccgccaccacccgtacaccctgttcgggaccagctgtcatctcagctggtacggccttctagaggcctcggtgcctatcgtacaatgtctgtttttggatctgggtggcggccgtgccgagccgcggcttcacacgttcgtggtgcgcggtgaccgtctaccgccggctgaggtgcgtgctgtgcatcgcgccagctacgctgcgctggcctcggccgtgactacggacgccgatgagcgtcggcgcggcctagagcagcgtagcgccgtgttggcgcgcgtgttgctagaaggcagcgcgttaatccgcgtgttggcgcgcaccttcacgccggtgcagattcagacggacgctagtggcgtggagattttggaggccgcaccggcactgggcgtggaaaccgcagcgctgtcgaacgcgcttagtcttttccacgtagccaagctagtggtcatcggctcgtatcccgaagtgcacgagccgcgtgtggtcacgcataccgcggaacgcgtctccgaagagtatggcacccacgcgcacaaaaaattgcgtcgcggttactacgcctacgatttggccatgtcgtttcgcgtcggcactcacaagtatgtgctggagcgcgacgacgaggccgtcctggcacgcctctttgaggtgcgcgaggtgtgttttttgcgcacctgtctgcgtctggtcacgcctgtcggtttcgtggccgtggcagtgaccgacgagcagtgttgtttattgctgcagtcggcctggactcacctttacgacgtgcttttccgtggtttcgctgggcagccgccgctacgcgactacctggggccggacctctttgagacgggcgccgcccgttctttcttttttcccggtttcccgcccgtgcccgtctacgcggtccacggtctgcacacgttaatgcgcgagacggcgttggacgcggcggctgaggtgctctcgtggtgcggcctgcccgacatcgtgggctcggccggcaagctggaggtggaaccctgcgcgctctcgctcggcgtgcccgaggatgagtggcaggtcttcggtaccgaggccggcggcggcgccgtgcgtctcaatgccacggcttttcgcgagcgaccggccggcggcgatcgtcgctggctgttgccgccgctgccacgtgacgacggcgacggtgaaaacaacgtcgtggaagtcagcagcagcaccggcggtgcgcacccgccgagcgacgacgccactttcaccgtgcacgttcgcgacgccacgctacatcgagtgctcatcgtggatttggtcgagcgcgtgctggccaagtgtgtacgcgcgcgcgacttcaatccctacgtgcgttatagtcatcgactccacacttatgcggtttgtgaaaagtttattgagaatctgcgttttcgctcgcgacgcgctttctggcagatccagagtctgctgggctacatctccgagcacgttacgtcagcctgcgcttcggccggccttttgtgggttctgtcgcgcggccaccgcgagttttatgtctacgacggctattcgggtcacggacccgtctcggccgaagtgtgcgtgcggactgtggtcgactgttattggcgcaaactttttggcggcgacgatccgggtcccacctgtcgtgttcaagagagcgcgcccggcgtgctgttggtctggggcgacgagcggttggtgggtcccttcaacttcttctacggcaacggcggcgccggtggtagtccgctccacggggtggtgggtggtttcgcggcgggacattgcggtggcgcttgttgcgcgggctgcgtcgtcactcaccgccattctagcggcggcggtggtagtggcgtgggcgacgcggaccacgcgagtggcggcggtctagatgccgctgccgggagtggtcataacggcggtagtgatcgggtttctccctccacgccgcccgcggcgttaggtggctgttgctgcgcagccggtggcgactggctctcggccgtgggtcatgtcctgggccggctgccggcgctgttacgggagcgcgtgagcgtgtccgagctggaagccgtgtaccgcgagatcctctttcgtttcgtggctcgccgcaacgacgtggacttttggttactgcgcttccagcccggtgaaaacgaagtaaggccgcacgctggggtgattgactgcgcgcccttccacggcgtgtgggccgagcagggccagatcatcgtacagtcacgcgatacggcgttggcggccgatatcggctacggcgtctatgtggacaaggcctttgccatgctcacggcttgcgtggaggtctgggcgcgagagttattgtcgtcctccaccgcttccaccaccgcttgttcttcttcttccgttctctcctccgccttgccgtccgtcacttcgtcctcttcgggcacggcgacggtgtctcctccgtcttgttcttcttcgtcggcgacttggctcgaggagcgcgacgagtgggtgcgctcgctggcggttgacgcgcaacacgctgctaagcgggtggcttccgagggcctgcggtttttccggctcaacgcttaacgagtcacgtaggggaactacgtgggtaagtgacgtggatactagtaaaaaaagtgcgtcaaagctcttagcgtgtgacgtggatactagtaaaagggacgtcaaagctcactacgtgttgcgtgttttttttttttctatgatatgcgtgtctagttcgcttctcactcttcctctcctcgttcccagcgcggcggcagcttggggggtgagggcaaattggggtagttggcgttgagcacgtctagcaggcccaggcccacgggccaaccgtccacggtcttgcgctcggtcagcttgaggctgaacgagtgtgcctcgtcctgaccggtaaggcggaaaaagaagcgtgctaccagctgcaggcaggtatgccgcgtctgctggaagagcacgaaggtagcgggcacgtactgcacaatgtgcggctctttttcctcaaagagcaggtagagcgcgctgcagatcagccgcctggcgctgtggtgcagcagccggccgaagctttcgcgcacgttcaccgcgtccaggtactggagcaggtcgtgcaggcacttgcgcgttaagttgcaattttccacgcacgaaataacggtacagagcgcgaagtgcagcaggttgtcggctttgacgatgccgcagcggtgtttgagccgcagatccgagagcctcacctgcgtgacggcgtcttcggtctcgagcaaaaacacggcggagtagcctagaaaggccgaggtgcacagcaactcgctgcggtactcggccatggagaccagcagcccgtgctccgtgtgcagccacagcttgtcgccgcgcaccgtaaagtcgagcacttgcggctccatgatcatcacattctgtctagtgaaatccgtatggacctccagcacgccgcggatcatcagggcctccatttcgaaatcggccgacacgctctgggccgcgccgctcctcgtctgccgtgatcaagcggcgcggcgcggacctttcaagtgttcctgggccgccgctcgaggcagttcccctttctggcactccgcccgccgcttcgcggctcatttggcgccgacgcgccttctcgcggctgcaaatcagctccacgtatcggcaaaacttgctgtcgtcgtaggcggcggccacgatctcgccgaaggagagctgcaggtaggcctcgggtacggggtccagcgtgcccagcgccaggatgtgacacagatagggcagggtcacgcgctctaccgtgtaattggagtagacgatggcctcttcggccccttgatgcgtgaccagacgccgtaggcgaaaggtacggaaatactcgttttcccacaactgcgtgaggaagcgttccagcgactcggtgccgggcacgaactgcgagaagaagctgttggccaccaggcggttgtcttccaccgccagcggacggaagggcgccgcgtcgcgcgccttgcgcacggcctccaacacgggcaggtggtagagttcggcgtcgcgcgcgcccaggctcatggagtcctcgcgccgcgaggcgtagcgcgtgagcaggtcgcgcagttcgcgcacgcgattctcccaggtctggttaagcgtgcgcaggtcctggatctcgtccacctgcgactggatctgctcctccaggcacttgataacctgcttcttaaacaggtcgcggatgtcccgctcgggcgccgccgggccgggtggcggcggcagcagcccgacgtggcccgcgggtcctcccaccacggcgccgccgggtcccaccacgccgggtccgcccggaccacgcgcgggtagtagacggttttggtccaccagcgagggggtcaggtcctgcagaaaggactcgacgctgtcctcgatgccgatgcgcgatttgctgtccgagacgttaagcaaaaacttcataatggactttttggcgtcgctgccccggtcgtgctgctccatcatctccaccagcttcttgcagttgagctcgtggcggctggcggtcaccactttcacaggaaaggtattgagcaactggcagatcttttggtggcggcagagcccgtcgtagcgcagaatctcctcgtgcaggtgtgccaccggcgtggtgaacagcagcttgtcgcgctcataagccagcggttcggccgccacgtacaagcggatgtgcttgccgcgcagctgcgcctccagccgctccgagcgcaccttcttgaagacgcgtacctcgggcgcgttggctacgcgcacggcgcccaggcgctcggccacctgcagcagcagcgccaggttagcctgcagcaggtcctgcgccagcgggtgtgtctcggtggcccgctgcacggccgcgcgtacaaattgcgcccgctcggccgcctcgctcggcttggtcttcacgtccagcagcggtaccagtcccaccgttacgcaccaatccacgtagagaccatagtcgtcgttatcggcgtactgatataaaatgtcgcggagcgcgcccagcacgcccgtttgcacgctctggcgcaacgaggcgctccacaccaacagatactgctccaggtcctcttcgtccagcgcgcggtagggaaatagcgccgcgtgcaacttccactcctcggccacgcgccgcaccgtgatggtgtcaaagagcgttttgcacactccgtagagcagctgcttgcgcagcacgcacgggtcgcgcagcacctggtgcatgctttggccgcgacacgtccccagaaagccgtgcagcaaccgcaggaagctcatcgtctgccccgtggggaaaatgtcgatgacggcctcgtcatccacgccgcggcccacgcccaagtacgacgacgccttgatcctcaacctctcgtcggccgccaagatcgaacggatcgtcgacaaggtcaagtccctctcgcgcgagcgctttgcgcccgaggatttttcgttccagtggtttcgctccatcagtcgcgttgaacgaacgacagataacaacccctctgccgcaactaccgccgcggcaacgacgaccgttcactcctccgcctcctcttctgccgccgctgccgcttcgtccgaggccggcggcacgcgcgtgccctgcgtcgaccgttggcccttctttcccttccgcgcgctgctcgtcaccggcacggcgggcgccggcaagacttccagcatccaggtgctggcggccaatctagattgcgtgatcaccggtaccacggtgatcgccgcgcagaacctcagcgcgatcctcaaccgcactcgctcggcgcaggtcaagaccatctaccgcgtcttcggcttcgtcagcaagcacgtgccgctggctgacagcgccgttagccacgagacgctggaacgctaccgcgtgtgcgagccgcacgaggagaccaccatccagcgcctgcagatcaacgatctgctcgcctactggccggtcatcgccgacatcgtggacaaatgcttaaatatgtgggagcgcaaggccgcttcggcctccgccgcggccgcagccgccgcctgcgaggacctctcggagctgtgcgagagcaatatcatcgtcatcgacgagtgcggccttatgctgcgctacatgctgcaggtggtggtgtttttttactacttttacaacgccctgggcgacacgcgactttaccgcgaacgccgcgtgccctgcatcatctgcgtcggttcgcccacgcagaccgaggcgctggagagccgctacgaccactacacgcaaaacaagagcgtgcgcaagggcgttgacgtgctctcggcgctgattcagaacgaggtgctcatcaactactgcgacatcgccgacaactgggtcatgtttattcacaacaagcgttgcaccgacctggactttggcgacctgctcaagtacatggagttcggtatcccgctcaaggaggagcacgtggcctacgtggatcgcttcgtgcggccgcccagctccatccgcaacccctcgtacgccgccgagatgacgcggctttttctctcacacgtcgaggtgcaggcttacttcaagcggctgcacgagcagatccgcctgagcgagcgccaccgtctctttgatctgcccgtctactgcgtggtcaacaaccgcgcgtaccaggagctctgcgagctggccgacccgctgggcgactcgccgcagcccgtcgagctctggttccgccagaacttggcgcgcatcattaactactcgcagtttgtcgaccacaacctctccagcgagatcaccaaggaggcgctgcgccccgcggccgacgtcgttgccaccaacaactcctccgtccaggctcacggagggggaggatctgtaatcgggagcaccggcggcaacgacgagacggcgtttttccaggacgatgataccaccactgcgcccgatagccgtgagacgctgctcaccttgcgcattacctacatcaagggcagttcggtgggagtcaactctaaggtgcgggcctgtgttatcggataccagggcacggtcgaacgtttcgtggacatcttgcaaaaggacacgtttatcgaacgcacgccctgcgagcaggcggcctacgcctactcgttagtttcgggcctgctcttctcggccatgtactacttctacgtgtcgccctacacgaccgaggagatgttgcgtgagctggcgcgcgttgagctgcccgacgtgagttcgctctgcgccgctgccgccgccacggccgccgctcccgcttggagcgggggagagaatccgataaataatcacgtcgacgcggattcttctcagggcggccagagcgtgccggtatctcaacggatggaacatggccaagaggagacccacgacatcccctgcctgtccaaccaccatgacgactcggacgccatcacggacgccgaactcatggatcacaccagtctgtacgcggatcccttttttctcaaatacgtcaagccacctagcctggcgctgctttctttcgaggagacggtgcacatgtacactaccttccgcgacatttttctcaagcgctaccagctcatgcagcgtctcacgggcggtcgcttcgccacgttgccgctcgttacctacaatcgccgtaacgtggtgttcaaggccaactgtcagatcagctcgcagaccggctccttcgtgggcatgctttcgcatgtgtcgccggcgcagacgtacacgctcgagggctacaccagcgacaacgtgctcagtctgcccagtgaccgccaccgcatccaccccgaggtggtgcagcgcggcctttcgcggctggtgctacgcgatgcgcttgggttcctctttgtgctcgacgttaacgtttcgcgcttcgtcgagtcggcgcagggcaagagtctgcacgtgtgcaccaccgtggactacggcctcacttcgcgcacggccatgaccatcgccaagagtcagggcctgtcgctcgagaaggtggccgtggactttggggaccatcccaagaacctcaagatgagccacatctacgtggccatgtcgcgagtcacggaccccgaacacctcatgatgaacgttaacccgttgcgactgccctatgagaagaacaccgctatcaccccctatatctgtcgcgcgctcaaagacaaacgcaccacgcttattttttgacacaacaccgtgtaaggaaaacgtgactttattgagcagggtaaaaaccacgtacaagaaccacgttgtctatccccaaaaaaacacacaccgtcagggaacacatcgcctatagatagcggcactttacataaaaccaccgtacctgcatcacggtggctcgatacactggaaattcaataaaaaccaccgtgtctccgtgacggtacttatcgggtcagcgtcttttgagatttctgttcgtaaacttatccgtttccccggtccgcggtgtctcctcgcgaggctgacagtcgacgggtggtacctgcaagagaagaaacccgggtgggagcgacgccgtcgctgggtatcaaccccgcggctgaccgtcgtccggtaaaggaacaacccgtcgtcgcaagccgggttcgaccaagagaaaaaacccgggtgcggggggagacgggtcgtcctttggttgttcgcggacggcgtacatgccgcgtgggtcagtcgacggcgtcgctccgtgcggtcggtcatcattctgcttcacatatatgggttgtttgtgtttttttttataatgaatacgcactgatcctatccgtgactgcgcgtgtggcagagaggatgccttataacatgtattttgaaaaattgccaacagctataatttctctcatgtagcagaatagagaccttttgtcgtctttttgtttgtcattacttgttttccagggaattagagagagggaaccgcgcctccggcggcggtgcccgcggaccccggccccttctcgcgtgcgcggtgtgactggttgagcgaatgagcagctaggcttggtggtgctccgcgtgcgggggagaagacgattaacaacaaaaaataagtggaagtggccggtgggtctttgtccgcgtgcgcgcccatccgtcgccgggaccgagcagaaagtgatgtggtggtacattgattttttccttgacaggaaagaaaaaaaagagttttgttttcctatgtgagaggagaaaggtatgtgaggagatgttcgatgatcgtatgttacagttatgctgtaaggaagcttttatcgtgcgtcctgtttttcatttgatgtatatgacacaattgaaacctatcgataggcgtatatcgaggattcatcaattcttagaatcgtcgtctttttggctaattggactttgcccatgttggttgtcattcgtggcctgaggtcatcgtcgtccacgacgacgtgtctatagcgtgcggtgtgatcattgtgtcgagccagagaaagcgcgcctcgcacgacgtttgcggatcggctcgcgggtgtgtggaattcctaagaacataatcagctggtcgtctttctttgatgtgttgttgtcgtcgaggtcttgcttcgttttcttttttctttttagtcgatggaacttttcttcggtacgggttcttgttatggaagcttgtgttttcgaacatgaattcgaaaaaataaaaaggcctatcttcgtttcaaaaaaaggacagatatcaatcttcttaacttatatcatggtaaattcagaatcctatggtgtcttattatctctaaagtagtcaacattatggtctaacttgtatttccctgacgagatatatatgatccttataacctggctactatcatgaacaacaatatccttacttacagtcatcttcgtgagttaatgaagtataatatcggtcatctatcaacttatctgctatgtaacgtacccttttaggtattttgcgtttcttaacgagtgtacccgcctgtgtgaggcgaaactctgagaagtctaccgagtcgagttacaagtcactaaaacacttacacgagttatctatactaaaatcactatctatgttgtttgcttacctaattattatcctacatgacgaagctacctcccaacgtaaggtagggggagaggagacagaacaataaaaagtaactaatgtttcttagaacttacccgctaaggacttaccaaactatattcaccaaaaaacaacagctacgtgtttcatttgttttaatctaccgaagtaaaaaaaaaaagatgattagctatccagaacctacttacttcttaatgttttaactaaggatgcctatgggattggaaaaaaaatcacagcaacttgctactaatcagttgacagcgaagagactcataacaaagatttctgggtaatacggttataataatgcttatggactaaaggatacttggaaaaaaagaacgggctatgactatagagattcgtcgagatatcaaacttcaaataggcggctatcattcatggttgtggtgactatatcgtggagaaaaaatgtgatcgttagttagctaggtgagacttacagctatccatccgtctagtttttcgttgtaatgatgatagtacgtctatggtggtgatcgattttggttaacaatttgttcgtttaaaggcttaatgtacttatgctacatgatgtattattctttgattcatcgttcctcctaagggggtgtatgtatgtatgtactagtcgtatagtgttcctaacatcatgactattcagactatggcttcatctatcgtgtctaaagttcacttattctactattactatatatatgcactactatgtaactaggatatggtcctataaggtgtcttctatcacggtggcttgtttatcgcttggcggttacgagcaagagttcatcacggaccagccgtgaggcagggcacacgcgggtcggcggcgatgatgtcccccgcgaaggggacaacgaaaacaagaggccgccggccgcggccacggatgcgtagcggttacacaatgtttggttgagcgttttgtttcatcgtcgtggtggttttgttgttctctgtatatatcgtgtggtggctttatcgtcatcattattatcatcattcttgtttccatcatcacgatgagttttctccgttttcctctcctccagtggtagtcgtgtatcatcatcaatcatcgtagtgacgtcgttgctgctgctgctcttgccttcatggcggtatttctcttcctcccccctaaccccatattaactcgtgagtgtgatggttagagtggctgcttgttttttttttcttttctctttggaacaacaaaagaggataaagatggtcggtgaatgtattattattatcatcattatgatacggtcgcggtcttcttctccgatgacgaaacctgcgcacatcgaagaaaagacgagcgcgcgaaccgatagccgtccgtctgggacgaaggagaagatgatggggagaggaggagagccccagaagccagagcgagaagggagacgacagacatacgtcgtcaccgtcctctggaggaggcacggcggcgctgtttgttgtttggatgcttgattatatcctgttctatggggtagattattatcaataggcttggttttcaaaggtcagcctgtgtattgtcgtgtctttttttttcgttctcatgatcgcggagaccacacagacgtgcgcgtctcccaatggctaggcgttctttttaggtagtaattttttgatcttttttttttcttaacaagtctggcttgatttcttttatctatgatcgattcttctttttctcgggggttgcatcttccgtgaaagtaaagtgacactactctaaatggtaaccatattatctgttgattaggagaaaaaataattttttcgcacgaaatcgatcctaagtgaggtgatttacttgctatcacacgaaatgattatcttttgctgctaacgtactgaattttttaacagaattgcttctccgtaactatttccgcagattcagacagattgtcaaaaaagaatacggcacagaaatagtgggtctgtggcttttggttcgtgtacattcgcgtttgcgtgtcgagatttctacggtatgtttattcttcctgcgatgatgtagggtccttggtgtaagtaggatttcgagtatctctcttagagcgaacaaaataatcaaaaaacaacagctaggaaatcgagggttactctacgataaagtgtctctacaaagtgaagaatgttacgttgtggtggaataataagactcgcgtgatcgatgagtgatcgagagcggctcgaaccttctttaagagctttgtttagtgcaactttaaattacaaggagtagaaagctgaaatgaatctatgaaggtgctattctttgaatatcttactttgtacgcttcacattcgttatttggatagagagttgtctagagaaaatctgtgattctctatgagtgttatttttattatccttttggggactacgatttttcttcttgttctacataccactactactcgtaatcacatacatggacgaaaaaaaaattcgtcaggcagtagataccagattctccgacgttacggcgtcttttttttcttttgagagagtatctgctgagattgtccgtggtgtatctagtcgctatttttgttgttactagtagttttgcacacagtttattcagtatagtttttcttcttgccatgatcaattaagcccaccacctttttttttagagaggaggaatttcgtcttgatctccagccggagacaacggcggtggtggtggtggcgggagagacttcaaggcaatgaaaaaaaaaatttcgttttgccatcaagtggtgacgataacccgtcagattgataattggttcctacagaaactattctaaccgcggaagaaagaaattgaaaaaaaaaattgacaaaaacatcataacataaaggaccacctacctgggacgcgcagttgggcggcggactgggacggcatgctgcggcgatgctgtcggtgatggtctcttcctctctggtcctgatcgtcttttttctaggcgcttccgaggaggcgaagccggcgacgacgacgataaagaatacaaagccgcagtgtcgtccagaggattacgcgaccagattgcaagatctccgcgtcacctttcatcgagtaaaacctacgttggtaggtcacgtaggtacggtttattgcgacggtctttcttttccgcgtgtcgggtgacgtagttttcctcttgtagcaacgtgaggacgactactccgtgtggctcgacggtacggtggtcaaaggctgttggggatgcagcgtcatggactggttgttgaggcggtatctggagatcgtgtttcccgcaggcgaccacgtctatcccggactcaagacggaattgcatagtatgcgctcgacgctagaatccatctacaaagacatgcggcaatgtgtaagtgtctctgtggcggcgctgtccgcacagaggtaacaacgtgttcatagcacgctgttttacttttgtcgggctcccagcctctgttaggttgcggagataagtccgtgattagtcggctgtctcaggaggcggaaaggaaatcggataacggcacgcggaaaggtctcagcgagttggacacgttgtttagccgtctcgaagagtatctgcactcgagaaagtagcgttgcgatttgcagtccgctccggtgtcgttcacccagttactttaataaacgtactgtttaaccacgttgcgtcgtgacgttgtttgtgggtgttgctaggcgggctggaaagatgatgtataaatagagtctgcgacggggttcggcgctctgccggctgcggcggcactcgctccacggcctccgacgagcgttgcgctcgcgctttgcgccgccgcgtcatggatctccctactaccgtcgtgcgaaaatactggacttttgcgaatcctaatcgcatcctgcatcaaagcgtcaatcagactttcgacgtgcgccagttcgtcttcgataccgcgcgtctggtcaactgcgtggacggcgatggcaaggtgctgcacctcaacaagggctggctctgcgctaccattatgcagcacggcgaggcttcggccggcgccaagacgcagcagggcttcatgtctattgacattacgggcgacggggagctgcaggagcacctctttgtacgcggcggtatcgtcttcaacaaatccgtctcctcggtggtgggctccagcggacccaatgagagcgcgctgctcaccatgatttccgagaacggtaatttgcaagtgacttacgtgcggcattacctgaaaaaccacggcgaatcctccagcggaggcggtggttgcggcgccgcgtctaccgcttccgccgtctgcgtgtcctcgctgggtggcagcggcgggactcgcgacggcccttctgcggaggaacagcaacggcgaaggcaggaacagcgtcacgaagaacggcgcaaaaagtcgtcctcgtctgccggtggtggtggaggcggcggcgctggtggtggcggtggcggcggcgggagcggcggtcagcactcctcggactccgccaacggactgctgcgggatccccggttgatgaaccggcagaaggagcggcggccgcctccctcctccgagaacgacggtgagtcccggccctcctcgcgtcacggtgctttccgagtggactcgtgagcctcccgtagcgcacgagcgagcaggcgagcggtgttggtgcgctggtggttgtgtggatgataaccatgtgctttttcgtgcgctatgtgtcgtcccgtctgtaggctctcctcccctccgggaggcgaagagacaaaagaccaccgcacagcacgaaggccatggcggcggcggcaagaacgagacggagcagcagtccggtggtgctggcggtggtggtggcggcggcagcggccgcatgtcgctgccgctggacacgtctgaagcggtggcctttctcaattactcgtcctcatcctccgcggtctcttcttcctccaacaaccaccaccaccatcatcaccaccataacgccgtgacggacgtggccgccggcaccgacggtgcgttacttctacccattgagcgcggagcggtggtttcgtcgccgtcgtcgacgtcgccgtcgtcacttctttcgctccctcgacccagcagcgcccacagcgcgggcgagacggtgcaggagtccgaggcggcggcgacggcggcggctgcggggttaatgatgatgaggaggatgaggagggctccggctgaggcggcggaggcaccaccgcagtcggaggaggagaatgattccaccactccagtctctaactgccgtgttcctccgaattcgcaggaatccgcggcgcctcagcctcctcgcagtccgcgttttgatgacattatacagtcattgaccaaaatgctcaatgattgtaaggagaaaagattgtgcgatctccccctggtttccagcagactcttgccagagacgtcgggcgggactgtcgtcgtcaaccacagcagcgtcgcgaggaccgccgcagctgtctccgcagccggcgttggccccccagcagccgcatgtccgccactcgtcaccaccggtgttgtaccctcaggttccgtcgccggtgtcgcgcccgttgccgccgcaatcgaaacaccagctgctcctccccggcccgtgtgtgaaatcaagccctacgtggtaaaccccgttgtcgccaccgccgcggctgccagtaactcttcctcgtcttcttcggctccactgccgccgccgccaccaccgtcgggcggacgtcggggtcgggcccggaacaatactcgaggaggcggcggtggtggcggtggtagaaacagccggcggcaggctgcatcgtcgtcgtcctcctcctctcggagatcgcgacggagaaacaaccgccacgaggacgaggaggacaacgaccctctgctccggttgtcgcaagttgccggcaacggccgccggcgagggccctcgttcctcgaggacggactcgaaattatcgatcccagcgaggaggctgcgatcgccgccgcctcgatcgcggcgtttttcgacgattaaaaaaccgagccgagaccggaaaaaatatgaaacaggacgcgcttggacatttgggtttccaccccttttggtgtgtgtctatatatattggtcactgattttttttacaataaagagatagacatcacagttcaccaccttgtctccccggtgtgtctattatcatcaatcacccacagagtcgccagtccatggtctctcggtaatgcgtgtccagatacgcgttggccagtataaaatggtcgttgcccacgaaggcgcgggtggtgttgcgcggcgacgggtggcaggacttgagtaccaagtgccgccgtcggtcgatcaggtactcgcaggtgtgcgcgtcggcgccccacagcatgaacaccagatgctcccggcgctctgacagcctccggatcacatggttactcagcgtctgccagcctaagtgacggtgagatccaggctgtccgtgcaccacggtgaacacggtgttgagcagcagcacgccgcgtcgcgcccaggcgtccaggcaacccgaggccggacgctgaaacccgtccaccgtacgcgccagttcgcgaaacacgttgttgagggagggcggcggcggtcggcccgccagcgtgccgaaggccaggccgctggcgctgccgtcgcagtacgggtcctggcccacgatcaccacgcgcacctgctcgggcggacacagatagctccagcggtgtacgtgctcgggtgccgggtacaccatctcgagttgccgcgcgccctccaccgccgccaccgtgtcgcgcagcagcaccgtgtcgtggtcgggcaagctgaggaagcggatccagtcggcgctcagacaaaacacgcgagcctgctcgtcgggggttaacagagagcctttattatcagcaatgttagcgagcatccactgcttgagggccatagcgcgagtgagccggcaggttgacgcgcgtctgcttcagctcgggcggcagtccggcgtagtatttatctaggtggcgtagtagcggcgggtccagctggtgacgcaggcagaattccttcactgcgttgtacaggccgtaaaagagtgtgatgccctcgggcgcggcagcggtgctcacgggcagacgcacggcgcggttggtacgcgtggcttcgttgcgtatggccaccaccacgttaaagagagacggtggcaccagctcgaagcctaacacgtgttccgtgaagatgctgcgcccgtatgacagtcgcgtgaggtcgtagccgcggcacaggtcgtccacgcacgtgtacacggccggcgagccatcgccgcactcgctgtagccgcgcatcaccgtcatccagcgcggcgctgtgtccgagctcaacagcgtcagcagggcccgcaattgatccggattgttgtacagcagggccagagtgtccaggaaagcatcgtccaacagcacggagttggcggcctccggcgtaacgggacggtaacgaataagttgcgatagcgggccatcgcgtctggtaacattcaccaacgggcgcagccaactttcatacttgtcaccctgaaacacctcacccaacaggcatcgacgcgttagttcggggcactccgcggggactttctcggcggcggtaggagcgacgctgacggcgactgaggaaacaatgggcagcagaaggcaacaccacagcagtaccaccggtccaggtgagaaagagaagccgcaatccgggcggcggcacatcaagtctgcggcacgatgagagtgtgacggtaaggagccagttggcgccgaaagttggcgctcaggtcttcgatccctaaaacgttatatattgcatccagcaggtgagccaggctaaacggattcacgtaccaggtttggttacccgcgacgatgacggccagaccgtgggcgctacagttggagaggttcctgggtacgaaggtaactgagtcgatgtcgcgccacggggggaatgagacagacgactggcgcacgctgtaatcacaactgtgattgacgtgtagcgtgtaatttcggttgcactcagcctcgaagtagagggggaaccacagttcgtcgtactcgtcgtcgtcctccagttctggctcttcttcatccaccgcaatgtctacgctgctctgagattcctcttcgtacaggatgattgacaggttatggctacacaggtcctgggcgggaggacgcgtgggagcgcgggtggtggtaatgttttccagatcgtcaaaagtcggagtgtagtctgacgccgtgacgacaccgtcgacggagatggtagaagttgcggccggtgtcacggtggtaagtatggatacagaaggggagggggaagtagcgttcgtaccgatggttgtggtattattattccctgtgtttcttgttccagaaaccgttgacgttgagatgggaatcgacgtggtgctggacgtcagattgctgaccgaggaaaccgtggtgggagtggtgacggtgttactcgtggttgaagtgacgttaggggaggtagtagtggtaccggtggtggcgacggtagtgtttgtcgtggcggcggcagcggtggtactggtaacggtggtcgcgttggtttccaccgcttcacacagtaagcaaaagcacagggccgggaaaagcaaccagccccgccatcgccgccgccgcttcatgaggtgggcaggcgaaagctggtgaattcgttgtacagcggcaagtggggcgccgcgatcgaagggtacgtcaacaagctgacgttgatattaaatacgtctggctgcttttctacgatggaagcgcacagggttacggcgtcaaacaggtctttcttggtggcgcccgagacccacatctggtatacacccgtctcgtggtacgaagtagagcgcggcaccaccggacggatgcagtccagaacgcggttgggatcctggtgaaagaatttgaacgtggctacggcctgtggcgtgtgcggcatcgtctgcgtgatgagctgctggcccgctaacacggtgacgttgtgcaacttgagcagggcactcttgagggcctggaaagcgttgccgcacgaggcgctgatctgcagctgcacggccgtggagtcgtgcagccgcatgagacgtgatacctcttcgaagacgtacttgtatttgctggcaaaaagtggcgcgtaccgacagtcggccggcaaaatgtaggtggcgttaccgccgttggtggccacggcgggcgcagcggccgcggaggccggcgtaaacagcgtcagcggccggtggtggctggtaaggtcgatcatgggcggcgtggtgaccgtggcggtggcgggcatgacggggtttgcggcgacgggcactccggccacagcggcggccgcggcggccacggcggcacttgccgagcccacacccgccggcagtcctccgccacccatgacgccgccgggcagagcgtcgcccagacagacttccgcagtggcgggcgcgctctcagcggtcagtacggtttgccgatcgacctcgcgacgaaagctggtgaggaactcactgtgatccatggccgcagggcccgagatcccgggattctgcgggtgctgaccgagtgcggggcgagttatatggaagacgattagcttggagcggagttttgcgtccctagctgacctgcggatcagcgacgtgccatagggatagactgtgagcggcggccgcaacggcggggtcggccgccgctcgtcgtcacggggcggcgcgagggaggaggaggtggtgggtacgatcttgacgtggttgacgtcctgcccgtccgggggaatacgcaaaaaaccccgtcgcggcgctaccacgatggtgcgatgggtctttctcttgttggccggggccagggacttgcagatgcgtgtggagccgtagacgatctggacgtggtcctgggagaacatgaccatcgccgccaacgctcagcggggggacgtgttgggaacacagaggctgagggaaaactccgtagaagtcagcgaaataaagacaacacagcagccactcctctcgtctcgggccctaccactgcttgaagtagggcaccgggtgtttcttttcctcaacgggctcctccagtctcttataggaccagtcccgccggcgcgccagcatgtaggtcacgtacaaaagaataatcaccatgaacaccaggaaagccagcacgccgtaggccagcagccggtcctcgaacagcgggtcgctcttgataaacacgtaggtggtggtaaaacttcggcccgcaatctgaacgtggagacgcacgacagtatacgtgccgttgaggtagaagacaaactcgcgtaaccgttgtccgttatacgtcacgttactaatattccacggcggaatgagctggttgccctgatgcagatgcacggtgctgttggggtgatagaggctgctaccgttgagcaagcagtgttcgtgttcctgaagcagcacgcggacccgcatcgtggtggcgttcaggcgagtcccgtacacggcgtagatgggataggtgaaaaggtcccaagtggcgttgtgatggcggccccagctgaagaaagagcacgtgtactcagtggtctcctgcggcctgagtcccgagataagcagctcttgagcagtagcgttgtaggagagatgtagttttcctgtggataaaattcataagttgtttattttgttggcaggttggcgggggaggaaaaggggttgaacagaaaggtaggtgctacttaccttcattatcgggggggaaggcgctaagataccccacctgagtgaagggacccttgcagtctgtccgtgcataacaagtaactgataaaatgtctggatttttggtgttattcaacaggataactttgcaggtggcgtttagagacacttggtcgtggctgtagctggcttcgcaattcacagtatacaggtgcccctctttctgcgtcgtggctatcacggaagtggaggcggacgaggtagaggtttgtaccgtggtggtgacagcagaagtgacgttgttagaggtacttattgacgtagtagacgtgacggtggtattactaggggaagtgacggcgcttgtggtgctacttttcacccccgggtgcatgtcgcccaagagcgcaactacgagcgcgatcgccagtacggaacacatgttgccgtgtgacgagacggcgtgtggacgagctatatgtggcaggaggtcgcgtcacctcttgtgacgcctaaacgtccagctccagataaaagaggcgttaataatgaagaccacaaaaaccacttgcgtcagtatgacaatcataaaggctcggtgattgctacgcctaaagtacgcgggattatccaccagttcatcctgctgaacaaagtggatgattgacgtattggtgttactatccgtgttgttgaccatggatttgactaagaaagtcttggcgccaaaagtcccgttagagccccagcaggtgacgctgctattcacataagttcccggtgcccccgctagcatgtatttcagttggtgggtataattttttctgtcgttatccatgtcattgctgtagttgaccttgctggtgagaaagcgtgttttcaacggggcacttatcatcatccctgaggccaaaaagggcgaattgcaagctgtagtgttacaaaaaatagtcaagttagtgtcattgtattgataaatgtaagccatgcttacttcaggctcataccacccgattccaatcgcggccgccatcgttaccacgtcccatcttcccagacttaccaccgccaccactaacagcgtcacccccgcacggtacatagttaccctctcgacgtcgccggctgtcaatgacgtgcctgcgtcagtggctatgatttatagcttttgggcccaaccgcaacggatctgtcgtaatctaccttccacagggccgccgcgacgatgctgaacgacaggatcagacagacggcgtacaaaagtcctaggtcggcgtcgacgcggcaggtgcggatgtctcgcagggtgggtagatgggcgatgcacaactctttctccccccgcccgtacatcccatcttgtatcagcagccgtagcgtggcattgatggtcagcggggtaaccaaagaaatcacatagggatgtgtacaggaagtgcagtgacgggtatccgtgagatgtaagtcaccaccctcctcaccgtcatcatgaaagaccaggactcgggtgagacgacccgatgaatactggatctcccaccacagtctttggtccaacaccgagagggcgcaagagattctaagtctccctgggttgggggagcagatgtaagtcccgtatgtgcctctcgccatcagggccatacacatgagggggagaaggacaattatccgggaccacccgcacccccacatcacgagaccagagacggagatgtataaaaaaagctacttttattaaacagcattctcaccacacgttaatactgtcacggggaatcactatgtacaagagtccatgtctctctttccagtttttcacttactgagacttgttcctcaggtcctggatggctgcctcgatggccaggctcagggtgtccaggtcttcgggaggggtctcggtgggctgctcaaactgccccacggcgtaggccttcgcggccgtctcgtagataggcagcatgaacccaccctggttggtggagaagatgcgcaccatgacctgtttgggaaacttttgcatcaggggcaggcacaggttgagagcgcccaacaggtccacgggggtggcagcgtggatgatcatgttgcggtaatcggaggaacgggggcataattggtgggtgtgcaattctttgaggctccacgcggccttgacgccttcgttacaagcatcggctgtgcgctgcgccacttcgggtggatgtgtcacgggcatggtgtgctccatgaggaagggagtggagagggccaggttgcacatggtgcccaggcgacaccgcaccgcatccacctcactcttcacctcatgattgcgggtgtagataatctggatgcccttgttgttcacctgcatggttttgcaggctttgatggcctcatctaacacctggtgcatactgggaatcgtgaagggcaggttcttgtactcaagagagcgattggtgttgcggaacatgcggctcacctcgtcaatcttgacgcgaccccgccgagtctgcacgttgggtgtgcagaagggggtgttcttatctttcatgatattgcgcaccttctcgttgtccaactcggagatgcgtttgctcttcttcttgcggggtccggtgctcgccccgccgctgctctgatggccgcagctcagcagagaggaggaggccgcgccaccaaaaccgccgcgcccatggtggctcgaggtcacggatgctcctccgccactgctgcatttcatctcctcggactcactctccgagtccgaagccgaactgcaggaggaggaagacgaagaggaactatcttcatcgggccggcccaagggatcgggaagaggagggtggttcatctgggagagcgggtgcgtgggagaggtcactcgcggcgtgccgctgccggtggaaggggaagacgcggtagcaccgcgggtttcgacttcttcaccctgttcttcctcgctatcagagatcacgatacagccggcggtatcgataatcttgttgcggtactggatggtaaagtcgggctcgggcttgatgtcttcctgtttgatgaggggcagcatgataggcgcgggaggcacgggcggtttaataatcaccttgaaaggacgcgtggttttgcgcggtttcttacgcgggctgagctcgggagtagcggatgccccggggagaggagtgttagtaaccgcgacgctggtgggggtcggcttgttaagaggggcgctgctaacgctgcaagagtgggttgtcagcgtggggccggtgctactggaatcgataccggcatgattgacagcctgggcgaggatgtcacctgatggtgataagaagacacgggagacttagtacggtttcacaggcgtgacacgtttattgagtaggattacagagtataacatagagtataatatagagtatacaatagtgacgtgggatccataacagtaactgatatatatatacaatagtttactggtcagccttgcttctagtcaccatagggtgggtgctcttgcctccagaggcggtgggttcctcagcaccatcctcctcttcctctggggcaacttcctctatctcagacactggctcagacttgacagacacagtgtcctcccgctcctcctgagcaccctcctcctcttcctcatcactctgctcactttcttcctgatcactgttctcagccacaattactgaggacagagggatagtcgcgggtacaggggactctgggggtgacaccagagaatcagaggagctgacaccagcggtggccaaagtgtaggctacaatagcctcttcctcatctgactcctcggcgatggcccgtaggtcatccacactaggagagcagactctcagaggatcggcccccagaatgtactgggcaaagaccttcatgcagatctcctcaatgcggcgcttcattacactgataacctcaggcttggttatcagaggccgcttggccagcatcacactagtctcctctaagacatagcagcacagcacccgacagaactcacttaagagagagatgcccccgtacatggtcatcatacaagcgtcactagtgaccttgtactcattacacattgtttccacacatgtagtgaggatatccataaatatgtgatcaatgtgcgtgagcaccttgtctctctcctcatccaaaatcttaaatattttctgggcataagccataatctcatcaggggagcactgaggcaagttctgcagtgccgccatggcctgactgcagccattggtggtcttagggaaggctgagttcttggtaaagaactctatattcctgtagcacatatacatcatctttctcctaagttcatcctttttagcacgggccttagcctgcagtgcaccccccaacttgttagcggcgcccttgctcacatcatgcagctccttaatacaagccatccacatctcccgcttatcctcaggtacaatgtagttctcatacatgctctgcatagttagcccaatacacttcatctcctcgaaaggctcatgaaccttatctaagatatctaaggcattctgcaaacatcctcccatcatattaaaggcgccagtgaatttctcttccgtctgggtatattttttcagcatgtgctccttgattctatgccgcaccatgtccactcgaaccttaatctgtttgactgtagaggaggataacaacacatataagtatccgtcctcctgactcatttatcgctatctcgatgccccgctcacatgcaagagttaatctttactctatctgacatacacaagtaaatccacgtcccatgcaggttagtatacatcacatacatgtcaacagacttaccgagttctgccaggacatctttctcggggttctcgttgcaatcctcggtcacttgttcaaaagttttgagggattcttcggccaactctggaaacagcgggtctcccagactcagctgactgttaacctccttcctcaacatagtctgcaggaacgtcgtggccttggtcacgggtgtctcgggcctaaacacatgagaaatagagtcataagcacatgggtcacatacaggagatatgtatataacattaatacaattttattaaaaaaaaagggggggcacaaaccccgacacgtaccgtggcaccttggaggaagggccctcgtcaggattatcagggtccatctttctcttggcagaggactcttccggttttagaagctccacatcgaagacgagagtggcatgtggtgggatgatgcctgggtgcccagtggcaccataggcataatctggagatatagtcagtttggctctctgacccacactcatctgggcaaccccttcttcccagcctcggatcacctcctgcttgcctagcataaacttaaagggcttgtttctgtcccgggaggaatcgactttctttccatcttcaagcatcccggtgtagtgcaccacacaggtctggccgcgcttggggaaggtgcgcccgtctcctggggagatggtttccacctgcactcccatcgtgtcaaggacggtgactgcagaaaagacccatggaaaggaacagtctgttagtctgtcagctattatgtctggtggcgcgcgcggcagcaacgagtactgctcagactacactgccctccaccgttaacagcaccgcaacgggagttacctctgactcttatcagaacacaacaactcagctgcctgcatcttcttctgccgctgccttaagtcttccaaatgcgtcagcggtgcaagcccgctccccgagctcattttcagacacataccctaccgccacggccttgtgcggcacactggtggtggtgggcatcgtgctgtgcctaagtctggcctccactgttaggagcaaggagctgccgagcgaccatgagtcgctggaggcatgggagcagggctcggatgtagaagctccgccgctaccggagaagagcccatgtccggaacacgtacccgagattcgcgtggagatcccacgttatgtttaataaaaactgcgggcactggggacggtggtgttgtatatgtgaatttgtaaataataaatgagaccccatcctgtaaaaatacagagtccgtgtcagtctctgaaggacagtgtattggcatatagccaataaagagagttgtggcaaagagccatgttatggattagtaatggaaagtatcgtcaccaataggggagtggtcaataatggtcaataacccacacctataggctaagctataccatcacctataacatgaggaagcgggggtgtatagaccccaagccaaaaacagtatagcatgcataagaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatggccaatagccaatattgatttatgctatataaccaatgaataatatggctaatggccaatattgattcaatgtatagatcgatatgcattggccatgtgccagcttgatgtcgcctctatcggcgatatagcctcatatcgtctgtcacctatatcgaaactgcgatatttgcgacacacagaatcgcccaagtcaccaaagtcgtctatcgccatcccccgtaaacgatataagcgctatcgccagatatcgcgtatgcccaaaaatcacttttggaaaaatggcgatatcagttacacagaaactcacatcggcgacattttcaatatgccatattttcaaatatcgatttttccaatatcgccatctctatcggcgataaacaccactatcgcgcgacatgaatttagtcggcgacagaaatctcaaaacgcgtatttcggacaaacacacattttattattcactgcagcatatagcccattttagcgcggcacacatccagccgtttgtgttttttaacgctctccaggtactgatccaggcccacgatccgggttatcttgtcgtattccaggttgatccatcgatagggaacgctgccagcggcgcccagcaggtactgcgccttgtcgttcactttgccgcagcgtattcgcccgtcagcttcgaggtataacctacaacacggaggggaagggggggtacaaaacgtgaaattagactttttttttaatgatgttttgtccctctgtcttactttcccataggctgtaaggccctcgaggaagagacttacggattgtagttgcagctcgtcagtttgttgtgtacgacctggcgtgtcaatgaatgggtcatggtggtgacgatcccgcgaatctcagccgttttctcgggactgtagcagacttcgccgtccggacaccgcagcctgtggattcatgaaaatctactctggcattcccgaggatcgtcgatggaacatggctatcagaaacgtcgagagacaaatccagacgcaccacagaacgcagacaatcataaaaatacgtacgcgacggtgaagcgattgcacattttgaaatcgtaacagcgttccggcgggtggttgacgtttatgaattcgcaacattcttctgcgcgcacccgcggcacgcggctgtgacccaatagcagccacaacgtcgtcaagaacggcgtcaggtctttgggactcatgacgcgcggttttcaaaattccctgcgcgcgcgacgggctcaaacgatgagattgggatgggtacagaaggtgtaagtctggttattggcctcggtgaacgtcaatcgcacctgaaaagacacgctgtagtcccggaagacgtgggcccagctctccagcttcatcacacacatctgataacgcgtgccatcgttgacgacgaagcgtagcagcttggtctgcttgggcaccatgtgcgctccaaaaatcttggcgtcttccacgctgatctgcacgtttccgtcgctcggtttcgaagccgtttggggcatccgttggaggatggtctggttgcgaccgctcagataccagatcacctttttcacccaggtggagcttctctccaccaaggtctggccttcccggttgtacagcagatacagggtctcgttgcgacactcgggacccgttgatacctgctggaaccccgagaattgcaagggggaccgtgggggcgagggatagagaaaaggacagtaaaacgtcgccgcgtcatgcggtttggaatacgtcagtttagaccatggcggggacggattctggtttgccgttagcgtcgaccacggagacgccagacagggcgttgcccaaaccgcgcacagaagcaggcagtgaaagtggtgacgaagcagaagccgcagcatattatttcccgtgacgcaggctagttggcaaagagccgcacgctgaactcgaggctccgggcgtgtggcgccagcgaaccggcggcgttgaacgtggtccttttgttggtgccgccgcgacggttctgacgtctaaagtcgctgatgagcaacgacacctcggtcacgttgattctgcaagcacaggttccaaacgtcatttcataccccatgcggttacttagccgttacccgttcgcccttaccttcccgttgtcatgcacctttagcgcgtaccctcacctcttgagcacgtcaaagttgtccaagccgtggctcgcatcgtagtggtagttcaacgtgaggtccacgagctgttccacatacttgtaacgggtttggtcgggcagcgcgcgagagcacgcgtcccagtaatgcggtactcggtaataatcgtttttttccgcggtttcccgctggcactgacccagcaccacggcgcacagacaaacagacagccacacccgacacagccgcatgttgcagactgagaaagaaagctttattatgagacatcatacacatagtataggcgaggtgatggggcggggaaagagttggaaccgaaagacaaaaaaaaaagcctagtcgtactcgggatctctgagcgagacgggttgcatggcaactttcattagtttgggaatctgccagctggtgctgttcgaaggttcttccatttccgaggcggtcagttcatcgtacaccgaaacgtagtacctgatggggtcctcctcattgtccgagaggtgagattcgatggtcaaaggcgagcctctcccataattgggattcacgaacgacgtgtccaagttgccatcctttctgaaatagatgacgttctcaggatcatgtttcatgcgctcgcgggccgcggacgcctcctcctcctcgtcccagtcccgagtttccaaccgctgataagggctcgaggaacaaaatccggcggggatctgagaacctcgtcgggaaccgctgccaaacgggctgctgccgccactgtcgtccgtgtcgtccaacaggttgacggcctcttcgtcggcgaaacgaaagcggcccgggtgcttgcaacacgaggagtaaactaccgcgatcagtaccgctatgaagctgaaaatggaggtgcctgtcacgatgtagaagaggatagccagcactttcatgatttcgtcattgcgcgcgtcgtgaacggaagattcgcgggcagtggtcatgttggtttcggttgtaggttcgctactcgtggtgctctcgacggtatttctgctgctggtgctagtagggacgtttgtgctgctggtcatatttgtagcgtcgctgaagtcgatgtgaagcagcaacccgaacgcgaccaggaccaggaatgttgcgcgaaggagaccccgcggggccggcattcttgagacgtggcgacgtggatttcttgttatgtccgcgaacgacgtgtaacgaggacgtggtttccgcaagcctctaccgacgccgcgacaccaggtaggttatcaaaacgcgagcccatatcgccgccatcattgtaatcagcaatgtgttgaggtactgcacgatgaatctgtctagtgacaccagccaaccctctgcttttgcgggcaagcgcgctttcggtgacagggtgtatcgtacgtagccgcgggtcaggcgcgcgttgtagcggtacacgcagaaatctatccacaggccaacgcccggctgtagcttcggatggtggataatagcgcggtgacgtacgccgcgtggctttagaatctccacctgtaaggccatctcctccaggtagtgggtctgactgcgacgcagcgtccagttcatgtaaaagtcggtctcgccgtgtccggccacgaagaggctgcttactaatccagtctacattgtgccatttctcagtctgattgcattttttagagttatgttgccaccaacgtatcctgttacgttgattacctcgtaactgcggtcgcatcttttatggactgtataattgaaaccatcacaagtaacggtcgtggtggtgttggtacatgtggtagtctcaacgtttgtatttgtcgttgtggatatctgtgtggttgttttcgacggttttgtagaaacggtggttgctggtgcagttgcagtagagcaatttatagattctgaagtgcttttattgctgataactgttgtactgtatgttgatgtggctgtctcagtactagtggaatagttaacggtagtactacagggacattgacaggaacaggtttgattgcagctttctgataacgcggatattagtatcgtccacataaccgtaaatcgccagtccattgcaatattagttctcgctcaatgggcattaatattcctttgaacgctgagccttacagaatgttttagtttattgttcagcttcataagatgtctgcccggaaacgtagctcaatcttcatgttctgtgtgatatcgaacaatgaattctgattcactgacggtgtcttgcaacatatggtactttttgttaaaggctcgtcgtgcaaaaaacagaactatgcaggccatacaaaccaacacgatggtccatacggtgcggcttctttgcgagctatgatagagattacgtttgtggtgatgtgtattgttagtatgttgacttcctttctctctatcttcattttcgatatcggtgttgtatctagggcaaacgaaagtagcattaatagcttcagtatgattttttggtgttactaataggtagaaattttcatcttcgtgatgtcctgtgaagtaattttctttaaaacaacgtctgctgtacctgccggaattggtgatatttagatcgtacagatgtagttctgtgttgttgcacgaacgacataagtcatggtacagtgaaggtctttcatgttgagaatgacatacagtatgagaggtaaggatgcgtaaccaataccaagattgggtatgtgcgttcttacgatgacctagatgatgtccgtgtgtggatcgattgtaatgtcgtatccaggcgactgaaagacaatcccacgtagaattaccttttatggtgacattactcccttctattcctgttgtattagtttctttgaaacgtatgattgttgtctctgtgtgacaagcgttggaagagttagtacggttgtacgtggtgtacgttgtggcgctgcaatttgtaagccatggcgtgcttataagtgcagtattagtggatacgttgtgcgaagtttcatctgacgtgatagttacggtgattgttgtgttataagatgatgtagcgtttgctgttacgttggtaaaagataatatagtgttggtatttgttgaaatcaattctgtagtggcagccgtattggatatattagcatatgatgtattgaatgtagaatatacggttgtgaaagtactcaagtcggaggtaacgttggtgatgttgccaatggttgacgcttgtgaggtgacagatgtgtgtggcgtcgttgatgtgttgttattcggagtagaaaatacgctggtcacaaaggtggtagaagcagtgttgggtgatgtgatggatgcagtattggtagtagtactgttgcatgtaactctatgcagaatatagaatattatgattgtatacgccgtatgcctgtacgtgagatggtgaggtcttcggcaggcgacacgcatcttttactgtaaatccccgtccaccgtcaacaacaaaggttccgtatctaggtccgtccgcagatgttcagcgtcctgttccccgattcgttgcgatcgcaggaagcagatgaccagcgcgccaacaaagatcatcattcccgaaacccaggcgcaatggagtgagaggccggaccactggcgttttaaatccgagataattgcccggtctgcctcttgggaatccgtaaccacaactctccctggtcccggataaaagcatcgacgcgtttccaaggctcggcagaagctacgtgggtggatgatgaggtagaaagcctcgacatcgccggtatactgatcctgcaggaggtagactcccgtatctttaaccgtgagattgtacagcgtcagattttggcgcgtgcacgcgaacgccgcaccgccctgacgcgtggtttctttataggcgtctgtaatgatacaaagtggcggcatacgacgcatgtatctgctgtagatatcataacgctgccagactacgctgtgatggctagtgttaagcctggtaaccagcgtgcgtgtacggtcctcgcaggtggcacggtagttggcgagctttaggggttttttggttggttcgacggcgttcgatgaacttccctgagttgtgaacaaaaacagcgacgtgactatgacaagcgtgaggggggtgctgtaggtctgcatggtgcaaaacacgttctcgccttccttatcagacgttgtcgtcctcgtcctcttcgtcgtctgtgcccgtcggttcgatcaacggggagttatctttctgtctggagggtcggtatggaatccgttcgtagatgttctgctttttagccgcgtgttgttccagctttttgcgtgtcaggctccgataggccagacattgatctacctcggtgcccgtgttgtttttctcctcctcgcgcgcgtaaattacaaagaagaccaccagcaggactatcagcgtagccacgaacgagcccgcgccccaggccgagtatgcgcctagcatggtaatgggttctgtgatccggcatttgcacatcgcgtggcacttgctgccattgccggtattagatgatgtgttattcggactgcacttgcacgtcaaatgggtattttctgatttcacgagacagttggtggcgactttggtttcggcgcagacggccacatagcttaccaagctgagtgccagaaagcacaccgcgtgcattacacgcggatacatattaaaacaccgtgttccacaagcaccgcacacgtcaatcctccccgcacggtcttcagcccgcccatgacatgatctccctcacgttacccttcaacaccctgtagtactctgtctcggcttccggtccccatgtcctaattataacaaaacaccgtgacactgtccatctccctgtctttttgcgccgccggtcccccccaaatcatgtctctagatgccgccggccaccaaccggaggcacggcggctattggattcggcattggtgcgccgcgtcttggcctgcatgatcatcgtcatcatgattgccattagcatctggatcctgacctacgtgctgtttctctaataagaaccccggcccctgacggtaattttcctttcttctccgtttctcctcagctgccgtacgtgatgcctcacggccatctccgacaggccctctccccgacctcctggacatgtgagggcttgttgctcctcctgggattgctggtgctcttctttcaccaccacaaccagtcggccgtggagaggcgtcgccgcgtctcgttcgtcgaggccgatcgactgccgcatgagagcgggtggtattcttccgatgacgacggagaccgggacggtgatgaggaaactggagagagccacaacagaaacagcgtgggactgtccgctgtttttagctgactggcgtgcgacctgtaaaccgttactcgggtctcaagatggtttggaagttgtgactcatcttcctgtgggtgatacccaaccggacgcgagtgttccataaaagccgggcgctccggcgagaccatgccatcctcgccttcggacgccccgctcctcttctctctcctctcctccccgctgccgcggccattgccgccgccgcccataccatcggcatgtcggccgacaaatcgcagctgtcttcgccgccgcagctgtagcagttaacgtcgccggcctccaggaggagatggcgctgtgcgtcgtctcttcgtcccgtctccctctgtggtcgtgggtggtgcgagagtacacgatgggtggctctcgtctcgggggaccacagggggaggggggtaatttattattcgtattactgtaattttgtatcgcttaatttgtttagagccgcacgcttgacaacgccttgtatagccttatttatcccgatgacttttttctccgtacaagaaatggacgtcacttgagcagacacagtttcatcgaccacgacagtctcatgatctgactacctctgacccgccaacgagaaaaccgaaaagtaaaagatgaccgcgccctcggagtccttttttccttttcaatcatgaaagcaagaggcagccgagagaatgccagtaagagacgaccatcgcagacacagtacgatactcatcttagaacgaaccagcgaataaccatcacacgtacagcagaatctcatgaactagtcaaccaacgtcataaaatcttcacacaatcgtttttgcgaacttttaggaaccagcaagtcaacaaaagactaacaaagaaaaaccatcttggaattaaaaaaagtagcatcgttaccttatgaaccagcagcattcagtatatacaccagatataatatatttattaatgtatcctctctttctcctgatgtaattttgtttttgtaaattcaattgttgaaagtctctccctgggggaattgcatatcttattgatgaagaagaaatccctgccatatgtgttgtcaaactatcattatttctctatatgggtattttttttctaagaagcaaaagactagcagcagccaaaataaacctgatgaaatctttaactgaactcccagtggtctgtgtgtatatttctgttggtggtcggttgtctgaacccgggtgggttgttcggaaacggcgggacggggaaacggatggaaacagcgtcgctatatacgtgacttttgatctaaacggacgtcgctaggctgacagtttacgaattgctaaacaagataggaacaaaacaagcggggctttgcctggtaggatttcctgtggaaacaataaccggatgtgattgtggctggtacataagctggttctggctgcaagcgcttttcactgcattaggtttggcgtttgcttttgcctgggaacgctatggctataacgggaaagaaccggtttggcaacattccattgtggggggggggtacttatagcgtgcctagctatgacgttgatatatgtggatgcggataatactcgtaatgagctaaaagcgacgactggtagtaattttaccattacgcataggaaagatccgttgacaactaagtggaaaaccgtttttggtaacaatggtgatcagtggttgtgcaacgttacgggtataggtaatgctactgtgaatagtaacgcaactatttgtgtgtcgagctgtggtcataatacgttggatttatgtaatttaaagtcgggagattctggcttcttcgatctgtctcgttggttcggtgaaaacatggatgaatacagtggtgatgtgtggcacttggaagtcagctaaatgttgtatcgcttagtgaattggtgttcttacagttttcatgtaataaactacgtgtaattcgttaaatttgtgtgtttttttgttagtattctgcgtaacggtggaataaaattgcgttgacctagttagatttcctgtgtagaacaatgaccggacgtgcttggactggtacatacgcaggggctggacgtggttaccggtcactggactcggtttcgctgtagctgtggttcaacctgaacatggctcccagagctgctaggaaccggtccagtcacattttttggtgggtggggggtactaaaaaagtgtttaatatttgggtttaatgataaaatccaggttatggatatgaggaaactgaatacctcgcagggtcgaaatcttaccacagttgatgatagaagacggttttccatcgggtgggaaacatgggataacggtggtgactaataatggtacaacggtcgtcaatacaacagcctgtgtttcaagttgttcgcatacgtcgcttgtgctttgcaatatgacgcagcagactgattcgttgtacggagtgggtcatcggttgaatgacgaagaagatggtgaactgtggagagtttcggtttcttaataatcccatacgacatgtgttcatttatatctgaattttaggatgatgactatagtataactctggggaacaaatatcatacgttaatcactttaagttacgccgttaggaaaagaaaatcagtccgaatgaagcatagtcagccgaatgatacagcaatagcttgtttacaacgtgttcttttttacattatgaacgtgccttgctttttatacacacatggagacagaggtccctcagcccttgtcacgacaactccctttttctaaaccgtatgtgctccaaaccgtatctcctcatcgtcacgtgaaataccatgggaccccttttcgtcacacacgtctttccgcttacccaacgcgtcagcccgcgctcggcagagctaccatataaaaacgcaggggtttagcagcttccccagatcgctgctgccccggcgttctccagaagccccggcgggcgaatcggccggctggtcggtcggcgctcggacggatggggagaacggcggtgacttagccgcccgtggccgggagaagacggaggagccgagatgacaacagcagtcgtggaagggtcgccaagccccggtccttctcttctgtctggtcgaatcttgttttcttttttcaaccgctctttttgtcacctttttatgtgagtttctcttccgcgtctcccggccgtaccatccacccatgcagcatgcacgcgtgtatgtatgcatcgcctctcctccgtcccgactaccatcagcagtaccactgccgccacccccagcgccaccaccgctgccgtcgccaccgcgttatccgttcctcgtaggctggtcctggggaacgggtcggcggccggtcggcttctgttttattatttttttttattttttatcttctcctttccttaatctcggattatcatttccctctcctacctaccacgaatcgcagatgataaacaagagggtaaaaagaaaaaagctacagacatttgggtacctcagctttccgataactcgaagaattcaaagtcgacgattcccaacaagagaaaacagaacaaaaacaaggtcatttttatttatcctcatcgtcaacaacaactaccgacaacaacgaaacaccaccaagaatgtcaatccgcaagggtgttcctgccccctcgacgcgcctgtcgcgatcctcatggcgaggaccgcgatctccgtataggtagatgaaattatcccgtgtccggtcctgattccccgcatgccctgcacatcctgacgcgtcggtcagcagccaaacaatcataggaaatgaaccagaagaacaaaaagatcatctctctcggtgtatagcaacaccaacaacaaccgcatcgcaacatcttcatccgcaagacggaaagaaaacaacaataatgagaatgaaatcaccacaaccaagccagatttcacgtccatgagtttttattatattattatcaaaacgaaaaacagaaaaactgtcatagataaatataaaaaaaaatagaaaccacaaacgactactagtactccaatcttagatgtatatgctcctagataagatttagtattaccataatcatcgaagaatgaaagacgacgatgattccttaccgctcctgccacccggtctgtatgtagagagagaagagagaaaacggtgaatccaagatccccgggtcggcgtcggcatgccgctgatcgcagtggccccacctcggcatgccggcgccgggcgaggaattgctcatgaaaaaaagtatctttctgtaaaaaaagaaaacaatacatgattaaccgaaaagaaaccaacaaaaagaacccgagatcagtcgatttcgatcactacgataaacacatggaagatttcttgaaaaaagaaaagagaaagagaccaccttcccggcggcggacacgctcctctccgtcgccgttctgcaccatgattcgatcaataacaacatcatcatcggagaccatcttttaatcaatcagcgttgcagtagtcgactccctggacacgaaggagtcatccatttttatcctcgcacttcttcgctctcaaagccgcctttaaagttgaaatgaaaggatggaaacatggaatacagttttaattgcacgtatcaccattttactacaaaaagaaaaaaaaacaacttacacatagtattaccttaggtttacggataagtagagtgtaggcgtttttgaaacagttcagccaatgcaatcttgtctcggcataatcactctttctgcatataatagtagtagtagatttattcacatcaacacagcgaaaaactccagcatcaaagtacacctagagacagcccttaaaatatagtttgcagcttttagatgtacttacaccaaagaagattaccgtccttacgagaaaacagatactcggatataggaatcaagacagctctgcactgaaaacacactctcctgtcacgacaccgcgccacaccagaggcgtacgcgtgacttcatcgcaacgatccatcgtgatgtccctcgcagaacctaaaaagaccaaaaaaaaatcttggaccacagttgtcgatacttgaagacaatattctcgtgagaactttgagattcgcacttgaaacctcttaggatccacaaaaacaacaacctctgtatggaaaatgcgctattttatctcagcttttctcccaaacctcggtttcttcctattcttatgttttccctagtatatttgcctccttataagaaaagaagcacaagctcggtcgcacggattattccttctgctaatctattattttgttccttttttttttctttgccttcaccctcttcactccctgtagcaacacagagtagtagacacaataaatgagaagtttgcatgcatttgtcgtgtccgtggtttgttatggcgtgtggagtgctcgggatgggtggacgtggggacggattcttgaggctacaaagatacgcggagacgtcgtggcgaggggatgggtttattggatatcggtgaagcagcgtggcggcgaaagacgcgatccctgggctggtagatccccctaccccgtctaccagggacgtttatcctttggacacgtaaatgtctcggccggcatccacgcgccacgttcaccgcgttgtgcccagcgccatgtgcgggtcgtttcggcgtgaagttggacggcgtagtttcggggattgtgaaccgtggctgagggtgtagatgggacaggaaaaagcgtgtgatctgaccgaggcgaagcatgtgggtggtgcgatgcggtggatgtggcggggtgcggcggtttccgacgtggagatgtggagatgggggtgatccggatgcgtggcaagaggcctcgagcttgggcttctcccgcggatggacgttctaactgtacacggcggccgtggcctccgagtaaaaaaaccaggtgctgacgccagacagagacgccgtcctcggaatcgtgtgcgcgaaagcctgtgccgcggcagcgtacgacgttccagtcagcgaggccgtcgcgttggcgcgccaacagtaaggtgacgacaggttggcggcccatggttccgaagcgtccccacatgcaccagcagtcggcgtcaaagtcgcttgcgctgtcggcccagtcgccaccgccgcggcggatttccgcgcgggggacggggtagccgagtgctgcgccctcgccaatgttgtgaagtggatgcgtgagttgatgttgattctctgtgggaaaatgagcgctgtcctgtgggttggtgttggggtatgcgagtagtaggggttgtgtttgatcgtagaggtgttggcgggcctgtgcgcaagcagcgtagtctgcggcgtcgagctccatctgtgtgcggtgttcttcgtcggcgtgtttgtccgaggtttggacatgcggttgtgtgttgctgtggtgtaagggtaacgtgtgttgggcgtctgggtgaagcggcgtggtgtgggtgctgtttgtgtctgtggctggcatgattgtgcggcatgtgtgtgttgtagtgggtggaggttaaataggtgaggtgggttccctggtccgcgccgcaaactgtccccgtccccaacgtaacctcccctacgcggcgcgaacagccccggccccagcgcaacccccgtccccggccccaacaccgtcccgcacaccccccgtctccgcaacaccccggcatcgccggcggccagaacgctcgaaaacccccgacaagcgcagcgccgaaacgacacaggcaaggaccgtggaacgcaccggcagcgcgccgaaacaccgtcccgaagcccggtgccgacaacaaataccgtgggacgacacgcaccggcagtgcgcaggcagcgtcggacacaacacgcttacggccctcaacactccctcgaggacccaccacgcggccccgcaccggcggtgttttgggtgtgtcggggcgcggccgggtgggtgtgtgccgggtgtgtcgcgggcgtgtgttgggtgtgtcgggggtgtgttggcagggtgtgtcagggtgtgtcgcgggcgtgtgccgggtgtgtcgtgccgggtgtgtcgcgggcgtgtggcgggtgtgccggcggggtgtggtggcggggtgtgtcggcggtgtgcgcggcctcggggtgtgcggcttcgcaggaacgagtgtgtggcctcgcggccgttatttcccccgcggtccccagggccgtcgtccctcgcccccgggcgttgcttttcgtgtgtccccagggacccatgctgccgtcccccgggaacttcctcttttccccggggaatcacacagacacagacacgcgtcttcttttcgccgtgcgcgccgcacgtcgcttttattcgccgtcgccgtcctccgcaccacacgcaactagtcgccgtccacacacgcaactccaagtttcacccccccgctaaaaacacccccccgcccctcgaggacccaccacgcggcccggaatggatgtcgggcgtccacctagatgggtgcgcgcccgggaggcggctgtgcgctccagtggtacgcgcctgccgcgcgtcttccttcgggtagctgcctttcccagtccacggccttccagactgcgtggcgccaaggcggcgccagcacgcgccgtgcacgtcgctgcctataaaagccagctgcgtgtcgcccgcggcacacgggcgacgaaggcgtccgcgtgtctaaaccgcgtgctcgctgacgcgggtttgcttcctatatagtggacgtcggaggtgtccggcgcccatggcccagcgcaacggcatgtcgccgcgccccccgccccttggtcgcggccgcggggccggagggccttcgggggttggttcctctcctccttcttcttgtgtgccgatgggagcgccgtcaacagcgggcactggtgcgagtgctgcggctacgacgacgccgggccacggcgtccaccgggtagaaccccgcgggccgccgggcgcccctccgagtagcggcaacaatagcaacttttggcacggcccggagcgcctgttgctgtctcagattccggtggagcgccaggcgctgacggagctggaataccaggccatgggcgccgtgtggcgcgcggcgtttttggccaacagcacgggccgcgccatgcgcaagtggtcgcagcgcgacgcgggcacgctgctgccgctcggacggccgtacggattctacgcgcgggtgacgccgcgcagccagatgaacggcgtgggcgcgacggacctgcgtcaactgtcgccgcgggacgcgtggatcgtactggtggctaccgtggtgcacgaggtggaccccgcagccgacccgacggtgggcgacaaggccggccatcccgagggtctgtgcgcgcaggacggactgtacctggcgctgggcgccgggttccgcgtgttcgtgtacgacctggcaaacaacacgctgatcctagcggcgcgcgacgcggacgagtggtttcggcacggcgcgggcgaggtggtgcggctgtaccgctgcaaccggctgggcgtgggcaccccgcgcgcgacgctgctgcctcagccggcgctccgacagacgttgctgcgcgccgaggaggcgacggcgctcggacgggagctgcgccggcggtgggccggcacgacggtggcgctgcagacgccgggcaggcgactgcagccgatggtactgctgggcgcgtggcaggagctggcgcagtacgagccgttcgcgtcggcgccgcaccccgcgtcgctgctgacggccgtgcgtcggcacctgaaccagcgtctgtgctgcggctggctggcgctgggcgcggtgctgcccgcgcggtggctgggctgcgcggcggggccggcgacggggacggcggcggggacgacgtcgccgccagcggcgagcggcacggagacggaggccgccggcggggacgcgccgtgcgcgatagcgggagccgtggggtccgctgtacctgtgcctccgcagccgtacggcgccgccggcgggggcgcgatttgcgtgcctaacgcggacgcgcacgcggtggtcggggcggacgcggcagcagcagcggcgccgacggtgatggtgggttcgacagcgatggcgggtccggcggcgtcggggaccgtgccgcgcgccatgctggtggtgctgctggacgagctgggcgccgtgttcgggtactgcccgctggacgggcacgtgtacccgctggcggcggagctgtcgcactttctgcgcgcgggcgtgctgggcgcgctggcgctgggacgcgagtcggcgcccgccgccgaggccgcgcggcggctgctgcccgagctggaccgcgagcagtgggagcggccgcgctgggacgcgctgcacctgcacccgcgcgccgcgctgtgggcgcgcgagccgcacgggcagtgggagttcatgtttcgcgaacaacgcggtgaccccataaatgatcccctcgcatttcgtctttcggacgctcgaactctcggtctcgacctcaccaccgtcatgacagagcgtcaaagtcaattgcccgaaaagtatatcggtttctatcagattaggaaacctccttggctcatggaacaacctccacccccatctcgccaaaccaaaccggacgctgcaacgatgcccccaccgctcagtgctcaggcaagcgtcagctacgcgctccgatacgatgacgagtcctggcgcccgctcagcacagttgacgaccacaaagcctggttggatctcgacgaatcacattgggtcctcggggacagccgacccgacgatataaaacaacgcagactgctgaaggccactcaacgacgaggcgccgaaatcgacagacccatgcctgtcgtgcctgaagaatgttacgaccaacgcttcactaccgaaggccaccaggtcatcccgttgtgcgcgtccgaacccgaggatgacgacgaagatcctacctacgacgaattgccgtcgcgcccaccccagaaacataagccgccagacaaacctccgcgcttatgcaaaacgggccccggcccacctccgctgccgccaaagcaacggcacggttccaccgacggaaaagtttctgcgccccgacagtcggagcatcataaaagacagacccgaccgccaaggccgccaccgcccaaattcggggatagaaccgcggcccatctctcgcaaaatatgcgggacatgtacctcgatatgtgtacatcttcgggccacaggccacggccgccagcacctccgcggccgaaaaaatgtcaaacacacgcccctcaccacgttcatcattgaaagtctctccagtccatatgttgtcaggacgtgctgtcgttctccgcttgctgcgaagcccgttcttccgagtcgtgtcgctgcgtccagcgtcgcgcccaagatgggaatttgggtcttttcacgcgtagcctcctccaccacggctgctgatcgccgtcactaaggaccgacacggaggatgacgaggagcttctccccgactccgcggtccgcgaccggctacgtagcgcgtgtccctgccagtctccgcagttacaccacacgtcgtgagcagcgtgcacctgctgccgccactgggcctcggcgtgctcaggccacccgccggagcccggtctgagctccgacgcaggatgcgcgtactcaacgtgcgccttccagtccatacagcaacaccataggtcgtgcgagtcgtcggctacccgccgccaggccagttcccgcatgggaaggctggacacgccgaccgagaggtcaccgagcccggacgccatctcttcttcctctccgtcgctgtcattaagcagccaggtcacctcctccgctccgcggtccgccggtctcgacggaccgcgccgccgtcggcaacacggaaaacagcacgccagcccgagccgctaaggccgcatgcccctgccgcccaactgaacacgcataccccgctcaactgcgttttgccacccctgtcagtgctctcgctcgagcaccaccccgcatctcccaacctttttccaataaacgaaaccgacatgacacacgtaatgggtactcgtggctagatttattgaaataaaccgcgatcccgggcgtctcagcacacgaaaaaccgcatccacatcatagacaagttacagtccacagtcacatacacgataaacaataccaacagggtaatgtttatggagtaaaacactattgtccaggccacatgcgtgtatgacttccgcaccatcccgtactgcatgttccacatgtacgcgctagacgtgtaatccactcgcagttcggggacgcaacgcagccagatcacatccccttgcagtaccagacgcagggctagcgtctcgaagatcggcatcacatctaagttccgcacgttccactttaacgactccccgggaacgaactccacgtcgtcggcgtgtacgtacaggttctctcccacgccgccataatcggccttcggatcgaagacgaaccgactcatgttgcccacgatgctcccccgagcaaacaacttgccgttgtcaatgtagcaccggttgtcctcgatttgaaaccagggatgcttggccgtggacttccagggccggagcgcgtcttccccggctttagtgattccatcgggcaggcggatcaagggacccatggaggtccaaagacccacccaggctttccagagattgttcatggtgaaacagcgtgtggactgtacgctctttcccaatttatatcccagagtagtgacgtgagcccagccacctcccagattcctgacgttttggttgtctttcctgccaattcctcccgtaaacttatgattatcctagcccattcccgataaaaatacacggagacagtagatagagttacgaataaaccggtttatttattcaagtgtctcaggagattattgaacgagcgtggataccacgccgtcgtcagttcatggtggcattgagcagccatagcaccagagtcccggcgcccggtatcagacacgctgacctaccgggcgccttcgagtccgtaccccgcggcctgggtgttagagtccgtaccttgcagcccaggtaggtttcaggtaccagctggttcgtacctgttaaataaatcgcagacgggcgctcacccctacggtcaggagcacaagaacaaccagagagaacagatatacgagcagggttctgaacagcagaccccaattgtcgtctctcatgcttcgctgaaggtaccagttgatggtctgagagctatagtccatcctcacctgaggaacacacgcggcatatttcttggggtctccccacctcgtagacaacgtgatgtccaccatatccacggtgtgcgtcaccgggtgcccaccgatgttccactcgaaataggctccgcgctcatcatggtggtactgctcaccggacacctgcagtctgtccatgtaagattgagagacgatacccacgttcacaaagtgtttctcggtgaagttgcccgacatcctccccttgaagtacagcatgcccatatggaaccagcattggttctcctccactcgaaagtgggccgatctgatctccgataccaccacatccaggggccggggcaccgagtccgcgagtctcaggaacaagacggccaggatcgcgagcaccaacaccggcttcatggctccgaaggtccgctgctcggctccgctcaccgctccggtctggctgcagcagtgcttcgctgagaagtagcgtgtggactgaacggtgtttttgaatatatagcgtttcttggtgacgttgtttcccctacgtagtaggcaactacgtgccaaaagaggcgttacggtactttccgtactgggatttccaaaccgggactttccacacggcggtttcaacaccgggacttttcacacggtgatttcggcaccgggactttccgcacggcggtttcgccaccgctgacgttctcatcgccgcccacgtcaacggtggcgacaccgtactttcccatgcggtttataaacgtcaagagtcacgtcagtcgcccacccccattacacggcgatatcccgatagggcatgaggggacccgggtgtcgcgacatgtcgacgacaggtgcggattagtggtcgtgtcgcgacatggacgtgcagggggatgtctgtcgcgatagagttgatgtgacagcccgctacacctctctgtcgcgacatgcatacacaacgggccggcttgtcggcgattgtcgcgacatatcgttatcagttagcgaccggagttgtctatcgcgacatatcgtcgactatcgcgacagaaaaaataccgttcgtagagaatgccgtgttgaaggaacgcgcttttattgagacgataaaacagcatcaggagccacaacgtcgaatcccacgtccagtcgattcgtatgttatgctgcacagcaatgctagaataacaaccagcagggtaatcccgcaacataaatacaaagtcacagcgaagaatccgtgtcgttctatcaagcgaaacgcgttccaaacggccccgtcacagacgcagttattcataagcgttaacaaccggtggctaggatgaatatccaaatcacagggcagtagccgacggactcgttgacaggtcagcctaccctcaaggttcctatcgttcggacgggatttgtgcgttttaggcctctttttcgccgcctgcaagcattggtgcgcaaagtcctcacccagctgtttccagctatcatctgcatctgtgcagtcccctgtatcgttgtaacaaacgggtctgtgcgacttcgttctcggaacacaagcttgttgtcgcggagacagagagagaagggttttcgggtcacgcgaagaccgctcaccgggggtcggcaacgcacacatcaacagaaaaccgagacgaatcaagagatccatagtgaaggagtgatatcgacgtgcttacgaaacggcgattatatatgttctcaacaataccgccctacgttgtatgatgtaacgtgtgacgtgagtctgatccaacactgaacgctttcgtcgtgtttttcatgcagcttttacagaccatgacaagcctgacgagagcgttcatcggggcatgaagtacgcattacacaaactccatatatttgttacgatagaatacggaacggaggaggctttcgccacacctatcctgaaagcgttgcattctttatgataggtgtgacgatgtctttaccattcccacggctgctttgcgtgatgatgacattcatcatgtatttccattcacacataccttttgtgcatacggtttatatatgaccatccacgcttataacgaacctaacagtttattagcccttgacaggataggtcaaaagattatatgtaggttttccggtaaaccgaattgtgatatttctctgcaggaaatagaacagcctggtacctataaaacggacaatgcagtactgtagcagcgtaaccaagtaggtccacatgaacacgtacaaaattatggtaagccatcgtttttcataccacagcctgtagctgtcgtacatgaatgaggacggtcgaggaacccagggtagttgtaattgggggcgacattcgtactgtccagaagacaattgcacgggtttcagtgagatgagtactttagcgatgtcggcgggggcgctacgtttcaccgtgacggtgagaacttgaccgtcgttttgtatttcatgaggcacgttatacaagccactggtatcatgaaggatgacctctgatgcgatgtgaggattaaattgtccctcaaaccgccaaacgctggtcatgtttccaccgtcaattacgcagctgacggtgtgagataccacgatgttggacttaggtttgggggctaattgcctttttacaaattcccttctgtattgcaggtcctgctgccactgcttttccgtgcggaaagtcgccatgtcttccacacgtgtggcgacgatagacgccaccaaggtagctaccagaagcagctggatccgcatggcattaccgtatgtcaattagaaagttgagcggacacggttatcgttcctggcggatataagtatataaacgcgagttagcctttcccgtccgttttgtacacccgttccccacacaaatgacgaatacgacctttttttttataaaaataaaccacgtgtattatataaaaacatttacatagaaaagagacacacggatcaacataaggacttttcacacttttggggtacacaggcgtgccaccgcagatagtaagcgctggatacacggtacacagtcctggccagcacgtatcccaacagcagcaccatcgccatacagatggcgatcacgaccccgagctctaagtgtctgtattcatagtgtagtcgccgcaggttatccactgaattcccgtaactgaaataacgtatatggtaccgaggctggcaccacatgggtttgcatttggtgcacggcaccaaatgcagagtgagatggtccaagtccgtgggcacccactggcgcaaacggaatacggcttcggtggtctccacgaggcactccggggcgtgcagacggccccactttcgtccgcgacggcccgaccagccgacccgagccactatccctttctcgggatagaacgtaccctgtacacgccacacagcgtccaacacgccgtccttgacgacgcagctggcctgatagctggacacgttgttaagcggcggaaagcgaaactgacgtgccggcggagccacatagttcggttcaccgtgttgtcgcggttcgtcctccctatagtaatagtagtcgtcgtcctcataggggttgccggcgtgagccagcgttacccaacagcagcccaggccgacgaggaggcgcagccaccgcctcatggcggcttcgccagtcaatcgtctttagcctcttcttcccgtgaggtccttccggtggcgcggtgccgacctcggacccagggacgtatccacctcaggtacacacagcaggctacctggacaccgaagctgaacaaggctacgtgtttcacaaactgcaccagtaccacatagaggaatgtcaggtagcgtctctccgcaaacagccgttccaagtctgagggcgttacccgcagcggcaaccagggcagcctggacgccggccggcaatggagcacgctccggttacaggcactgcaggggtaaacggttaacatcacgtaagagagtcgtgcgtccacctgtgggagctcagtttcgtaacgtagagccccgtcattttccagctggggtgcgccgaccttgaaatgggtcgcgctccgctcgttaccccaggtgccgtaggctctcggggccgtatcggagaagttgccacgcacaagccaggcggccacgagtaccccgtgctggacgtaacattcggacacggaactggagacacggtagccggacacgtccccaaacccgcgagggtactggggcagacggacggacttgctatttgacaacggacagatacgagacgacgaggacgcagacgactcgtcgctggaccacgacaaccggagcgactccttggagcggctcgagagtacacttactgcgatcagacaccagtgccagaagaaggaacaggtggacggggaccacaggatcatagccgccggcaccgcggccggccgcaggaagccgcccggcgcgtcgtctgtgtgcgggagccgaaacaccgtgcctctttatatcgtcccgacgtgacgcgagtattacgtgtcaggggaaacccccgtcacgacgaacgtgatttgtaagtgacgcggggtgctgacggggttcggcccgagaggtgacggagcgcctcacgtcagtatgatgtccgatccgcgtcagccccgacgtggttgtggtcaccgaaacccacgtttatatggacgttgagagcagcgcctgaccacatgattcatcataccatttctcggaatcgggcccatgccgggaaagcacattccttttcagtaaacaacaatgacatcataacaaatcattttattcgcgaggtggataataaccgcatatcaggaggagggatcgggtgatgacgcaggccccgcagaacagtccgaaataaatttttagtattgccccatagtcgcctagataccagaggtacgttaagttcatcaaaacgcccatcggcgtcccggaatcgtataccgggcacacgaagcgttcataacaatcccgggaggcgagtgttagggtagcagagtagtttcggggtcggtttccttccggcgacgacagttccgtgggcagcagaatgtacagcgcctcggtagctgtcgcggtgccttccacgaggatgggctgccggtgcctttcgtgattttccccgtcgtgtagccaagccgaggcccgcaaagtcttaggcgaggggaattgtccatagagtttcaccgcacccttcagtacatggttctgaataacacagccgcacgtgaagtaggtaggttctctcgtctcctccgtggctgccgccaccactcccagccaccacaacaggcagatcgccagagggttccggaggcttccccggcgtagcatggttttgggttaaagcaaaaagtctggtgagtcgtttccgagcgactcgagatgcactccgcttcagtctatatatcaccactggtccgaaaacatccagggaaaatgtcggtgcagccaacctttcacatacagcccccaaaacacttgaatcactgccaccatcatcagcgtatactgcgccgacttaatcgtgagcgcgtagtacgccattagacggcgatcttcgaacaatagtcgttcgatgtcctctaacgagctccacaggggaacccaaggcacgaggcaccggggttcgcactctacataataagtttggcattggtggcagggggaaaagtagaacaacacgagttttgtgcgttggggaacacgatagtcccggagccagtagcgttttgcgacgaggctttcggagacgtcctccaccggcgtcggcactcgatccgcgtagccctccagcgtctggtagtacacccggggtgtcggcgtgggcacggacaggttcccgcgcagggtccacagagcctccagtcgaccgcccgatcggagcacgcagcgcgcctcggaatactctactcggtactccgaaacatcggacagaggcggtaacggctccgtctccaccaagggcggaggttcatcgaaaagagtcaaggataattcaggcatactacccgcgaccggggcccagagggctagaataagcattacaaggttcattctgtcttacaagggaaggctgttaccctgtctagactcaaaagctgtaaggctgtcttatagcatgtagtcttgcacgtcacggggaacagggtggtgatctagtgacgtcgggagaacacggtgttttagggtgcgggggacaaaggacagtacgacagattaggtgatagaaacgtttttttttatttatgaaaaagccagtgtgccgtgcggcctagggccccggcgtagtttggataccagatgggggccgtcaggggtactaccacgagcagaaacataatgacttggtccatgtatagcagcatagcggtgcgcagcaggtcgccgtccgtgtagcaatttgacggtgagcgataaagcaccgttaatgtgtcgcggataagcacgatcttgaggccgtagatgaagctcacagtcagtgctaaaatgatgcgttggtatggttcccaggactgcacggcgatgaagagccagagtatgggaagcatgaagcttagcaaacagaggatggctaaccgtcgttgcatgttccaggccatgagccaggctaggcccgtacaccagacgcagagcatggatgacaggacataggcctggattaccacggtgcgatcgaaacacagcccgatggtggacacggatatcgtagtgagggtggtatataccatgaccagcatcagggtcccgggtcggcgccgacgttccagccagtacgcgtggcaacgcagagcgcagggtagcagtgtgctccagaagggcaatgtatcgcgcaggtagggggccgtcacgcgccacggtatgagcatgaaaaggatggtagtggctatggtggcgctggtctggaacacgacagtgccgtagagacgtaccatccagagaaagtgttgaacgctccgcagggtgtcttcatctttggtgattacggtgactcgacggatcggcggtggtgacggcggcgacacgggtgggggtttctctttcttatggccgagtggctcgccttggtgaaactggatctgtaccatgacgggtgctcgacgaacagtcgtgggggctttaggtacccggcaagttttatagagaaagggggacgatgggtggtggctacgagccaccgccaccttcgcaatacgaggatctgaaggcggcaaagacggtcgtccagggcaggcgccagaggttgggactgagcacgatcagcgtgattttaaacatggtcaccagtcctacgtagatcagcagcgagccgcgtaacgtctgagcagccggcagttcgtcgcggatgtaacgcgtgccgtagaaagtcacggtcatcataaggaagacgatggcgccgtagccgtagagtagaatacgctgatgatggaacacggtctggtcgccgataacccagagcgtgatgaaaaaaacgctggtgagtacccgtgagcatatgagctcccaacgcttagcgcgaaagctgtccccaaccatgacagcgccggtgcaagctatccacagcgtgaggaccagtgtgtagtcgatgaggatggcgggcaggtcggagcaccaggtgtagaaaaccgtggtaacggagaggaggcctacgtagcccatggtcaataccacgtcgtcggggtgcctttcgccctgtatcaagaccaaacaccagagaagggagggggcaaaaaccagcagcagaggggaagattcatgttgacatatgttgtgggaatcggggatacccagccaaatcattccgcagaaagccgtactgatggcgatgtgaaagaccactagggcgtagacccggacgaggacagcaaaacggcgcagccacataaggccgtggtgcagctgcaggagggaagcccattgcggcgaatgtagcgacggtagcggcgggtccatgaggcgggtgatgcgcccgagtgaacgggtgagcgtctcggtggagtcttcttataaaccagcggagctcaggcagccttgctctggaacgtcgcagtggtggtgttgaggatgacgctgagcgtgccgttgtcaatcaggtaatgatgataggtgccgagcttggccaggtagctgaacatttggtcccagcgtgccgaccacaccacgggcgtgagcatcaggagtgtggtgtgatagattagtgtttcggtggcgtaaagtatcagcgagctgcggatgacgtggctcacgggcattttggtggcgatgtagcgcacgtcttggaaaaggacggccaggatgcagcccacgaacacggtgtagagacacagcaaagtcttatgtaaccaggtgtaagtagaagccaggacgctgaccatcaccgtcaaaagtgtggaggtaaaaagcgcgtcacgccacacggagctgagacggtgctcccaagccacgccgttgcaggccacgaacaacgtccacgttaggatgaggctagaaatgccgatgggcgctgtggcgcacaggttgagcccggcggtggtgaacgagagaagcgccacatacagcgcaaacaccaggccgttgctggggtgtctgtgatcggtgagctccagcgcgcccagaaccaatactggtgtgcagctaagcaatagcggcgagggatcgtcgctgcacttgtagcccagcgaggggtaacccagccaaaccagcgcgctaatgagtacgctgaaagcggtttccagcgtcagcaatccgtagacacgcatgacaatcgcggtccgccgtagccaacacacggcatcttcggaaactgtggacgctgtttccgaataccgggaggagatcgtgcttccctcttccaaggatcggaaagtagcgtccgtcgtttccgcggacgcggcttccctggtacgctccgtttccgacgacgcggtttcccgctgcgtggaaactgtctccatgtcgggaccgcagcgcccggcggcgtatccgcaaggtctcgaagctacagcttgtcagaggaaaagtaggtttgcaaaaaggtgcgcagggtcatgattctcagcaccatcagcagagtgaaaaccagactgagaaacaccttgacggccgccaaaagcgcgcgttccagcggcgtctcgtagcgtacagccagggccgcttcgtggaaatgcgagacggctagacaggtaatgagcacgctgaaggacaagacgatcttaaagcaccaggaccaaccacgcctcaagatgaccaccacgattgccgtgaaggtcaacgtgatcaaagcatggacgaccacgatctgacggcggacggtacgttcgggagccaacaacgctacgccggtgcagctgagaaaggccagtaaggtgaacaacgcggccgagatgaccaacgtaccgtccaggcagagacatatcacgatcaacggcggcacgtgaagcagcgtgtaaaagagcagaacgccgatattgctgggatgcgatgtttcgtaacagtgaatgaagatcactgacgtgacgggtatgacaaagacgaggctgggcgaggactccgtgagacacagacgagaatggtgaaaccacgtcgcgggcgccgcgtagcagaaggcgctcaacaacgcggtcaagccggccagctgccaacccacggcgccataggtgtgcagcgccacgcggcaacagtcgacccaagccagactgcgggtcgccagccgggtctcttggatcccggggggcacgtagatgaccgtgccatcggtgggtacttgaaaccctttttctcttctcatggtgcgctgcgttctctggaaacggctgctctgtccgaaaaccagttccgaacgaaaatctagggcgagagggtggacaacggcgtcgacgacgaagcatgggacaggtcgttcggcgttaacgtcatcgcgtcggacgacggtagttctaagagacgtagatcgctcagcaggtcctgacagttgcggattcgcaagatcagaaaaaaaagggaaatgaacgtaataaagagctgtagcgacgtatgcgccacatcgcgtggcataagaacgtgacggacgaaaaggacctgctgcgaaaagtgaccggcgaagataaggcccaccgtgctgtagaagcccaaaagcagccgcaggggccaagtccagggccgcgtgaagacgatgagaacgttgaccagaaagaccacgacccagacgccgttgatgagggtaaattgatcggacagggtgcagttgtcgcgacagatgaagactacttccgcgcagagcaaggtgatgaccaacgtgagcacaaacgacgtcaacacctcgcggggctcctggcaggcacacgtgacacctagcgccgggatgtgcgccaggaggccggcgagtaatagcaccagctgtcggaacggacgacggcagcgcgggtgccggtttcgctgagcgagaaccggtcgctcatagcggaaatacacgaagagcgcggaggccacaggcaccaggaggagcacctcgggcgcccagacaacgtgacaaggaaagcccggacgcgacttgagagtcgctgtagggaagaccagagagaagctacccaagacggccaccgccgcggagatttggaagaggagcaagccggcgattcggacgacaacctcgaagcgatgcacccagcccagcacggccaccacggccgcttcatcatagtcgtcgttgttgccgctgtcgaacagccgccgaaacacgatctgtcgctgggtcgcggtgggaaagcgcagacccatgacagccggaggctatatgaccgcgcgtctaagacgcgagatccgtggggggacttttagatgtttgggcggcccgcggttctaacaggcttgattggtggagacggccggcgcggcgggtgggggaaacgacgagtttttccgttacgccatggttcgcgtgaggtttctctgtacctcccgcaaaaggtcacagcccgaaatggaggccgcgttggtggccccggtggcgcgtgacgataaccaggtcatccaagcgatgagtttgtctaatgagtcctcggtggtgaagaggatgagaatgagcaggtacaggtacaccaggttctcatagagacacaaggtgagcaggtcagcctcggaccacgcgatctcaaacaggcgcgtggtgtcaaagaccgtgacgaccagcatgaagctgagcgccatggcgtaatagcccaaaaaaagtttgtgccccaacggtacgggctgcaggtaaagtgcgatcaagaacgcgataacgccgatcacaaacagcgtgacgatgacctgccatcgacggtgattatggccggctagacccgtgacgcagctgcagaggctaaaaagcacgcaagccaagaggcccgagaaggtcactagcgtagaggaggagcaggcgctggccacgatcaccgaaagcgtcgtgagcacgctataaatggtgagcaggccagggctcggtggcgacgtgaacgatccttcatcgcgtttgccgtgcagcagggccaaacagatggtgggcaccatcaaacttaagggcggcataaagccggtgcaacagagaaagacggtgcctttaagatgcggaaaagccagcaccaggcccagacagagcaagaaggtgcaggtgccctgcacggccacggtgctgtagacccgcatacaaagtaaaaagcgacgtacgtcgttcgtcgacacggaggaaatcataatgactccgcgcgagggtcgcgggggtgggggcgcccaggccgtcccggtggcctctgagttcggagacatgacggcggtggcgatcaaaaggcgcgtatgagaaaccgtttatagagtgtaatagaatcaccgtcattcccacacggcgttcccccataaagtcacgtaacactcgagtaagcgtgaaaaagctttattgttgaataaaaaacacgagtacaacaccgagttgcggtgtcctgtctgtctactgggtggggaaggttcatcgtctgtctctagagggaaggtggggaatgtctaagcgagcgggagcgtgtcatctcccccatctttttacaacaagctgaggagactcacgccgtcgatgcgtccgccgtgtttctcggcgtactgctgcacccagacgtggccgctaaatatggcgacgctcatgtttaggagactcatgacgatggtgtacaacacgacgctgacacagacgctgtttttagacaacgttccacgctggtagatgagatccagggtctcgtaaataagcacggccgaagcggcggtcaccaccaggacgtagagtccgctgtagatcttgctgacccacagcacgggcgaaaagtaaagcaataggtaaaagacgatgacggaccagccgtagccaatcccgatgactttccagcgcgtgggattgttgccggccaggtaggtgagaccgctgcagagaacgaaaaagaccatcaccagggcaaacgacagaccgatgacgcgcctttctccgcaaaagcccgtgcacacggtgatgccggtgttgatcagcaagcacgccaccgtgagatgagcaaaattggtggtgtgtgggcgaaactcggcgaaaccgcgtagcatagccagcgtggacacgggtacgatggaggatagggctggcactatgccgttggcgcactgtccctgcacatcggggaaggcgagccaagccagcaagcagaccgtgagggtacaagccagctgccacacgagcccgtgatagacctccatgagcagcttaaagcgtttcaaccattggaagagctgctgttcggccaccagcgcgtggctgcgatggagcggcacgatggtgaccgtcggcgactcatggtgttcggaaaccgaggcggtgtcgcccatgctgccgcttacgaccgctgtcggtctaaggtaggcgtcgatgaaacagtccgtcttatcagcacccggttaccgcggatttgattgacgtcacgagtgtggtcaaaccgtggcggcaccctgtatccgacccgtcgtcatgggctccacaaccagagcctcagaagatggtacatgccgatgaataaagccacattttcgacatagaggcgtagcgagggctgaaaactctccgggaaagaactctgacaggtgatcagggacagatcgtgaattagcatcagcgtcaccgtcaacagcgtcgtcgcgtgtaaaccgagaaagaacggggccgcggcccgcagcagccaaagtcccagcgccgtagcgcagagcagagacaggaccgacggtagccacagccgccggagagacgcgccaggatcgcaacccaaaagcgaggcccccaggcagctgagatctaccgccagggcgagaagagccgcgccgacaaaggcctgcggcgacggctggcacatcagcaaggtcagaaaggctagcgcgtgcggcaggcagtaagccaacaggagtgggagtttgcggggacaacggtcgatcgacggaccgcgtagcagcaggaacaggcagccgacgggcacgacgaggctgagatgagaaagcggcggtgggtcgtcgtcccgtccccgctcgcatagctcggccaccggtggcggcatgagccaccagctgagcacgctgagggcgacggtggcggtaagctggaaggcgacgaggacggaggcgcgcagccataccgccagcctctctaggtaggggactacctcctcgacggtccattctagcgggacgacatgaagcatggcgacaagcgcggctgctgtgaaaacgggcacggttttataggcattaggacttccccgtcgtactggcggctgtcaaagtcccgttgtccaaaggcgcgccgtccgaaagactaatccaacggggacccgagagcatgagcaacaacgtgagaaagatggccatgctgtccaggtagagacagacggcgtgacggatgcattggttaggtgggcagaaaaagatgaccataagactgtcgtaggccagaatacccaaaaagaagctgatagagaaggcgcacaacgtcaccactatcttctgcagccaatcggcgtcgcttagcagagcgagcgtgaggaacgaaagcagcattaccacgtagacgcagctgatgcatttccagcgacgtcggtcacggccacctagaaacgccagccccgtaaaggagataaacaacgccagggtcatcacgtaggaacctactagtacgcggctttcagagcacatttggaagatggccgccgtcaggctgttggccaacagatagatgaaaagcaccgtggcgttactagggtgttcgttgcccaacgtgtacgtgatgaacatgcagacgatgggcacgagcacggtgagaaagaagctgtagttctcgacgcaaaagttgcggttttgtgggaaccccaaccaaaaaacgcttcccaagccgaagctgaaagccagctgaaagatgaagatggcgtacacgcgcagccatacggtgaactttttgaaccactcgagagcctccatgcgggagagcagcagcgcgttagcctcctgcgcctgcatggtggcgacggtctcggcacaaagccgctgcggcgcacctacccttctcttatacacaagcgagcgagtggggcacggtgacgtggtcacgccgcggacacgtcgattaggagacgaactggggcgacgccgctgctgtggcagcgaccgtcgtctgagcagtgtgggcgctgccgggctcggagggcatgaagtagagcacggagacaaagaggtacatgaggtccatgtacaagcagagcgcgcccgggatataactctcatactcgatgtcgtgcaggatgtcctgcgtatcgcacaccaccgaggtcacgatgacggccaaaccggctatcatcaccaggatctcacttaccgcctcgggaaaaagagaaaatacggcgaacagtaagagaatcagcgtggatgcgcccgtcaatagggaacgctgtaattccacgtcgcgggcaaacagatacgtagcgagcgtgaggaaacaaaatagcgtcactgtggccaccatggcataaatgactgaacgatgactaaagtggaagcctgacgccgtgacagccacgctggtaagcaacgtgtacgtcagtaagatccatacgtttttgggaaagttgggctcggcccaacgcaacagacctaggcacacgatggagatcattaagcaagacagcgtcagacgcacgctggaaaagagctgctccaaccggtgcggcaacaccagccagcaaaaggcgcagacgctcataaggatgaggcattgcacccagataaggatgtagatgcgcagcaggaagaccgaccgggctatctggacctgaccgcggagcgacatggcggcaacgccggcggttatcgccgagattcgtctaaatacacgaagcgaactagaaaacgcacacacgtgatttgcaaaaagaaagcagctgccggcttattattttattaaaaatttatctgtgcagaatcataagtttatgatgaataaaaacggggaaagggaatctgcttttagggacccgggtctggtccgtcgtctcccatctggtcgggttcggggatggggacctgtttcagcgtgtgtccgcgggcgtgcatggcttttgctcgccggccgcgctgtaaccaggcctctttctctgtggtcggcgagtcttccgacgggtagggagcctgggagtccatcgcttcaggcccaccgctcgttccctcgaccgtcgtgtcgtcctcgttttcgctattacacggggtttctggagtatcgcctatacggttggcgattctccgggggcggccgctctcgtcctcgtcgctgctatcgccgcccggtaattcgacgccgcattcgttgtacggaacgcggcacatgggcggcggaaagaacttgggcatgcgaaagcagcgttgtccatccacggtctgcgtggtttcatcgttatcctcccataatcccccctgtagcgccggcagcgtttcgacgctgtgagaggggaaggcccagttctggttgtcttgcagcgcgcccgtgggcagtaggtccgtgcggccccatgcgctgctgttgttgggtaccttgtcagtgccgcgagtaggtcgcagaaaccagtccagagcgctctctagctgcgagcgtgtgatggtgcccagtgcgccgtgccagcgcagcacgtctcttttcagcgtgtggtgacagacgggcagctcctccaaccgacactcgccgcgcaatccgcggtcgaagcggcagagaccacgcagtttaagcagaccgcacttgagaaacatgtgaaaattatcggcaatgcgatataggtccgagtcctcgatcttgtgtaggtagaccacgccaaacttgtcgagcagcaccaggccgctgggcacaaaaggcccgtaggccaggtaatagcccacgaggccgacgacgtaccactcgcagcacaagcgttgacgaataaagttcagaagatcgcgaaagtccgcggccggcatgtggtcaaaaggccggcaggcgcgcaggccctcgatggagcccagcatgagcaacggctccacctcggtgcgacccggcgtgcggatgaccaggttgagaccgctcatttcgcgggccgtcttggccacggccgcagcgtcagtggggtcggtgcagaggaatttttgcacatgatagcgcggttcggtggtggcgaacggcgtttgtgggtgccgatacacatattcgcaccagagtaggccgttcttggaaaaggctttgatatcactggccacctcgtagagcccgtcggtctcccagtcgtagacgtagacggtgccgtaatgacttagcatgagcacgcagggcagttcctgcgcctgcttggtgtttcgtgttagatcgctgtcgggtggacgcacggctagtacaccgacggcttccagggtgtcatcgcagcagagatagtcggcggccagagaacgtgcgtaaatctgcgggatggcggcctgttcgcgcatcactaggaaccagttggcggggttgcgcagtgctacggtggttccttggtggcgttgcacgtaggttctcagcgccggaggatcgtactggcgcagatagaggccttgcagcatcgataacgtcttttgaaagacggtgtttctaaattgaaaaacgccgtagtcgcagcggatagcatcttcgcagcgctcgtcgcgctgtcggagataggtgccccaggcttcggcggcggctttggtgagtagggacatgccggcggagccgtctcgacagcgagtcggataaagcgcgctgcgcgaaagcttaatataggagcagcgtcagacgaatcgcggctggtggcccggggggtgggacgcgccgcctacacaaagtgctcccgaaaatcgaaactcttgacccactccggagacaaatccgtattcagattgatgcgtcgagcttccacttcggcttccgaaacctcggcctccgtccggtaggcgttaacaatacgctgacccaggtgccaacgctctttctctgccaaacgccgttgctcaaaccattcgtctacgtccttgaggtcaaagacagtgtcctcctcaaggtcaaagcctaggtcttcccactcgtcgtcatcgctctcgtggccggcggccatacgcgcggcaaccgcgtcttcccctcctcttctttcaacgttgggtaccacgttgttttcttcgggttccataggttctgcgccgctgtcgtcatcatcctctccctgctcctcatcgtccgccaaggcgtcgtggattacctccaggttctgattgtcgggtacgacgtggttatcttcgtcgtcgtcgcgtggcatgggcggcggccgacggcggacgaccggcatggcgcggccgtcgtttccttcgtcttcctcttcaccgtctcccaaggaacgcggtcgacgacgttccgcgaagtcgccgcggaccacgcgcgcctgccaaatggtaaacgcgtcccaaccgtcccagttattgagcatttcggcgcgaaaacggtcgcctcgacagagccagcgaaactgccgcgcgtagtcgcggtctacgccgctgtcgaacatggtaaagtgcagacgcgccgcctcgcccatgtgtacgcagcctccgttgcgttccagcctggccgcgcgccgtagaccgtgttcgtagcggcgacgcacgtacaccttcatgaggccggcgcgaaaaagttcctctaggctgtcggccagccggtagatttcaccggctagacgctgcaggggcggcgagcggtccagatgcgacttgacaatcaccacgtaaaaacgacagaaacggtcgaagatgatgaggaaggacgtgtcaaaaaaaccaccggcgcggtaagagcccacggcacccagcaggtaccagcggcaacgcagttgcagcgtgacgtacatttcgcactcggccaagcgggcggctggcgctacctcgaagggccagcagtccgtcaagcagccgaaactggtcaggagtttcaacgttttggcatggcgtccaggtgtatgaaagttcacgtcgcgtccgtggtgttcgccaacgcaggcggccaacgcgtcggcgtcatgaccgtgacgcagcagcatcgctaccacgtcgtgcggtacccgcgtagcaaatggcgtctgtggctgacggtatacggcttcggtgtacatcataccgtaacgcgccagctcgtccagatgacgcgcgcacagcagcagaatctcttgcgagggttcgtagatgtagaggcgcgtaccgccacccatgcagagcaccagctccgtctcttcgtagtgatcttccaccatgatcacgcacttgcctagcacgataaggcgttcggggcaacaaatcacgtcgtccagcagctggtcgcgtagctccggcatggtgctgccgggccgtacctgcaggaaccagttgtgcggaatgccgagcgacagcacctggtcgacgtggttacggacccagtcgcgaagcacgtcggcgctgtactggcactcgaagatgccctgaaagtcgcccatgacccgcagaaaagtttcgtagcgcgtgtggcaatagaggaattcatcgtttcgcgtaaacgtgggagctccgtcttcccaacgtgtacgccacatgtcaaaagaggccgccagctagacaccccagaaaagaagcagagaaagagacttctttgtgcgacacgttttattctgcgtcctccgctcgacgttcaaatctggatgtactcgcgcacacccgtcaggctctttaagggaaaagggtccgagtacgtcactaaccgcgactgatgcaccagggcggtaatcacccgctccgcgccctcgcgcgtcgacgaacgcgtcgtcaccaggcaatgcagccgcgggcccgtatcgtcctgatgaccagcggcctcgcgctcggctgcttccacaccgacaatgtcgggatccaacacgtagctctgcgagttggtgtcgtagcggtgtagcaccaacgtgttggggtccagacgctcccacgcgccctcgtgcgggtcaaaacgctccgttaaacagagccagtcatactgctgctgcagaatacgccgctcgcgctcgcgtcgctcatcgggcaacgcggcgtcttcgttgaagagaatgtcccgcttgtggtctacggcacgctcgtggtggtgcgggcacaggtgacggtgttccatacgcgtctgacgttgacgctcgcgctcaaaacgccggtgtcgaaagaccattttcagcaaccccatgcggaaaaactccgtgatggtgttggcaacgcgccgcacatagtggttggggtcgtccatctggatggcgtacacggcaccgaaccagtccagcagtaccagcacttcggccacaaaactgcgtcccggtcgcggacgtcccgtcacgcctagcacataccacggcgtggccagattagcacggacagcccaccaccaacgacggctctccacctcggtgagcgcacaaaagggccaaatgcggtgtaactgctgtaccgttttcatcaaccgcataatcaccgtaccgtaacccggtgtatgcaactttacgtcgcaacccaggattcgttcggccgtggcgtacgagccctcgggcgtggtgtcattgagaaacaaaacatgcatggtacgcgcgcccttaggatatcgtcgcggaacgggtaccgtcattctccgcagagtggtgtgaatcacgtcgcgatacgcaatctccgaacgtgacacaccgtaacgtgccagttcgtccaggttgtgcgataccaacaccatgtacttttcacgagtgtcgtaggcgtagacgcgagaaaagcgacccataaaaaccacgtacggagtagccaccatgccatcatggtgatcgcgacgtggctcgggcaacaaaataacagcgtatcccaacggcgtcaacggctcgcggcaacagatgagctttgacgccgcctgtttggcggcggtaatgatcccgtcctccgtacgtaacatcacatgccagcccttggggggacccaaggacagacagcgtccctcgttacgatgaacgtaacgcgtgatttccattggctccaggcaaaagaacagttccttaaaatcccgcaacacttgtcggtataacgccatgggatcctcggccgccacaggcagcgcggggagctccggcggcataactgcagcgccgtcagggccagaacccgcagccggatccatcattgagcgacactctcagccggacaaccggcgtcactgacagaagccgagccaaatacagagaaagcaacgctacaccgtcaccccgctcccaagcgccgcggaaagtgctccgatttttcaccgtcgttcgcgacgttgatttgcctcggtctgagaaccgacctagcgttcggaccggtgcgcagaaacagccggcggtccgagccactgagcggttcacagccccggccgccgatagttaccggagagacgttcgagctgcaggtacatcagcgcttcccgcttcgccaccccgcgcccgccccagtttatactctccgacgccccgtccaacgcgcctgtggagggccaatcggaccgcgggagctctccaagtggatgacaggcacagccgagtgcccgaccgtgaagagccctcatccacctgaacagaccgctaaccgaaggaccccgagtcgcgtccgtcggtcccgacgtccgtcgccatctggctccctgctgttggctacctctcggatttcaaaaaagagcacgtgccgatgacggtgcacaggaaagagccaaagtgtcacggcgtctttttttatttgtattcctttcctgttttgtactcgtaaactgttgatgttgtttttacatccaaaagggcaagtaagaaacaggatgaggcatggtaggtttgggcgtggggcggccctccagcacggcggcccgggccgcccggcgggtgagcacccggcgttgcgccgtgtctatcttgtgtttcttctgtgtctttttcctatcttgttccgcgacggcctctttcatcacgttcagcatgcgttcctcgacgccctccagggatcctggggaggagggagtcctagtgaggcttccaatgttgttttgtggattttcggtttccttttcttggtcgtcatcgtcggacgtgtcgtcttcctcttgatcctcttcttcgtccgagtagtagacgcatagtccttggttcatcaggctgggattcatcaggttctgacggggaatccgctgttgtagacgtttaaccgcccgttccaggcgagagctcatgccgcaccagacgctgtaacgccgcacgggcccgtagcgggctgtttgttcgcgtacatgatcgttgagctcttgccaatattgtttggcacactccagatcggaggtttgtggatagtcgggtcggatccgcggatcccaactgacatcggcggtgccggagacttcgtccagactgttacgcatagagcaccagtcgggtcggacgataaacctgtccttgcggattaaccatttataacgtagttcgtgatggcgtgtagaggcccgtacacgctccacggtcccaaagcggtcccagaagggaaagttttcgtgggggcagcgacccggcacttccagacgttcggcgtcgtccacggcgtagtgaaaacgccggccggcctggtaaattttgagcagacccactgttaacaacatatccacgctgtcagccaaccgccagatctcgcggcgagacacgtcaaaatagaaaaattcgcaggctcggtcgaccaggatcacgaaatcggcgtgaaagacgccggagggtagcgattcgcccaccacacccattatcatggtttcacagcataagcggtccacaaagaacttcaacaggtcgttgaattgctccgtctccatacagatgaagggccagacgcctttgaggttctcggcctggccgcagagcagtagcggacgtgtcatctcgcccggagtgcgcagaggcacgcattcgccgcgataacgacaggtcacacgctgtagttcgctgatgctgttgtcgtgcaggcgaaggtcgcagataatatgatccggttgcgtggttagcagcggcgtgcgcatttgctcgccgtagatggcctcgcagtgcaacagcccgtgtcgcgcaaaatcgtccaaactgtgcgccaggtagtaaagcaccccgcgatcgcggtctagacaccacacggtttcgtaacgtcctaacaggagcaccagacgggcctggctaggtggctcaatttcctctacatacacgaaaaagtcgtcatcgtccgagtcctcgtcctcagaagaggaccgcggcccgtgtactctgggcaacacggtggtagagaactgcaggacgcccagagactcgagcgattcttcgcagcagatgagctgaccccagggcgtttcgggcccgtcggtgacagccgcgctgccaaagatgtcctcaaactctacaaaatctagacgccatccgggtggcgctgaaatgggaaggctaatgttcatatcagcatagctacgaactaagtggcggatgtcctgccgcaagtcttggcagagaatgagctttcgtaaacccttgagggtcctccgaacaacggccccagacgcgtagcgataggactggcgcatggtgccgcggcgtggagcggcacttggcagcctattttatggagtttcttcagtgacgtggcttgttcacgtcgttcgtgggctgcggttggcagctccggtctgtaaaccacccgaaaagactgacatcgacgtcaaagactcacgtaatttggaacatgtgcgaccgcaaagtgcgtcagaatagcacgtggctttaggacataaaaagtaccgtgaggtctagacgtgggttttgtgattgacacttacaccaggtaagccaagggacggtgaaactgtatgtgaggaatctgggtgcttagacgactaacgtgtaatgctttttacaggactgttcgacaggtgatagtacctgtaaggtgatgaccacctctacaaataatcaaaccttaacgcaggtgagcaacatgacaaaccacaccttaaacagcaccgaaatttatcagttgttcgagtacactcggttgggggtatggttgatgtgcatcgtgggcacgtttctgaacgtgctggtgattaccaccatcctgtactaccgtcgtaagaaaaaatctccgagcgatacctacatctgcaacctggctgtagccgatctgttgattgtcgtcggcctgccgttttttctagaatatgccaagcatcaccctaaactcagccgagaggtggtttgttcgggactcaacgcttgtttctacatctgtctttttgccggcgtttgttttctcatcaacctgtcgatggatcgctactgcgtcatcgtttggggtgtagaattgaaccgcgtgcgaaataacaagcgggccacctgttgggtggtgattttttggatactagccgtgctcatggggatgccacattacctgatgtacagccataccaacaacgagtgtgttggtgaattcgctaacgagacgtcgggttggttccccgtgtttttgaacaccaaagttaacatttgcggctacctggcgcccatcgcgctgatggcgtacacgtacaaccgtatggtgcggtttatcattaactacgttggtaaatggcacatgcagacgctccacgttcttttggttgtggttgtgtcttttgccagcttttggtttcctttcaacctggcgctatttttagaatccatccgtcttctggcgggagtgtacaatgacacacttcaaaacgttattatcttctgtctatacgtcggtcagtttttggcctacgttcgcgcttgtctgaatcctgggatctacatcctagtaggcactcaaatgaggaaggacatgtggacaaccctaagggtattcgcctgttgctgcgtgaagcaggagataccttaccaggacattgatattgagctacaaaaggacatacaaagaagggccaaacacaccaaacgtacccattatgacagaaaaaatgcacctatggagtccggggaggaggaatttctgttgtaattcgatcctctctcacgcgtccgccgcacatctatttttgctaattgcacgtttcttcgtggtcacgtcggctcgaagaggttggtgtgaaaacgtcatctcgccgacgtggtgaaccgctcatatagaccaaaccggacgctgcctcagtctctcggtgcgtggaccagacggcgtccatgcaccgagggcagaactggtgctatcatgacaccgacgacgacgaccgcggaactcacgacggagtttgactacgatgaagacgcgactccttgtgttttcaccgacgtgcttaatcagtcaaagccagttacgttgtttctgtacggcgttgtctttctcttcggttccatcggcaacttcttggtgatcttcaccatcacctggcgacgtcggattcaatgctccggcgatgtttactttatcaacctcgcggccgccgatttgcttttcgtttgtacactacctctgtggatgcaatacctcctagatcacaactccctagccagcgtgccgtgtacgttactcactgcctgtttctacgtggctatgtttgccagtttgtgttttatcacggagattgcactcgatcgctactacgctattgtttacatgagatatcggcctgtaaaacaggcctgccttttcagtattttttggtggatctttgccgtgatcatcgccattccacactttatggtggtgaccaaaaaagacaatcaatgtatgaccgactacgactacttagaggtcagttacccgatcatcctcaacgtagaactcatgcttggtgctttcgtgatcccgctcagtgttatcagctactgctactaccgcatttccagaatcgttgcggtgtctcagtcgcgccacaaaggtcgcattgtacgggtacttatagcggtcgtgcttgtctttatcatcttttggctgccgtaccacctaacgctgtttgtggacacgttaaaactcctcaaatggatctccagcagctgcgagttcgaaagatcgctcaaacgtgcgctcatcttgaccgagtcgctcgccttttgtcactgttgtctcaatccgctgctgtacgtcttcgtgggcaccaagtttcggcaagaactacactgtctgctggccgagtttcgccagcgactcttttcccgcgatgtatcctggtaccacagcatgagcttttcgcgtcggagctcgccgagtcgaagagagacatcttccgacacgctgtccgacgaggtgtgtcgcgtctcacaaattataccgtaatataacttcgtatagcatacattatacgaagttattaaaaaagcgctacctcggccttttcatacaaaccccgtgtccgcccctcttttccccgtgcccgatatacacgatattaaacccacgaccatttccgtgcgattagcgaaccggaaaagtttatggggaaaaagacgtaggaaaggatcatgtagaaaaacatgcggtgtttccgatggtggctctacagtgggtggtggtggctcacgtttggatgtgctcggaccgtgacggtgggtttcgtcgcgcccacggtccgggcacaatcaaccgtggtccgctctgagccggctccgccgtcggaaacccgacgagacaacaatgacacgtcttacttcagcagcacctctttccattcttccgtgtcccctgccacctcagtggaccgtcaatttcgacggaccacgtacgaccgttgggacggtcgacgttggctgcgcacccgctacgggaacgccagcgcctgcgtgacgggcacccaatggagcaccaacttttttttctctcagtgtgagcactaccctagtttcgtgaaactcaacggggtgcagcgctggacacctgttcggagacctatgggcgaggttgcctactacgggggttgttgtatggtgggcgggggtaatcgtgcgtacgtgatactcgtgagcggttacgggaccgccagctacggcaacgctttacgcgtgaattttgggcgcggcaactgcacggcgccgaaacgcacctaccctcggcgcttggaactgcacgatggccgcacagaccctagccgttgcgatccctaccaagtgtatttctacggtctgcagtgtcctgagcaactggttatcaccgcccacggcggcgtgggtatgcgccgctgtcctaccggctctcgtcccaccccgtcccggccccaccggcatgacttggagaacgagctacatggtctgtgtgtggatcttctggtgtgcgtccttttattagctctgctgctgttggagctcgttcccatggaagccgtgcgtcacccgctgcttttctggcgacgcgtggcgttatcgccgtccacttccaaggtggatcgcgccgtcaagctgtgtcttcggcgcatgtttggtctgccgccgccaccgtcagtcgcaccacctggggaaaagaaggagctaccggctcaggcggccttgtcgccgccactgaccacctggtcactaccgccgtttccgtccacgcggatacctgacagtccgccgccaccgtaccagcttcgtcacgccacgtcactagtgacggtacccacgctgctgttatatacgtcatccgacatcggtgacacagcttcagaaacaacgtgtgtggcgcacgctacttatggggaacccccggagcccgctcgatcgacggctacggttcaggaatgtaccgttcttaccgccccgaattgcggcatcgtcaacaacgacggcgcggtctctgaaggccaagaccatggagatgcggttcaccatagcctggatgtggtttcccagtgtgctgctgatactggggttgttgacacctccgagtaacgggtgcaccgtcgatgttggacgaaacgtatccattggagaacagtgccgccttcgaaacggtgcgacgttctccaagggagacatcgaaggtaacttcagtgggcccgtcgtcgtggagttggactacgaagatatcgatattactggcgaacggcagcgacttcggttccatctcagcggactcgggtgtcctacaaaggaaaatataagaaaagacaatgaaagcgacgtcaacggtggaattcgctgggctctatatatacaaaccggcgacgccaagtacggtattcgtaaccagcatttgagtatacggttaatgtatcctggggaaaaaaatacacaacagctgttggattctgatttcagttgcgaacgtcaccggagaccgtccacgccgttgggaaagaacgccgaagtgcctcccgcgacccgcacgtcttctacatacagcgtcctcagcgcttttgtagtgtggatcggatccggcctcaatatcatctggtggaccggcatcgtgcttctggcggtggacgctctcggacttggcgagcgttggctgaggttagcactgtctcaccgggacaaacatcacgcatcgcgaaccgcggcgctccagtgtcaacgcgacatgttacttcggcaacgtcgacgggctcggcggctgcacgccgtttctgaaggcaaactgcaggaagagaagaaacgacagtctgctctggtctggaacgttgaggcgcgaccctttccgtccacacatcagctgattgtgctgccccctcctgtagcgtcagctcctcctgcggttccctcgcagccccccgagtattcgtctgtgtttccgcctgtataaaaataaagagacgggaggctgatcgcggccttcagcgtctcatttgtctttactctcgagtgcggtcggtgtctcgtcggtgagacgaggccgccgcccgacaagttcgatctcatgtcgctcttggagcgcgaagagagttggcgtcgcgtagtcgactactcgcacaacctgtggtgtacgtgcggtaactggcagagccacgttgagattcaggacgaggagccaaactgcgagcagccggagcccgcacactggctggaatacgtggcggtccagtggcaggcccgggttcgcgattctcacgatcgctggtgtctctgcaacgcctggcgtgatcacgccttgcgcggccgttggggtacggcgtattcctcgggttcctcggcctcttcctccggtttcgtcgcggagagcaagttcacctggtggaaacgactgcgccacagtacccggcgctggttgtttcgccgccggcgagctcgatacactccgtctaactgtggggaaagtagcactagcagcggccagagtagcggtgacgagagtaactgcagtctacgcacccacggcgtgtacacacggggtgaacaacactaatcgataagtcgcgtgtaggcgactggctacatcaaccggatatctgcggggatttaaaaagacgacccgttgtcatccggcttagaccaaaccgtccttttatcatcttccgtcgccatggctatgtacacatccgaatccgaacgcgactggcgtcgtgtaatccacgactcgcacggcctgtggtgcgactgcggcgactggcgagagcacctctattgtgtgtacgacagccattttcagcgacgacccacgacccgagccgaacggagggccgccaattggcggcgacagatgcggcggttacaccgtttgtggtgtttttgtcaggactggaagtgtcacgcgttatacgccgagtgggacggcaaagaatccgacgacgagtcgtcggcgtcttcctcgggcgaagcgccagagcaacaggtccccgcttggaagaccgtgcgggccttctcgcgggcctaccaccaccgcattaaccggggtctgcggggcacgcccccaccgcgcaacttgccgggatacgagcacgcctccgagggctggcggttttgcagtcgacgggaacggcgagaggacgatcttcgcacgcgggctgagccggaccgcgtggtgttccagttagggggagtaccccctcgccgtcaccgggaaacttacgtgtaagaacacggcgtgacaataaacaacatagcgtaaatccccgtgtgatgtgtgtgattgacgttcgggaaacatgtccccatcatcagcgtcacaattgacgtgggttggtcactgacgtgcaggatgttacgcgagtcagagaatcgcataagaacggagtggtgagcgggttcccacaggagtctctggcgcaaaagcaccatgagcctcaggttccccgagagggtgggttacgagaaactgggataccgcccgcatgccaaacgcgtgcgggtgcatgactcgttgggattgacgcggtttatcatgaggcaactcatgatgtacccgctggtgttgccgttcacttttccgttttacgtgccgcggtcctagcacgtcagtggtgacgctgataattgcaacatggcccatgacgaacccgcttgggacgaacgtcaataccacgtcaaaccaccgtgacttggctgaacgttgaaacataaagccaaagcgccgtcggcacttggcttcagagcagcgcctcggggcgatgcgacggcgatgaacttagagcaactcatcaacgtccttggtctgctcgtctggattgccgctcgtgctgtcagccgcgttggtccgcatggctccggactcgtttatcgtgagcttcatgatttctacgggtatctgcagctggaccttctgggaccagtggtggcggggaatcgctcagtccggacctggagagagcaggcggaccgagccagagggaccttcgcttggcgttcaggccttaatactagccgcatcttacctgtcggcagcatgtatcggggctccgacgccttacccgccggcctgtatcgtcccgaagaagaggtgttcctcctcttgaatcgctgccatgggccactgtcaacgccgaaaaatgcttgtctggctgaggttggtgtcgctaatgccacttttttgtctcgcttcaatgtcggtgattttcacggagcgtcatgggaaaacggtaccgctcccgatggagagcccggggtatgctgaaattcctcttaaaatttcgtaaacgacgtcgtccagtcgttgtgccgcgattcgtacggttcatcgtctacgtcgttttgttcaccgtcgctgtgcaacgcgtgaaacaagagcgtgatgcgcaccttcggcggtatgaagaacgattacggaaaaaccgcgcacggcgtcggcagtcttttccgtgacttggggcgatgggtccgagctgcggtatgggtcacggcggcgtgtgttttattgacgaagatgccgatgtgtgactaaaaacgtcccagccccagagcgatatgtttcaataaaaaaaatatgtagtatcatattatgcgtgtcctggtttttcatttttggatgtatgtatcgcataaagggtggcgaggtgtgaggatgaaacatatgcagatacgcagtgttgttatccgaacgaaacccgtgtaatgcgtacaacggtacttcagtatgaaagtcccgtgtgtggggggggggggcaaatagttgcgtttgccgttgggcgtacgctacgtttgtatttctggctataatatgtgcggtcatgtgtcgatgttcctattgggaagggtgtgactgtagggtgtataaagtacggtgggacgcagagggacattgatagaaacaggttgagcgctgtacgagtttcacacgctgaatcggcgccaagaataaaacagtggttattcgtaaaagtatgggggggggggggatgttgtcgacggttgttagatgcattgcgtatctgtattagtagttttgcaagccgtggtgcgtgttattgtgacgtagcaattatcgtattgtgcatgtgtcgttcatcacagagtttagtatactaatatgaagcgtcgcgagtattaaagcaattggtgtctctgtgctagtctaacaacacctgtgtaatgcgtacaacgagaaaaaagacgcgaaagcaacgtgtatgggggggggggggaataatattgctaatcatgcgtcttgcagtacagatagccgctgtatcttacgcgtattgtcgcaacagttccacatcggtgtaattggatgtctggtacttatcactggcgtcgttataacattgtaaaacaagttttcgaaacataacgacagctgcaaaagaaaaccagtttattgagcattgtaatggtagtgtgtggctatattagaaaacgtgacgcgtcgcatgtcgcggcacaatctggcagcggggtcggggtagggtacggtgggaggcatgtacacagatggaacaaaagcagaagtaacgtgagaaggagcatacagtccagtatccagcggttcctgagtagcaccacccatcaactgaatgccctcatgagtaaaagtctgcgggcgacagcccttggggaccgttggcatgggacgatcaatctccaaaccacagcgtaacaccgttttcttccaacgtcgttgatagacgtcgtttttacggttactcccaagaacccagaaagtctcgtccaagtcgtaccaggaatcttctccggggagacgcgacggtttccaatcctcgtcgtctcgtctcaaagcacgtcccaaactggcttgaggagtcaacggtggttctgtgggtcgggtgtagcgcgagtgttttcccttcatgagcgattcatcctccttgcctttaggctttttggtctttttgtgtatcatctggccgccggcctccataaccaccgtggccaagtccagtcccagagcttgagcgtcggcgcggcgtcgggcgtcttgcaggtagtcttccacatttgcacagatggccgggtgtttggtggctagggtgaggacctcagcctcgccgcgacccggacgtagcaaaaaagccaactgcccgtgcggctcgcgcgcccacagcgcggcgcgcgggtgcaggtgcagcgcgtcccagcgcggccgctcccactgctcgcggtccagctcgggcagcagccgccgcgcggcctcggcggcgggcgccgactcgcgtcccagcgccagcgcgcccagcacgcccgcgcgcagaaagtgcgacagctccgccgccagcgggtacacgtgcccgtccagcgggcagtacccgaacacggcgcccagctcgtccagcagcaccaccagcatggcgcgcggcacggtccccgacgccgccggacccgccatcgctgtcgaacccaccatcaccgtcggcgccgctgctgctgccgcgtccgccccgaccaccgcgtgcgcgtccgcgttaggcacgcaaatcgcgcccccgccggcggcgccgtacggctgcggaggcacaggtacagcggaccccacggctcccgctatcgcgcacggcgcgtccccgccggcggcctccgtctccgtgccgctcgccgctggcggcgacgtcgtccccgccgccgtccccgtcgccggccccgccgcgcagcccagccaccgcgcgggcagcaccgcgcccagcgccagccagccgcagcacagacgctggttcaggtgccgacgcacggccgtcagcagcgacgcggggtgcggcgccgacgcgaacggctcgtactgcgccagctcctgccacgcgcccagcagtaccatcggctgcagtcgcctgcccggcgtctgcagcgccaccgtcgtgccggcccaccgccggcgcagctcccgtccgagcgccgtcgcctcctcggcgcgcagcaacgtctgtcggagcgccggctgaggcagcagcgtcgcgcgcggggtgcccacgcccagccggttgcagcggtacagccgcaccacctcgcccgcgccgtgccgaaaccactcgtccgcgtcgcgcgccgctaggatcagcgtgttgtttgccaggtcgtacacgaacacgcggaacccggcgcccagcgccaggtacagtccgtcctgcgcgcacagaccctcgggatggccggccttgtcgcccaacgtcgggtcggctgcggggtccacctcgtgcaccacggtagccaccagtacgatccacgcgtcccgcggcgacagttgacgcaggtccgtcgcgcccacgccgttcatctggctgcgcggcgtcacccgcgcgtagaatccgtacggccgtccgagcggcagcagcgtgcccgcgtcgcgctgcgaccacttgcgcatggcgcggcccgtgctgttggccaaaaacgccgcgcgccacacggcgcccatggcctggtattccagctccgtcagcgcctggcgctccaccggaatctgagacagcaacaggcgctccgggccgtgccaaaagttgctattgttgccgctactcggaggggcgcccggcggcccgcggggttctacccggtggacgccgtggcccggcgtcgtcgtagccgcagcactcgcaccagtgcccgctgtggacggcgctcccatcggcacacaagaagaaggaggagaggaaccaacccccgaaggccctccggccccgcggccgcgaccaaggggcggggggcgcggcgacatgccgttgcgctgggccatgggcgccggacacctccgacgtccactatataggaagcaaacccgcgtcagcgagcacgcggtttagacacgcggacgccttcgtcgcccgtgtgccgcgggcgacacgcagctggcttttataggcagcgacgtgcacggcgcgtgctggcgccgccttggcgccacgcagtctggaaggccgtggactgggaaaggcagctacccgaaggaagacgcgcggcaggcgcgtaccactggagcgcacagccgcctcccgggcgcgcacccatctaggtggacgcccgacatccattccgggccgcgtggtgggtcctcgaggggcgggggggtgtttttagcgggggggtgaaacttggagttgcgtgtgtggacggcgactagttgcgtgtggtgcggaggacggcgacggcgaataaaagcgacgtgcggcgcgcacggcgaaaagaagacgcgtgtctgtgtctgtgtgattccccggggaaaagaggaagttcccgggggacggcagcatgggtccctggggacacacgaaaagcaacgcccgggggcgagggacgacggccctggggaccgcgggggaaataacggccgcgaggccacacactcgttcctgcgaagccgcacaccccgaggccgcgcacaccgccgacacaccccgccaccacaccccgccggcacacccgccacacgcccgcgacacacccggcacgacacacccggcacacgcccgcgacacaccctgacacaccctgccaacacacccccgacacacccaacacacgcccgcgacacacccggcacacacccacccggccgcgccccgacacacccaaaacaccgccggtgcggggccgcgtggtgggtcctcgaggg

SEQ ID NO: 15 - DNA, 20 bp, synthetic construct
  misc_feature 1..20: note = Synthetic Sequence
  source 1..20: mol_type = other DNA
ccgcggaaaaaaacgattat

SEQ ID NO: 16 - DNA, 20 bp, synthetic construct
  misc_feature 1..20: note = Synthetic Sequence
  source 1..20: mol_type = other DNA
ccgcggaaaaaacgattatc